(19) United States
(12) Patent Application Publication
(10) Pub. No.: US 2026/0200900 A1
(43) Pub. Date: Jul. 16, 2026

(54) PROCESS FOR THE SYNTHESIS OF SUBSTITUTED TETRAHYDROFURAN MODULATORS OF SODIUM CHANNELS

(71) Applicant: VERTEX PHARMACEUTICALS INCORPORATED, Boston, MA (US)

(72) Inventors: Cristian HARRISON, Beverly, MA (US); Paloma BENITO GALLO, Didcot, Oxfordshire (GB); Shujauddin M. CHANGI, Needham, MA (US); Christopher John DAVIS, Salisbury, Wiltshire (GB); Simon Robert EVERITT, Beaconsfield, Bucks (GB); Enrico EMER, Oxford, Oxfordshire (GB); Kevin J. GAGNON, Acton, MA (US); Bérénice L. LEWANDOWSKI, Braintree, MA (US); Andrew McTIERNAN, Framingham, MA (US); Ales MEDEK, Winchester, MA (US); Michael Edward O'DONNELL, Abingdon, Oxfordshire (GB); Simon Adam O'NEIL, Belmont, MA (US); Maninder PANESAR, Didcot, Oxfordshire (GB); Mireia SIDERA PORTELA, Abingdon, Oxfordshire (GB); Stephen Edward SHANAHAN, Wokingham, Berkshire (GB); Bruno Artur SOUSA, Reading, Berkshire (GB); David E. STEPHENS, Dorchester, MA (US); Tharanga K. WIJETHUNGA, Quincy, MA (US)

(73) Assignee: VERTEX PHARMACEUTICALS INCORPORATED, Boston, MA (US)

(21) Appl. No.: 19/136,202

(22) PCT Filed: Dec. 5, 2023
(86) PCT No.: PCT/US2023/082576

§ 371 Date: Jun. 5, 2025

Related U.S. Application Data
(60) Provisional application No. 63/430,568, filed on Dec. 6, 2022.

(51) Int. Cl. C07D 405/12; A61K 9/20; A61K 31/443; C07D 307/68; C07D 405/14; C07D 471/08

(52) U.S. Cl. CPC C07D 405/12; A61K 9/2013; A61K 9/2054; A61K 31/443; C07D 307/68; C07D 405/14; C07D 471/08; C07B 2200/13

(57) ABSTRACT

Provided in this application is a process for making a compound of formula (I) and pharmaceutically acceptable salts thereof, which are useful as inhibitors of sodium channels. Also provided in this application is a process for making a compound of formula (II) and pharmaceutically acceptable salts thereof, which are useful as inhibitors of sodium channels. Processes for making various intermediate products, and suitable salts thereof, are also provided.

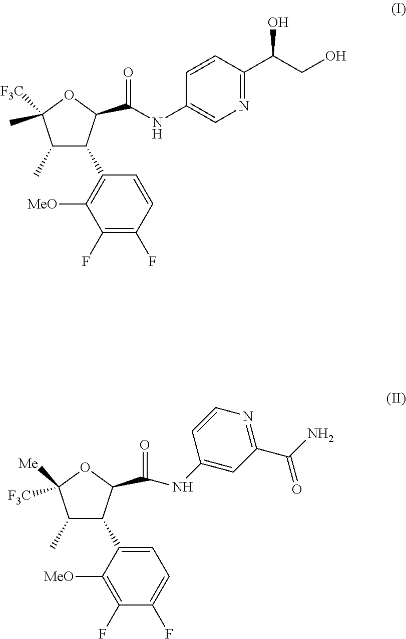 [SMILES: COc1c([C@H]2[C@H](C(=O)Nc3ccc([C@@H](O)CO)nc3)O[C@@](C)(C(F)(F)F)[C@H]2C)ccc(F)c1F ; COc1c([C@H]2[C@H](C(=O)Nc3ccnc(C(N)=O)c3)O[C@@](C)(C(F)(F)F)[C@H]2C)ccc(F)c1F]

### CROSS-REFERENCE TO RELATED APPLICATIONS

This application claims the benefit of U.S. Provisional Application No. 63/430,568, filed Dec. 6, 2022, which is incorporated by reference in its entirety.

### BACKGROUND

Pain is a protective mechanism that allows healthy animals to avoid tissue damage and to prevent further damage to injured tissue. Nonetheless, there are many conditions where pain persists beyond its usefulness, or where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137 (3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain.

Voltage-gated sodium channels (Na_{V}s) are involved in pain signaling. Na_{V}s are biological mediators of electrical signaling as they mediate the rapid upstroke of the action potential of many excitable cell types (e.g., neurons, skeletal myocytes, cardiac myocytes). The evidence for the role of these channels in normal physiology, the pathological states arising from mutations in sodium channel genes, preclinical work in animal models, and the clinical pharmacology of known sodium channel modulating agents all point to the central role of Nays in pain sensation (Rush, A. M. and T. R. Cummins, Painful Research: Identification of a Small-Molecule Inhibitor that Selectively Targets Na_{V}1.8 Sodium Channels. Mol. Interv., 2007. 7 (4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Na_{V}s mediate the rapid upstroke of the action potential of many excitable cell types (e.g., neurons, skeletal myocytes, cardiac myocytes), and thus are involved in the initiation of signaling in those cells (Hille, Bertil, Ion Channels of Excitable Membranes, Third ed. (Sinauer Associates, Inc., Sunderland, MA, 2001)). Because of the role Na_{V}s play in the initiation and propagation of neuronal signals, antagonists that reduce Na_{V} currents can prevent or reduce neural signaling and Na_{V} channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Na_{V} channels. The local anesthetic drugs such as lidocaine block pain by inhibiting Na_{V} channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J. Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Na_{V}s form a subfamily of the voltage-gated ion channel super-family and comprises 9 isoforms, designated Na_{V}1.1-Na_{V}1.9. The tissue localizations of the nine isoforms vary. Na_{V}1.4 is the primary sodium channel of skeletal muscle, and Na_{V}1.5 is primary sodium channel of cardiac myocytes. Na_{V}s 1.7, 1.8 and 1.9 are primarily localized to the peripheral nervous system, while Na_{V}s 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Na_{V}1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379 (6562): p. 257-62). Since then, Na_{V}1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22 (23): p. 10277-90). Na_{V}1.8 is involved in spontaneous firing in damaged neurons, like those that drive neuropathic pain (Roza, C., et al., The tetrodotoxin-resistant Na⁺ channel Na_{V}1.8 is essential for the expression of spontaneous activity in damaged sensory axons of mice. J. Physiol., 2003. 550 (Pt 3): p. 921-6; Jarvis, M. F., et al., A-803467, a potent and selective Na_{V}1.8 sodium channel blocker, attenuates neuropathic and inflammatory pain in the rat. Proc. Natl. Acad. Sci. USA, 2007. 104 (20): p. 8520-5; Joshi, S. K., et al., Involvement of the TTX-resistant sodium channel Na_{V}1.8 in inflammatory and neuropathic, but not post-operative, pain states. Pain, 2006. 123 (1-2): pp. 75-82; Lai, J., et al., Inhibition of neuropathic pain by decreased expression of the tetrodotoxin-resistant sodium channel, Na_{V}1.8. Pain, 2002. 95 (1-2): p. 143-52; Dong, X. W., et al., Small interfering RNA-mediated selective knockdown of Na_{V}1.8 tetrodotoxin-resistant sodium channel reverses mechanical allodynia in neuropathic rats. Neuroscience, 2007. 146 (2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64 (6): p. 644-53; Coward, K., et al., Immunolocalization of SNS/PN3 and NaN/SNS2 sodium channels in human pain states. Pain, 2000. 85 (1-2): p. 41-50; Yiangou, Y., et al., SNS/PN3 and SNS2/NaN sodium channel-like immunoreactivity in human adult and neonate injured sensory nerves. FEBS Lett., 2000. 467 (2-3): p. 249-52; Ruangsri, S., et al., Relationship of axonal voltage-gated sodium channel 1.8 (Na_{V}1.8) mRNA accumulation to sciatic nerve injury-induced painful neuropathy in rats. J. Biol. Chem. 286 (46): p. 39836-47). The small DRG neurons where Na_{V}1.8 is expressed include the nociceptors involved in pain signaling. Na_{V}1.8 mediates large amplitude action potentials in small neurons of the dorsal root ganglia (Blair, N. T. and B. P. Bean, Roles of tetrodotoxin (TTX)-sensitive Na′ current, TTX-resistant Na′ current, and Ca²′ current in the action potentials of nociceptive sensory neurons. J. Neurosci., 2002. 22 (23): p. 10277-90). Na_{V}1.8 is necessary for rapid repetitive action potentials in nociceptors, and for spontaneous activity of damaged neurons. (Choi, J. S. and S. G. Waxman, Physiological interactions between Na_{V}1.7 and Na_{V}1.8 sodium channels: a computer simulation study. J. Neurophysiol. 106 (6): p. 3173-84; Renganathan, M., T. R. Cummins, and S. G. Waxman, Contribution of Na(_{V})1.8 sodium channels to action potential electrogenesis in DRG neurons. J. Neurophysiol., 2001. 86 (2): p. 629-40; Roza, C., et al., The tetrodotoxin-resistant Na⁺ channel Na_{V}1.8 is essential for the expression of spontaneous activity in damaged sensory axons of mice. J. Physiol., 2003. 550 (Pt 3): p. 921-6). In depolarized or damaged DRG neurons, Na_{V}1.8 appears to be a driver of hyper-excitablility (Rush, A. M., et al., A single sodium channel mutation produces hyper- or hypoexcitability in different types of neurons. Proc. Natl. Acad. Sci. USA, 2006. 103 (21): p. 8245-50). In some animal pain models, Na_{V}1.8 mRNA expression levels have been shown to increase in the DRG (Sun, W., et al., Reduced conduction failure of the main axon of polymodal nociceptive C-fibers contributes to painful diabetic neuropathy in rats. Brain, 135 (Pt 2): p. 359-75; Strickland, I. T., et al., Changes in the expression of Na_{V}1.7, Na_{V}1.8 and Na_{V}1.9 in a distinct population of dorsal root ganglia innervating the rat knee joint in a model of chronic inflammatory joint pain. Eur. J. Pain, 2008. 12 (5): p. 564-72; Qiu, F., et al., Increased expression of tetrodotoxin-resistant sodium channels Na_{V}1.8 and Na_{V}1.9 within dorsal root ganglia in a rat model of bone cancer pain. Neurosci. Lett., 512 (2): p. 61-6).

The inventors have discovered that some voltage-gated sodium channel inhibitors have limitations as therapeutic agents due to, for example, a poor therapeutic window (e.g., due to a lack of Na_{V} isoform selectivity, low potency, and/or other reasons). Accordingly, there remains a need to develop selective voltage-gated sodium channel inhibitors, such as selective Na_{V}1.8 inhibitors, and methods for making the same.

### SUMMARY

In one aspect, the invention relates to a method of preparing a compound of formula (I):

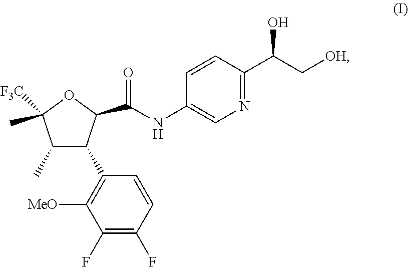 [SMILES: COc1c([C@H]2[C@H](C(=O)Nc3ccc([C@@H](O)CO)nc3)O[C@@](C)(C(F)(F)F)[C@H]2C)ccc(F)c1F]

or a pharmaceutically acceptable salt thereof.

In some embodiments, the method comprises converting any of compounds of formulae (III), (IV), (VI)-(XV), and (XXII)-(XIX) to the compound of formula (I) via the chemical processes described herein.

In a second aspect, the invention relates to a method of preparing a compound of formula (II):

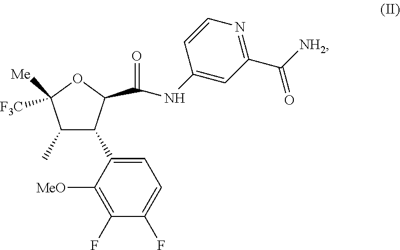 [SMILES: COc1c([C@H]2[C@H](C(=O)Nc3ccnc(C(N)=O)c3)O[C@@](C)(C(F)(F)F)[C@H]2C)ccc(F)c1F]

or a pharmaceutically acceptable salt thereof.

In some embodiments, the method comprises converting any of compounds of formulae (IV), (VI)-(XV), (XX), and (XXII)-(XIX) to the compound of formula (II) via the chemical processes described herein.

### BRIEF DESCRIPTION OF THE DRAWINGS

FIG. 1 depicts an XRPD pattern characteristic of Compound (VII), Form A.

FIG. 2 depicts an XRPD pattern characteristic of Compound (VI), Form A

FIG. 3 depicts an XRPD pattern characteristic of Compound (I), Form A.

FIG. 4 depicts a solid-state ¹³C NMR spectrum characteristic of Compound (I), Form A.

FIG. 5 depicts a solid-state ¹⁹F NMR spectrum characteristic of Compound (I), Form A.

FIG. 6 depicts an XRPD pattern of the spray-dried dispersion of Compound II of Example 11.

FIG. 7A depicts an XRPD pattern of the SDD tablet composition of Example 11 over the range of about 3° to about 40° 20.

FIG. 7B depicts an XRPD pattern of the SDD tablet composition of Example 11 over the range of about 14° to about 16° 2θ.

FIG. 8A depicts a solid state ¹⁹F NMR spectrum characteristic of the SDD tablet composition of Example 11.

FIG. 8B depicts a solid state ¹³C NMR spectrum characteristic of a powder of the SDD tablet composition of Example 11.

### DETAILED DESCRIPTION

Disclosed herein are synthetic processes and methods for preparing a compound of formula (I) and a compound of formula (II). In some embodiments, the skilled artisan may prepare a compound of formula (I) from any one of a compound of formulae (III), (IV), and (VI)-(XV), as described in Scheme A, below. In other embodiments, the skilled artisan may prepare a compound of formula (I) from any one of a compound of formulae (III), (IV), and (XXII)-(XIX), as summarized in Schemes A and C, below. In some embodiments, the skilled artisan may prepare a compound of formula (II) from any one of a compound of formulae (IV), (VI)-(XV), and (XX), as described in Scheme B, below. In other embodiments, the skilled artisan may prepare a compound of formula (II) from any one of a compound of formula (IV), (XX), and (XXII)-(XIX), as summarized in Schemes B and C, below.

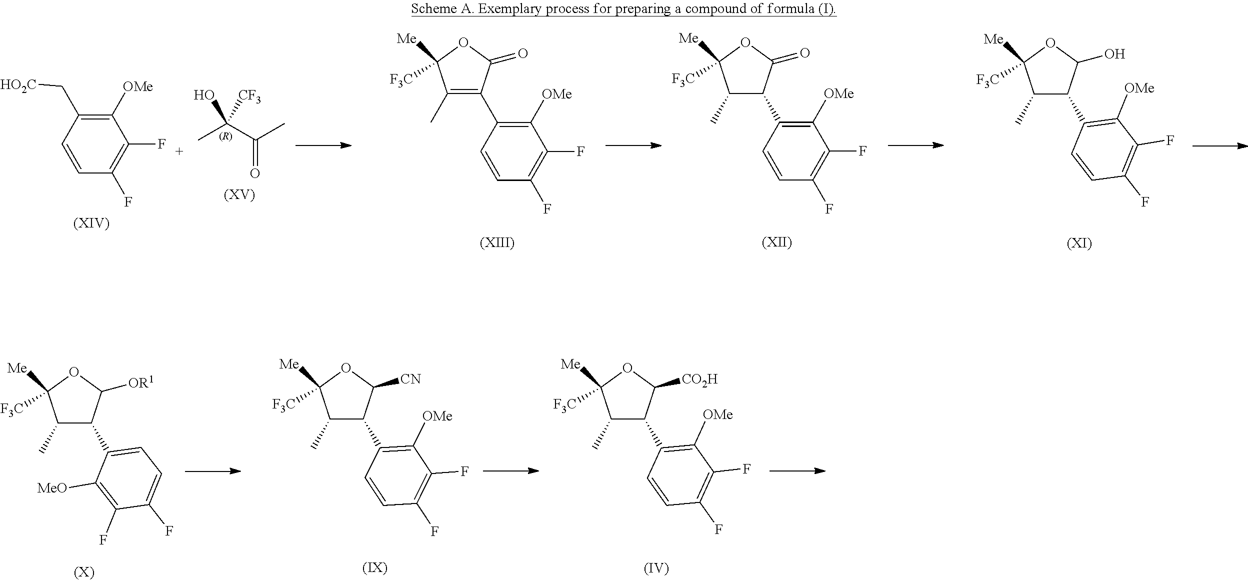

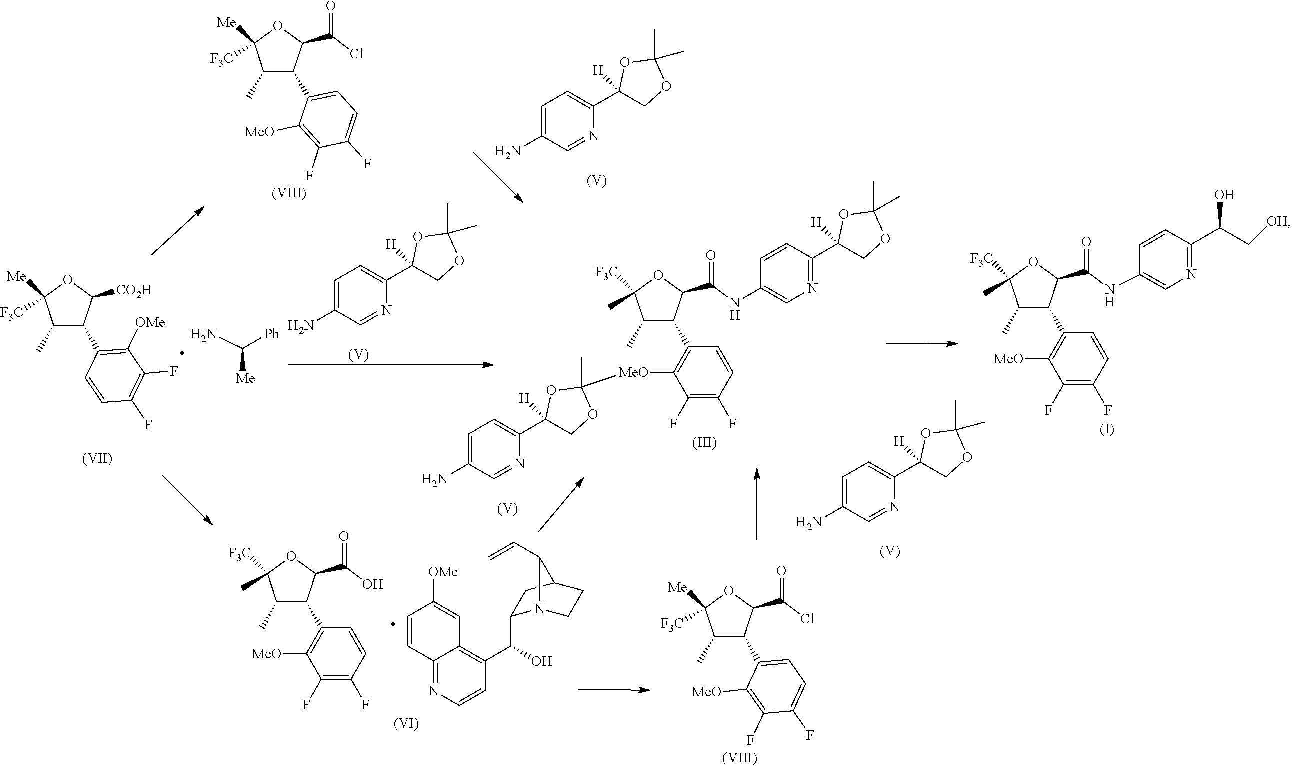

wherein:
R¹ is —C(O)—Z, —C(O)OZ, —C(O)CH═CH—Z, or —P(O)Z₂; and
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl.

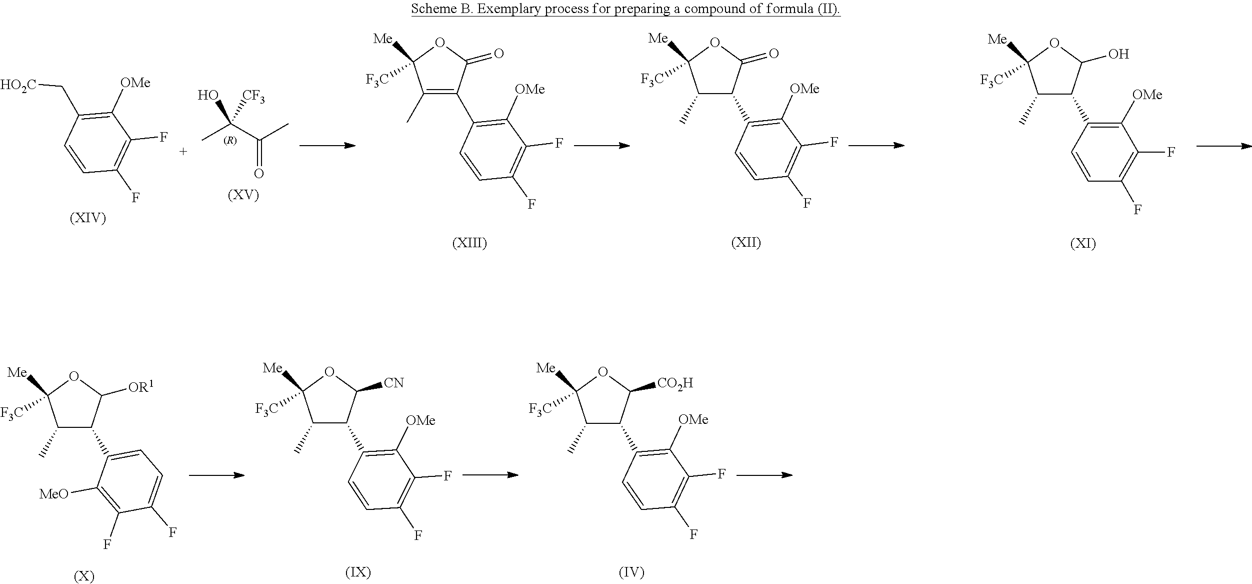

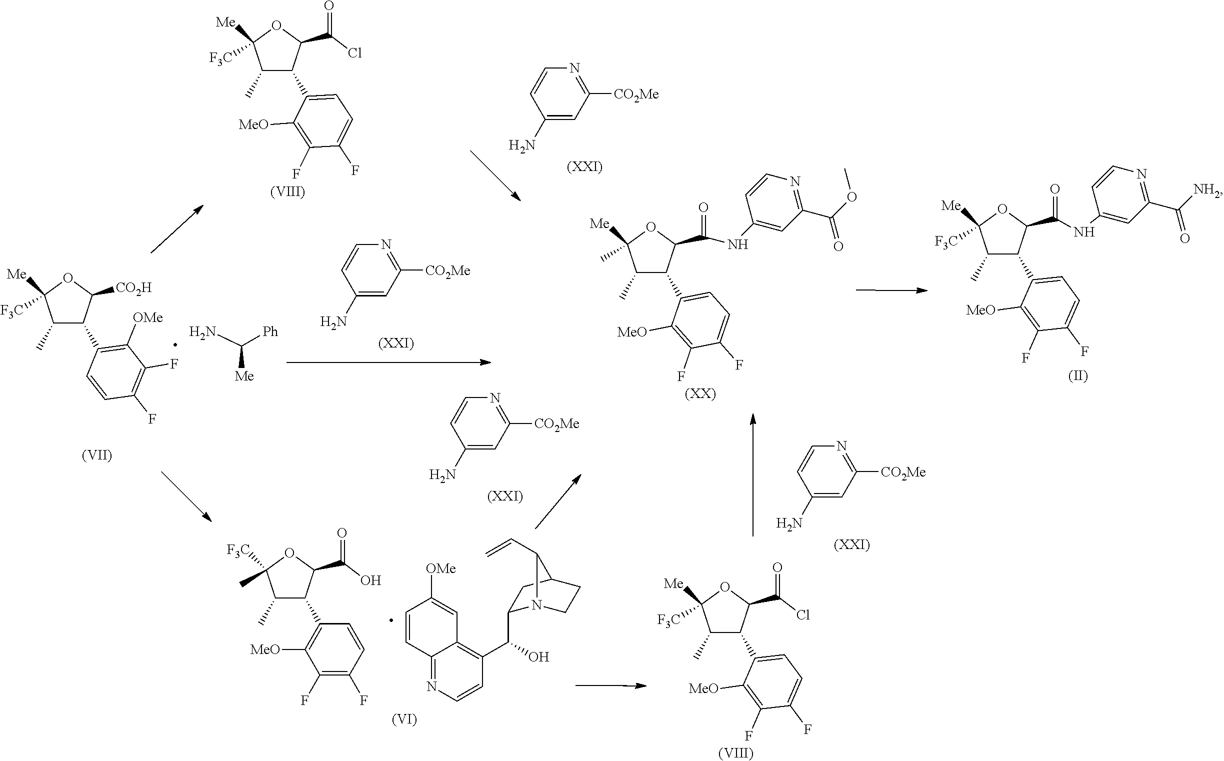

wherein:
R¹ is —C(O)—Z, —C(O)OZ, —C(O)CH═CH—Z, or —P(O)Z₂; and
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl.

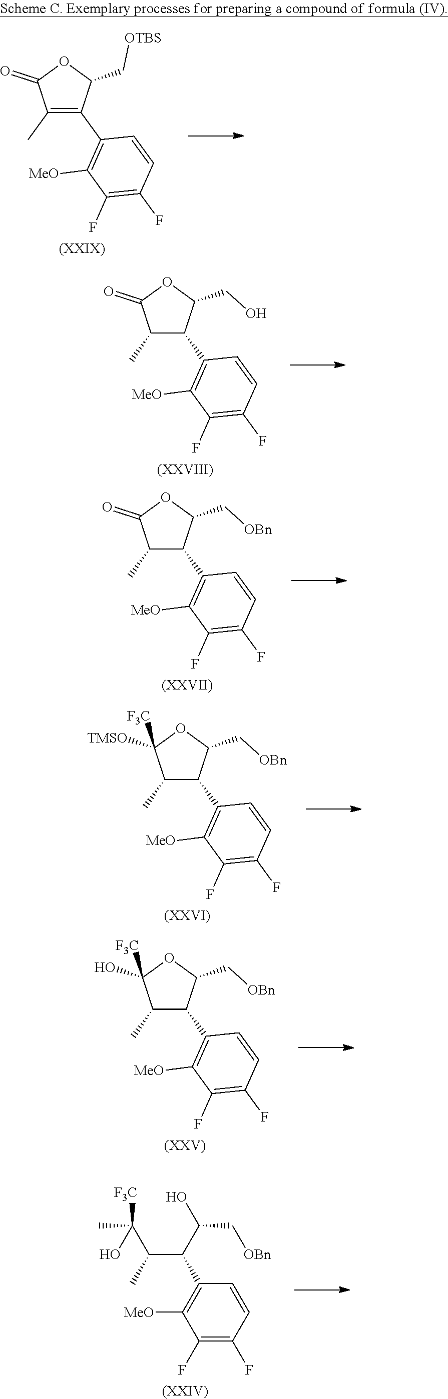

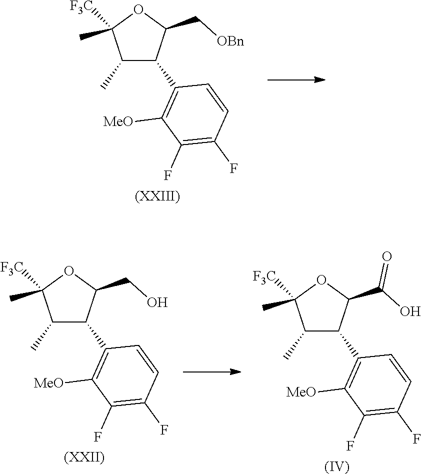

In one aspect, the method steps described herein may refer to conversion of a starting compound of formulae (III), (IV), (VI)-(XV), and (XXII)-(XIX) to the compound of formula (I). The skilled artisan would understand that such methods can also be used to prepare any intermediate between any starting compound and the compound of formula (I). For example, in some embodiments, conversion of the compound of formula (IV) to the compound of formula (I) goes through intermediate compounds (VII) and (III). As such, the skilled artisan would understand that the methods described for converting the compound of formula (IV) to the compound of formula (I) can be used to prepare either of intermediate compounds (VII) and (III) from the compound of formula (IV). Similarly, in some embodiments, conversion of the compound of formula (XIII) to the compound of formula (I) goes through preparation of intermediate compounds (III), (VII), (IV), and (IX)-(XII). As such, the skilled artisan would understand that the methods described for converting the compound of formula (XIII) to the compound of formula (I) can be used to prepare any of intermediate compounds (III), (VII), (IV), and (IX)-(XII) starting with the compound of formula (XIII). In other embodiments, conversion of the compound of formula (IV) to the compound of formula (I) goes through intermediate compounds (III), (VI), and (VII). In yet other embodiments, conversion of the compound of formula (IV) to the compound of formula (I) goes through intermediate compounds (III), (VII), and (VIII). In other embodiments, conversion of the compound of formula (IV) to the compound of formula (I) goes through intermediate compounds (III), (VI), (VII), and (VIII).

In another aspect, the method steps described herein may refer to conversion of a starting compound of formulae (IV), (VI)-(XV), (XX), and (XXII)-(XIX) to the compound of formula (II). The skilled artisan would understand that such methods can also be used to prepare any intermediate between any starting compound and the compound of formula (II). For example, in some embodiments, conversion of the compound of formula (IV) to the compound of formula (II) goes through intermediate compounds (VII) and (XX). As such, the skilled artisan would understand that the methods described for converting the compound of formula (IV) to the compound of formula (II) can be used to prepare either of intermediate compounds (VII) and (XX) from the compound of formula (IV). Similarly, in some embodiments, conversion of the compound of formula (XIII) to the compound of formula (II) goes through preparation of intermediate compounds (IV), (VII), (IX)-(XII), and (XX). As such, the skilled artisan would understand that the methods described for converting the compound of formula (XIII) to the compound of formula (II) can be used to prepare any of intermediate compounds (IV), (VII), (IX)-(XII), and (XX), starting with the compound of formula (XIII). In other embodiments, conversion of the compound of formula (IV) to the compound of formula (II) goes through intermediate compounds (VI), (VII), and (XX). In yet other embodiments, conversion of the compound of formula (IV) to the compound of formula (II) goes through intermediate compounds (VII), (VIII), and (XX). In other embodiments, conversion of the compound of formula (IV) to the compound of formula (I) goes through intermediate compounds (VI), (VII), (VIII), and (XX).

Thus, the present application contemplates preparing intermediate compounds (III), (IV), (VI)-(XV), (XX), and (XXII)-(XIX) starting with any intermediate or starting material that precedes the intermediate that is being prepared. For example, intermediate compound (III) may be prepared starting with any of compounds (IV), (VI)-(XV), and (XXII)-(XXIX). Similarly, compound (XX) may be prepared starting with any of compounds (IV), (VI)-(XV), and (XXII)-(XXIX).

In another aspect, the invention provides a process for preparing a compound of formula (I) that includes transforming a compound of formula (IV):

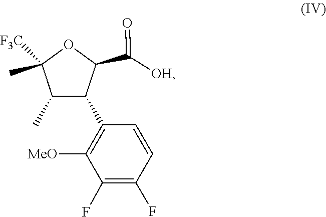 [SMILES: COc1c([C@H]2[C@H](C(=O)O)O[C@@](C)(C(F)(F)F)[C@H]2C)ccc(F)c1F]

or a salt thereof, to the compound of formula (I).

In some embodiments, the process of transforming a compound of formula (IV) to the compound of formula (I) includes preparing a compound of formula (VI):

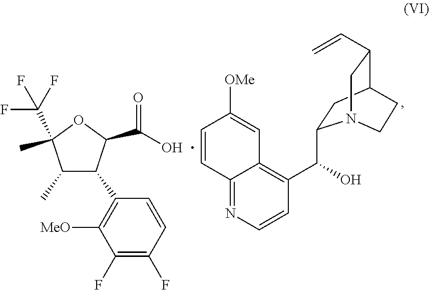

which may also be referred to as a quinine salt of the compound of formula (IV).

In some embodiments, the compound of formula (VI) may be prepared directly from the compound of formula (IV) by contacting the compound of formula (IV) within quinine in an appropriate solvent. In some embodiments, the compound of formula (VI) may be prepared by dissolving the compound of formula (IV) and quinine in an appropriate solvent. In some embodiments, the appropriate solvent is a polar solvent. One of ordinary skill will appreciate that there are many solvents compatible with transforming the compound of formula (IV) to the compound of formula (VI), including without limitation: isopropanol, n-heptane, DCM, toluene, EtOAc, MTBE, acetonitrile, 2-MeTHF, MEK, and combinations thereof. In some embodiments, the compound of formula (IV) is converted to the compound of formula (VI) in the presence of DCM, isopropanol, n-heptane, and/or combinations thereof.

In some embodiments, the process of preparing a compound of formula (VI) includes preparing a compound of formula (VIII):

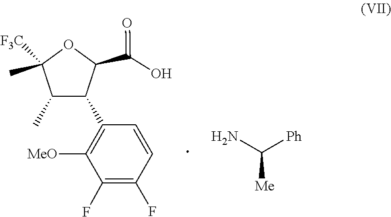 [SMILES: COc1c([C@H]2[C@H](C(=O)O)O[C@@](C)(C(F)(F)F)[C@H]2C)ccc(F)c1F ; C[C@@H](N)c1ccccc1]

and contacting the compound of formula (VII) with quinine. The compound of formula (VII) may also be referred to as an (R)-AMB or an (R)-α-methylbenzylamine salt of the compound of formula (IV). The compound of formula (VII) can be converted to the compound of formula (VI) using any method known to those skilled in the art.

In some embodiments, the compound of formula (VI) may be prepared from the compound of formula (VII) by contacting the compound of formula (VII) within quinine in an appropriate solvent. In some embodiments, the compound of formula (VI) may be prepared by dissolving the compound of formula (VII) and quinine in an appropriate solvent. In some embodiments, the appropriate solvent is a polar solvent. One of ordinary skill will appreciate that there are many solvents compatible with transforming the compound of formula (VII) to the compound of formula (VI), including without limitation: isopropanol, n-heptane, DCM, toluene, EtOAc, MTBE, acetonitrile, 2-MeTHF, MEK, and combinations thereof. In some embodiments, the compound of formula (VII) is converted to the compound of formula (VI) in the presence of DCM, isopropanol, n-heptane, and/or combinations thereof.

In some embodiments, the compound of formula (VI) may be prepared from the compound of formula (VII) by first converting the compound of formula (VII) to a free base, i.e. the compound of formula (IV):

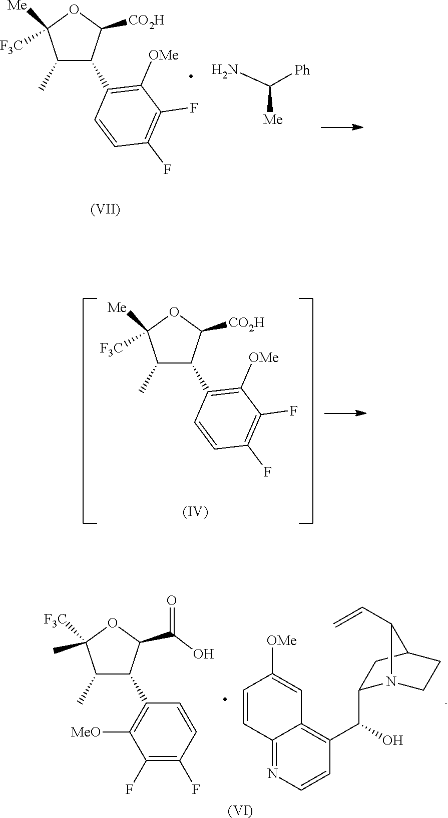

The compound of formula (VII) may be converted to the free base compound of formula (IV) by any method known to one of ordinary skill. In some embodiments, the compound of formula (VII) is converted to the free base compound of formula (IV) by treating the compound of formula (VII) with an aqueous acid. In some embodiments, the aqueous acid is aqueous hydrochloric acid.

In some embodiments, the process of transforming a compound of formula (IV) to the compound of formula (I) includes preparing a compound of formula (VII):

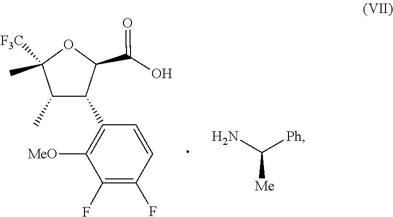 [SMILES: COc1c([C@H]2[C@H](C(=O)O)O[C@@](C)(C(F)(F)F)[C@H]2C)ccc(F)c1F ; C[C@@H](N)c1ccccc1]

which may also be referred to as an (R)-AMB or an (R)-α-methylbenzylamine salt of the compound of formula (IV).

In some embodiments, the compound of formula (VII) may be prepared directly from the compound of formula (IV) by contacting the compound of formula (IV) with (R)-α-methylbenzylamine. In some embodiments, the compound of formula (VII) may be prepared directly from the compound of formula (IV) by contacting the compound of formula (IV) with (R)-α-methylbenzylamine in an appropriate solvent. In some embodiments, the compound of formula (VII) may be prepared directly from the compound of formula (IV) by dissolving the compound of formula (IV) and (R)-α-methylbenzylamine in an appropriate solvent. One of ordinary skill will appreciate that there are many solvents compatible with transforming the compound of formula (IV) to the compound of formula (VII). In some embodiments, the appropriate solvent is toluene.

In some embodiments, the process of transforming the compound of formula (IV) to the compound of formula (I) includes contacting the compound of formula (IV), the compound of formula (VI), or the compound of formula (VII) with a compound of formula (V):

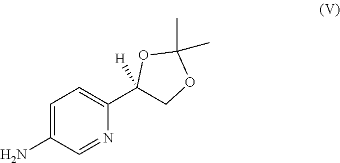

to afford a compound of formula (III):

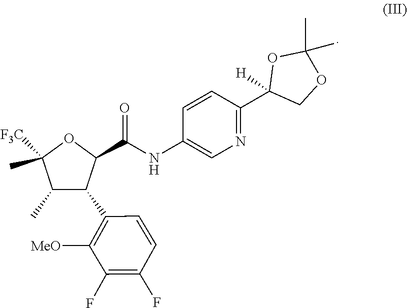

In some embodiments, the compound of formula (IV), the compound of formula (VI), or the compound of formula (VII) is contacted with a compound of formula (V) in the presence of a coupling reagent and a base. In some embodiments, the compound of formula (IV) is contacted with a compound of formula (V) in the presence of a coupling reagent and a base. In some embodiments, the compound of formula (VI) is contacted with a compound of formula (V) in the presence of a coupling reagent and a base. In some embodiments, the compound of formula (VII) is contacted with a compound of formula (V) in the presence of a coupling reagent and a base. One of ordinary skill in the art will appreciate that many coupling reagents may be compatible with the process of coupling a compound of formula (V) to the compound of formula (IV), the compound of formula (VI), or the compound of formula (VII). For example, in some embodiments, the coupling reagent is 1,1′-carbonyldiimidazole (CDI), 1-ethyl-3-(3-dimethylaminopropyl)carbodiimide (EDCl), or propylphosphonic acid anhydride (T3P). In some embodiments, the coupling reagent is T3P. One of ordinary skill in the art will appreciate that many bases may be compatible with the process of coupling a compound of formula (V) to the compound of formula (IV), the compound of formula (VI), or the compound of formula (VII). In some embodiments, the base is a non-nucleophilic base. In some embodiments, the base is selected from trimethylamine, N-methylimidazole, pyridine, 4-methylmorpholine, Hunig's base, DABCO, and NaOH, and the like. In other embodiments, the base may be any C₁-C₄ alkyl tertiary amine, such as triethylamine, ethyldimethyl amine, ethyldipropyl amine and various other alkyl combinations thereof. In some embodiments, the base is triethylamine.

In some embodiments, the compound of formula (VI) or (VII) is first converted to a free base, i.e. the compound of formula (IV), before being contacted by the compound of formula (V). The compound of formula (VI) or (VII) may be converted to the free base compound of formula (IV) by any method known to one of ordinary skill. In some embodiments, the compound of formula (VI) or (VII) is converted to the free base compound of formula (IV) by treating the compound of formula (VI) or (VII) with an aqueous acid. In some embodiments, the aqueous acid is aqueous hydrochloric acid.

In some embodiments, the process of transforming the compound of formula (IV) to the compound of formula (I) includes transforming the compound of formula (IV), the compound of formula (VI), or the compound of formula (VII) to a compound of formula (VIII):

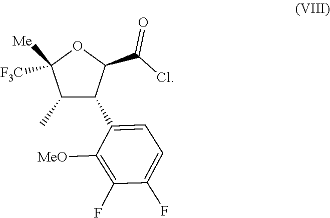 [SMILES: COc1c([C@H]2[C@H](C(=O)Cl)O[C@@](C)(C(F)(F)F)[C@H]2C)ccc(F)c1F]

In some embodiments, the process of transforming the compound of formula (IV) to the compound of formula (I) includes transforming the compound of formula (IV) to the compound of formula (VIII). In some embodiments, the process of transforming the compound of formula (IV) to the compound of formula (I) includes transforming the compound of formula (VI) to the compound of formula (VIII). In some embodiments, the process of transforming the compound of formula (IV) to the compound of formula (I) includes transforming the compound of formula (VII) to the compound of formula (VIII)

In some embodiments, the compound of formula (VI) or (VII) is first converted to a free base, i.e. the compound of formula (IV), before being transformed to the compound of formula (VIII). The compound of formula (VI) or (VII) may be converted to the free base compound of formula (IV) by any method known to one of ordinary skill. In some embodiments, the compound of formula (VI) or (VII) is converted to the free base compound of formula (IV) by treating the compound of formula (VI) or (VII) with an aqueous acid. In some embodiments, the aqueous acid is aqueous hydrochloric acid.

In some embodiments, transforming the compound of formula (IV), the compound of formula (VI), or the compound of formula (VII) to the compound of formula (VIII) includes treating the compound of formula (IV), the compound of formula (VI) or the compound of formula (VII) with a chlorinating reagent. Any chlorinating agent suitable for chlorinating compound IV, or a salt thereof (i.e. compound (VI) or (VII)), may be used. In some embodiments, the chlorinating agent is thionyl chloride, methanesulfonyl chloride, phosphorus oxychloride, phosphorus pentachloride, phosgene, oxalyl chloride, isobutyl chloroformate (IBCF), pivaloyl chloride (PivCl), or diphenylphosphinic chloride (DPPCl). In some embodiments, the chlorinating agent is oxalyl chloride. In some embodiments, the compound of formula (VIII) is carried on to the next reaction in the process for preparing a compound of formula (I) without further purification or isolation.

In some embodiments, treating the compound of formula (IV), the compound of formula (VI) or the compound of formula (VII) with a chlorinating reagent is performed in the presence of a non-nucleophilic base. Without being bound to theory, the non-nucleophilic base may be included to scavenge byproduct HCl that is generated during the chlorination reaction. Accordingly, any suitable non-nucleophilic base known by one of ordinary skill in the art may be used. Suitable non-nucleophilic bases are typically tertiary or aromatic amines where the nitrogen of the amine base does not carry an H atom. The non-nucleophilic base may be bulky bases that are non-nucleophilic because of steric hindrance. Examples of suitable bases include Hunig's base, triethylamine, diisopropyl ethylamine, N-methylmorpholine, 1,8-diazabicyclo[5.4.0]undec-7-ene, pyridine, butylamine, or 1,5-diazabicyclo(4.3.0) non-5-ene, or a mixture thereof. In some embodiments, the reaction between compound IV and the chlorinating agent is conducted at a temperature of no more than about 90° C.

In some embodiments, transforming a compound of formula (IV) to the compound of formula (I) includes contacting the compound of formula (VIII) with the compound of formula (V) to afford the compound of formula (III). In some embodiments, contacting the compound of formula (VIII) with a compound of formula (V) is performed in the presence of a base. One of ordinary skill in the art will appreciate that many bases would be compatible with the reaction between the compound of formula (VIII) and the compound of formula (V). In some embodiments, the base is a non-nucleophilic base. In some embodiments, the base is triethylamine.

In some embodiments, the process of transforming a compound of formula (IV) to the compound of formula (I) further includes transforming the compound of formula (III) to the compound of formula (I). In some embodiments, transforming the compound of formula (III) to the compound of formula (I) includes deprotecting the compound of formula (III) to afford the compound of formula (I). In some embodiments, transforming the compound of formula (III) to the compound of formula (I) includes treating the compound of formula (III) with an acid and water, e.g. an aqueous acid. One of ordinary skill in the art will appreciate that many acids would be compatible the process of deprotecting the compound of formula (III) to afford the compound of formula (I). In some embodiments, the acid is aqueous trifluoroacetic acid.

In some embodiments, the process for preparing the compound of formula (I) further includes recrystallizing the compound of formula (I) from a suitable solvent to afford a crystalline form of the compound of formula (I). In some embodiments, the crystalline form is Form A of the compound of formula (I), as described more fully below. In some embodiments, the suitable solvent includes ethyl acetate and n-heptane. In some embodiments, Form A of compound (I) is obtainable by dissolving the compound in ethyl acetate and then crystallizing the compound by adding n-heptane as an antisolvent. In other embodiments, Form A is obtainiably by the procedure described in Example 7.

Intermediate compound (V) may be prepared, for example, by the process depicted in Scheme D, below.

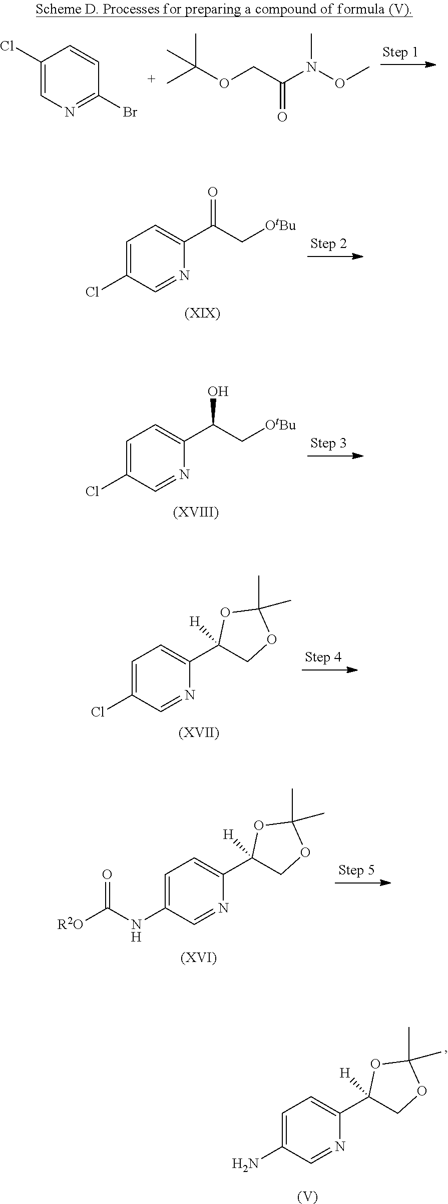

wherein R² is C₁-C₆ alkyl.

In some embodiments, the process for preparing the compound of formula (I) further includes transforming a compound of formula (XVI):

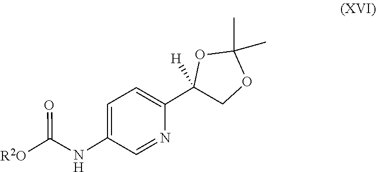

to the compound of formula (V), wherein R² is C₁-C₆ alkyl. In some embodiments, R² is ethyl. In other embodiments, R² is tert-butyl. In some embodiments, the compound of formula (XVI) is a compound of formula (XVI-A):

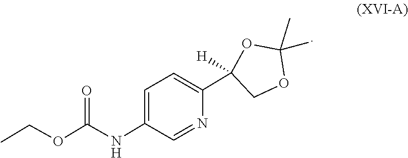

In other embodiments, the compound of formula (XVI) is a compound of formula (XVI-B):

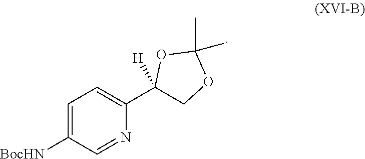

In some embodiments, transforming the compound of formula (XVI) to the compound of formula (V) includes treating the compound of formula (XVI) with water at reflux. In other embodiments, transforming the compound of formula (XVI) to the compound of formula (V) includes treating the compound of formula (XVI) with a base and water. One of ordinary skill in the art will appreciate that many bases would be compatible with the process of transforming the compound of formula (XVI) to the compound of formula (V). In some embodiments, the base is NaOH.

In some embodiments, the compound of formula (V) prepared from the compound of formula (XVI) is recrystallized from a suitable solvent prior to use in the process for preparing a compound of formula (I). In some embodiments, the suitable solvent includes MTBE and n-heptane.

In some embodiments, the process for preparing a compound of formula (V) further includes transforming a compound of formula (XVII):

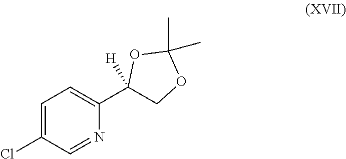

to the compound of formula (XVI).

In some embodiments, transforming the compound of formula (XVII) to the compound of formula (XVI) includes treating the compound of formula (XVII) with NH₂—C(O)—(C₁-C₆ alkyl), a base, a palladium catalyst, and a ligand. In some embodiments, the base is Cs₂CO₃ or K₃PO₄. In some embodiments, the base is Cs₂CO₃. In other embodiments, the base is K₃PO₄. In some embodiments, the palladium catalyst is Pd(OAc)₂. In some embodiments, the ligand is XPhos or BrettPhos. In some embodiments, the ligand is XPhos. In other embodiments, the ligand is BrettPhos.

In some embodiments, the process of preparing a compound of formula (V) further includes transforming a compound of formula (XVIII):

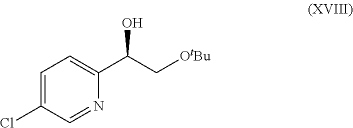 [SMILES: CC(C)(C)OC[C@H](O)c1ccc(Cl)cn1]

to the compound of formula (XVII).

In some embodiments, the process of transforming a compound of formula (XVIII) to the compound of formula (XVII) comprises treating the compound of formula (XVIII) with 2,2-dimethoxypropane in the presence of an acid. One of ordinary skill in the art will appreciate that many acids would be compatible with the process of transforming a compound of formula (XVIII) to the compound of (XVII). In some embodiments, the acid is methanesulfonic acid.

In some embodiments, the process of preparing a compound of formula (V) further includes transforming a compound of formula (XIX):

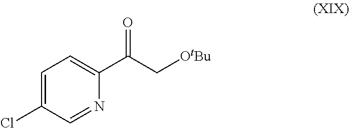 [SMILES: CC(C)(C)OCC(=O)c1ccc(Cl)cn1]

to the compound of formula (XVIII).

In some embodiments, transforming the compound of formula (XIX) to the compound of formula (XVIII) comprises contacting the compound of formula (XIX) with a reducing agent. In some embodiments, the reduction of the compound of formula (XIX) to the compound of formula (XVIII) is completed enzymatically. In some embodiments, the compound of formula (XIX) is contacted with a reducing agent in the presence of an enzyme. In some embodiments, the enzyme includes Ketone reductase. In some embodiments, the enzyme includes Glucose dehydrogenase. In some embodiments, the enzyme includes Ketone reductase 117297 and Glucose dehydrogenase 117446.

In some embodiments, the process of preparing a compound of formula (V) further includes contacting 2-bromo-5-chloropyridine with 2-(tert-butoxy)-N-methoxy-N-methylacetamide to afford the compound of formula (XIX). In some embodiments, the 2-bromo-5-chloropyridine is contacted with 2-(tert-butoxy)-N-methoxy-N-methylacetamide in the presence of a Grignard reagent. In some embodiments, the Grignard reagent is isopropyl magnesium chloride or isopropyl magnesium chloride lithium chloride complex.

In a second aspect, the present invention relates to a process for preparing a compound of formula (II):

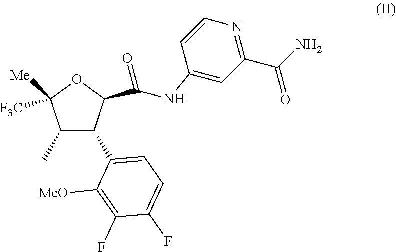 [SMILES: COc1c([C@H]2[C@H](C(=O)Nc3ccnc(C(N)=O)c3)O[C@@](C)(C(F)(F)F)[C@H]2C)ccc(F)c1F]

that includes transforming a compound of formula (VII):

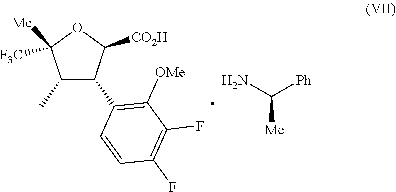 [SMILES: COc1c([C@H]2[C@H](C(=O)O)O[C@@](C)(C(F)(F)F)[C@H]2C)ccc(F)c1F ; C[C@@H](N)c1ccccc1]

to the compound of formula (II).

In some embodiments, transforming the compound of formula (VII) to the compound of formula (II) includes contacting the compound of formula (VII) with a compound of formula (XXI):

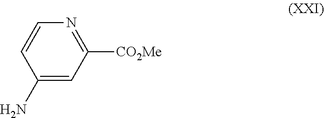 [SMILES: COC(=O)c1cc(N)ccn1]

to afford a compound of formula (XX):

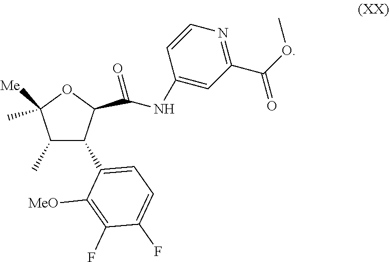

In some embodiments, contacting the compound of formula (VII) with a compound of formula (XXI) is performed in the presence of a coupling reagent and a base. One of ordinary skill in the art will appreciate that many coupling reagents may be compatible with the process of coupling a compound of formula (V) to the compound of formula (IV), the compound of formula (VI), or the compound of formula (VII). For example, in some embodiments, the coupling reagent is 1,1′-carbonyldiimidazole (CDI), 1-ethyl-3-(3-dimethylaminopropyl)carbodiimide (EDCl), diphenylphosphinic chloride (DPPCL), isobutyl chloroformate (IBCF), or propylphosphonic acid anhydride (T3P). In some embodiments, the coupling reagent is T3P. One of ordinary skill in the art will appreciate that many bases may be compatible with the process of coupling a compound of formula (V) to the compound of formula (IV), the compound of formula (VI), or the compound of formula (VII). In some embodiments, the base is a non-nucleophilic base. In some embodiments, the base is selected from trimethylamine, N-methylimidazole, pyridine, 4-methylmorpholine, Hunig's base, DABCO, and NaOH, and the like. In other embodiments, the base may be any C₁-C₄ alkyl tertiary amine, such as triethylamine, ethyldimethyl amine, ethyldipropyl amine and various other alkyl combinations thereof. In some embodiments, the base is triethylamine, 4-methylmorpholine (NMM), or 1-methylimidzole (NMI). In some embodiments, the base is triethylamine.

In some embodiments, the compound of formula (VII) is first converted to a free base, i.e. the compound of formula (IV), before being contacted by the compound of formula (XXI). The compound of formula (VII) may be converted to the free base compound of formula (IV) by any method known to one of ordinary skill. In some embodiments, the compound of formula (VII) is converted to the free base compound of formula (IV) by treating the compound of formula (VII) with an aqueous acid. In some embodiments, the aqueous acid is aqueous hydrochloric acid.

In some embodiments, transforming the compound of formula (VII) to the compound of formula (II) includes transforming the compound of formula (VII) to a compound of formula (VIII):

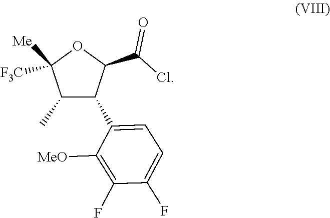 [SMILES: COc1c([C@H]2[C@H](C(=O)Cl)O[C@@](C)(C(F)(F)F)[C@H]2C)ccc(F)c1F]

In some embodiments, the compound of formula (VII) is first converted to a free base, i.e. the compound of formula (IV), before being transformed to the compound of formula (VIII). The compound of formula (VII) may be converted to the free base compound of formula (IV) by any method known to one of ordinary skill. In some embodiments, the compound of formula (VII) is converted to the free base compound of formula (IV) by treating the compound of formula (VII) with an aqueous acid. In some embodiments, the aqueous acid is aqueous hydrochloric acid.

In some embodiments, transforming the compound of formula (VII) to the compound of formula (VIII) includes treating the free base compound of formula (IV) or the compound of formula (VII) with a chlorinating reagent. Any chlorinating agent suitable for chlorinating compound IV, or a salt thereof (i.e. compound (VII)), may be used. In some embodiments, the chlorinating agent is thionyl chloride, methanesulfonyl chloride, phosphorus oxychloride, phosphorus pentachloride, phosgene, oxalyl chloride, isobutyl chloroformate (IBCF), pivaloyl chloride (PivCl), or diphenylphosphinic chloride (DPPCl). In some embodiments, the chlorinating agent is oxalyl chloride. In some embodiments, the compound of formula (VIII) is carried on to the next reaction in the process for preparing a compound of formula (I) without further purification or isolation.

In some embodiments, treating the free base compound of formula (IV) or the compound of formula (VII) with a chlorinating reagent is performed in the presence of a non-nucleophilic base. Without being bound to theory, the non-nucleophilic base may be included to scavenge byproduct HCl that is generated during the chlorination reaction. Accordingly, any suitable non-nucleophilic base known by one of ordinary skill in the art may be used. Suitable non-nucleophilic bases are typically tertiary or aromatic amines where the nitrogen of the amine base does not carry an H atom. The non-nucleophilic base may be bulky bases that are non-nucleophilic because of steric hindrance. Examples of suitable bases include Hunig's base, triethylamine, diisopropyl ethylamine, N-methylmorpholine, 1,8-diazabicyclo[5.4.0]undec-7-ene, pyridine, butylamine, or 1,5-diazabicyclo(4.3.0)non-5-ene, or a mixture thereof. In some embodiments, the reaction between the free base of compound IV or the compound of formula (VII) and the chlorinating agent is conducted at a temperature of no more than about 90° C.

In some embodiments, transforming the compound of formula (VII) to the compound of formula (II) comprises contacting the compound of formula (VIII) with the compound of formula (XXI) to afford the compound of formula (XX). In some embodiments, contacting the compound of formula (VIII) with a compound of formula (XXI) is performed in the presence of a base. One of ordinary skill in the art will appreciate that many bases would be compatible with the reaction between the compound of formula (VIII) and the compound of formula (XXI). In some embodiments, the base is a non-nucleophilic base. In some embodiments, the base is triethylamine. One of ordinary skill in the art will appreciate that many solvents would be compatible with the reaction between the compound of formula (VIII) and the compound of formula (XXI). In some embodiments, the solvent is toluene. In some embodiments, the compound of formula (XX) is obtained by partitioning the reaction mixture between an organic layer comprising ethyl acetate and toluene and an aqueous layer.

In some embodiments, the compound of formula (XX) (prepared either by way of a free base compound of formula (IV), a compound of formula (VII), or a compound of formula (VIII)) may be purified, for example, by recrystallizing it from a solvent comprising methanol or water or a mixture thereof. Other suitable combination of two solvents include ethanol/water, toluene/heptane, IPA/water, etc. In any of these combinations, the compound of formula (XX) is dissolved in one solvent at boiling or near boiling temperature followed by addition of the second solvent until the solution becomes turbid. The turbid suspension is allowed to cool down to room temperature (or cooled with an ice bath) followed by filtration of the solid.

In some embodiments, transforming a compound of formula (VII) to the compound of formula (II) further includes transforming the compound of formula (XX) to the compound of formula (II). In some embodiments, transforming the compound of formula (XX) to the compound of formula (II) comprises treating the compound of formula (XX) with ammonia to afford the compound of formula (II). In some embodiments, the compound of formula (XX) may be treated with ammonia in the presence of a suitable solvent. In some embodiments, the suitable solvent is methanol, ethanol, IPA, MeCN, THF, 2-MeTHF, water, or a mixture thereof. In some embodiments, treating the compound of formula (XX) with ammonia may be performed in the presence of a weak, non-nucleophilic base. In some embodiments, the base is selected from Mg(OMe)₂, CaCl₂, DIPEA, and K₂CO₃. In some embodiments, the ammonia is in the form of a solution of ammonia in a solvent, ammonia in gas form in which an ammonia gas is bubbled into the reaction mixture, or in the form of ammonium hydroxide or an ammonium salt where ammonia is generated in situ. In some embodiments, the ammonia is in the form of a solution of ammonia in methanol. In some embodiments, the ammonia is in the form of a solution of ammonia in methanol and tetrahydrofuran. In some embodiments, the in situ generation of ammonia includes reacting ammonium hydroxide or the ammonium salt with an acid. In some embodiments, treating the compound of formula (XX) with ammonia is performed in a solvent mixture comprising methanol and tetrahydrofuran.

In some embodiments, the process for preparing a compound of formula (II) further includes recrystallizing the compound of formula (II) from a suitable solvent. In some embodiments, the suitable solvent includes MeOH, THF, water, or a combination thereof. In other embodiments, the suitable solvent includes acetone and water.

In some embodiments, the compound of formula (VII) useful for preparing a compound of formula (II) is obtained from a compound of formula (IV), as described above with respect to the process for preparing a compound of formula (I).

Although the skilled artisan may devise a method of making the compound of formula (IV), or a salt thereof (i.e. a compound of formula (VI) or (VII)), used to prepare the compound of formula (I) or (II), the inventors of the present application contemplate preparing the compound of formula III using the following process.

In some embodiments, the process for preparing a compound of formula (I) or (II) further includes transforming a compound of formula (IX):

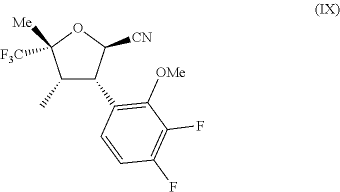 [SMILES: COc1c([C@H]2[C@H](C#N)O[C@@](C)(C(F)(F)F)[C@H]2C)ccc(F)c1F]

to the compound of formula (IV).

In some embodiments, transforming the compound of formula (IX) to the compound of formula (IV) includes hydrolyzing the CN group of the compound of formula (IX). Any base or acid suitable for hydrolyzing the CN group without affecting other functional groups in the compound of formula VII may be used. In some embodiments, a strong base (such as NaOH, KOH, and the like) or strong acid (HCl, sulfuric acid, or the like) may be used. In other embodiments, the CN group in the compound of formula (IX) is enzymatically hydrolyzed using a nitrilase. The CN hydrolysis of the compound of formula (IX) may be conducted in a solvent or solvent mixture. For example, ethanol, methanol, 1-propanol, 2-propanol, dioxane, water, THF, or a mixture thereof may be used. The hydrolysis reaction may be conducted at about 25-75° C., about 30-70° C., about 35-65° C., about 40-60° C., about 45-60° C., about 50-60° C., or about 55° C. As used in this paragraph, the term “about” in front of a temperature range applies to both ends of the range. It also means±2.5° C. In some embodiments, transforming a compound of formula (IX) to the compound of formula (IV) includes treating the compound of formula (IX) with a base. One of ordinary skill will appreciate that many bases would be compatible with the process of transforming the compound of formula (IX) to the compound of formula (IV). In some embodiments, the base is potassium hydroxide.

In some embodiments, the process for preparing a compound of formula (I) or (II) further includes transforming a compound of formula (X):

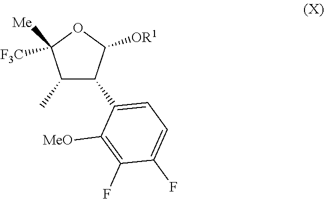

to the compound of formula (IX),
where:
R¹ is —C(O)—Z, —C(O)OZ, —C(O)CH═CH—Z, or —P(O)Z₂; and
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl.

In some embodiments, the compound of formula (X) is a compound of formula (X′):

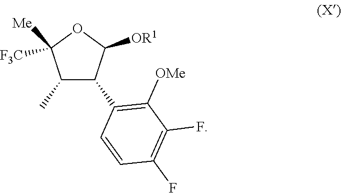

In some embodiments, the compound of formula (X) is a compound of formula (X″):

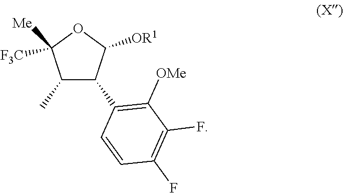

In some embodiments, R¹ is —C(O)—Z. In some embodiments, Z is methyl or 4-nitrophenyl. In some embodiments, Z is 4-nitrophenyl.

In some embodiments, the compound of formula (X) is selected from:

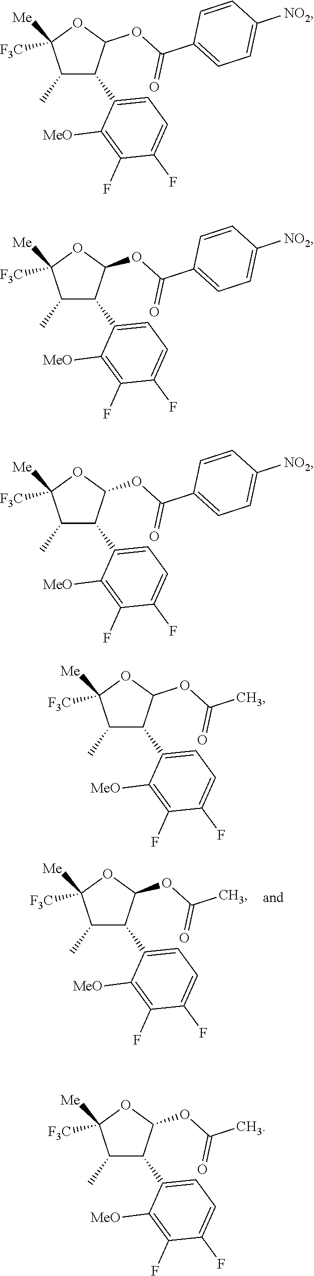

In some embodiments, the compound of formula (X) is a compound of formula (X-A):

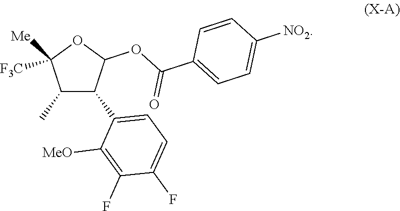 [SMILES: COc1c([C@H]2C(OC(=O)c3ccc([N+](=O)[O-])cc3)O[C@@](C)(C(F)(F)F)[C@H]2C)ccc(F)c1F]

In some embodiments, the compound of formula (X) is a compound of formula (X-B):

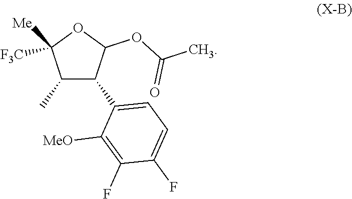 [SMILES: COc1c([C@H]2C(OC(C)=O)O[C@@](C)(C(F)(F)F)[C@H]2C)ccc(F)c1F]

In some embodiments, the compound of formula (X) is a compound of formula (X′-A):

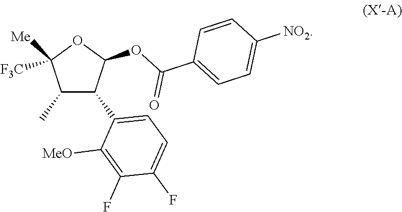 [SMILES: COc1c([C@H]2[C@H](OC(=O)c3ccc([N+](=O)[O-])cc3)O[C@@](C)(C(F)(F)F)[C@H]2C)ccc(F)c1F]

In some embodiments, the compound of formula (X) is a compound of formula (X′-B):

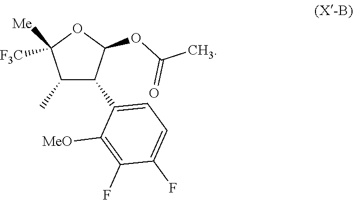 [SMILES: COc1c([C@H]2[C@H](OC(C)=O)O[C@@](C)(C(F)(F)F)[C@H]2C)ccc(F)c1F]

In some embodiments, transforming a compound of formula (X) to the compound of formula (IX) includes treating the compound of formula (X) with a cyanating reagent. In some embodiments, the cyanating agent is selected from the group consisting of trimethylsilyl cyanide, diethylaluminum cyanide, KCN, NaCN, TBACN, and HCN. In some embodiments, the cyanating reagent is trimethylsilyl cyanide. In some embodiments, the compound of formula (X) is treated with 1.35-1.65 equivalents of trimethylsilyl cyanide. In some embodiments, treating the compound of formula (X) with a cyanating reagent is performed in the presence of a Lewis acid. In some embodiments, the Lewis acid is selected from boron trifluoride diethyl etherate (BF3·OEt2), TiCl₄, InCl₃, AgSbF₆, iodine, ZnBr₂, Al(OiPr)₃, MgCl₂, Mn(acac)₂, MnCl₂, TMSOTf, and SnCl₄. In some embodiments, the Lewis acid is BF₃·OEt₂. In some embodiments, 0.9-1.1 equivalents of the BF₃OEt₃ are present, based on the compound of formula (X). The cyanation reaction may be conducted in an organic solvent, for example toluene, dichloromethane, 2-methyl THF, acetonitrile, methanol, 1,2-dichloroethane, nitromethane, CPME, MTBE, DMAc, t-BuOAc, and the like. In some embodiments, the cyanation reaction is conducted at a temperature between −28 and 0° C., or between −28 and −12° C. In some embodiments, the cyanation reaction is quenched with acetone (e.g., about 1.0 equivalents of acetone). In some embodiments, the quench is conducted at a temperature between −28 and 0° C., or between −28 and −12° C. In some embodiments, the compound of formula (IX) is obtained by partitioning the reaction mixture between an organic layer comprising ethanol and an aqueous layer comprising aqueous potassium hydroxide.

Other non-limiting examples of compounds of formula (X) include:

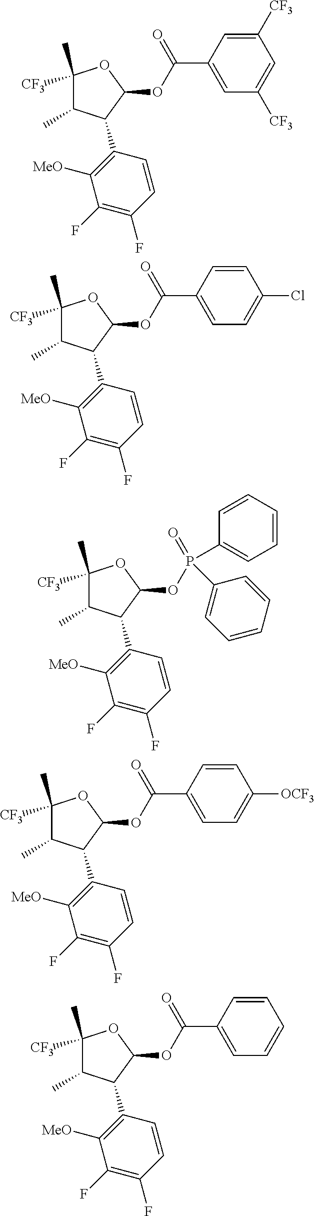

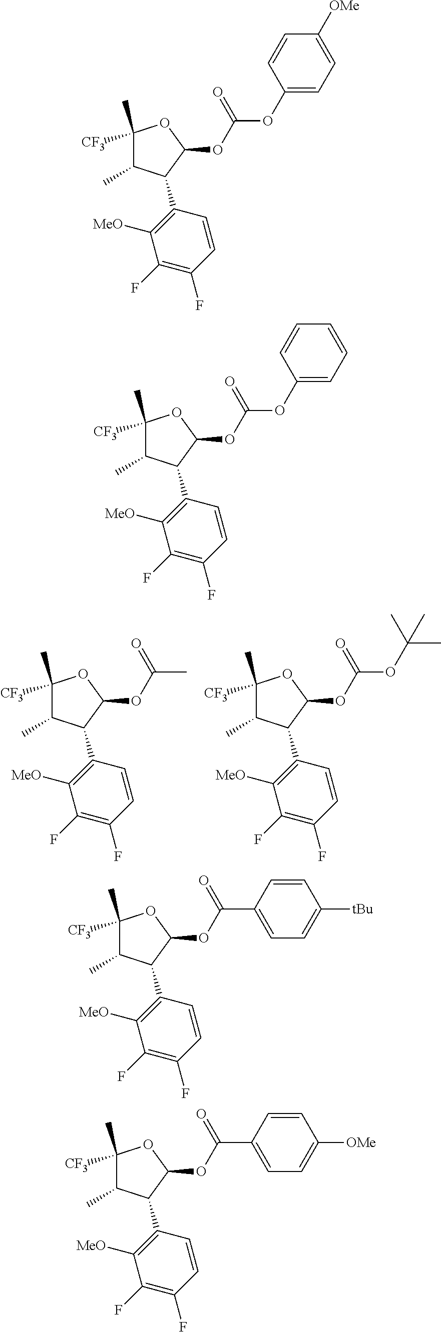

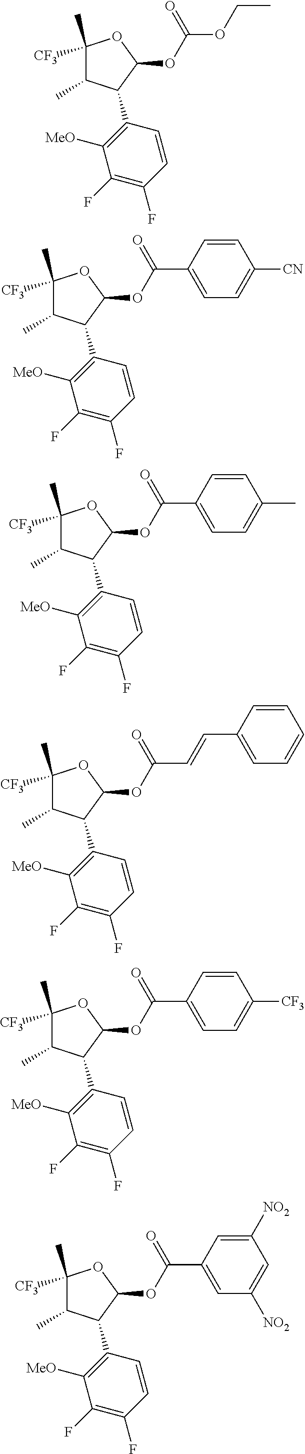

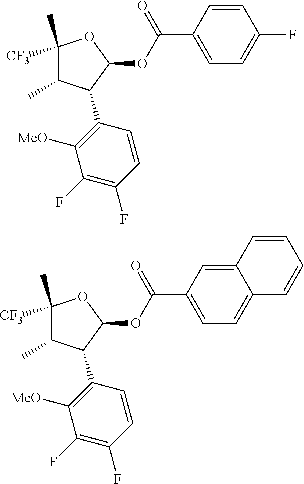 [SMILES: COc1c([C@H]2[C@H](OC(=O)c3ccc(F)cc3)O[C@@](C)(C(F)(F)F)[C@H]2C)ccc(F)c1F ; COc1c([C@H]2[C@H](OC(=O)c3ccc4ccccc4c3)O[C@@](C)(C(F)(F)F)[C@H]2C)ccc(F)c1F]

In some embodiments, the process for preparing a compound of formula (I) or (II) further includes transforming a compound of formula (XI):

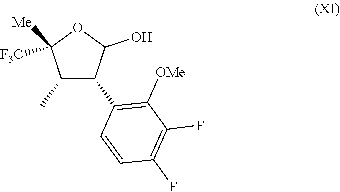 [SMILES: COc1c([C@H]2C(O)O[C@@](C)(C(F)(F)F)[C@H]2C)ccc(F)c1F]

to the compound of formula (X).

In some embodiments, the compound of formula (XI) has the formula (XI′):

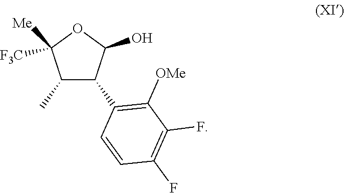 [SMILES: COc1c([C@H]2[C@H](O)O[C@@](C)(C(F)(F)F)[C@H]2C)ccc(F)c1F]

In some embodiments, the compound of formula (XI) has the formula (XI″):

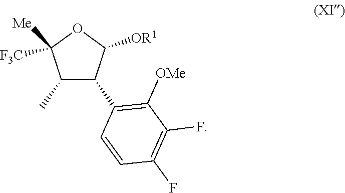

In some embodiments, transforming a compound of formula (XI) to the compound of formula (X) includes contacting the compound of formula (XI) with an acid anhydride or an acid halide to afford the compound of formula (X). In some embodiments, contacting the compound of formula (XI) with an acid anhydride or an acid halide is performed in the presence of a base and a catalyst. One of ordinary skill in the art will appreciate that there are many bases that would be compatible with the process of transforming the compound of formula (XI) to the compound of formula (X). In some embodiments, the base is a non-nucleophilic base. In some embodiments, the base is selected from TEA, pyridine, Hunig's base, K₂CO₃, Na₂CO₃, NaHCO₃, 2,6-Lutidine, NMM, and DABCO. In some embodiments, the base is triethylamine and the catalyst is 4-dimethylaminopyridine (DMAP). In some embodiments, the acid anhydride is acetic anhydride. In some embodiments, the acid halide is 4-nitrobenzoyl chloride. In some embodiments, the transformation of the compound of formula (XI) to the compound of formula (X) is performed in a polar solvent. In some embodiments, the polar solvent is selected from toluene, cyclopentyl methyl ether (CPME), dichloromethane, dichloroethane, pyridine, chloroform, acetonitrile, THF, 2-MeTHE, EtOAc, IPAC or combinations thereof.

In some embodiments, the process for preparing a compound of formula (I) or (II) further includes recrystallizing the compound of formula (X) from a suitable solvent. In some embodiments, the suitable solvent includes acetone, water, and combinations thereof.

In some embodiments, the process for preparing a compound of formula (I) or (II) further includes transforming a compound of formula (XII):

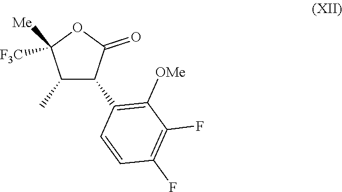 [SMILES: COc1c([C@H]2C(=O)O[C@@](C)(C(F)(F)F)[C@H]2C)ccc(F)c1F]

to the compound of formula (XI).

In some embodiments, transforming the compound of formula (XII) to the compound of formula (XI) includes treating the compound of formula (XII) with a reducing reagent. In some embodiments, the reducing reagent is selected from diisobutylaluminum hydride, Red-Al, NaBH₄/BF₃, titanocene with polymethylhydrosiloxane and phenylsilane. In some embodiments, the reducing reagent is diisobutylaluminum hydride. The reduction reaction may be conducted in an organic solvent or solvent mixture. Suitable solvents include toluene, dichloromethane, 2-methyl THF, THF, TFT, MTBE, CPME, heptane, or a mixture thereof. The reaction may be conducted at below room temperature, for example, about −78° C. to 0° C., about −60° C. to 0° C., about −50° C. to −10° C., about −40° C. to −10° C., about −30° C. to −10° C., about −30° C. to −15° C., about −25° C. to −15° C., or about −20° C. The reduction reaction may be conducted in the presence of CuCl, CuI, CuTol, CuBr, CuF, Cu(II)Cl₂, DMAP, 2,6-lutidine, LiI, or pyridine.

In some embodiments, the process for preparing a compound of formula (I) or (II) further includes transforming a compound of formula (XIII):

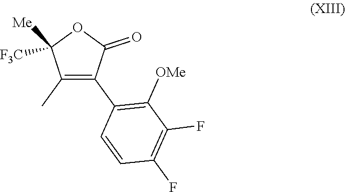 [SMILES: COc1c(C2=C(C)[C@](C)(C(F)(F)F)OC2=O)ccc(F)c1F]

to the compound of formula (XII).

In some embodiments, transforming a compound of formula (XIII) to the compound of formula (XII) comprises a hydrogenation of the compound of formula (XIII). In some embodiments, the hydrogenation is performed in the presence of a hydrogenation catalyst. In some embodiments, the hydrogenation catalyst is selected from Pd/C, Pd/Al₂O₃, Pt/C, Ni (Raney), Co (Raney), Rh/C, Ir/C, Ru/C, Pd(OH)₂, homogeneous chiral Ru and Rh. In some embodiments, the hydrogenation is performed in the presence of a suitable hydrogen source. In some embodiments, the hydrogen source is selected from H₂ gas, NiCl₂/NaBH₄ in methanol, and Et₃SiH. In some embodiments, the hydrogenation is conducted in the presence of H₂ gas using Pd/C (e.g., Johnson Matthey Catalog No. A503032-5 or A503014-5) as a catalyst. The hydrogenation reaction may be conducted in an organic solvent at between about 20 to 40 bar. In some embodiments, the hydrogenation reaction also may be conducted in an organic solvent at between about 5 to 40 barg. In some embodiments, the hydrogenation reaction may be performed at a temperature of 10-50° C. A lower pressure may be use with high temperature and vice versa. For instance, about 5 bar may be suitable at about 40° C. Conversely, about 15-20 bar may be suitable at about 30° C. The skilled artisan can match pressure, temperature and reaction time to obtain desirable results. The hydrogenation reaction may be conducted in an organic solvent or a solvent mixture. In one embodiment, the organic solvent is IPA, EtOAc, MeOH, nBuOH, THF, MTBE, CPME, IPAc, nBuAc, Toluene, Ethanol, or a mixture thereof. In some embodiments, the hydrogenation is performed in a solvent mixture comprising 2-propanol, tetrahydrofuran, and catalytic trifluoroacetic acid. The asymmetric hydrogenation reaction may be conducted in the presence of citric acid, benzoic acid, TFA, AcOH, H₂SO₄, H₃PO₄, MSA, Cs₂CO₃, CuCl, MgF₂, LiBr, CsF, ZnI, LiOTf, imidazole, KF, Bu₄NOAc, and/or NH₄BF₄.

In some embodiments, the process for preparing a compound of formula (I) or (II) further includes contacting a compound of formula (XIV):

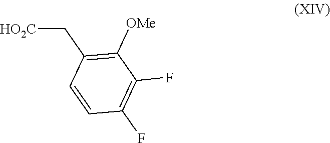 [SMILES: COc1c(CC(=O)O)ccc(F)c1F]

with a compound of formula (XV):

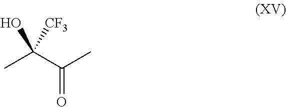

to afford the compound of formula (XIII).

In some embodiments, contacting a compound of formula (XIV) with a compound of formula (XV) is performed in the presence of a coupling agent or a chlorinating agent. In some embodiments, the coupling agent is selected from CDI and T3P. The coupling reaction between compounds of formulae (XIV) and (XV) may be conducted in the presence of a mild or a non-nucleophilic base. Examples of mild or non-nucleophilic bases suitable for the coupling reaction between compounds of formulae (XIV) and (XV) include imidazole, DIPEA, TEA, NMM, TBD, Na₂CO₃, K₃PO₄, DBU, DABCO, and MTBD. In some embodiments, the mild or non-nucleophilic base is imidazole, DIPEA, TEA, NMM, or TBD. In some embodiments, the base is K₂CO₃. The coupling reaction between compounds of formulae (XIV) and (XV) may be conducted in a polar aprotic solvent. Examples of polar aprotic solvents that may be suitable for the claims of the present application include solvents comprising MTBE, toluene, EtOAc, MeCN, THF, DMC, MeOAc, NMP, DMF, DMSO, THE, 2-Me THF, and combinations thereof. The coupling reaction between compounds of formulae (XIV) and (XV) may be conducted at between about 20° C. and about 60° C., between about 25° C. and about 55° C., between about 30° C. and about 50° C., between about 30° C. and about 45° C., between about 30° C. and about 40° C., or about 35° C. As used in this paragraph, the term “about” means±2.5° C. In some embodiments, the chlorinating agent converts the compound of formula (XIV) to an acid chloride, which is not isolated before reacting with the compound of formula (XV). Examples of chlorinating agents suitable in coupling the compound of formula (XIV) with the compound of formula (XV) include oxalyl chloride, thionyl chloride, phosgene, and the like. In some embodiments, the chlorinating agent is selected from oxalyl chloride and thionyl chloride.

Alternatively, the compound of formula (IV) may be prepared by transforming a compound of formula (XXII):

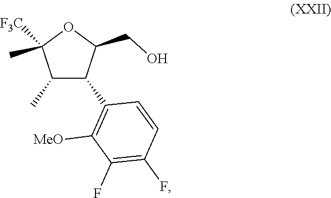 [SMILES: COc1c([C@H]2[C@H](CO)O[C@@](C)(C(F)(F)F)[C@H]2C)ccc(F)c1F]

to the compound of formula (IV).

In some embodiments, transforming a compound of formula (XXII) to the compound of formula (IV) comprises treating the compound of formula (XXII) with an oxidant. In some embodiments, the oxidant comprises TEMPO and NaOCl.

In some embodiments, the compound of formula (XXII) can be obtained by transforming a compound of formula (XXIII):

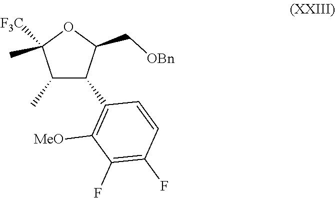 [SMILES: COc1c([C@H]2[C@H](COCc3ccccc3)O[C@@](C)(C(F)(F)F)[C@H]2C)ccc(F)c1F]

to the compound of formula (XXII).

In some embodiments, transforming the compound of formula (XXIII) to the compound of formula (XXII) comprises hydrogenating the compound of formula (XXIII). In some embodiments, hydrogenating the compound of formula (XXIII) is performed in the presence of hydrogen and a palladium on carbon catalyst.

In some embodiments, the compound of formula (XXIII) can be obtained by transforming a compound of formula (XXIV):

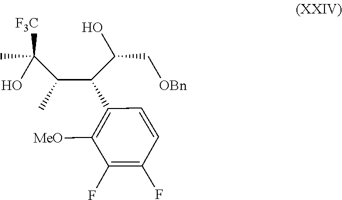 [SMILES: COc1c([C@@H]([C@H](O)COCc2ccccc2)[C@H](C)[C@@](C)(O)C(F)(F)F)ccc(F)c1F]

to the compound of formula (XXIII).

In some embodiments, transforming the compound of formula (XXIV) to the compound of formula (XXIII) comprises treating the compound of formula (XXIV) with mesyl chloride in the presence of a base. In some embodiments, the base is triethylamine.

In some embodiments, the compound of formula (XXIV) is obtained by transforming a compound of formula (XXV):

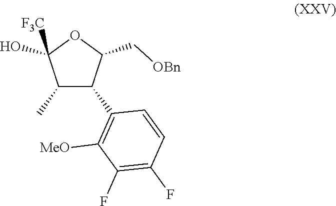 [SMILES: COc1c([C@H]2[C@@H](COCc3ccccc3)O[C@](O)(C(F)(F)F)[C@H]2C)ccc(F)c1F]

to the compound of formula (XXIV).

In some embodiments, transforming the compound of formula (XXV) to the compound of formula (XXIV) comprises treating the compound of formula (XXV) with methyl magnesium chloride.

In some embodiments, the compound of formula (XXV) is obtained by transforming a

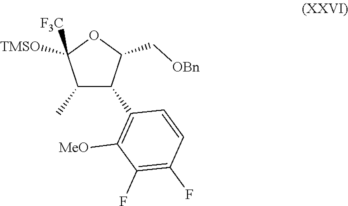

to the compound of formula (XXV).

In some embodiments, transforming the compound of formula (XXVI) to the compound of formula (XXV) comprises treating the compound of formula (XXVI) with tetra-N-butylammonium fluoride (TBAF).

In some embodiments, the compound of formula (XXVI) is obtained by transforming a compound of formula (XXVII):

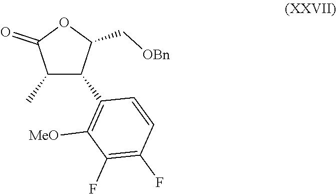 [SMILES: COc1c([C@@H]2[C@H](C)C(=O)O[C@@H]2COCc2ccccc2)ccc(F)c1F]

to the compound of formula (XXVI).

In some embodiments, transforming the compound of formula (XXVII) to the compound of formula (XXVI) comprises treating the compound of formula (XXVII) with trimethyl(trifluoromethyl) silane in the presence cesium fluoride.

In some embodiments, the compound of formula (XXVII) is obtained by transforming a compound of formula (XXVIII):

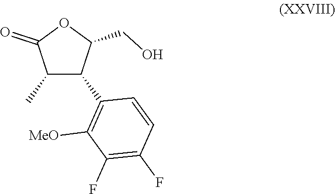 [SMILES: COc1c([C@@H]2[C@H](C)C(=O)O[C@@H]2CO)ccc(F)c1F]

to the compound of formula (XXVII).

In some embodiments, transforming a compound of formula (XXVIII) to the compound of formula (XXVII) comprises treating the compound of formula (XXVIII) with benzyl-2,2,2-trichloroethan-imidate in the presence of an acid. In some embodiments, the acid is triflic acid.

In some embodiments, the compound of formula (XXVIII) is obtained by transforming a compound of formula (XXIX):

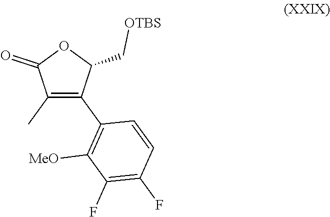 [SMILES: COc1c(C2=C(C)C(=O)O[C@@H]2CO[Si](C)(C)C(C)(C)C)ccc(F)c1F]

to the compound of formula (XVIII).

In some embodiments, transforming the compound of formula (XXIX) to the compound of formula (XXVIII) includes a hydrogenation of the compound of formula (XXIX). In some embodiments, the hydrogenation of the compound of formula (XXIX) is performed in the presence of NiCl₂/NaBH₄ in methanol.

In some embodiments, the present invention is directed to a method for preparing a compound of formula (I), or a salt thereof, comprising converting a compound of formula (XIII) to the compound of formula (I) using the methods described herein for converting the compound of formula (XIII) to the compound of formula (I).

In some embodiments, the present invention is directed to a method for preparing a compound of formula (I), or a salt thereof, comprising converting a compound of formula (XII) to the compound of formula (I) using the methods described herein for converting the compound of formula (XII) to the compound of formula (I).

In some embodiments, the present invention is directed to a method for preparing a compound of formula (I), or a salt thereof, comprising converting a compound of formula (XI) to the compound of formula (I) using the methods described herein for converting the compound of formula (XI) to the compound of formula (I).

In some embodiments, the present invention is directed to a method for preparing a compound of formula (I), or a salt thereof, comprising converting a compound of formula (X) to the compound of formula (I) using the methods described herein for converting the compound of formula (X) to the compound of formula (I).

In some embodiments, the present invention is directed to a method for preparing a compound of formula (I), or a salt thereof, comprising converting a compound of formula (IX) to the compound of formula (I) using the methods described herein for converting the compound of formula (IX) to the compound of formula (I).

In some embodiments, the present invention is directed to a method for preparing a compound of formula (I), or a salt thereof, comprising converting a compound of formula (VIII) to the compound of formula (I) using the methods described herein for converting the compound of formula (VIII) to the compound of formula (I).

In some embodiments, the present invention is directed to a method for preparing a compound of formula (I), or a salt thereof, comprising converting a compound of formula (VII) to the compound of formula (I) using the methods described herein for converting the compound of formula (VII) to the compound of formula (I).

In some embodiments, the present invention is directed to a method for preparing a compound of formula (I), or a salt thereof, comprising converting a compound of formula (VI) to the compound of formula (I) using the methods described herein for converting the compound of formula (VI) to the compound of formula (I).

In some embodiments, the present invention is directed to a method for preparing a compound of formula (I), or a salt thereof, comprising converting a compound of formula (III) to the compound of formula (I) using the methods described herein for converting the compound of formula (III) to the compound of formula (I).

In some embodiments, the present invention is directed to a method for preparing a compound of formula (I), or a salt thereof, comprising converting a compound of formula (XXII) to the compound of formula (I) using the methods described herein for converting the compound of formula (XXII) to the compound of formula (I).

In some embodiments, the present invention is directed to a method for preparing a compound of formula (I), or a salt thereof, comprising converting a compound of formula (XXIII) to the compound of formula (I) using the methods described herein for converting the compound of formula (XXIII) to the compound of formula (I).

In some embodiments, the present invention is directed to a method for preparing a compound of formula (I), or a salt thereof, comprising converting a compound of formula (XXIV) to the compound of formula (I) using the methods described herein for converting the compound of formula (XXIV) to the compound of formula (I).

In some embodiments, the present invention is directed to a method for preparing a compound of formula (I), or a salt thereof, comprising converting a compound of formula (XXV) to the compound of formula (I) using the methods described herein for converting the compound of formula (XXV) to the compound of formula (I).

In some embodiments, the present invention is directed to a method for preparing a compound of formula (I), or a salt thereof, comprising converting a compound of formula (XXVI) to the compound of formula (I) using the methods described herein for converting the compound of formula (XXVI) to the compound of formula (I).

In some embodiments, the present invention is directed to a method for preparing a compound of formula (I), or a salt thereof, comprising converting a compound of formula (XXVII) to the compound of formula (I) using the methods described herein for converting the compound of formula (XXVII) to the compound of formula (I).

In some embodiments, the present invention is directed to a method for preparing a compound of formula (I), or a salt thereof, comprising converting a compound of formula (XXVIII) to the compound of formula (I) using the methods described herein for converting the compound of formula (XXVIII) to the compound of formula (I).

In some embodiments, the present invention is directed to a method for preparing a compound of formula (I), or a salt thereof, comprising converting a compound of formula (XXIX) to the compound of formula (I) using the methods described herein for converting the compound of formula (XXIX) to the compound of formula (I).

In some embodiments, the present invention is directed to a method for preparing a compound of formula (II), or a salt thereof, comprising converting a compound of formula (XIII) to the compound of formula (II) using the methods described herein for converting the compound of formula (XIII) to the compound of formula (II).

In some embodiments, the present invention is directed to a method for preparing a compound of formula (II), or a salt thereof, comprising converting a compound of formula (XII) to the compound of formula (II) using the methods described herein for converting the compound of formula (XII) to the compound of formula (II).

In some embodiments, the present invention is directed to a method for preparing a compound of formula (II), or a salt thereof, comprising converting a compound of formula (XI) to the compound of formula (II) using the methods described herein for converting the compound of formula (XI) to the compound of formula (II).

In some embodiments, the present invention is directed to a method for preparing a compound of formula (II), or a salt thereof, comprising converting a compound of formula (X) to the compound of formula (II) using the methods described herein for converting the compound of formula (X) to the compound of formula (II).

In some embodiments, the present invention is directed to a method for preparing a compound of formula (II), or a salt thereof, comprising converting a compound of formula (IX) to the compound of formula (II) using the methods described herein for converting the compound of formula (IX) to the compound of formula (II).

In some embodiments, the present invention is directed to a method for preparing a compound of formula (II), or a salt thereof, comprising converting a compound of formula (VIII) to the compound of formula (II) using the methods described herein for converting the compound of formula (VIII) to the compound of formula (II).

In some embodiments, the present invention is directed to a method for preparing a compound of formula (II), or a salt thereof, comprising converting a compound of formula (VI) to the compound of formula (II) using the methods described herein for converting the compound of formula (VI) to the compound of formula (II).

In some embodiments, the present invention is directed to a method for preparing a compound of formula (II), or a salt thereof, comprising converting a compound of formula (IV) to the compound of formula (II) using the methods described herein for converting the compound of formula (IV) to the compound of formula (II).

In some embodiments, the present invention is directed to a method for preparing a compound of formula (II), or a salt thereof, comprising converting a compound of formula (XX) to the compound of formula (II) using the methods described herein for converting the compound of formula (XX) to the compound of formula (II).

In some embodiments, the present invention is directed to a method for preparing a compound of formula (II), or a salt thereof, comprising converting a compound of formula (XXII) to the compound of formula (II) using the methods described herein for converting the compound of formula (XXII) to the compound of formula (II).

In some embodiments, the present invention is directed to a method for preparing a compound of formula (II), or a salt thereof, comprising converting a compound of formula (XXIII) to the compound of formula (II) using the methods described herein for converting the compound of formula (XXIII) to the compound of formula (II).

In some embodiments, the present invention is directed to a method for preparing a compound of formula (II), or a salt thereof, comprising converting a compound of formula (XXIV) to the compound of formula (II) using the methods described herein for converting the compound of formula (XXIV) to the compound of formula (II).

In some embodiments, the present invention is directed to a method for preparing a compound of formula (II), or a salt thereof, comprising converting a compound of formula (XXV) to the compound of formula (II) using the methods described herein for converting the compound of formula (XXV) to the compound of formula (II).

In some embodiments, the present invention is directed to a method for preparing a compound of formula (II), or a salt thereof, comprising converting a compound of formula (XXVI) to the compound of formula (II) using the methods described herein for converting the compound of formula (XXVI) to the compound of formula (II).

In some embodiments, the present invention is directed to a method for preparing a compound of formula (II), or a salt thereof, comprising converting a compound of formula (XXVII) to the compound of formula (II) using the methods described herein for converting the compound of formula (XXVII) to the compound of formula (II).

In some embodiments, the present invention is directed to a method for preparing a compound of formula (II), or a salt thereof, comprising converting a compound of formula (XXVIII) to the compound of formula (II) using the methods described herein for converting the compound of formula (XXVIII) to the compound of formula (II).

In some embodiments, the present invention is directed to a method for preparing a compound of formula (II), or a salt thereof, comprising converting a compound of formula (XXIX) to the compound of formula (II) using the methods described herein for converting the compound of formula (XXIX) to the compound of formula (II).

In some embodiments, the present invention is directed to a method for preparing an intermediate compound of formulae (III), (IV), and (VI)-(XIII) using the methods described herein.

In some embodiments, the present invention is directed to a method for preparing a compound of formula (III), or a salt thereof, including converting any of the compounds of formulae (IV) and (VI)-(XIII) to the compound of formula (III) using the methods disclosed herein.

In some embodiments, the present invention is directed to a method for preparing a compound of formula (VIII), or a salt thereof, including converting any of the compounds of formulae (IV) and (VI), (VII), and (IX)-(XIII) to the compound of formula (VIII) using the methods disclosed herein.

In some embodiments, the present invention is directed to a method for preparing a compound of formula (VI), or a salt thereof, including converting any of the compounds of formulae (IV) and (VII), and (IX)-(XIII) to the compound of formula (VI) using the methods disclosed herein.

In some embodiments, the present invention is directed to a method for preparing a compound of formula (VII), or a salt thereof, including converting any of the compounds of formulae (IV) and (IX)-(XIII) to the compound of formula (VII) using the methods disclosed herein.

In some embodiments, the present invention is directed to a method for preparing a compound of formula (IV), or a salt thereof, including converting any of the compounds of formulae (IX)-(XIII) to the compound of formula (IV) using the methods disclosed herein.

In some embodiments, the present invention is directed to a method for preparing a compound of formula (IX), or a salt thereof, including converting any of the compounds of formulae (X)-(XIII) to the compound of formula (IX) using the methods disclosed herein.

In some embodiments, the present invention is directed to a method for preparing a compound of formula (X), or a salt thereof, including converting any of the compounds of formulae (XI)-(XIII) to the compound of formula (X) using the methods disclosed herein.

In some embodiments, the present invention is directed to a method for preparing a compound of formula (XI), or a salt thereof, including converting any of the compounds of formulae (XII)-(XIII) to the compound of formula (XI) using the methods disclosed herein.

In some embodiments, the present invention is directed to a method for preparing a compound of formula (XII), or a salt thereof, including converting a compound of formula (XIII) to the compound of formula (XII) using the methods disclosed herein.

In some embodiments, the present invention is directed to a method for preparing an intermediate compound of formulae (XX) using the methods described herein.

In some embodiments, the present invention is directed to a method for preparing a compound of formula (XX), or a salt thereof, including converting any of the compounds of formulae (IV), and (VI)-(XIII) to the compound of formula (XX) using the methods disclosed herein.

In some embodiments, the present invention is directed to a method for preparing an intermediate compound of formulae (III), (IV), (VI)-(VIII), and (XXII)-(XXIX) using the methods described herein.

In some embodiments, the present invention is directed to a method for preparing a compound of formula (III), or a salt thereof, including converting any of the compounds of formulae (IV), (VI)-(VIII), and (XXII)-(XXIX) to the compound of formula (III) using the methods disclosed herein.

In some embodiments, the present invention is directed to a method for preparing a compound of formula (VIII), or a salt thereof, including converting any of the compounds of formulae (IV), (VI)-(VII), and (XXII)-(XXIX) to the compound of formula (VIII) using the methods disclosed herein.

In some embodiments, the present invention is directed to a method for preparing a compound of formula (VI), or a salt thereof, including converting any of the compounds of formulae (IV), (VII), and (XXII)-(XXIX) to the compound of formula (VI) using the methods disclosed herein.

In some embodiments, the present invention is directed to a method for preparing a compound of formula (VII), or a salt thereof, including converting any of the compounds of formulae (IV) and (XXII)-(XXIX) to the compound of formula (VII) using the methods disclosed herein.

In some embodiments, the present invention is directed to a method for preparing a compound of formula (IV), or a salt thereof, including converting any of the compounds of formulae (XXII)-(XXIX) to the compound of formula (IV) using the methods disclosed herein.

In some embodiments, the present invention is directed to a method for preparing a compound of formula (XXII), or a salt thereof, including converting any of the compounds of formulae (XXIII)-(XXIX) to the compound of formula (XXII) using the methods disclosed herein.

In some embodiments, the present invention is directed to a method for preparing a compound of formula (XXIII), or a salt thereof, including converting any of the compounds of formulae (XXIV)-(XXIX) to the compound of formula (XXIII) using the methods disclosed herein.

In some embodiments, the present invention is directed to a method for preparing a compound of formula (XXIV), or a salt thereof, including converting any of the compounds of formulae (XXV)-(XXIX) to the compound of formula (XXIV) using the methods disclosed herein.

In some embodiments, the present invention is directed to a method for preparing a compound of formula (XXV), or a salt thereof, including converting any of the compounds of formulae (XXVI)-(XXIX) to the compound of formula (XXV) using the methods disclosed herein.

In some embodiments, the present invention is directed to a method for preparing a compound of formula (XXVI), or a salt thereof, including converting any of the compounds of formulae (XXVII)-(XXIX) to the compound of formula (XXVI) using the methods disclosed herein.

In some embodiments, the present invention is directed to a method for preparing a compound of formula (XXVII), or a salt thereof, including converting any of the compounds of formulae (XXVIII)-(XXIX) to the compound of formula (XXVII) using the methods disclosed herein.

In some embodiments, the present invention is directed to a method for preparing a compound of formula (XXVIII), or a salt thereof, including converting a compound of formula (XXIX) to the compound of formula (XXVIII) using the methods disclosed herein.

In some embodiments, the present invention is directed to a method for preparing an intermediate compound of formulae (IV), (VI)-(VIII), (XX), and (XXII)-(XXIX) using the methods described herein.

In some embodiments, the present invention is directed to a method for preparing a compound of formula (XX), or a salt thereof, including converting any of the compounds of formulae (IV), (VI)-(VIII), and (XXII)-(XXIX) to the compound of formula (XX) using the methods disclosed herein.

In some embodiments, the present invention is directed to a method for preparing an intermediate compound of formulae (V) and (XVI)-(XIX) using the methods described herein.

In some embodiments, the present invention is directed to a method for preparing a compound of formula (V), or a salt thereof, including converting any of the compounds of formulae (XVI)-(XIX) to the compound of formula (V) using the methods disclosed herein.

In some embodiments, the present invention is directed to a method for preparing a compound of formula (XVI), or a salt thereof, including converting any of the compounds of formulae (XVII)-(XIX) to the compound of formula (XVI) using the methods disclosed herein.

In some embodiments, the present invention is directed to a method for preparing a compound of formula (XVII), or a salt thereof, including converting any of the compounds of formulae (XVIII)-(XIX) to the compound of formula (XVII) using the methods disclosed herein.

In some embodiments, the present invention is directed to a method for preparing a compound of formula (XVIII), or a salt thereof, including converting a compound of formula (XIX) to the compound of formula (XVIII) using the methods disclosed herein.

In a third aspect, the present invention relates to an intermediate compound selected from Table A, below.

TABLE A
Intermediate compounds of the invention.

| | |
|---|---|
| 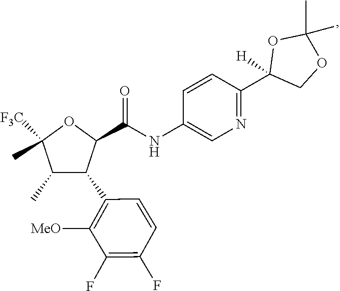 | (III) |
| 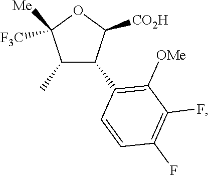 [SMILES: COc1c([C@H]2[C@H](C(=O)O)O[C@@](C)(C(F)(F)F)[C@H]2C)ccc(F)c1F] | (IV) |
| 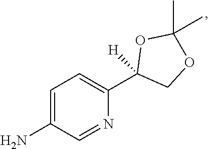 | (V) |
| 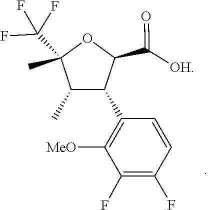 [SMILES: COc1c([C@H]2[C@H](C(=O)O)O[C@@](C)(C(F)(F)F)[C@H]2C)ccc(F)c1F] 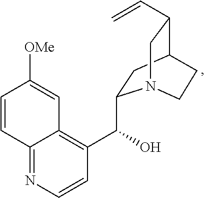 [SMILES: C=CC1CN2CCC1CC2[C@H](O)c1ccnc2ccc(OC)cc12] | (VI) |
| 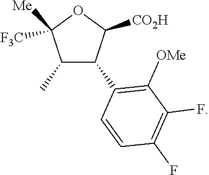 [SMILES: COc1c([C@H]2[C@H](C(=O)O)O[C@@](C)(C(F)(F)F)[C@H]2C)ccc(F)c1F] 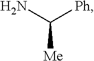 | (VII) |
| 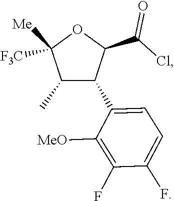 [SMILES: COc1c([C@H]2[C@H](C(=O)Cl)O[C@@](C)(C(F)(F)F)[C@H]2C)ccc(F)c1F] | (VIII) |
| 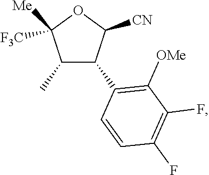 [SMILES: COc1c([C@H]2[C@H](C#N)O[C@@](C)(C(F)(F)F)[C@H]2C)ccc(F)c1F] | (IX) |
| 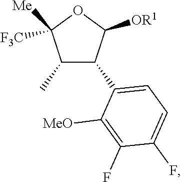 | (X) |
| 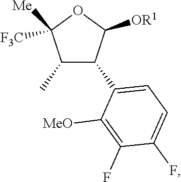 | (X′) |
| 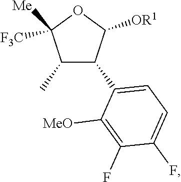 | (X″) |
| 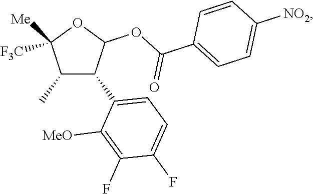 [SMILES: COc1c([C@H]2C(OC(=O)c3ccc([N+](=O)[O-])cc3)O[C@@](C)(C(F)(F)F)[C@H]2C)ccc(F)c1F] | (X-A) |
| 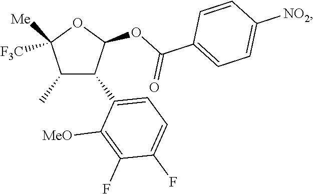 [SMILES: COc1c([C@H]2[C@H](OC(=O)c3ccc([N+](=O)[O-])cc3)O[C@@](C)(C(F)(F)F)[C@H]2C)ccc(F)c1F] | (X′-A) |
| 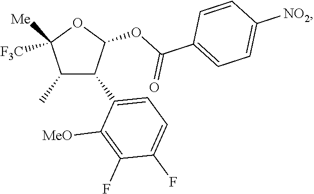 [SMILES: COc1c([C@H]2[C@@H](OC(=O)c3ccc([N+](=O)[O-])cc3)O[C@@](C)(C(F)(F)F)[C@H]2C)ccc(F)c1F] | (X″-A) |
| 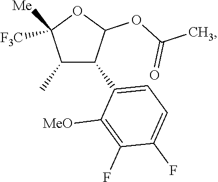 [SMILES: COc1c([C@H]2C(OC(C)=O)O[C@@](C)(C(F)(F)F)[C@H]2C)ccc(F)c1F] | (X-B) |
| 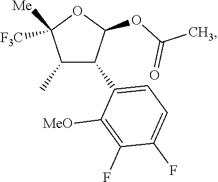 [SMILES: COc1c([C@H]2[C@H](OC(C)=O)O[C@@](C)(C(F)(F)F)[C@H]2C)ccc(F)c1F] | (X′-B) |
| 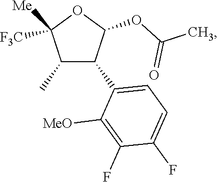 [SMILES: COc1c([C@H]2[C@@H](OC(C)=O)O[C@@](C)(C(F)(F)F)[C@H]2C)ccc(F)c1F] | (X″-B) |
| 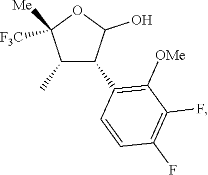 [SMILES: COc1c([C@H]2C(O)O[C@@](C)(C(F)(F)F)[C@H]2C)ccc(F)c1F] | (XI) |
| 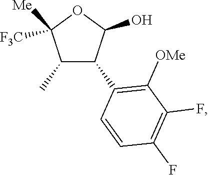 [SMILES: COc1c([C@H]2[C@H](O)O[C@@](C)(C(F)(F)F)[C@H]2C)ccc(F)c1F] | (XI′) |
| 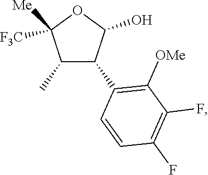 [SMILES: COc1c([C@H]2[C@@H](O)O[C@@](C)(C(F)(F)F)[C@H]2C)ccc(F)c1F] | (XI″) |
| 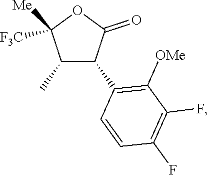 [SMILES: COc1c([C@H]2C(=O)O[C@@](C)(C(F)(F)F)[C@H]2C)ccc(F)c1F] | (XII) |
| 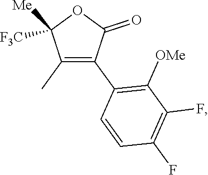 [SMILES: COc1c(C2=C(C)[C@](C)(C(F)(F)F)OC2=O)ccc(F)c1F] | (XIII) |
| 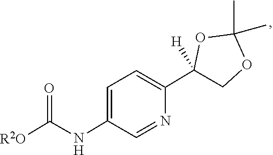 | (XVI) |
| 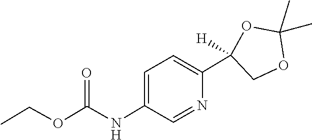 [SMILES: CCOC(=O)Nc1ccc([C@@H]2COC(C)(C)O2)nc1] | (XVI-A) |
| 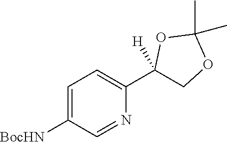 [SMILES: CC(C)(C)OC(=O)Nc1ccc([C@@H]2COC(C)(C)O2)nc1] | (XVI-B) |
| 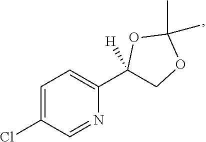 | (XVII) |
| 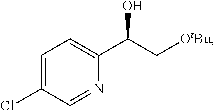 [SMILES: CC(C)(C)OC[C@H](O)c1ccc(Cl)cn1] | (XVIII) |
| 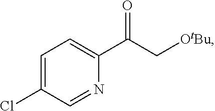 [SMILES: CC(C)(C)OCC(=O)c1ccc(Cl)cn1] | (XIX) |
| 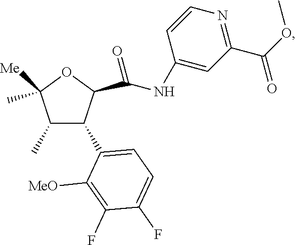 | (XX) |
| 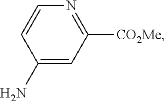 [SMILES: COC(=O)c1cc(N)ccn1] | (XXI) |
| 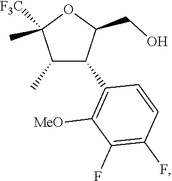 [SMILES: COc1c([C@H]2[C@H](CO)O[C@@](C)(C(F)(F)F)[C@H]2C)ccc(F)c1F] | (XXII) |
| 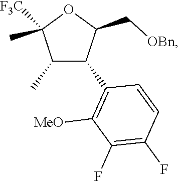 [SMILES: COc1c([C@H]2[C@H](COCc3ccccc3)O[C@@](C)(C(F)(F)F)[C@H]2C)ccc(F)c1F] | (XXIII) |
| 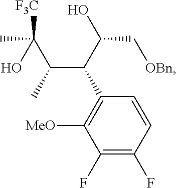 [SMILES: COc1c([C@@H]([C@H](O)COCc2ccccc2)[C@H](C)[C@@](C)(O)C(F)(F)F)ccc(F)c1F] | (XXIV) |
| 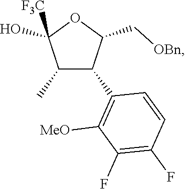 [SMILES: COc1c([C@H]2[C@@H](COCc3ccccc3)O[C@](O)(C(F)(F)F)[C@H]2C)ccc(F)c1F] | (XXV) |
| 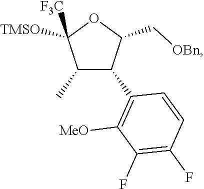 | (XXVI) |
| 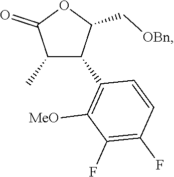 [SMILES: COc1c([C@@H]2[C@H](C)C(=O)O[C@@H]2COCc2ccccc2)ccc(F)c1F] | (XXVII) |
| 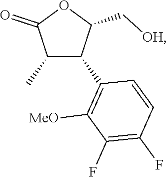 [SMILES: COc1c([C@@H]2[C@H](C)C(=O)O[C@@H]2CO)ccc(F)c1F] or | (XXVIII) |
| 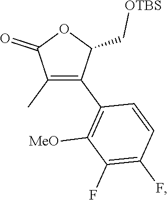 [SMILES: COc1c(C2=C(C)C(=O)O[C@@H]2CO[Si](C)(C)C(C)(C)C)ccc(F)c1F] | (XXIX) |

wherein:
R¹ is —C(O)—Z, —C(O)OZ, —C(O)CH═CH—Z, or —P(O)Z₂;
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl; and
R² is C₁-C₆ alkyl.

In some embodiments, the present invention relates to a compound selected from Table B, below.

TABLE B
Intermediate compounds of the invention.

| | |
|---|---|
| 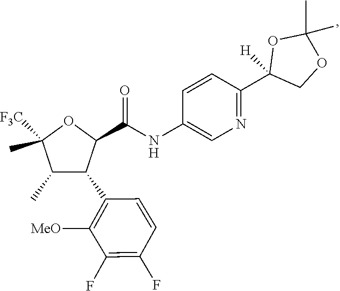 | (III) |
| 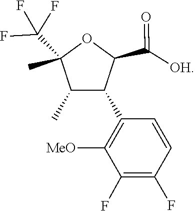 [SMILES: COc1c([C@H]2[C@H](C(=O)O)O[C@@](C)(C(F)(F)F)[C@H]2C)ccc(F)c1F] 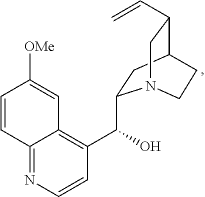 [SMILES: C=CC1CN2CCC1CC2[C@H](O)c1ccnc2ccc(OC)cc12] | (VI) |
| 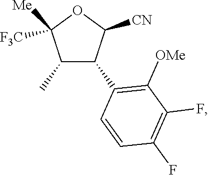 [SMILES: COc1c([C@H]2[C@H](C#N)O[C@@](C)(C(F)(F)F)[C@H]2C)ccc(F)c1F] | (IX) |
| 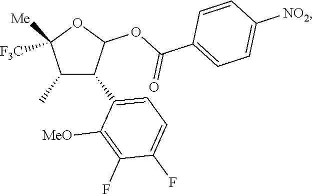 [SMILES: COc1c([C@H]2C(OC(=O)c3ccc([N+](=O)[O-])cc3)O[C@@](C)(C(F)(F)F)[C@H]2C)ccc(F)c1F] | (X-A) |
| 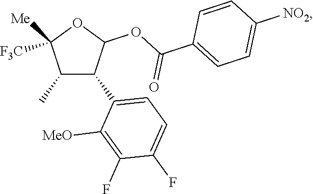 [SMILES: COc1c([C@H]2C(OC(=O)c3ccc([N+](=O)[O-])cc3)O[C@@](C)(C(F)(F)F)[C@H]2C)ccc(F)c1F] | (X′-A) |
| 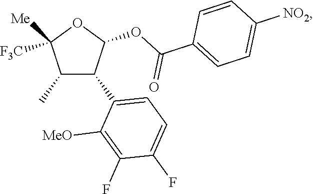 [SMILES: COc1c([C@H]2[C@@H](OC(=O)c3ccc([N+](=O)[O-])cc3)O[C@@](C)(C(F)(F)F)[C@H]2C)ccc(F)c1F] | (X″-A) |
| 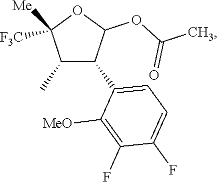 [SMILES: COc1c([C@H]2C(OC(C)=O)O[C@@](C)(C(F)(F)F)[C@H]2C)ccc(F)c1F] | (X-B) |
| 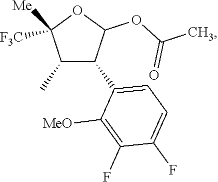 [SMILES: COc1c([C@H]2C(OC(C)=O)O[C@@](C)(C(F)(F)F)[C@H]2C)ccc(F)c1F] | (X′-B) |
| 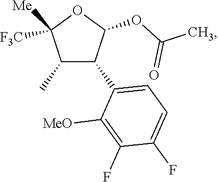 [SMILES: COc1c([C@H]2[C@@H](OC(C)=O)O[C@@](C)(C(F)(F)F)[C@H]2C)ccc(F)c1F] | (X″-B) |
| 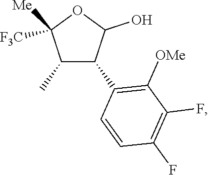 [SMILES: COc1c([C@H]2C(O)O[C@@](C)(C(F)(F)F)[C@H]2C)ccc(F)c1F] | (XI) |
| 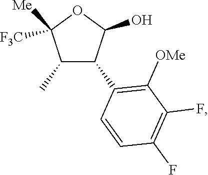 [SMILES: COc1c([C@H]2[C@H](O)O[C@@](C)(C(F)(F)F)[C@H]2C)ccc(F)c1F] | (XI′) |
| 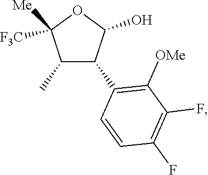 [SMILES: COc1c([C@H]2[C@@H](O)O[C@@](C)(C(F)(F)F)[C@H]2C)ccc(F)c1F] | (XI″) |
| 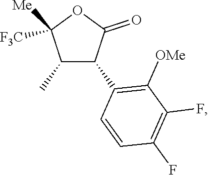 [SMILES: COc1c([C@H]2C(=O)O[C@@](C)(C(F)(F)F)[C@H]2C)ccc(F)c1F] | (XII) |
| 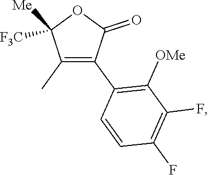 [SMILES: COc1c(C2=C(C)[C@](C)(C(F)(F)F)OC2=O)ccc(F)c1F] | (XIII) |
| 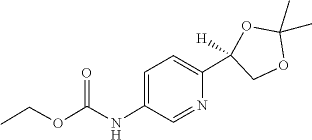 [SMILES: CCOC(=O)Nc1ccc([C@@H]2COC(C)(C)O2)nc1] | (XVI-A) |
| 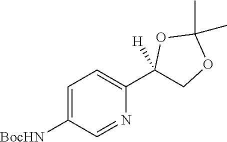 [SMILES: CC(C)(C)OC(=O)Nc1ccc([C@@H]2COC(C)(C)O2)nc1] | (XVI-B) |
| 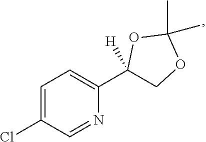 | (XVII) |
| 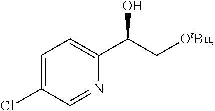 [SMILES: CC(C)(C)OC[C@H](O)c1ccc(Cl)cn1] | (XVIII) |
| 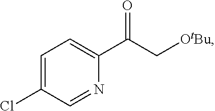 [SMILES: CC(C)(C)OCC(=O)c1ccc(Cl)cn1] | (XIX) |
| 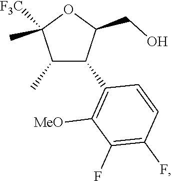 [SMILES: COc1c([C@H]2[C@H](CO)O[C@@](C)(C(F)(F)F)[C@H]2C)ccc(F)c1F] | (XXII) |
| 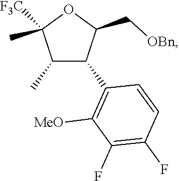 [SMILES: COc1c([C@H]2[C@H](COCc3ccccc3)O[C@@](C)(C(F)(F)F)[C@H]2C)ccc(F)c1F] | (XXIII) |
| 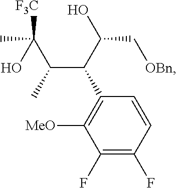 [SMILES: COc1c([C@@H]([C@H](O)COCc2ccccc2)[C@H](C)[C@@](C)(O)C(F)(F)F)ccc(F)c1F] | (XXIV) |
| 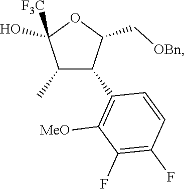 [SMILES: COc1c([C@H]2[C@@H](COCc3ccccc3)O[C@](O)(C(F)(F)F)[C@H]2C)ccc(F)c1F] | (XXV) |
| 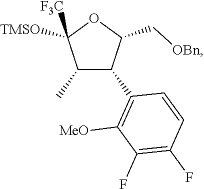 | (XXVI) |
| 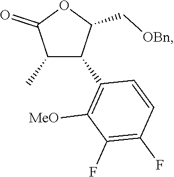 [SMILES: COc1c([C@@H]2[C@H](C)C(=O)O[C@@H]2COCc2ccccc2)ccc(F)c1F] | (XXVII) |
| 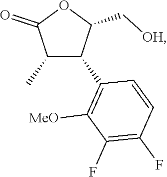 [SMILES: COc1c([C@@H]2[C@H](C)C(=O)O[C@@H]2CO)ccc(F)c1F] or | (XXVIII) |
| 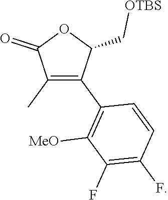 [SMILES: COc1c(C2=C(C)C(=O)O[C@@H]2CO[Si](C)(C)C(C)(C)C)ccc(F)c1F] | (XXIX) |

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, in any chemical structure or formula, a bold or hashed straight bond ( [symbol] or [symbol] , respectively) attached to a stereocenter of a compound, such as in

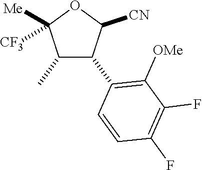 [SMILES: COc1c([C@H]2[C@H](C#N)O[C@@](C)(C(F)(F)F)[C@H]2C)ccc(F)c1F]

denotes the relative stereochemistry of the stereocenter, relative to other stereocenter(s) to which bold or hashed straight bonds are attached.

As used herein, in any chemical structure or formula, a bold or hashed wedge bond ( [symbol] or [symbol] , respectively) attached to a stereocenter of a compound, such as in

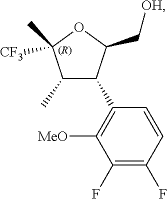

denotes the absolute stereochemistry of the stereocenter, as well as the relative stereochemistry of the stereocenter, relative to other stereocenter(s) to which bold or hashed wedge bonds are attached.

As used herein, the prefix “rac-,” when used in connection with a chiral compound, refers to a racemic mixture of the compound. In a compound bearing the “rac-” prefix, the (R)- and(S)-designators in the chemical name reflect the relative stereochemistry of the compound.

As used herein, the prefix “rel-,” when used in connection with a chiral compound, refers to a single enantiomer of unknown absolute configuration. In a compound bearing the “rel-” prefix, the (R)- and(S)-designators in the chemical name reflect the relative stereochemistry of the compound, but do not necessarily reflect the absolute stereochemistry of the compound.

As used herein, the term “compound,” when referring to the compounds described in this application, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

As used herein, the term “halo” means F, Cl, Br or I.

As used herein, the term “alkyl” refers to a straight or branched hydrocarbon chain radical group consisting solely of carbon and hydrogen atoms, containing no unsaturation, and having the specified number of carbon atoms, which is attached to the rest of the molecule by a single bond. For example, a “C₁-C₆ alkyl” group is an alkyl group having between one and six carbon atoms.

As used herein, the term “alkoxy” refers to a radical of the formula —ORₐ where Rₐ is an alkyl group having the specified number of carbon atoms. For example, a “C₁-C₆ alkoxy” group is a radical of the formula —ORₐ where Rₐ is an alkyl group having the between one and six carbon atoms.

As used herein, the term “haloalkyl” refers to an alkyl group having the specified number of carbon atoms, wherein one or more of the hydrogen atoms of the alkyl group are replaced by halo groups. For example, a “C₁-C₆ haloalkyl” group is an alkyl group having between one and six carbon atoms, wherein one or more of the hydrogen atoms of the alkyl group are replaced by halo groups.

As used herein, the term “haloalkoxy” refers to an alkoxy group having the specified number of carbon atoms, wherein one or more of the hydrogen atoms of the of the alkyl group are replaced by halo groups.

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance concentration of hydrogen.

As used herein, “H” refers to protium. Where an atom in a compound of the invention, or a pharmaceutically acceptable salt thereof, is designated as protium, protium is present at the specified position at at least the natural abundance concentration of protium.

As used herein, “D,” “d,” and “²H” refer to deuterium.

In some embodiments, the compounds described in the present application include each constituent atom at approximately the natural abundance isotopic composition of the specified element.

In some embodiments, the compounds described in the present application, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (²H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the Examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (~45% deuterium incorporation). In some embodiments, the isotopic enrichment factor is at least 3500 (~52.5% deuterium incorporation), at least 4000 (~60% deuterium incorporation), at least 4500 (~67.5% deuterium incorporation), at least 5000 (~75% deuterium incorporation), at least 5500 (~82.5% deuterium incorporation), at least 6000 (~90% deuterium incorporation), at least 6333.3 (~95% deuterium incorporation), at least 6466.7 (~97% deuterium incorporation), at least 6600 (~99% deuterium incorporation), or at least 6633.3 (~99.5% deuterium incorporation).

The term the “compound of formula” followed by a number (typically Roman number) and the term “compound” followed by the same number (Roman or otherwise) may interchangeably be used. For example, the “compound of formula V” and “compound V” denote the same compound.

The term “reacting,” when referring to a chemical reaction, means to add or mix two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “conducted in a solvent,” or “performed in the presence of a solvent,” when referring to a reaction, means that the substrate(s) and reagent(s) are dissolved or suspended in the specified solvent or in a mixture of solvents comprising the specified solvent.

The term “chromatographic purification” refers to any method of purification based on differential retention by a stationary phase. Methods of chromatographic purification include flash chromatography, medium pressure liquid chromatography, preparative thin layer chromatography, and high performance liquid chromatography.

The term “converting,” as used herein to refer to a step of converting a first compound or salt to a second compound or salt, refers to a process of transforming the first compound or salt to the second compound or salt in one or more chemical steps.

The term “acid” refers to a chemical species having a pKa (in water) of less than 7. The term includes inorganic (mineral) acids, such as hydrochloric acid, hydrobromic acid, hydroiodic acid, sulfuric acid, phosphoric acid, nitric acid, and the like. The term also includes organic acids such as acetic acid, propionic acid, n-butyric acid, i-butyric acid, n-valeric acid, i-valeric acid, n-hexanoic acid, succinic acid, glutaric acid, adipic acid, aspartic acid, formic acid, citric acid, o-chlorobenzoic acid, chloroacetic acid, dichloroacetic acid, trichloroacetic acid, nicotinic acid, lactic acid, oxalic acid, picric acid, picolinic acid, fluoroacetic acid, difluoroacetic acid, trifluoroacetic acid, phthalic acid, isophthalic acid, terephthalic acid, maleic acid, malonic acid, and the like.

The term “base” refers to a chemical species whose conjugate acid has a pKa (in water) of greater than 7. The term includes “inorganic bases,” such as sodium hydroxide, potassium hydroxide, sodium bicarbonate, potassium bicarbonate, sodium carbonate, potassium carbonate, cesium carbonate, potassium phosphate (mono-, di-, or tri-basic), sodium hydride, and potassium hydride. The term also includes “anionic organic bases,” such as methyl lithium, butyl lithium, lithium diisopropyl amide, and sodium acetate. The term also includes “neutral organic bases,” such as trimethylamine, dimethylethylamine, diethylmethylamine, triethylamine, di-n-propylmethylamine, dimethylcyclohexylamine, diisopropylethylamine, tri-n-propylamine, diisopropylisobutylamine, dimethyl-n-nonylamine, tri-n-butylamine, di-n-hexylmethylamine, dimethyl-n-dodecylamine, tri-n-pentylamine, 1,4-diazabicyclo[2.2.2]octane (DABCO), dimethylaminopyridine (DMAP), 1,5-diazabicyclo[4.3.0]non-5-ene (DBN), 1,8-diazabicyclo[5.4.0]undec-7-ene (DBU), pyridine, 2,3-lutidine, 2,4-lutidine, 2,5-lutidine, 2,6-lutidine, 3,4-lutidine, 3,5-lutidine, 2,3,4-collidine, 2,4,5-collidine, 2,5,6-collidine, 2,4,6-collidine, 3,4,5-collidine, and 3,5,6-collidine.

The term “alcohol protecting group” refers to a chemical moiety suitable to protect an alcohol group against undesirable side reactions during synthetic procedures. Common alcohol protecting groups include methyl, ethyl, isopropyl, benzyl, 2-tetrahydropyranyl, acetyl, trifluoroacetyl, trialkylsilyl, aryldialkylsilyl, alkyldiarylsilyl, or triarylsilyl. Other alcohol protecting groups also are well known in the art. See, e.g., P. G. M. Wuts et al., Greene's Protective Groups in Organic Synthesis (4th ed. 2006).

The term “deprotecting” refers to a step of reacting a compound or salt containing a protecting group, such as an alcohol protecting group, under conditions suitable to remove the protecting group and reveal the protected moiety. For example, where a compound or salt contains an alcohol protecting group, the term “deprotecting” refers to reacting the compound or salt under conditions suitable to remove the alcohol protecting group and reveal the alcohol. Conditions for removing various protecting groups are well known in the art. See, e.g., P. G. M. Wuts et al., Greene's Protective Groups in Organic Synthesis (4th ed. 2006).

The term “hydrogenation catalyst” refers to any homogeneous or heterogeneous catalyst that catalyzes the hydrogenolysis of benzylic carbon-oxygen single bonds. Suitable hydrogenation catalysts are well-known in the art and include palladium on activated carbon, platinum oxide, and Raney Nickel.

The term “coupling,” when referring to a reaction between a carboxylic acid or acid halide and an amine, refers to a net transformation linking the carboxylic acid or acid halide and the amine to form an amide. The term includes a direct reaction between the carboxylic acid and the amine, as well as a reaction between an activated derivative of the carboxylic acid (such as the derivative formed by the reaction between the carboxylic acid and a coupling reagent) and the amine.

The term “coupling reagent” refers to a reagent suitable to react with a carboxylic acid to activate the carboxylic acid for coupling with an amine to form an amide bond. Coupling reagents are well known in the art. Coupling reagents include, but are not limited to, thionyl chloride, oxalyl chloride, 1,1′-carbonylbis-(4,5-dicyanoimidazole) (CBDCl), 1,1′-carbonyldiimidazole (CDI), propylphosphonic anhydride (T3P), 1-ethyl-3-(3-dimethylaminopropyl)carbodiimide (EDCI), N,N′-dicyclohexylcarbodiimide (DCC), 1-[Bis(dimethylamino)methylene]-1H-1,2,3-triazolo[4,5-b]pyridinium 3-oxid hexafluorophosphate (HATU), and 1-hydroxybenzotriazole (HOBt).

The term “monovalent cation” refers to any cation with a charge of +1, such as alkali metal cations, NH₄⁺, and tetraalkylammonium.

The term “alkali metal cation” refers to a cation derived from a Group I metal atom, including without limitation lithium (Li⁺), sodium (Na⁺), potassium (K⁺), rubidium (Rb⁺), and cesium (Cs⁺).

The term “substituted benzyl” refers to a benzyl group that is substituted with 1-3 substituents selected from the group consisting of C₁-C₃ alkyl, C₁-C₃ alkoxy, halogen, and cyano.

The term “about” means that the stated number can vary from that value by ±10%. Where the term defines a temperature, the stated temperature can vary by ±10%. For example, about 80° C. means between 72° C. and 88° C. Where the term defines pressure, the term “about” means the pressure can vary by ±10%. Thus, about 100 bars means between 90 and 110 bars. Where the term defines quantity (such as equivalents or weight), the term means the quantity can vary by ±10%. For example, about 1 equivalent means between 0.9 and 1.1 equivalents. Where the term defines time, the term means the stated time can vary by ±10%. For example, about 1 hour means between 0.9 and 1.1 hours.

The term “leaving group” is a chemical group that is readily displaced by a desired incoming chemical moiety. Thus, the choice of the specific suitable leaving group is predicated upon its ability to be readily displaced by the incoming chemical moiety such as a CN group. Suitable leaving groups are well known in the art, e.g., see, “Advanced Organic Chemistry,” Jerry March 5.sup.th Ed., pp. 351-357, John Wiley and Sons, N.Y.

As used here, the term “cyanating agent” (such as trimethylsilyl cyanide, diethylaluminum cyanide, KCN, NaCN, TBACN, HCN and the like) to afford the compound of formula IX. In one embodiment, the reaction between the cyanating agent (e.g., trimethylsilyl cyanide) and the compound of formula IX may be conducted in the presence of a Lewis acid. In some embodiments, the Lewis acid is boron trifluoride ethyl etherate (BF₃OEt₂), TiCl₄, InCl₃, AgSbF₆, iodine, ZnBr₂, Al(OiPr)₃, MgCl₂, Mn(acac)₂, MnCl₂, TMSOTf, SnCl₄, and the like. In further embodiments, the Lewis acid is BF₃OEt₂. The cyanation reaction may be conducted in an organic solvent, for example toluene, dichloromethane, 2-methyl THF, acetonitrile, methanol, 1,2-dichloroethane, nitromethane, and the like.

### Solid Forms of the Compounds of the Compounds of the Invention

In another aspect, the invention relates to a compound of the invention, or a pharmaceutically acceptable salt thereof, in solid form. In some embodiments, the compound of the invention, or pharmaceutically acceptable salt thereof, is in crystalline solid form.

### Solid Forms of Compound (I)

In some embodiments, the invention relates to a compound of formula (I)

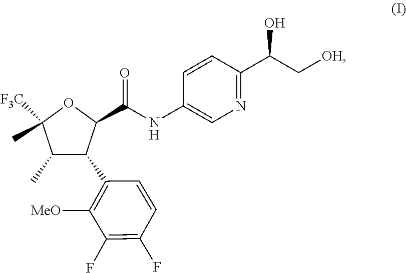 [SMILES: COc1c([C@H]2[C@H](C(=O)Nc3ccc([C@@H](O)CO)nc3)O[C@@](C)(C(F)(F)F)[C@H]2C)ccc(F)c1F]

wherein the compound is in crystalline solid form.

In some embodiments, the invention relates to a composition comprising a compound of formula (I), wherein the compound of formula (I) is in a crystalline solid form. In some embodiments, at least 85% of the compound of formula (I) present in the composition is in a crystalline solid form. In some embodiments, at least 90% of the compound of formula (I) present in the composition is in a crystalline solid form. In some embodiments, at least 95% of the compound of formula (I) present in the composition is in a crystalline solid form. In some embodiments, substantially all of the compound of formula (I) present in the composition is in a crystalline solid form. In some embodiments, 100% of the compound of formula (I) present in the composition is in a crystalline solid form.

In some embodiments, the invention relates to a pharmaceutical composition comprising a compound of formula (I) and one or more pharmaceutically acceptable carriers or vehicles, wherein the compound of formula (I) is in a crystalline solid form. In some embodiments, at least 85% of the compound of formula (I) present in the pharmaceutical composition is in a crystalline solid form. In some embodiments, at least 90% of the compound of formula (I) present in the pharmaceutical composition is in a crystalline solid form. In some embodiments, at least 95% of the compound of formula (I) present in the pharmaceutical composition is in a crystalline solid form. In some embodiments, substantially all of the compound of formula (I) present in the composition is in a crystalline solid form. In some embodiments, 100% of the compound of formula (I) present in the pharmaceutical composition is in a crystalline solid form.

In some embodiments, the crystalline solid form of the compound of formula (I) is Form A.

In some embodiments, Form A is characterized by an X-ray powder diffraction pattern (XRPD) pattern comprising at least one approximate peak position, at least two approximate peak positions, at least three approximate peak positions, at least four approximate peak positions, at least five approximate peak positions, at least six approximate peak positions, at least seven approximate peak positions, at least eight approximate peak positions, at least nine approximate peak positions, at least ten approximate peak positions, at least eleven approximate peak positions, at least twelve approximate peak positions, at least thirteen approximate peak positions, at least fourteen approximate peak positions, at least fifteen approximate peak positions, at least sixteen approximate peak positions, at least seventeen approximate peak positions, at least eighteen approximate peak positions, at least nineteen approximate peak positions, or at least 20 approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.

In some embodiments, Form A is characterized by an X-ray powder diffraction pattern (XRPD) pattern comprising at least one approximate peak position, at least two approximate peak positions, or at least three approximate peak positions (degrees 2-theta±0.2) when measured using Cu K_{α} radiation, selected from 13.4, 17.2, and 18.9.

In some embodiments, Form A is characterized by an X-ray powder diffraction pattern (XRPD) pattern comprising at least one approximate peak position, at least two approximate peak positions, or at least three approximate peak positions (degrees 2-theta±0.2) when measured using Cu K_{α} radiation, selected from 9.0, 11.4, and 20.1.

In some embodiments, Form A is characterized by an X-ray powder diffraction pattern (XRPD) pattern comprising at least one approximate peak position, at least two approximate peak positions, or at least three approximate peak positiosn (degrees 2-theta±0.2) when measured using Cu K_{α} radiation, selected from 13.7, 13.8, and 24.8.

In some embodiments, Form A is characterized by an X-ray powder diffraction pattern (XRPD) pattern comprising at least one approximate peak position, at least two approximate peak positions, or at least three approximate peak positions (degrees 2-theta±0.2) when measured using Cu K_{α} radiation, selected from 13.4, 17.2, and 18.9; and at least one approximate peak position, at least two approximate peak positions, or at least three approximate peak positions (degrees 2-theta±0.2) when measured using Cu K_{α} radiation, selected from 9.0, 11.4, and 20.1.

In some embodiments, Form A is characterized by an X-ray powder diffraction pattern (XRPD) pattern comprising at least one approximate peak position, at least two approximate peak positions, or at least three approximate peak positions (degrees 2-theta±0.2) when measured using Cu K_{α} radiation, selected from 13.4, 17.2, and 18.9; at least one approximate peak position, at least two approximate peak positions, or at least three approximate peak positions (degrees 2-theta±0.2) when measured using Cu K_{α} radiation, selected from 9.0, 11.4, and 20.1; and at least one approximate peak position, at least two approximate peak positions, or at least three approximate peak positions (degrees 2-theta±0.2) when measured using Cu K_{α} radiation, selected from 13.7, 13.8, and 24.8.

In some embodiments, Form A is characterized by a solid state ¹⁹F NMR spectrum having at least one peak, at least two peaks, or at least three peaks at a chemical shift selected from −74.5, −139.5, and −161.5 ppm.

In some embodiments, Form A is characterized by a solid state ¹³C NMR spectrum having at least one peak, at least two peaks, at least three peaks, at least four peaks, at least five peaks, at least six peaks, at least seven peaks, at least eight peaks, at least nine peaks, at least ten peaks, at least eleven peaks, at least twelve peaks, at least thirteen peaks, at least fourteen peaks, at least fifteen peaks, at least sixteen peaks, at least seventeen peaks, at least eighteen peaks, at least nineteen peaks, at least twenty peaks, or at least twenty-one peaks at a chemical shift selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.

In some embodiments, Form A is characterized by an orthorhombic crystal system, as determined by single-crystal X-ray analysis. In other embodiments, Form A is characterized by a P2₁2₁2₁ space group, as determined by single-crystal X-ray analysis. In other embodiments, Form A is characterized by a unit cell, as determined by single-crystal X-ray analysis, of the following dimensions: a=7.0(1) Å; b=8.3(1) Å: c=38.8(1) Å: α=90°; β=90°; and γ=90°.

Further embodiments of the disclosure are set out in the following numbered clauses:

1. A compound of formula (I), a composition comprising a compound of formula (I), or a pharmaceutical composition comprising a compound of formula (I) and one or more pharmaceutically acceptable carriers or vehicles,
wherein:
the compound of formula (I) is in a crystalline solid form;
the crystalline solid form is Form A; and/or
Form A is characterized by an orthorhombic crystal system, as determined by single-crystal X-ray analysis.
   2. The compound, the composition, or the pharmaceutical composition of clause 1, wherein Form A is characterized by a P2₁2₁2₁ space group, as determined by single-crystal X-ray analysis.
   3. The compound, the composition, or the pharmaceutical composition of clause 1 or 2, wherein Form A is characterized by a unit cell, as determined by single-crystal X-ray analysis, of the following dimensions: a=7.0(1) Å; b=8.3(1) Å; c=38.8(1) Å; α=90°; β=90°; and γ=90°.

In some embodiments, Form A of compound (I) is obtainable by dissolving the compound in ethyl acetate and then crystallizing the compound by adding n-heptane as an antisolvent. In other embodiments, Form A is obtainiable by the procedure described in Example 7.

### Solid Forms of Compound (II)

In some embodiments, the invention relates to a compound of formula (II)

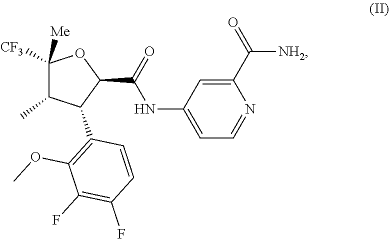 [SMILES: COc1c([C@H]2[C@H](C(=O)Nc3ccnc(C(N)=O)c3)O[C@@](C)(C(F)(F)F)[C@H]2C)ccc(F)c1F]

wherein the compound is in crystalline solid form.

In some embodiments, the crystalline solid form is Form B.

In some embodiments, Form B is characterized by an XRPD pattern having diffractions at angles (degrees 2 theta±0.2) of 12.8, 14.1, 15.2, 18.5, and 20.3. In other embodiments, Form B is characterized by an XRPD pattern having at least one, at least two, at least three, or at least four diffractions at angles (degrees 2 theta±0.2) of 12.8, 14.1, 15.2, 18.5, and 20.3. In other embodiments, Form B is characterized by an XRPD pattern having diffractions at angles (degrees 2 theta±0.2) of 12.0, 12.8, 14.1, 15.2, 16.9, 18.4, 18.5, 18.7, 19.3, and 20.3. In other embodiments, Form B is characterized by an XRPD pattern having at least one, at least two, at least three, at least four, at least five, at least six, at least seven, at least eight, or at least nine diffractions at angles (degrees 2 theta±0.2) of 12.0, 12.8, 14.1, 15.2, 16.9, 18.4, 18.5, 18.7, 19.3, and 20.3. In other embodiments, Form B is characterized by an XRPD pattern having diffractions at angles (degrees 2 theta±0.2) of 7.6, 9.2, 12.0, 12.8, 14.1, 15.1, 15.2, 16.2, 16.9, 17.6, 18.4, 18.5, 18.7, 19.3, 20.3, 21.7, 22.0, 22.2, 22.9, 23.6, 24.0, 24.2, 25.2, 26.9, 27.0, 27.4, 28.6, and 28.9. In other embodiments, Form B is characterized by an XRPD pattern having at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, or at least twenty-seven diffractions at angles (degrees 2 theta±0.2) of 7.6, 9.2, 12.0, 12.8, 14.1, 15.1, 15.2, 16.2, 16.9, 17.6, 18.4, 18.5, 18.7, 19.3, 20.3, 21.7, 22.0, 22.2, 22.9, 23.6, 24.0, 24.2, 25.2, 26.9, 27.0, 27.4, 28.6, and 28.9.

In some embodiments, Form B is characterized by a solid state ¹³C NMR spectrum having peaks at chemical shifts of 172.5, 172.1, 168.5, 168.3, 168.0, 151.5, 148.3, 147.8, 127.7, 122.7, 116.6, 115.1, 110.6, 86.5, 80.2, 63.2, 44.3, 23.0, and 13.1 ppm.

In some embodiments, Form B is characterized by a solid state ¹⁹F NMR spectrum having peaks at chemical shifts of −137.1 and −152.8 ppm.

In some embodiments, Form B is characterized by a DSC thermogram having a melting onset of 182° C. with a peak at 183° C.

In some embodiments, Form B is characterized by an IR spectrum having peaks at 3501, 3356, 1684, 1565, 1505, and 1122 cm⁻¹.

In some embodiments, Form B is characterized by an orthorhombic crystal system, as determined by single-crystal X-ray analysis. In other embodiments, Form B is characterized by a P2₁2₁2₁ space group, as determined by single-crystal X-ray analysis. In other embodiments, Form B is characterized by a unit cell, as determined by single-crystal X-ray analysis, of the following dimensions: a=7.3929(2) Å; b=14.5827(4) Å; c=18.9312(6) Å; α=90°; β=90°; and γ=90°.

### Uses of Compounds, Pharmaceutically Acceptable Salts, and Compositions of the Invention

### Pharmaceutically Acceptable Salts and Compositions

As discussed herein, the invention provides compounds, and pharmaceutically acceptable salts thereof, that are inhibitors of voltage-gated sodium channels, and thus the present compounds, and pharmaceutically acceptable salts thereof, are useful for the treatment of diseases, disorders, and conditions including, but not limited to chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia. Accordingly, in another aspect of the invention, pharmaceutical compositions are provided, wherein these compositions comprise a compound as described herein, or a pharmaceutically acceptable salt thereof, and optionally comprise a pharmaceutically acceptable carrier, adjuvant or vehicle. In certain embodiments, these compositions optionally further comprise one or more additional therapeutic agents. In some embodiments, the additional therapeutic agent is a sodium channel inhibitor.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof. The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the compound of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺ (C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In another aspect, the invention relates to a pharmaceutical composition comprising a compound of the invention (e.g., a compound of formula (I) or (II)), or a pharmaceutically acceptable salt thereof, and a pharmaceutically acceptable carrier, adjuvant or vehicle.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the invention features a pharmaceutical composition comprising a compound of the invention, or a pharmaceutically acceptable salt thereof, and a pharmaceutically acceptable carrier.

In another aspect, the invention features a pharmaceutical composition comprising a therapeutically effective amount of a compound, or a pharmaceutically acceptable salt thereof, and one or more pharmaceutically acceptable carriers or vehicles.

### Dosage Forms and Routes of Administration

A compound of the invention (e.g., a compound of formula (I) or (II)), or a pharmaceutically acceptable salt thereof, may be formulated in dosage unit form for ease of administration and uniformity of dosage. The term “dosage unit form,” as used herein, refers to a physically discrete unit of agent appropriate for the subject to be treated.

The compounds of the invention, or pharmaceutically acceptable salt thereof, can be administered to humans and other animals orally, rectally, parenterally, intracisternally, intravaginally, intraperitoneally, topically (as by powders, ointments, or drops), bucally, as an oral or nasal spray, or the like, depending on the condition being treated.

Liquid dosage forms for oral administration include, but are not limited to, pharmaceutically acceptable emulsions, microemulsions, solutions, suspensions, syrups and elixirs. In addition to the compound of the invention, or a pharmaceutically acceptable salt thereof, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the therapeutic effect of the compound of the invention, it may be desirable to slow the absorption of the compound, or a pharmaceutically acceptable salt thereof, from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound of the invention, or a pharmaceutically acceptable salt thereof, with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the compound of the invention, or a pharmaceutically acceptable salt thereof, is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or, in a certain part of the intestinal tract, optionally, in a delayed manner. Examples of embedding compositions that can be used include polymeric substances and waxes.

The active compound or salt can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of the compound of the invention, or a pharmaceutically acceptable salt thereof, include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Solid Dispersion and Tablet

In another aspect, the invention relates to a solid dispersion (e.g., a spray dried dispersion) comprising a compound of the invention (e.g., a compound of formula (I) or (II)), or a pharmaceutically acceptable salt thereof, and at least one polymer. Spray drying converts a liquid feed to a dried particulate form. Spray drying generally involves bringing into contact a highly dispersed liquid suspension or solution and a sufficient volume of hot air to promote drying of the liquid droplets. For example, a liquid solution containing a compound of the invention (e.g., a compound of formula (I) or (II)), or a pharmaceutically acceptable salt thereof, and at least one polymer can be sprayed into a current of warm filtered gas that evaporates the solvent and conveys the dried product to a collector. Evaporated solvent and spent gas are removed from the collector and can be sent to a condenser to capture the solvent. For example, commercial spray dryers are manufactured by Buchi Ltd. and Niro (e.g., the PSD line of spray driers manufactured by Niro) (see, US 2004/0105820, US 2003/0144257).

Techniques and methods for spray drying may be found in Perry's Chemical Engineering Handbook, 6th Ed., R. H. Perry, D. W. Green & J. O. Maloney, eds.), McGraw-Hill book co. (1984); and Marshall “Atomization and Spray-Drying” 50, Chem. Eng. Prog. Monogr. Series 2 (1954). All three references are incorporated herein in their entirety by reference.

Additional drying steps may be required after spray-drying to ensure removal of the solvent. Other drying techniques include, but are not limited to, tray drying, fluid bed drying (e.g., from about room temperature to about 100° C.), vacuum drying, microwave drying, rotary drum drying or biconical vacuum drying (e.g., from about room temperature to about 200° C.).

In some embodiments, the solvent(s) used in spray-drying is a volatile solvent. A volatile solvent, for example, can have a boiling point less than 100° C. A mixture of volatile solvents may be used or a mixture of volatile and non-volatile solvents.

Exemplary solvents that could be tested include acetone, cyclohexane, dichloromethane, N,N-dimethylacetamide (DMA), N,N-dimethylformamide (DMF), 1,3-dimethyl-2-imidazolidinone (DMI), dimethyl sulfoxide (DMSO), dioxane, ethyl acetate, ethyl ether, glacial acetic acid (HOAc), methyl ethyl ketone (MEK), N-methyl-2-pyrrolidinone (NMP), methyl tert-butyl ether (MTBE), tetrahydrofuran (THF), pentane, acetonitrile, methanol, ethanol, isopropyl alcohol, isopropyl acetate, DCM, and toluene. Exemplary co-solvents include acetone/DMSO, acetone/DMF, acetone/water, MEK/water, THF/water, dioxane/water. In a two solvent system, the solvents can be present in of from about 0.1% to about 99.9%. In some embodiments, water is a co-solvent with acetone where water is present from about 0.1% to about 15%, for example about 9% to about 11%, e.g., about 10%. In some embodiments, water is a co-solvent with MEK where water is present from about 0.1% to about 15%, for example about 9% to about 11%, e.g., about 10%. In some embodiments the solvent system includes three solvents. Suitable solvents include those described above, for example, DCM, water, methanol, IPA, and mixtures thereof. In some embodiments, the solvent comprises DCM and methanol.

In some embodiments, the at least one polymer is selected from: hydroxypropyl methylcellulose acetate succinate (HPMCAS), polyvinyl caprolactam-polyvinyl acetate-polyethylene glycol graft co-polymer, and any combination thereof.

In some embodiments, the at least one polymer is HPMCAS.

In another aspect, the invention relates to a pharmaceutical composition that includes the solid dispersion disclosed herein. In some embodiments, the pharmaceutical composition can include one or more excipients. Examples of excipients include, but are not limited to, fillers, disintegrants, and lubricants.

Examples of fillers include, but are not limited to, microcrystalline cellulose, lactose monohydrate, mannitol, and mixtures of the same. In some embodiments, the filler comprises microcrystalline cellulose. In some embodiments, the filler comprises lactose monohydrate. In some embodiments, the filler comprises mannitol. In some embodiments, the filler comprises a mixture of microcrystalline cellulose and lactose monohydrate. In some embodiments, the filler comprises microcrystalline cellulose, wherein the microcrystalline cellulose is Avicel PhH101. In some embodiments, the filler comprises microcrystalline cellulose, wherein the microcrystalline cellulose is Avicel PH102. In some embodiments, the filler comprises microcrystalline cellulose, wherein the microcrystalline cellulose is a combination of Avicel PH101 and Avicel PH102.

Examples of suitable disintegrants include, but are not limited to, croscarmellose sodium, crospovidone, and mixtures thereof. In some embodiments, the disintegrant comprises croscarmellose sodium. In some embodiments, the disintegrant comprises crospovidone.

Examples of suitable lubricants include, but are not limited to, sodium stearyl fumarate, magnesium stearate, and mixtures thereof. In some embodiments, the lubricant comprises sodium stearyl fumarate. In some embodiments, the lubricant comprises magnesium stearate.

In some embodiments, the pharmaceutical composition comprises a compound of the invention (e.g., a compound of formula (I) or (II), or a pharmaceutically acceptable salt thereof, at least one polymer, at least one filler, at least one lubricant, and at least one disintegrant.

In some embodiments, the invention relates to a pharmaceutical composition comprising 45-55 wt % of a solid dispersion comprising a polymer and a compound of formula (II); 42-50 wt % of microcrystalline cellulose; 2-4 wt % of croscarmellose sodium; and 0.5-1.5 wt % of magnesium stearate. In some embodiments, the pharmaceutical composition comprises about 50 wt % of the solid dispersion; about 46 wt % of microcrystalline cellulose; about 3 wt % of croscarmellose sodium; and about 1.0 wt % of magnesium stearate.

In some embodiments, the pharmaceutical composition is a tablet core composition. In some embodiments, the tablet core composition is coated with a tablet coating. In some embodiments, the tablet coating is Opadry Blue.

In some embodiments, the solid dispersion comprises 70-80 wt % of the polymer and 20-30 wt % of the compound of formula (II). In some embodiments, the solid dispersion comprises about 75 wt % of the polymer and about 25 wt % of the compound of formula (II). In some embodiments, the polymer is HPMCAS.

In some embodiments, the pharmaceutical composition comprises about 50 mg of the compound of formula (II).

In some embodiments, the pharmaceutical composition comprises an intragranular blend and extragranular blend. In some embodiments, the intragranular blend comprises about 200 mg of the solid dispersion, about 93.6 mg of microcrystalline cellulose, about 6.0 mg of croscarmellose sodium, and about 0.4 mg of magnesium stearate; and the extragranular blend comprises about 90.4 mg of microcrystalline cellulose, about 6.0 mg of croscarmellose sodium, and about 3.6 mg of magnesium stearate.

### Uses of Compounds and Pharmaceutically Acceptable Salts and Compositions

In another aspect, the invention features a method of inhibiting a voltage-gated sodium channel in a subject comprising administering to the subject a compound of formula (I) or a pharmaceutically acceptable salt thereof or a pharmaceutical composition thereof. In another aspect, the invention features a method of inhibiting a voltage-gated sodium channel in a subject comprising administering to the subject a compound of formula (II) or a pharmaceutically acceptable salt thereof or a pharmaceutical composition thereof. In some aspects, the voltage-gated sodium channel is Na_{V}1.8.

In yet another aspect, the invention features a method of treating or lessening the severity of pain in a subject comprising administering an effective amount of a compound of formula (I), a pharmaceutically acceptable salt thereof or a pharmaceutical composition thereof. In yet another aspect, the invention features a method of treating or lessening the severity of pain in a subject comprising administering an effective amount of a compound of formula (II), a pharmaceutically acceptable salt thereof or a pharmaceutical composition thereof.

In another aspect, the invention features a compound of formula (I), or a pharmaceutically acceptable salt or pharmaceutical composition thereof, for use as a medicament. In another aspect, the invention features a compound of formula (II), or a pharmaceutically acceptable salt or pharmaceutical composition thereof, for use as a medicament.

In another aspect, the invention features a compound of formula (I), or a pharmaceutically acceptable salt or pharmaceutical composition thereof, for use in a method of inhibiting a voltage-gated sodium channel in a subject. In another aspect, the invention features a compound of formula (II), or a pharmaceutically acceptable salt or pharmaceutical composition thereof, for use in a method of inhibiting a voltage-gated sodium channel in a subject. In some aspects, the voltage-gated sodium channel is Na_{V}1.8.

In another aspect, the invention features a compound of formula (I), or a pharmaceutically acceptable salt or pharmaceutical composition thereof, for use in a method of treating or lessening the severity of pain in a subject. In another aspect, the invention features a compound of formula (II), or a pharmaceutically acceptable salt or pharmaceutical composition thereof, for use in a method of treating or lessening the severity of pain in a subject.

In another aspect, the invention provides the use of a compound of formula (I), or a pharmaceutically acceptable salt or pharmaceutical composition thereof, for the manufacture of a medicament. In another aspect, the invention provides the use of a compound of formula (II), or a pharmaceutically acceptable salt or pharmaceutical composition thereof, for the manufacture of a medicament.

In another aspect, the invention provides the use of a compound of formula (I), a pharmaceutically acceptable salt thereof, or a pharmaceutical composition thereof for the manufacture of a medicament for use in inhibiting a voltage-gated sodium channel. In another aspect, the invention provides the use of a compound of formula (II), a pharmaceutically acceptable salt thereof, or a pharmaceutical composition thereof for the manufacture of a medicament for use in inhibiting a voltage-gated sodium channel. In some aspects, the voltage-gated sodium channel is Na_{V}1.8.

In yet another aspect, the invention provides the use of a compound of formula (I), a pharmaceutically acceptable salt thereof, or a pharmaceutical composition thereof for the manufacture of a medicament for use in treating or lessening the severity of pain in a subject. In yet another aspect, the invention provides the use of a compound of formula (II), a pharmaceutically acceptable salt thereof, or a pharmaceutical composition thereof for the manufacture of a medicament for use in treating or lessening the severity of pain in a subject.

In another aspect, the invention features a method of inhibiting a voltage-gated sodium channel in a subject comprising administering to the subject a compound of the invention or a pharmaceutically acceptable salt thereof or a pharmaceutical composition thereof. In another aspect, the voltage-gated sodium channel is Na_{V}1.8.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of gut pain, wherein gut pain comprises inflammatory bowel disease pain, Crohn's disease pain, irritable bowel syndrome, endometriosis, polycystic ovarian disease, salpingitis, cervicitis or interstitial cystitis pain wherein said method comprises administering an effective amount of a compound of the invention, a pharmaceutically acceptable salt thereof or a pharmaceutical composition thereof.

In yet another aspect, the invention features a method of treating or lessening the severity in a subject of neuropathic pain comprising administering an effective amount of a compound of the invention, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the invention features a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia wherein said method comprises administering an effective amount of a compound of the invention, a pharmaceutically acceptable salt thereof or a pharmaceutical composition thereof.

In yet another aspect, the invention features a method of treating or lessening the severity in a subject of musculoskeletal pain comprising administering an effective amount of a compound of the invention, a pharmaceutically acceptable salt thereof or a pharmaceutical composition thereof. In some aspects, the musculoskeletal pain comprises osteoarthritis pain.

In yet another aspect, the invention features a method of treating or lessening the severity in a subject of musculoskeletal pain, wherein musculoskeletal pain comprises osteoarthritis pain, back pain, cold pain, burn pain or dental pain wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain, ankylosing spondylitis or vulvodynia wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises fibromyalgia pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises reflex sympathetic dystrophy pain, wherein said method comprises administering an effective amount of a compound of the invention, a pharmaceutically acceptable salt thereof or a pharmaceutical composition thereof.

In yet another aspect, the invention features a method of treating or lessening the severity in a subject of pathological cough wherein said method comprises administering an effective amount of a compound of the invention, a pharmaceutically acceptable salt thereof or a pharmaceutical composition thereof.

In yet another aspect, the invention features a method of treating or lessening the severity in a subject of acute pain comprising administering an effective amount of a compound of the invention, a pharmaceutically acceptable salt thereof or a pharmaceutical composition thereof. In some aspects, the acute pain comprises acute post-operative pain.

In yet another aspect, the invention features a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain) comprising administering an effective amount of a compound of the invention, a pharmaceutically acceptable salt thereof or a pharmaceutical composition thereof.

In yet another aspect, the invention features a method of treating or lessening the severity in a subject of bunionectomy pain comprising administering an effective amount of a compound of the invention, a pharmaceutically acceptable salt thereof or a pharmaceutical composition thereof.

In yet another aspect, the invention features a method of treating or lessening the severity in a subject of shoulder arthroplasty pain or shoulder arthroscopy pain comprising administering an effective amount of a compound of the invention, a pharmaceutically acceptable salt thereof or a pharmaceutical composition thereof.

In yet another aspect, the invention features a method of treating or lessening the severity in a subject of herniorrhaphy pain comprising administering an effective amount of a compound of the invention, a pharmaceutically acceptable salt thereof or a pharmaceutical composition thereof.

In yet another aspect, the invention features a method of treating or lessening the severity in a subject of abdominoplasty pain comprising administering an effective amount of a compound of the invention, a pharmaceutically acceptable salt thereof or a pharmaceutical composition thereof.

In yet another aspect, the invention features a method of treating or lessening the severity in a subject of visceral pain comprising administering an effective amount of a compound of the invention, a pharmaceutically acceptable salt thereof or a pharmaceutical composition thereof. In some aspects, the visceral pain comprises visceral pain from abdominoplasty.

In yet another aspect, the invention features a method of treating or lessening the severity in a subject of a neurodegenerative disease comprising administering an effective amount of a compound of the invention, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the invention features a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility, comprising administering an effective amount of a compound of the invention, a pharmaceutically acceptable salt thereof or a pharmaceutical composition thereof.

In another aspect, the invention features a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain, comprising administering an effective amount of a compound of the invention, a pharmaceutically acceptable salt thereof or a pharmaceutical composition thereof.

In another aspect, the invention features a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain, comprising administering an effective amount of a compound of the invention, a pharmaceutically acceptable salt thereof or a pharmaceutical composition thereof.

Compounds. Pharmaceutically Acceptable Salts, and Compositions for Use

In another aspect, the invention features a compound of the invention, or a pharmaceutically acceptable salt or pharmaceutical composition thereof, for use as a medicament.

In another aspect, the invention features a compound of the invention, or a pharmaceutically acceptable salt or pharmaceutical composition thereof, for use in a method of inhibiting a voltage-gated sodium channel in a subject. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In another aspect, the invention features a compound of the invention, or a pharmaceutically acceptable salt or pharmaceutical composition thereof, for use in a method of treating or lessening the severity in a subject of gut pain, wherein gut pain comprises inflammatory bowel disease pain, Crohn's disease pain, irritable bowel syndrome, endometriosis, polycystic ovarian disease, salpingitis, cervicitis or interstitial cystitis pain.

In another aspect, the invention features a compound of the invention, or a pharmaceutically acceptable salt or pharmaceutical composition thereof, for use in a method of treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the invention features a compound of the invention, or a pharmaceutically acceptable salt or pharmaceutical composition thereof, for use in a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia.

In another aspect, the invention features a compound of the invention, or a pharmaceutically acceptable salt or pharmaceutical composition thereof, for use in a method of treating or lessening the severity in a subject of musculoskeletal pain. In some aspects, the musculoskeletal pain comprises osteoarthritis pain.

In another aspect, the invention features a compound of the invention, or a pharmaceutically acceptable salt or pharmaceutical composition thereof, for use in a method of treating or lessening the severity in a subject of musculoskeletal pain, wherein musculoskeletal pain comprises osteoarthritis pain, back pain, cold pain, burn pain or dental pain.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain, ankylosing spondylitis or vulvodynia.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises fibromyalgia pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises reflex sympathetic dystrophy pain.

In another aspect, the invention features a compound of the invention, or a pharmaceutically acceptable salt or pharmaceutical composition thereof, for use in a method of treating or lessening the severity in a subject of pathological cough.

In another aspect, the invention features a compound of the invention, or a pharmaceutically acceptable salt or pharmaceutical composition thereof, for use in a method of treating or lessening the severity in a subject of acute pain. In some aspects, the acute pain comprises acute post-operative pain.

In another aspect, the invention features a compound of the invention, or a pharmaceutically acceptable salt or pharmaceutical composition thereof, for use in a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In another aspect, the invention features a compound of the invention, or a pharmaceutically acceptable salt or pharmaceutical composition thereof, for use in a method of treating or lessening the severity in a subject of bunionectomy pain.

In another aspect, the invention features a compound of the invention, or a pharmaceutically acceptable salt or pharmaceutical composition thereof, for use in a method of treating or lessening the severity in a subject of shoulder arthroplasty pain or shoulder arthroscopy pain.

In another aspect, the invention features a compound of the invention, or a pharmaceutically acceptable salt or pharmaceutical composition thereof, for use in a method of treating or lessening the severity in a subject of herniorrhaphy pain.

In another aspect, the invention features a compound of the invention, or a pharmaceutically acceptable salt or pharmaceutical composition thereof, for use in a method of treating or lessening the severity in a subject of abdominoplasty pain.

In another aspect, the invention features a compound of the invention, or a pharmaceutically acceptable salt or pharmaceutical composition thereof, for use in a method of treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for use in a method of treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In another aspect, the invention features a compound of the invention, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a compound of the invention, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention features a compound of the invention, or a pharmaceutically acceptable salt or pharmaceutical composition thereof, for use in a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention features a compound of the invention, or a pharmaceutically acceptable salt or pharmaceutical composition thereof, for use in a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Manufacture of Medicaments

In another aspect, the invention provides the use of a compound of the invention, or a pharmaceutically acceptable salt or pharmaceutical composition thereof, for the manufacture of a medicament.

In another aspect, the invention provides the use of a compound of the invention, a pharmaceutically acceptable salt thereof, or a pharmaceutical composition thereof for the manufacture of a medicament for use in inhibiting a voltage-gated sodium channel. In another aspect, the voltage-gated sodium channel is Na_{V}1.8.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In yet another aspect, the invention provides the use of the compound, pharmaceutically acceptable salt, or pharmaceutical composition described herein for the manufacture of a medicament for use in treating or lessening the severity in a subject of gut pain, wherein gut pain comprises inflammatory bowel disease pain, Crohn's disease pain, irritable bowel syndrome, endometriosis, polycystic ovarian disease, salpingitis, cervicitis or interstitial cystitis pain.

In yet another aspect, the invention provides a compound of the invention, a pharmaceutically acceptable salt thereof, or a pharmaceutical composition thereof for the manufacture of a medicament for use in treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the invention provides the use of a compound of the invention, a pharmaceutically acceptable salt thereof, or a pharmaceutical composition thereof for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic neuropathy.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain. In some aspects, the musculoskeletal pain comprises osteoarthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain, wherein musculoskeletal pain comprises osteoarthritis pain, back pain, cold pain, burn pain or dental pain.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain, ankylosing spondylitis or vulvodynia.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises fibromyalgia pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises reflex sympathetic dystrophy pain.

In yet another aspect, the invention provides the use of a compound of the invention, a pharmaceutically acceptable salt thereof, or a pharmaceutical composition thereof for the manufacture of a medicament for use in treating or lessening the severity in a subject of pathological cough.

In yet another aspect, the invention provides the use of a compound of the invention, a pharmaceutically acceptable salt thereof, or a pharmaceutical composition thereof for the manufacture of a medicament for use in treating or lessening the severity in a subject of acute pain. In some aspects, the acute pain comprises acute post-operative pain.

In yet another aspect, the invention provides the use of a compound of the invention, a pharmaceutically acceptable salt thereof, or a pharmaceutical composition thereof for the manufacture of a medicament for use in treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In yet another aspect, the invention provides the use of a compound of the invention, a pharmaceutically acceptable salt thereof, or a pharmaceutical composition thereof for the manufacture of a medicament for use in treating or lessening the severity in a subject of herniorrhaphy pain.

In yet another aspect, the invention provides the use of a compound of the invention, a pharmaceutically acceptable salt thereof, or a pharmaceutical composition thereof for the manufacture of a medicament for use in treating or lessening the severity in a subject of bunionectomy pain.

In yet another aspect, the invention provides the use of a compound of the invention, a pharmaceutically acceptable salt thereof, or a pharmaceutical composition thereof for the manufacture of a medicament for use in treating or lessening the severity in a subject of shoulder arthroplasty pain or shoulder arthroscopy pain.

In yet another aspect, the invention provides the use of a compound of the invention, a pharmaceutically acceptable salt thereof, or a pharmaceutical composition thereof for the manufacture of a medicament for use in treating or lessening the severity in a subject of abdominoplasty pain.

In yet another aspect, the invention provides the use of a compound of the invention, a pharmaceutically acceptable salt thereof, or a pharmaceutical composition thereof for the manufacture of a medicament for use in treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for the manufacture of a medicament for use in treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In yet another aspect, the invention provides the use of a compound of the invention, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the invention provides the use of a compound of the invention, a pharmaceutically acceptable salt thereof, or a pharmaceutical composition thereof for the manufacture of a medicament for use in treating or lessening the severity of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound of the invention, a pharmaceutically acceptable salt thereof, or a pharmaceutical composition thereof for the manufacture of a medicament for use in treating or lessening the severity of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention provides the use of a compound of the invention, a pharmaceutically acceptable salt thereof, or a pharmaceutical composition thereof for the manufacture of a medicament for use in treating or lessening the severity of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Administration of Compounds, Pharmaceutically Acceptable Salts, and Compositions

In certain embodiments of the invention, an “effective amount” of a compound of the invention, a pharmaceutically acceptable salt thereof, or a pharmaceutical composition thereof is that amount effective for treating or lessening the severity of one or more of the conditions recited above.

The compounds, salts, and compositions, according to the method of the invention, may be administered using any amount and any route of administration effective for treating or lessening the severity of one or more of the pain or non-pain diseases recited herein. The exact amount required will vary from subject to subject, depending on the species, age, and general condition of the subject, the severity of the condition, the particular agent, its mode of administration, and the like. The compounds, salts, and compositions of the invention are preferably formulated in dosage unit form for case of administration and uniformity of dosage. The expression “dosage unit form” as used herein refers to a physically discrete unit of agent appropriate for the subject to be treated. It will be understood, however, that the total daily usage of the compounds, salts, and compositions of the invention will be decided by the attending physician within the scope of sound medical judgment. The specific effective dose level for any particular subject or organism will depend upon a variety of factors including the disorder being treated and the severity of the disorder; the activity of the specific compound or salt employed; the specific composition employed; the age, body weight, general health, sex and diet of the subject; the time of administration, route of administration, and rate of excretion of the specific compound or salt employed; the duration of the treatment; drugs used in combination or coincidental with the specific compound or salt employed, and like factors well known in the medical arts. The term “subject” or “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compound, salts, and compositions of the invention may be administered orally or parenterally at dosage levels of about 0.001 mg/kg to about 1000 mg/kg, one or more times a day, effective to obtain the desired therapeutic effect.

Liquid dosage forms for oral administration include, but are not limited to, pharmaceutically acceptable emulsions, microemulsions, solutions, suspensions, syrups and elixirs. In addition to the active compound or salt, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of the compounds of the invention, it is often desirable to slow the absorption of the compounds from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound or salt of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound or salt is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound or salt can also be in microencapsulated form with one or more excipients as noted above. The solid dosage forms of tablets, dragees, capsules, pills, and granules can be prepared with coatings and shells such as enteric coatings, release-controlling coatings and other coatings well known in the pharmaceutical formulating art. In such solid dosage forms, the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound or salt of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of voltage-gated sodium channels. In one embodiment, the compounds are inhibitors of Na_{V}1.8 and thus, without wishing to be bound by any particular theory, the compounds, salts, and compositions are particularly useful for treating or lessening the severity of a disease, condition, or disorder where activation or hyperactivity of Na_{V}1.8 is implicated in the disease, condition, or disorder. When activation or hyperactivity of Na_{V}1.8 is implicated in a particular disease, condition, or disorder, the disease, condition, or disorder may also be referred to as a “Na_{V}1.8-mediated disease, condition or disorder.” Accordingly, in another aspect, the invention provides a method for treating or lessening the severity of a disease, condition, or disorder where activation or hyperactivity of Na_{V}1.8 is implicated in the disease state.

The activity of a compound utilized in this invention as an inhibitor of Na_{V}1.8 may be assayed according to methods described generally in International Publication No. WO 2014/120808 A9 and U.S. Publication No. 2014/0213616 A1, both of which are incorporated by reference in their entirety, methods described herein, and other methods known and available to one of ordinary skill in the art.

### Additional Therapeutic Agents

It will also be appreciated that the compounds, salts, and pharmaceutically acceptable compositions of the invention can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin; naphthylalkanones such as Nabumetone; oxicams such as Piroxicam; para-aminophenol derivatives, such as Acetaminophen; propionic acids such as Fenoprofen, Flurbiprofen, Ibuprofen, Ketoprofen, Naproxen, Naproxen sodium, Oxaprozin; salicylates such as Aspirin, Choline magnesium trisalicylate, Diflunisal; fenamates such as meclofenamic acid, Mefenamic acid; and pyrazoles such as Phenylbutazone); or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp &Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat R) or valproate;
(12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g. oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT_{1B/1D} agonist such as eletriptan, sumatriptan, naratriptan, zolmitriptan or rizatriptan;
(22) a 5-HT_{2A} receptor antagonist such as R(+)-alpha-(2,3-dimethoxy-phenyl)-1-[2-(4-fluorophenylethyl)]-4-piperidinemethanol (MDL-100907);
(23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ER®), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (1[alpha],3[alpha],5[alpha])(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluR1) antagonist;
(29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-S-chloro-S-pyridinecarbonitrile: 2-[[(1R,3S)-3-amino-4-hydroxy-1-(5-thiazolyl)butyl]thio]-4-chlorobenzonitrile, (2S,4R)-2-amino-4-[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504);
(37) a sodium channel blocker, such as lidocaine, lidocaine plus tetracaine cream (ZRS-201) or eslicarbazepine acetate;
(38) a Na_{V}1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607); WO2012/106499 (US2012196869); WO2012/112743 (US2012245136); WO2012/125613 (US2012264749), WO2012/116440 (US2014187533), WO2011026240 (US2012220605), U.S. Pat. Nos. 8,883,840, 8,466,188, WO2013/109521 (US2015005304), CN111217776, WO2020/117626, WO2021/252822, WO2021/252818, WO2021/252820, WO2014/201173, WO2012/125973, WO2013/086229, WO2013/134518, WO2014/201206, or WO2016/141035 the entire contents of each application hereby incorporated by reference;
(38a) a Na_{V}1.7 blocker such as (2-benzylspiro[3,4-dihydropyrrolo[1,2-a]pyrazine-1,4′-piperidine]-1′-yl)-(4-isopropoxy-3-methyl-phenyl)methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-yl]ethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl)methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl)methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone.
(39) a Na_{V}1.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/144375, WO2020/261114, WO2020/140959, WO2020/151728, WO2021/032074, WO2021/047622 (CN112479996), WO2021/257490, WO2021/257420, WO2021/257418, WO2022/263498, WO2022/235558, WO2022/235859, WO2023/138599, CN112390745, CN111808019, CN112225695, CN112457294, CN112300051, CN112300069, CN112441969, and CN114591293, the entire contents of each application hereby incorporated by reference;
(39a) a Na_{V}1.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl)quinoline-3-carboxamide, 2-(4-fluoro-2-methoxyphenoxy)-N-(3-sulfamoylphenyl)quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy)quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxy]-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, or 4-[[2-fluoro-6-[3-fluoro-2-methoxy-4-(trifluoromethoxy)phenoxy]-3-(trifluoromethyl)benzoyl]amino]pyridine-2-carboxamide;
(40) a combined Na_{V}1.7 and Na_{V}1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®); and the pharmaceutically acceptable salts and solvates thereof;
(43) a nicotinic receptor antagonist, such as varenicline;
(44) an N-type calcium channel antagonist, such as Z-160;
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ=10234094 (Carisbamate), BMS-954561 or ARC-4558.

In another embodiment, the additional appropriate therapeutic agents are selected from N-(6-amino-5-(2,3,5-trichlorophenyl)pyridin-2-yl) acetamide; N-(6-amino-5-(2-chloro-5-methoxyphenyl)pyridin-2-yl)-1-methyl-1H-pyrazole-5-carboxamide; or 3-((4-(4-(trifluoromethoxy)phenyl)-1H-imidazol-2-yl)methyl) oxetan-3-amine.

In another embodiment, the additional therapeutic agent is selected from a GlyT2/5HT2 inhibitor, such as Operanserin (VVZ149), a TRPV modulator such as CA008, CMX-020, NEO6860, FTABS, CNTX4975, MCP101, MDR16523, or MDR652, a EGR1 inhibitor such as Brivoglide (AYX1), an NGF inhibitor such as Tanezumab, Fasinumab, ASP6294, MEDI7352, a Mu opioid agonist such as Cebranopadol, NKTR181 (oxycodegol), a CB-1 agonist such as NEO1940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Na_{V}1.7 blocker such as ST-2427, ST-2578 and those disclosed in WO2010/129864, WO2015/157559, WO2017/059385, WO2018/183781, WO2018/183782, WO2020/072835, and/or WO2022/036297 the entire contents of each application hereby incorporated by reference.

In another embodiment, the additional therapeutic agent is selected from ASP18071, CC-8464, ANP-230, ANP-231, NOC-100, NTX-1175, ASN008, NW3509, AM-6120, AM-8145, AM-0422, BL-017881, NTM-006, Opiranserin (Unafra™), brivoligide, SR419, NRD.E1, LX9211, LY3016859, ISC-17536, NFX-88, LAT-8881, AP-235, NYX 2925, CNTX-6016, S-600918, S-637880, RQ-00434739, KLS-2031, MEDI 7352, and XT-150.

In another embodiment, the additional therapeutic agent is selected from Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623/ODM-111, ETX-801, OLP-1002, ANP-230/DSP-2230, iN1011-N17, DSP-3905 and ACD440.

In another embodiment, the additional therapeutic agent is selected from HRS4800, ODM-111/JMKX000623, LX9211, LY3556050, LY3857210, CFTX01554/CFTX-1554, MEDI7352, MEDI0618, BAY3178275, BAY2395840, GSK3858279, STC-004, HALNEURON, OLP-1002, ATX01, ANP230, CC-8464, iN1011-N17, ST-2427, MSD199, FZ008, VYNAV-01, BL-017881, Profervia (Cilnidipine), LS-04, vixotrigine, FX301/PCRX-301, PF-04531083, PF-01247324, and DSP-3905.

In another embodiment, the additional therapeutic agent is a sodium channel inhibitor (also known as a sodium channel blocker), such as the Na_{V}1.7 and Na_{V}1.8 blockers identified above.

The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

The compounds and salts of this invention or pharmaceutically acceptable compositions thereof may also be incorporated into compositions for coating an implantable medical device, such as prostheses, artificial valves, vascular grafts, stents and catheters. Accordingly, the invention, in another aspect, includes a composition for coating an implantable device comprising a compound or salt of the invention as described generally above, and in classes and subclasses herein, and a carrier suitable for coating said implantable device. In still another aspect, the invention includes an implantable device coated with a composition comprising a compound or salt of the invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

Another aspect of the invention relates to inhibiting Na_{V}1.8 activity in a biological sample or a subject, which method comprises administering to the subject, or contacting said biological sample with a compound of the invention, a pharmaceutically acceptable salt thereof, or a pharmaceutical composition thereof. The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

Inhibition of Na_{V}1.8 activity in a biological sample is useful for a variety of purposes that are known to one of skill in the art. Examples of such purposes include, but are not limited to, the study of sodium channels in biological and pathological phenomena; and the comparative evaluation of new sodium channel inhibitors.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T. G. M. Wuts et al., Greene's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P, ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)- and/or carbon-14 (¹⁴C)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### Enumerated Embodiments

Further embodiments of the disclosure are set out in the following numbered clauses:

1. A process for preparing a compound of formula (I):

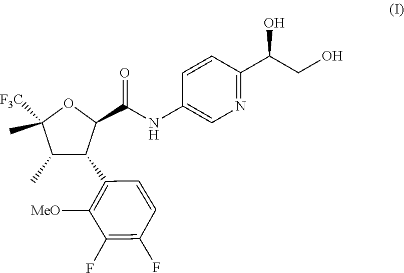 [SMILES: COc1c([C@H]2[C@H](C(=O)Nc3ccc([C@@H](O)CO)nc3)O[C@@](C)(C(F)(F)F)[C@H]2C)ccc(F)c1F]

comprising transforming a compound of formula (IV):

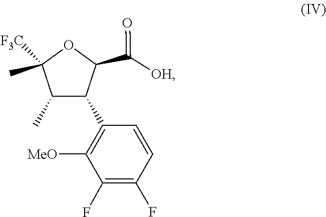 [SMILES: COc1c([C@H]2[C@H](C(=O)O)O[C@@](C)(C(F)(F)F)[C@H]2C)ccc(F)c1F]

or a salt thereof, to the compound of formula (I).
2. The process of embodiment 1, wherein transforming the compound of formula (IV) to the compound of formula (I) comprises preparing a compound of formula (VI):

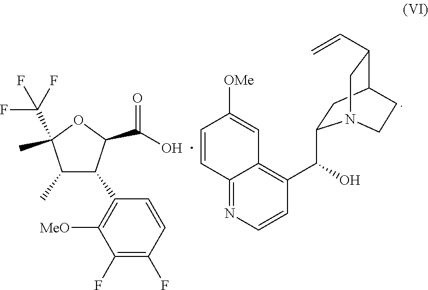

3. The process of embodiment 2, wherein preparing the compound of formula (VI) comprises contacting the compound of formula (IV) or a compound of formula (VII):

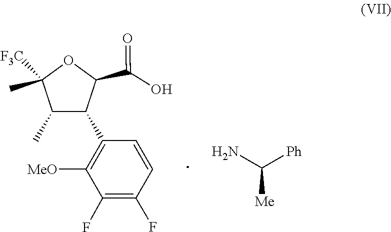 [SMILES: COc1c([C@H]2[C@H](C(=O)O)O[C@@](C)(C(F)(F)F)[C@H]2C)ccc(F)c1F ; C[C@@H](N)c1ccccc1]

with quinine.
4. The process of embodiment 1, wherein transforming the compound of formula (IV) to the compound of formula (I) comprises preparing a compound of formula (VII):

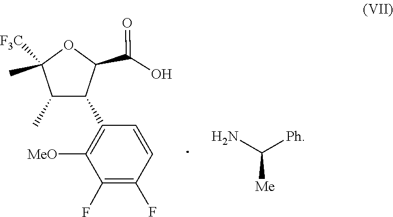 [SMILES: COc1c([C@H]2[C@H](C(=O)O)O[C@@](C)(C(F)(F)F)[C@H]2C)ccc(F)c1F ; C[C@@H](N)c1ccccc1]

5. The process of embodiment 3 or 4, wherein the compound of formula (VII) is obtained by contacting the compound of formula (IV) with (R)-α-methylbenzylamine.
6. The process of any one of embodiments 1 to 5, wherein transforming a compound of formula (IV) to the compound of formula (I) comprises contacting the compound of formula (IV), the compound of formula (VI), or the compound of formula (VII) with a compound of formula (V):

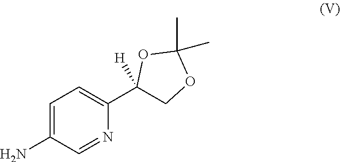

to afford a compound of formula (III):

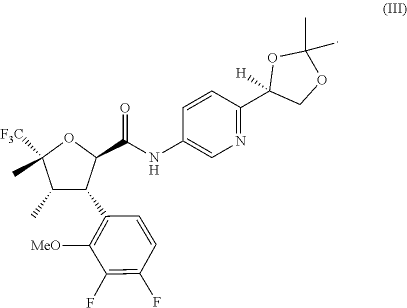

7. The process of embodiment 6, wherein contacting the compound of formula (IV), the compound of formula (VI), or the compound of formula (VII) with a compound of formula (V) is performed in the presence of a coupling reagent and a first base.
8. The process of embodiment 7, wherein the coupling reagent is propanephosphonic acid anhydride (T3P) and the first base is triethylamine.
9. The process of any one of embodiments 1 to 5, wherein transforming the compound of formula (IV) to the compound of formula (I) comprises transforming the compound of formula (IV), the compound of formula (VI), or the compound of formula (VII) to a compound of formula (VIII):

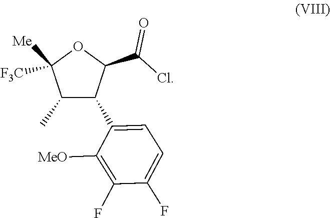 [SMILES: COc1c([C@H]2[C@H](C(=O)Cl)O[C@@](C)(C(F)(F)F)[C@H]2C)ccc(F)c1F]

10. The process of embodiment 9, wherein transforming the compound of formula (IV), the compound of formula (VI), or the compound of formula (VII) to a compound of formula (VIII) comprises treating the compound of formula (IV), the compound of formula (VI) or the compound of formula (VII) with a chlorinating reagent.
11. The process of embodiment 10, wherein the chlorinating reagent is selected from phosgene, thionyl chloride, methanesulfonyl chloride, phosphorus oxychloride, phosphorus pentachloride, oxalyl chloride, isobutyl chloroformate (IBCF), pivaloyl chloride (PivCl), and diphenylphosphinic chloride (DPPCl).
12. The process of embodiment 10 or 11, wherein the chlorinating reagent is oxalyl chloride.
13. The process of any one of embodiments 9 to 12, wherein transforming a compound of formula (IV) to the compound of formula (I) comprises contacting the compound of formula (VIII) with a compound of formula (V):

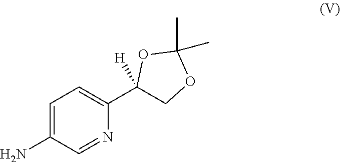

to afford a compound of formula (III):

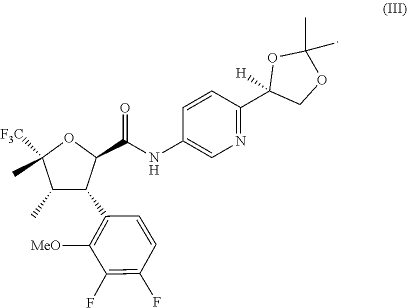

14. The process of embodiment 13, wherein contacting the compound of formula (VIII) with a compound of formula (V) is performed in the presence of a second base.
15. The process of embodiment 14, wherein the second base is triethylamine.
16. The process of any one of embodiments 6 to 8 or 13 to 15, wherein transforming a compound of formula (IV) to the compound of formula (I) further comprises transforming the compound of formula (III) to the compound of formula (I).
17. The process of embodiment 16, wherein transforming the compound of formula (III) to the compound of formula (I) comprises treating the compound of formula (III) with a first acid and water.
18. The process of embodiment 17, wherein the first acid is aqueous trifluoroacetic acid.
19. The process of any one of embodiments 1 to 18, further comprising recrystallizing the compound of formula (I) from a suitable solvent to afford crystalline Form A of the compound of formula (I).
20. The process of embodiment 19, wherein the suitable solvent comprises ethyl acetate and n-heptane.
21. The process of any one of embodiments 6 to 8 or 13 to 20, further comprising transforming a compound of formula (XVI):

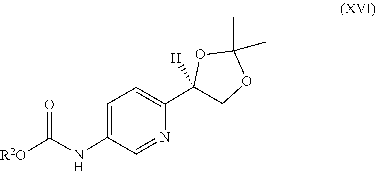

to the compound of formula (V),
wherein R² is C₁-C₆ alkyl.
   22. The process of embodiment 21, wherein transforming the compound of formula (XVI) to the compound of formula (V) comprises treating the compound of formula (XVI) with water at reflux.
   23. The process of embodiment 21, wherein transforming the compound of formula (XVI) to the compound of formula (V) comprises treating the compound of formula (XVI) with a third base and water.
   24. The process of embodiment 23, wherein the third base is NaOH.
   25. The process of any one of embodiments 21 to 24, wherein R² is tert-butyl or ethyl.
   26. The process of any one of embodiments 21 to 25, wherein the compound of formula (XVI) is a compound of formula (XVI-A):

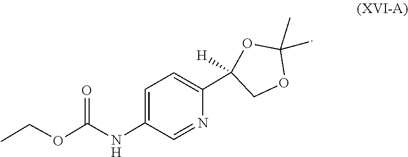

27. The process of any one of embodiments 21 to 25, wherein the compound of formula (XVI) is a compound of formula (XVI-B):

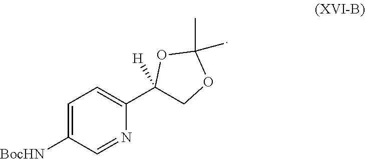

28. The process of any one of embodiments 21 to 27, further comprising recrystallizing the compound of formula (V) from a suitable solvent.
29. The process of embodiment 28, wherein the suitable solvent comprises MTBE and n-heptane.
30. The process of any one of embodiments 21 to 29, further comprising transforming a compound of formula (XVII):

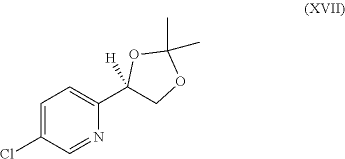

to the compound of formula (XVI).
31. The process of embodiment 30, wherein transforming the compound of formula (XVII) to the compound of formula (XVI) comprises treating the compound of formula (XVII) with NH₂—C(O)—(C₁-C₆ alkyl), a fourth base, a palladium catalyst, and a ligand.
32. The process of embodiment 31, wherein the fourth base is Cs₂CO₃ or K₃PO₄.
33. The process of embodiment 31 or 32, wherein the palladium catalyst is Pd(OAc)₂.
34. The process of any one of embodiments 31 to 33, wherein the ligand is XPhos or BrettPhos.
35. The process of any one of embodiments 30 to 34, further comprising transforming a compound of formula (XVIII):

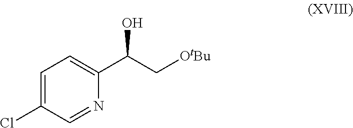 [SMILES: CC(C)(C)OC[C@H](O)c1ccc(Cl)cn1]

to the compound of formula (XVII).
36. The process of embodiment 35, wherein transforming a compound of formula (XVIII) to the compound of formula (XVII) comprises treating the compound of formula (XVIII) with 2,2-dimethoxypropane in the presence of a second acid.
37. The process of embodiment 36, wherein the second acid is methanesulfonic acid.
38. The process of any one of embodiments 35 to 37, further comprising transforming a compound of formula (XIX):

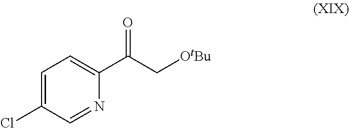 [SMILES: CC(C)(C)OCC(=O)c1ccc(Cl)cn1]

to the compound of formula (XVIII).
39. The process of embodiment 38, wherein transforming the compound of formula (XIX) to the compound of formula (XVIII) comprises contacting the compound of formula (XIX) with a reducing agent.
40. The process of embodiment 39, wherein the compound of formula (XIX) is contacted with a reducing agent in the presence of an enzyme.
41. The process of any one of embodiments 38 to 40, further comprising contacting 2-bromo-5-chloropyridine with 2-(tert-butoxy)-N-methoxy-N-methylacetamide to afford the compound of formula (XIX).
42. The process of embodiment 41, wherein the 2-bromo-5-chloropyridine is contacted with 2-(tert-butoxy)-N-methoxy-N-methylacetamide in the presence of a Grignard reagent.
43. The process of embodiment 42, wherein the Grignard reagent is isopropyl magnesium chloride lithium chloride complex.
44. A process for preparing a compound of formula (II):

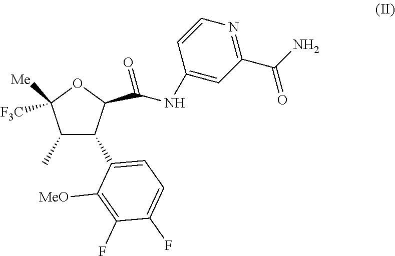 [SMILES: COc1c([C@H]2[C@H](C(=O)Nc3ccnc(C(N)=O)c3)O[C@@](C)(C(F)(F)F)[C@H]2C)ccc(F)c1F]

comprising transforming a compound of formula (VII):

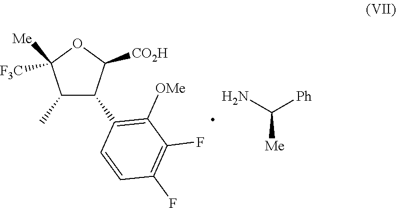 [SMILES: COc1c([C@H]2[C@H](C(=O)O)O[C@@](C)(C(F)(F)F)[C@H]2C)ccc(F)c1F ; C[C@@H](N)c1ccccc1]

to the compound of formula (II).
45. The process of embodiment 44, wherein transforming the compound of formula (VII) to the compound of formula (II) comprises contacting the compound of formula (VII) with a compound of formula (XXI):

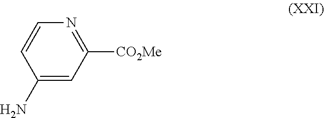 [SMILES: COC(=O)c1cc(N)ccn1]

to afford a compound of formula (XX):

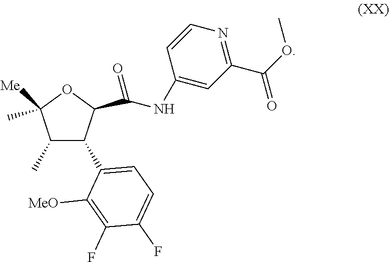

46. The process of embodiment 45, wherein contacting the compound of formula (VII) with a compound of formula (XXI) is performed in the presence of a coupling reagent and a first base.
47. The process of embodiment 46, wherein the coupling reagent is propanephosphonic acid anhydride (T3P) and the first base is triethylamine.
48. The process of embodiment 44, wherein transforming the compound of formula (VII) to the compound of formula (II) comprises transforming the compound of formula (VII) to a compound of formula (VIII):

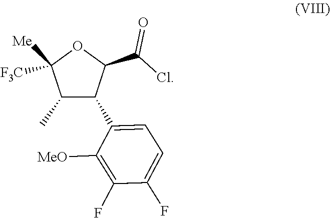 [SMILES: COc1c([C@H]2[C@H](C(=O)Cl)O[C@@](C)(C(F)(F)F)[C@H]2C)ccc(F)c1F]

49. The process of embodiment 48, wherein transforming the compound of formula (VII) to a compound of formula (VIII) comprises treating the compound of formula (VII) with a chlorinating reagent.
50. The process of embodiment 49, wherein the chlorinating reagent is selected from phosgene, thionyl chloride, methanesulfonyl chloride, phosphorus oxychloride, phosphorus pentachloride, oxalyl chloride, isobutyl chloroformate (IBCF), pivaloyl chloride (PivCl), and diphenylphosphinic chloride (DPPCl).
51. The process of embodiment 49 or 50, wherein the chlorinating reagent is oxalyl chloride.
52. The process of any one of embodiments 48 to 51, wherein transforming the compound of formula (VII) to the compound of formula (II) comprises contacting the compound of formula (VIII) with a compound of formula (XXI):

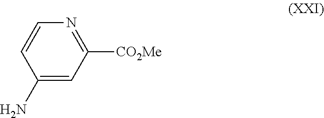 [SMILES: COC(=O)c1cc(N)ccn1]

to afford a compound of formula (XX):

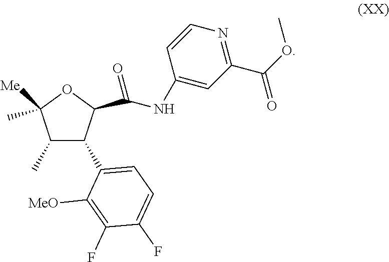

53. The process of embodiment 52, wherein contacting the compound of formula (VIII) with a compound of formula (XXI) is performed in the presence of a second base.
54. The process of embodiment 53, wherein the second base is triethylamine.
55. The process of any one of embodiments 52 to 54, wherein contacting the compound of formula (VIII) with a compound of formula (XXI) is performed in toluene.
56. The process of any one of embodiments 45 to 47 or 52 to 55, wherein transforming a compound of formula (VII) to the compound of formula (II) further comprises transforming the compound of formula (XX) to the compound of formula (II).
57. The process of embodiment 56, wherein transforming the compound of formula (XX) to the compound of formula (II) comprises treating the compound of formula (XX) with ammonia to afford the compound of formula (II).
58. The process of embodiment 57, wherein the ammonia is in the form of a solution of ammonia in a solvent, ammonia in gas form in which an ammonia gas is bubbled into the reaction mixture, or in the form of ammonium hydroxide or an ammonium salt where ammonia is generated in situ.
59. The process of embodiment 58, wherein the ammonia is in the form of a solution of ammonia in methanol.
60. The process of embodiment 58, wherein the ammonia is in the form of a solution of ammonia in methanol and tetrahydrofuran.
61. The process of embodiment 58, wherein the in situ generation of ammonia comprises reacting ammonium hydroxide or the ammonium salt with an acid.
62. The process of any one of embodiments 57 to 61, wherein treating the compound of formula (XX) with ammonia is performed in a solvent mixture comprising methanol and tetrahydrofuran.
63. The process of any one of embodiments 44 to 62, further comprising recrystallizing the compound of formula (II) from a suitable solvent.
64. The process of embodiment 63, wherein the suitable solvent comprises MeOH, THF, and water.
65. The process of any one of embodiments 44 to 64, further comprising transforming a compound of formula (IV):

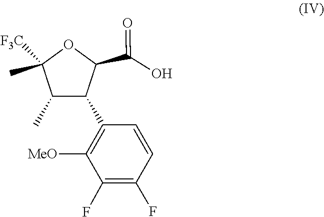 [SMILES: COc1c([C@H]2[C@H](C(=O)O)O[C@@](C)(C(F)(F)F)[C@H]2C)ccc(F)c1F]

to the compound of formula (VII).
66. The process of embodiment 65, wherein transforming the compound of formula (IV) to the compound of formula (VII) comprises contacting the compound of formula (IV) with (R)-α-methylbenzylamine.
67. The process of any one of embodiments 1 to 43, 65, or 66, further comprising transforming a compound of formula (IX):

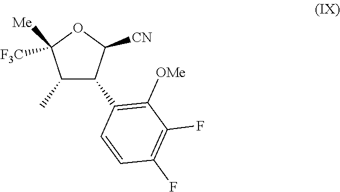 [SMILES: COc1c([C@H]2[C@H](C#N)O[C@@](C)(C(F)(F)F)[C@H]2C)ccc(F)c1F]

to the compound of formula (IV).
68. The process of embodiment 67, wherein transforming a compound of formula (IX) to the compound of formula (IV) comprises treating the compound of formula (IX) with a fifth base.
69. The process of embodiment 68, wherein the fifth base is potassium hydroxide.
70. The process of any one of embodiments 67 to 69, further comprising transforming a compound of formula (X):

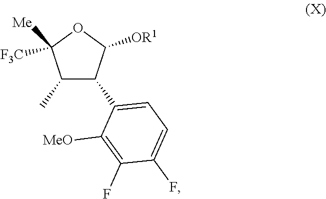

to the compound of formula (IX),
wherein:
R¹ is —C(O)—Z, —C(O)OZ, —C(O)CH═CH—Z, or —P(O)Z₂; and
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl.
   71. The process of embodiment 70, wherein the compound of formula (X) has formula (X′):

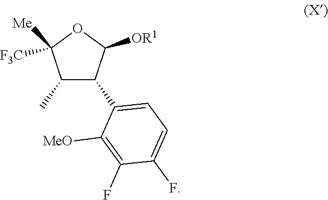

72. The process of embodiment 71, wherein transforming a compound of formula (X) to the compound of formula (IX) comprises treating the compound of formula (X) with a cyanating reagent.
73. The process of embodiment 72, wherein the cyanating agent is selected from the group consisting of trimethylsilyl cyanide, diethylaluminum cyanide, KCN, NaCN, TBACN, and HCN.
74. The process of embodiment 73, wherein the cyanating reagent is trimethylsilyl cyanide.
75. The process of embodiment 74, wherein the compound of formula (X) is treated with 1.35-1.65 equivalents of trimethylsilyl cyanide.
76. The process of any one of embodiments 70 to 75, wherein treating the compound of formula (X) with a cyanating reagent is performed in the presence of a Lewis acid.
77. The process of embodiment 76, wherein the Lewis acid is selected from boron trifluoride diethyl etherate (BF3·OEt2), TiCl₄, InCl₃, AgSbF₆, iodine, ZnBr₂, Al(OiPr)₃, MgCl₂, Mn(acac)₂, MnCl₂, TMSOTf, and SnCl₄.
78. The process of embodiment 77, wherein the Lewis acid is BF₃OEt₂.
79. The process of embodiment 78, wherein 0.9-1.1 equivalents of the BF₃OEt₃ are present, based on the compound of formula (X).
80. The process of any one of embodiments 70 to 79, wherein R¹ is —C(O)—Z.
81. The process of embodiment 80, wherein Z is methyl or 4-nitrophenyl.
82. The process of embodiment 81, wherein Z is 4-nitrophenyl.
83. The process of any one of embodiments 70 to 82, further comprising transforming a compound of formula (XI):

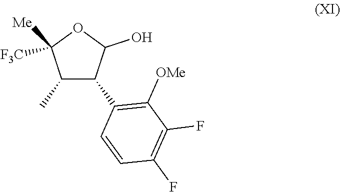 [SMILES: COc1c([C@H]2C(O)O[C@@](C)(C(F)(F)F)[C@H]2C)ccc(F)c1F]

to the compound of formula (X).
84. The process of embodiment 83, wherein the compound of formula (XI) has formula (XI′):

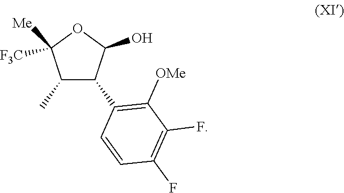 [SMILES: COc1c([C@H]2[C@H](O)O[C@@](C)(C(F)(F)F)[C@H]2C)ccc(F)c1F]

85. The process of embodiment 84, wherein transforming a compound of formula (XI) to the compound of formula (X) comprises contacting the compound of formula (XI) with an acid anhydride or an acid halide to afford the compound of formula (X).
86. The process of embodiment 85, wherein contacting the compound of formula (XI) with an acid anhydride or an acid halide is performed in the presence of a sixth base and a catalyst.
87. The process of embodiment 86, wherein the sixth base is triethylamine and the catalyst is 4-dimethylaminopyridine (DMAP).
88. The process of any one of embodiments 85 to 87, wherein the acid anhydride is acetic anhydride.
89. The process of any one of embodiments 85 to 87, wherein the acid halide is 4-nitrobenzoyl chloride.
90. The process of any one of embodiments 83 to 89, further comprising recrystallizing the compound of formula (X) from a suitable solvent.
91. The process of embodiment 90, wherein the suitable solvent comprises acetone and water.
92. The process of any one of embodiments 83 to 91, further comprising transforming a compound of formula (XII):

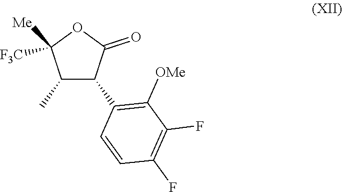 [SMILES: COc1c([C@H]2C(=O)O[C@@](C)(C(F)(F)F)[C@H]2C)ccc(F)c1F]

to the compound of formula (XI).
93. The process of embodiment 92, wherein transforming the compound of formula (XII) to the compound of formula (XI) comprises treating the compound of formula (XII) with a reducing reagent.
94. The process of embodiment 93, wherein the reducing reagent is selected from diisobutylaluminum hydride, Red-Al, NaBH₄/BF₃, titanocene with polymethylhydrosiloxane and phenylsilane.
95. The process of embodiment 93 or 94, wherein the reducing reagent is diisobutylaluminum hydride.
96. The process of any one of embodiments 92 to 95, further comprising transforming a compound of formula (XIII):

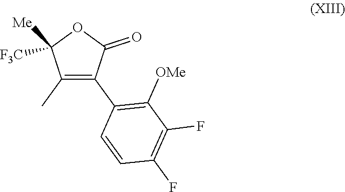 [SMILES: COc1c(C2=C(C)[C@](C)(C(F)(F)F)OC2=O)ccc(F)c1F]

to the compound of formula (XII).
97. The process of embodiment 96, wherein transforming a compound of formula (XIII) to the compound of formula (XII) comprises a hydrogenation of the compound of formula (XIII).
98. The process of embodiment 97, wherein the hydrogenation is performed in the presence of a hydrogenation catalyst.
99. The process of embodiment 98, wherein the hydrogenation catalyst is selected from Pd/C, Pd/Al₂O₃, Pt/C, Ni (Raney), Co (Raney), Rh/C, Ir/C, Ru/C, Pd(OH)₂, homogeneous chiral Ru and Rh.
100. The process of any one of embodiments 97 to 99, wherein the hydrogenation is performed in the presence of a suitable hydrogen source.
101. The process of embodiment 100, wherein the hydrogen source is selected from H₂ gas, NiCl₂/NaBH₄ in methanol, and Et₃SiH.
102. The process of any one of embodiments 97 to 101, wherein the hydrogenation is conducted in the presence of H₂ gas using Pd/C as a catalyst.
103. The process of embodiment 102, wherein the H₂ gas is present at a pressure of 5-40 barg.
104. The process of any one of embodiments 97 to 103, wherein the hydrogenation is performed in a solvent mixture comprising 2-propanol, tetrahydrofuran, and catalytic trifluoroacetic acid.
105. The process of any one of embodiments 97 to 104, wherein the hydrogenation is performed at a temperature of 10-50° C.
106. The process of any one of embodiments 96 to 105, further comprising contacting a compound of formula (XIV):

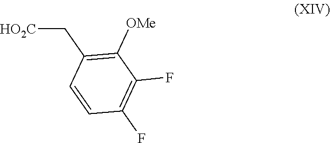 [SMILES: COc1c(CC(=O)O)ccc(F)c1F]

with a compound of formula (XV):

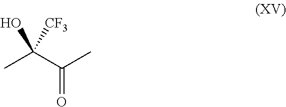

to afford the compound of formula (XIII).
107. The process of embodiment 106, wherein contacting a compound of formula (XIV) with a compound of formula (XV) is performed in the presence of a coupling agent or a chlorinating agent.
108. The process of embodiment 107, wherein the coupling agent is selected from CDI and T3P.
109. The process of embodiment 107, wherein the chlorinating agent converts the compound of formula (XIV) to an acid chloride, which is not isolated before reacting with the compound of formula (XV).
110. The process of embodiment 107 or 109, wherein the chlorinating agent is selected from oxalyl chloride and thionyl chloride.
111. The process of any one of embodiments 1 to 43, 65, or 66, further comprising transforming a compound of formula (XXII):

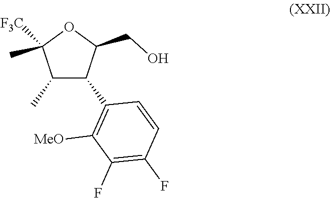 [SMILES: COc1c([C@H]2[C@H](CO)O[C@@](C)(C(F)(F)F)[C@H]2C)ccc(F)c1F]

to the compound of formula (IV).
112. The process of embodiment 111, wherein transforming a compound of formula (XXII) to the compound of formula (IV) comprises treating the compound of formula (XXII) with an oxidant.
113. The process of embodiment 112, wherein the oxidant comprises TEMPO and NaOCl.
114. The process of any one of embodiments 111 to 113, further comprising transforming a compound of formula (XXIII):

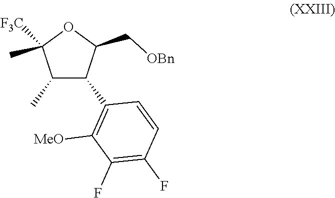 [SMILES: COc1c([C@H]2[C@H](COCc3ccccc3)O[C@@](C)(C(F)(F)F)[C@H]2C)ccc(F)c1F]

to the compound of formula (XXII).
115. The process of embodiment 114, wherein transforming the compound of formula (XXIII) to the compound of formula (XXII) comprises hydrogenating the compound of formula (XXIII).
116. The process of embodiment 115, wherein hydrogenating the compound of formula (XXIII) is performed in the presence of hydrogen and a palladium on carbon catalyst.
117. The process of any one of embodiments 114 to 116, further comprising transforming a compound of formula (XXIV):

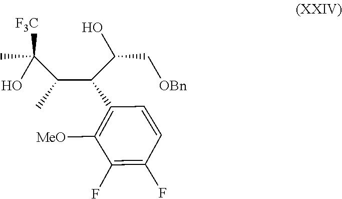 [SMILES: COc1c([C@@H]([C@H](O)COCc2ccccc2)[C@H](C)[C@@](C)(O)C(F)(F)F)ccc(F)c1F]

to the compound of formula (XXIII).
118. The process of embodiment 117, wherein transforming the compound of formula (XXIV) to the compound of formula (XXIII) comprises treating the compound of formula (XXIV) with mesyl chloride in the presence of a seventh base.
119. The process of embodiment 118, wherein the seventh base is triethylamine.
120. The process of any one of embodiments 117 to 119, further comprising transforming a compound of formula (XXV):

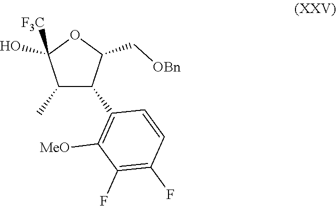 [SMILES: COc1c([C@H]2[C@@H](COCc3ccccc3)O[C@](O)(C(F)(F)F)[C@H]2C)ccc(F)c1F]

to the compound of formula (XXIV).
121. The process of embodiment 120, wherein transforming the compound of formula (XXV) to the compound of formula (XXIV) comprises treating the compound of formula (XXV) with methyl magnesium chloride.
122. The process of embodiment 120 or 121, further comprising transforming a compound of formula (XXVI):

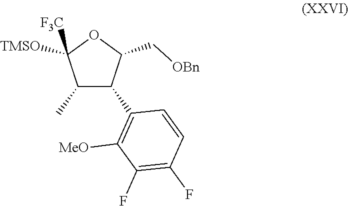

to the compound of formula (XXV).
123. The process of embodiment 122, wherein transforming the compound of formula (XXVI) to the compound of formula (XXV) comprises treating the compound of formula (XXVI) with tetra-N-butylammonium fluoride (TBAF).
124. The process of embodiment 122 or 123, further comprising transforming a compound of formula (XXVII):

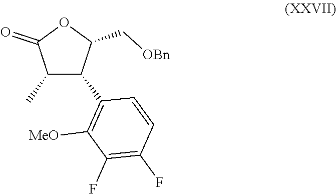 [SMILES: COc1c([C@@H]2[C@H](C)C(=O)O[C@@H]2COCc2ccccc2)ccc(F)c1F]

to the compound of formula (XXVI).
125. The process of embodiment 124, wherein transforming the compound of formula (XXVII) to the compound of formula (XXVI) comprises treating the compound of formula (XXVII) with trimethyl(trifluoromethyl) silane in the presence cesium fluoride.
126. The process of embodiment 124 or 125, further comprising transforming a compound of formula (XXVIII):

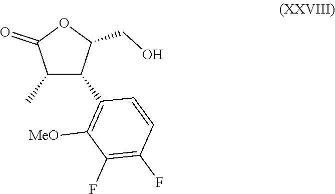 [SMILES: COc1c([C@@H]2[C@H](C)C(=O)O[C@@H]2CO)ccc(F)c1F]

to the compound of formula (XXVII).
127. The process of embodiment 126, wherein transforming a compound of formula (XXVIII) to the compound of formula (XXVII) comprises treating the compound of formula (XXVIII) with benzyl-2,2,2-trichloroethan-imidate in the presence of a third acid.
128. The process of embodiment 127, wherein the third acid is triflic acid.
129. The process of any one of embodiments 126 to 128, further comprising transforming a compound of formula (XXIX):

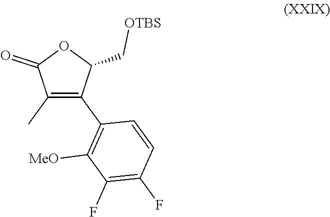 [SMILES: COc1c(C2=C(C)C(=O)O[C@@H]2CO[Si](C)(C)C(C)(C)C)ccc(F)c1F]

to the compound of formula (XXVIII).
130. The process of embodiment 129, wherein transforming the compound of formula (XXIX) to the compound of formula (XXVIII) comprises a hydrogenation of the compound of formula (XXIX).
131. The process of embodiment 130, wherein the hydrogenation of the compound of formula (XXIX) is performed in the presence of NiCl₂/NaBH₄ in methanol.
132. A compound of formula:

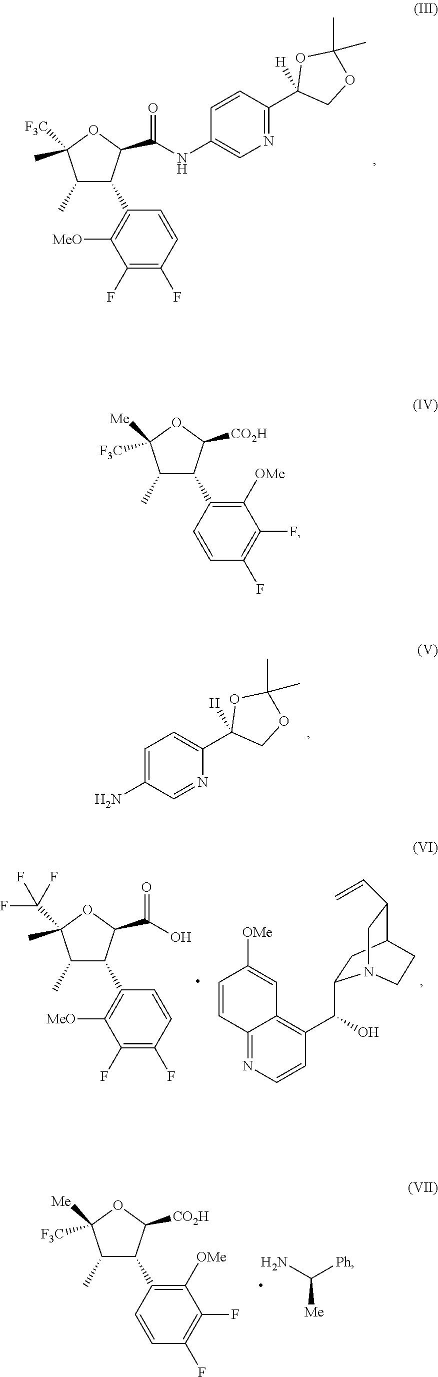

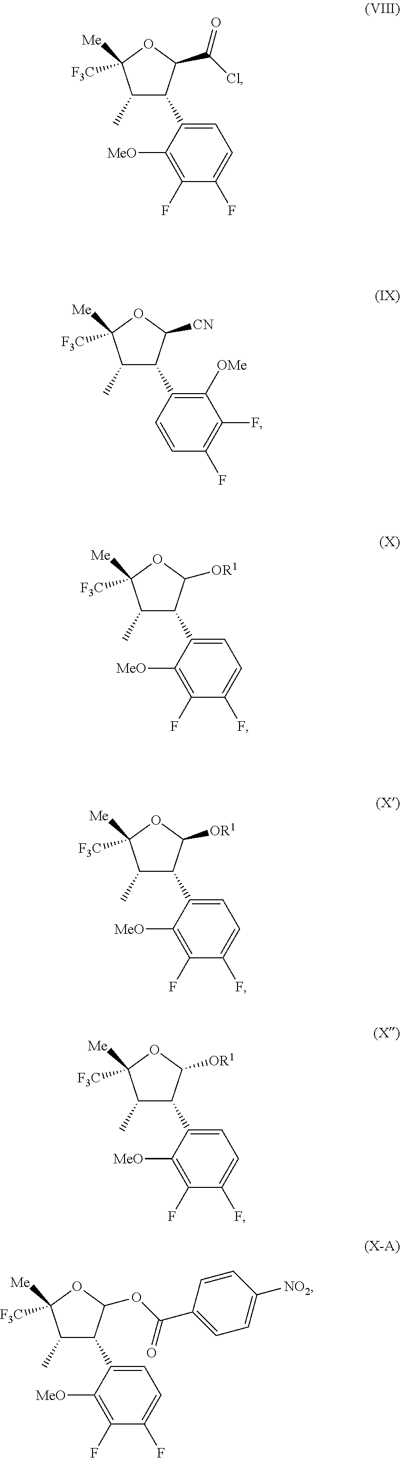

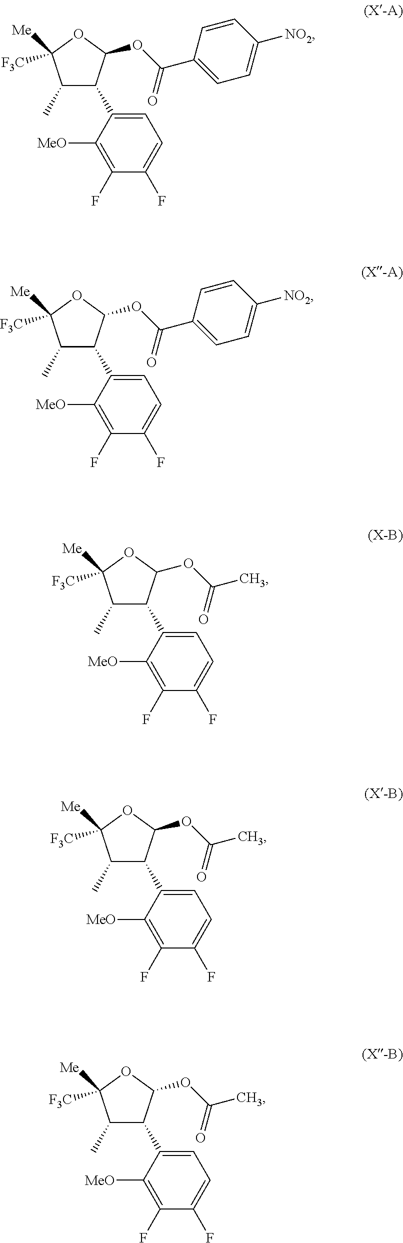

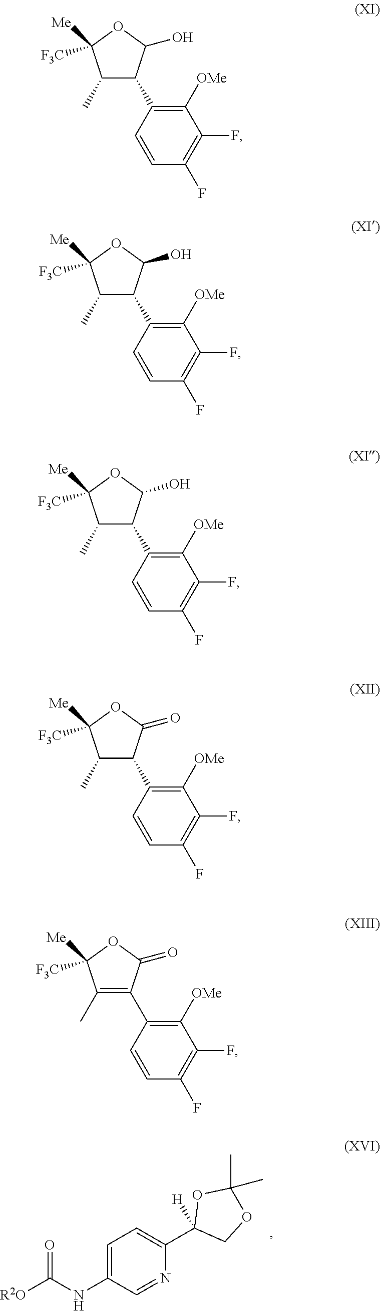

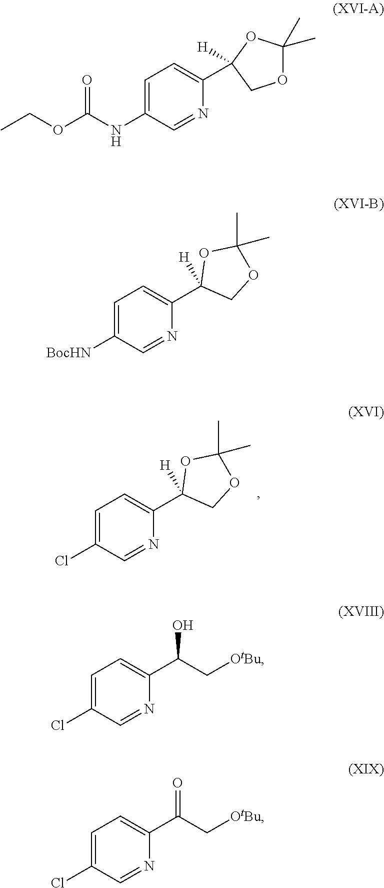

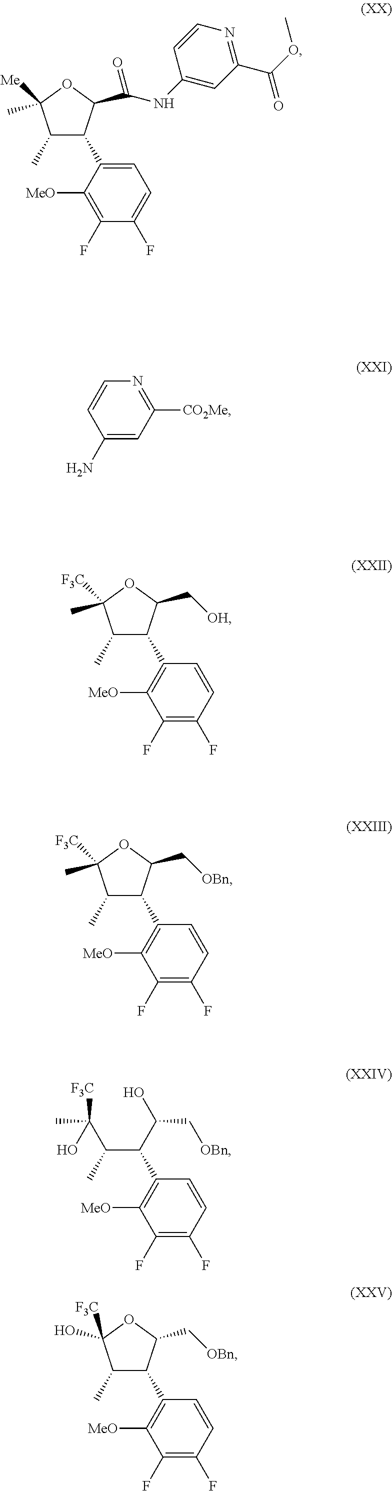

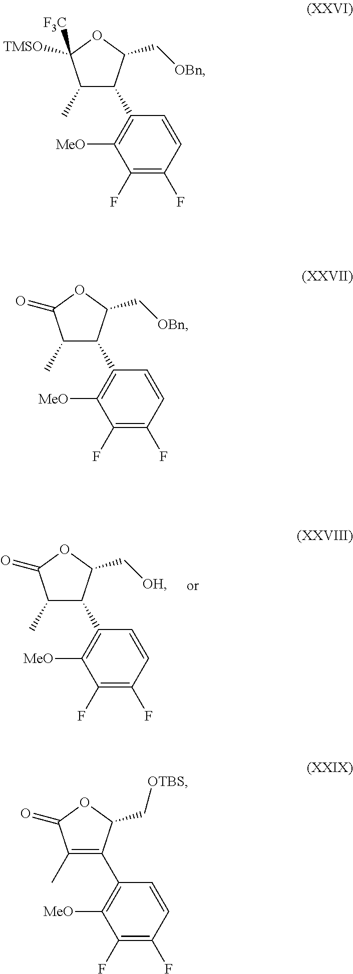

wherein:
R¹ is —C(O)—Z, —C(O)OZ, —C(O)CH═CH—Z, or —P(O)Z₂;
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl; and
R² is C₁-C₆ alkyl.
   133. A compound of formula:

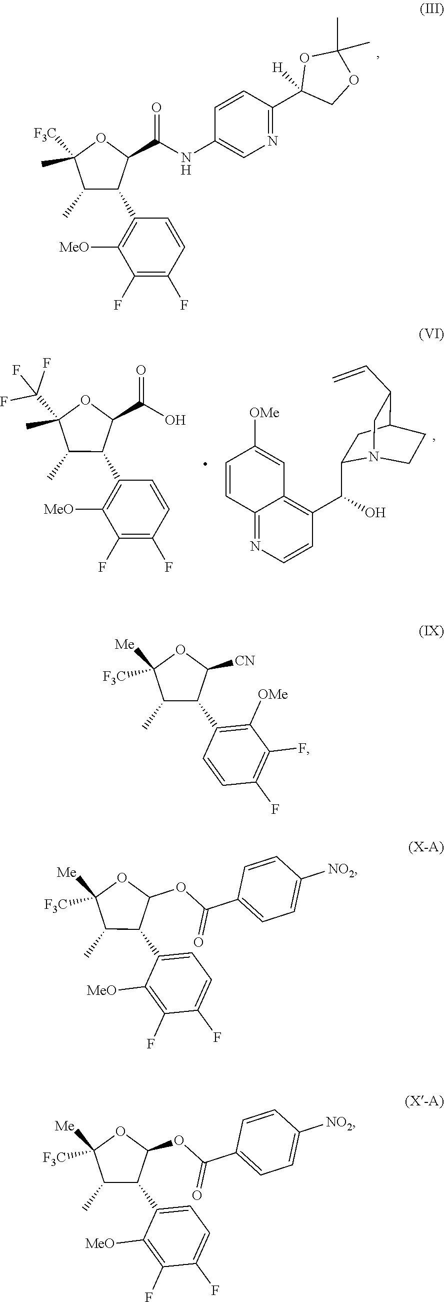

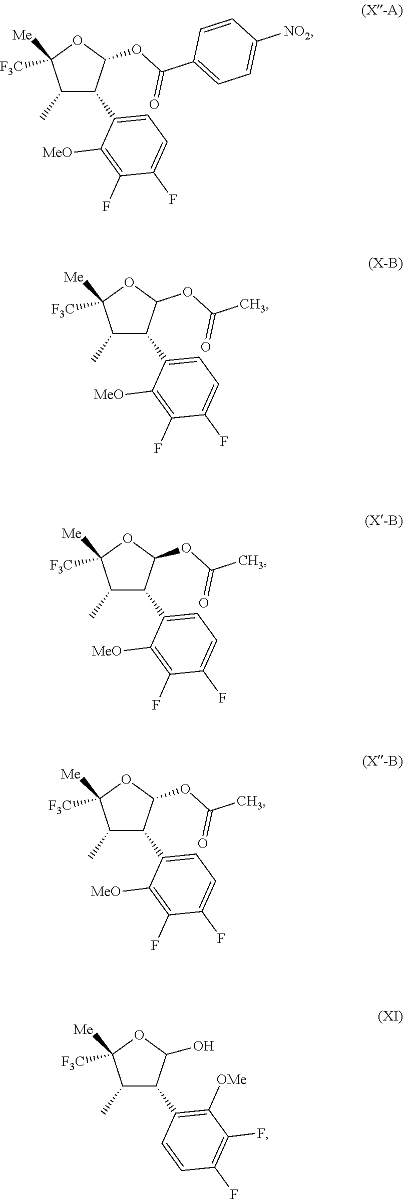 [SMILES: COc1c([C@H]2[C@@H](OC(=O)c3ccc([N+](=O)[O-])cc3)O[C@@](C)(C(F)(F)F)[C@H]2C)ccc(F)c1F ; COc1c([C@H]2C(OC(C)=O)O[C@@](C)(C(F)(F)F)[C@H]2C)ccc(F)c1F ; COc1c([C@H]2[C@H](OC(C)=O)O[C@@](C)(C(F)(F)F)[C@H]2C)ccc(F)c1F ; COc1c([C@H]2[C@@H](OC(C)=O)O[C@@](C)(C(F)(F)F)[C@H]2C)ccc(F)c1F ; COc1c([C@H]2C(O)O[C@@](C)(C(F)(F)F)[C@H]2C)ccc(F)c1F]

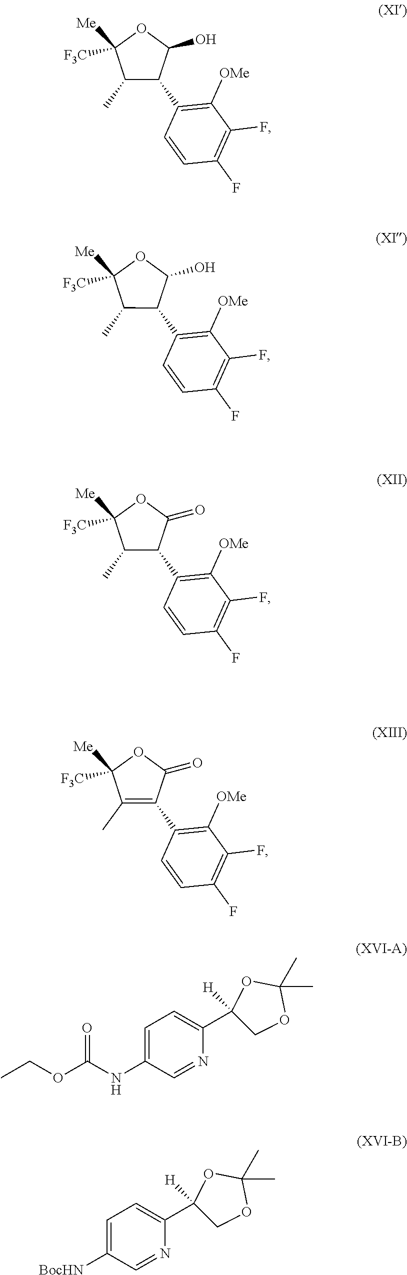

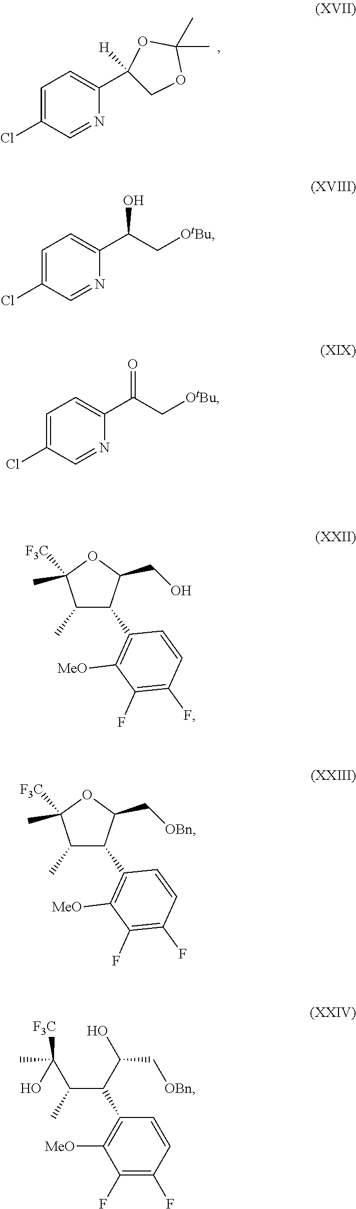

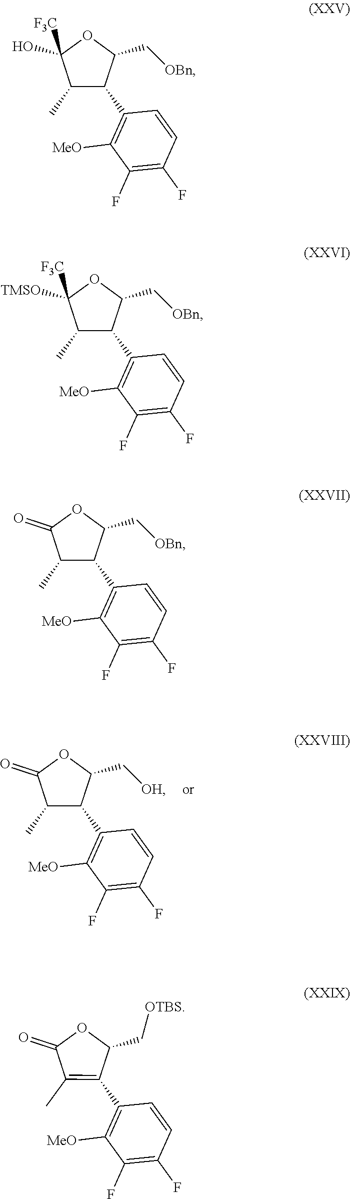

134. A compound of formula (I):

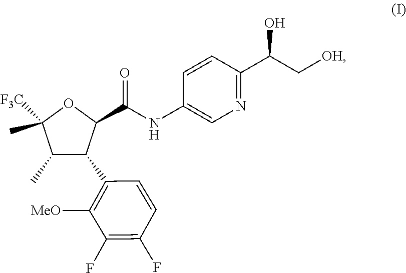 [SMILES: COc1c([C@H]2[C@H](C(=O)Nc3ccc([C@@H](O)CO)nc3)O[C@@](C)(C(F)(F)F)[C@H]2C)ccc(F)c1F]

wherein the compound is in a crystalline solid form.
135. A composition comprising a compound of formula (I):

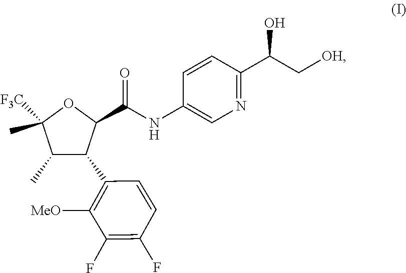 [SMILES: COc1c([C@H]2[C@H](C(=O)Nc3ccc([C@@H](O)CO)nc3)O[C@@](C)(C(F)(F)F)[C@H]2C)ccc(F)c1F]

wherein the compound of formula (I) is in a crystalline solid form.
136. The composition of embodiment 135, wherein at least 85% of the compound of formula (I) present in the composition is in a crystalline solid form.
137. The composition of embodiment 135, wherein at least 90% of the compound of formula (I) present in the composition is in a crystalline solid form.
138. The composition of embodiment 135, wherein at least 95% of the compound of formula (I) present in the composition is in a crystalline solid form.
139. The composition of embodiment 135, wherein substantially all of the compound of formula (I) present in the composition is in a crystalline solid form.
140. The composition of embodiment 135, wherein 100% of the compound of formula (I) present in the composition is in a crystalline solid form.
141. A pharmaceutical composition comprising a compound of formula (I):

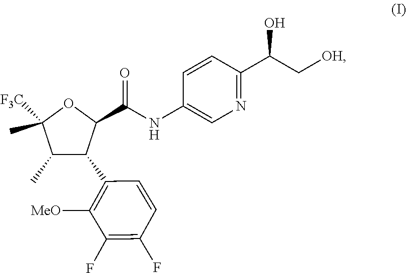 [SMILES: COc1c([C@H]2[C@H](C(=O)Nc3ccc([C@@H](O)CO)nc3)O[C@@](C)(C(F)(F)F)[C@H]2C)ccc(F)c1F]

and one or more pharmaceutically acceptable carriers or vehicles, wherein the compound of formula (I) is in a crystalline solid form.
142. The pharmaceutical composition of embodiment 141, wherein at least 85% of the compound of formula (I) present in the pharmaceutical composition is in a crystalline solid form.
143. The pharmaceutical composition of embodiment 141, wherein at least 90% of the compound of formula (I) present in the pharmaceutical composition is in a crystalline solid form.
144. The pharmaceutical composition of embodiment 141, wherein at least 95% of the compound of formula (I) present in the pharmaceutical composition is in a crystalline solid form.
145. The pharmaceutical composition of embodiment 141, wherein substantially all of the compound of formula (I) present in the composition is in a crystalline solid form.
146. The pharmaceutical composition of embodiment 141, wherein 100% of the compound of formula (I) present in the pharmaceutical composition is in a crystalline solid form.
147. The compound of embodiment 134, the composition of any one of embodiments 135 to 140, or the pharmaceutical composition of any one of embodiments 141 to 146, wherein the crystalline solid form is Form A.
148. The compound, the composition, or the pharmaceutical composition of embodiment 147, wherein Form A is characterized by an X-ray powder diffraction pattern (XRPD) pattern comprising at least one approximate peak position (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
149. The compound, the composition, or the pharmaceutical composition of embodiment 147, wherein Form A is characterized by an X-ray powder diffraction pattern (XRPD) pattern comprising at least two approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
150. The compound, the composition, or the pharmaceutical composition of embodiment 147, wherein Form A is characterized by an X-ray powder diffraction pattern (XRPD) pattern comprising at least three approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
151. The compound, the composition, or the pharmaceutical composition of embodiment 147, wherein Form A is characterized by an X-ray powder diffraction pattern (XRPD) pattern comprising at least four approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
152. The compound, the composition, or the pharmaceutical composition of embodiment 147, wherein Form A is characterized by an X-ray powder diffraction pattern (XRPD) pattern comprising at least five approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
153. The compound, the composition, or the pharmaceutical composition of embodiment 147, wherein Form A is characterized by an X-ray powder diffraction pattern (XRPD) pattern comprising at least six approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
154. The compound, the composition, or the pharmaceutical composition of embodiment 147, wherein Form A is characterized by an X-ray powder diffraction pattern (XRPD) pattern comprising at least seven approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
155. The compound, the composition, or the pharmaceutical composition of embodiment 147, wherein Form A is characterized by an X-ray powder diffraction pattern (XRPD) pattern comprising at least eight approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
156. The compound, the composition, or the pharmaceutical composition of embodiment 147, wherein Form A is characterized by an X-ray powder diffraction pattern (XRPD) pattern comprising at least nine approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
157. The compound, the composition, or the pharmaceutical composition of embodiment 147, wherein Form A is characterized by an X-ray powder diffraction pattern (XRPD) pattern comprising at least ten approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
158. The compound, the composition, or the pharmaceutical composition of embodiment 147, wherein Form A is characterized by an X-ray powder diffraction pattern (XRPD) pattern comprising at least eleven approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
159. The compound, the composition, or the pharmaceutical composition of embodiment 147, wherein Form A is characterized by an X-ray powder diffraction pattern (XRPD) pattern comprising at least twelve approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
160. The compound, the composition, or the pharmaceutical composition of embodiment 147, wherein Form A is characterized by an X-ray powder diffraction pattern (XRPD) pattern comprising at least thirteen approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
161. The compound, the composition, or the pharmaceutical composition of embodiment 147, wherein Form A is characterized by an X-ray powder diffraction pattern (XRPD) pattern comprising at least fourteen approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
162. The compound, the composition, or the pharmaceutical composition of embodiment 147, wherein Form A is characterized by an X-ray powder diffraction pattern (XRPD) pattern comprising at least fifteen approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
163. The compound, the composition, or the pharmaceutical composition of embodiment 147, wherein Form A is characterized by an X-ray powder diffraction pattern (XRPD) pattern comprising at least sixteen approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
164. The compound, the composition, or the pharmaceutical composition of embodiment 147, wherein Form A is characterized by an X-ray powder diffraction pattern (XRPD) pattern comprising at least seventeen approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
165. The compound, the composition, or the pharmaceutical composition of embodiment 147, wherein Form A is characterized by an X-ray powder diffraction pattern (XRPD) pattern comprising at least eighteen approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
166. The compound, the composition, or the pharmaceutical composition of embodiment 147, wherein Form A is characterized by an X-ray powder diffraction pattern (XRPD) pattern comprising at least nineteen approximate peak positions (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
167. The compound, the composition, or the pharmaceutical composition of embodiment 147, wherein Form A is characterized by an X-ray powder diffraction pattern (XRPD) pattern comprising approximate peak positions (degrees 2-theta±0.2) when measured using Cu K_{α} radiation of 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.
168. The compound, the composition, or the pharmaceutical composition of embodiment 147, wherein Form A is characterized by an X-ray powder diffraction pattern (XRPD) pattern comprising at least one approximate peak position (degrees 2-theta±0.2) when measured using Cu K_{α} radiation, selected from 13.4, 17.2, and 18.9.
169. The compound, the composition, or the pharmaceutical composition of embodiment 147, wherein Form A is characterized by an X-ray powder diffraction pattern (XRPD) pattern comprising at least two approximate peak positions (degrees 2-theta±0.2) when measured using Cu K_{α} radiation, selected from 13.4, 17.2, and 18.9.
170. The compound, the composition, or the pharmaceutical composition of embodiment 147, wherein Form A is characterized by an X-ray powder diffraction pattern (XRPD) pattern comprising approximate peak positions (degrees 2-theta±0.2) when measured using Cu K_{α} radiation of 13.4, 17.2, and 18.9.
171. The compound, the composition, or the pharmaceutical composition of embodiment 147, wherein Form A is characterized by an X-ray powder diffraction pattern (XRPD) pattern comprising at least one approximate peak position (degrees 2-theta±0.2) when measured using Cu K_{α} radiation, selected from 9.0, 11.4, and 20.1.
172. The compound, the composition, or the pharmaceutical composition of embodiment 147, wherein Form A is characterized by an X-ray powder diffraction pattern (XRPD) pattern comprising at least two approximate peak positions (degrees 2-theta±0.2) when measured using Cu K_{α} radiation, selected from 9.0, 11.4, and 20.1.
173. The compound, the composition, or the pharmaceutical composition of embodiment 147, wherein Form A is characterized by an X-ray powder diffraction pattern (XRPD) pattern comprising approximate peak positions (degrees 2-theta±0.2) when measured using Cu K_{α} radiation of 9.0, 11.4, and 20.1.
174. The compound, the composition, or the pharmaceutical composition of embodiment 147, wherein Form A is characterized by an X-ray powder diffraction pattern (XRPD) pattern comprising at least one approximate peak position (degrees 2-theta±0.2) when measured using Cu K_{α} radiation, selected from 13.7, 13.8, and 24.8.
175. The compound, the composition, or the pharmaceutical composition of embodiment 147, wherein Form A is characterized by an X-ray powder diffraction pattern (XRPD) pattern comprising at least two approximate peak positions (degrees 2-theta±0.2) when measured using Cu K_{α} radiation, selected from 13.7, 13.8, and 24.8.
176. The compound, the composition, or the pharmaceutical composition of embodiment 147, wherein Form A is characterized by an X-ray powder diffraction pattern (XRPD) pattern comprising approximate peak positions (degrees 2-theta±0.2) when measured using Cu K_{α} radiation of 13.7, 13.8, and 24.8.
177. The compound, the composition, or the pharmaceutical composition of embodiment 147, wherein Form A is characterized by an X-ray powder diffraction pattern (XRPD) pattern comprising:
at least one approximate peak position (degrees 2-theta±0.2) when measured using Cu K_{α} radiation, selected from 13.4, 17.2, and 18.9; and
at least one approximate peak position (degrees 2-theta±0.2) when measured using Cu K_{α} radiation, selected from 9.0, 11.4, and 20.1.
   178. The compound, the composition, or the pharmaceutical composition of embodiment 147, wherein Form A is characterized by an X-ray powder diffraction pattern (XRPD) pattern comprising:
at least two approximate peak positions (degrees 2-theta±0.2) when measured using Cu K_{α} radiation, selected from 13.4, 17.2, and 18.9; and
at least two approximate peak positions (degrees 2-theta±0.2) when measured using Cu K_{α} radiation, selected from 9.0, 11.4, and 20.1.
   179. The compound, the composition, or the pharmaceutical composition of embodiment 147, wherein Form A is characterized by an X-ray powder diffraction pattern (XRPD) pattern comprising:
approximate peak positions (degrees 2-theta±0.2) when measured using Cu K_{α} radiation of 13.4, 17.2, and 18.9; and
approximate peak positions (degrees 2-theta±0.2) when measured using Cu K_{α} radiation of 9.0, 11.4, and 20.1.
   180. The compound, the composition, or the pharmaceutical composition of embodiment 147, wherein Form A is characterized by an X-ray powder diffraction pattern (XRPD) pattern comprising:
at least one approximate peak position (degrees 2-theta±0.2) when measured using Cu K_{α} radiation, selected from 13.4, 17.2, and 18.9;
at least one approximate peak position (degrees 2-theta±0.2) when measured using Cu K_{α} radiation, selected from 9.0, 11.4, and 20.1; and
at least one approximate peak position (degrees 2-theta±0.2) when measured using Cu K_{α} radiation, selected from 13.7, 13.8, and 24.8.
   181. The compound, the composition, or the pharmaceutical composition of embodiment 147, wherein Form A is characterized by an X-ray powder diffraction pattern (XRPD) pattern comprising:
at least two approximate peak positions (degrees 2-theta±0.2) when measured using Cu K_{α} radiation, selected from 13.4, 17.2, and 18.9;
at least two approximate peak positions (degrees 2-theta±0.2) when measured using Cu K_{α} radiation, selected from 9.0, 11.4, and 20.1; and
at least two approximate peak positions (degrees 2-theta±0.2) when measured using Cu K_{α} radiation, selected from 13.7, 13.8, and 24.8.
   182. The compound, the composition, or the pharmaceutical composition of embodiment 147, wherein Form A is characterized by an X-ray powder diffraction pattern (XRPD) pattern comprising:
approximate peak positions (degrees 2-theta±0.2) when measured using Cu K_{α} radiation of 13.4, 17.2, and 18.9;
approximate peak positions (degrees 2-theta±0.2) when measured using Cu K_{α} radiation of 9.0, 11.4, and 20.1; and
approximate peak positions (degrees 2-theta±0.2) when measured using Cu K_{α} radiation of 13.7, 13.8, and 24.8.
   183. The compound, the composition, or the pharmaceutical composition of any one of embodiments 147 to 182, wherein Form A is characterized by a solid state ¹⁹F NMR spectrum having at least one peak at a chemical shift selected from −74.5, −139.5, and −161.5 ppm.
   184. The compound, the composition, or the pharmaceutical composition of any one of embodiments 147 to 182, wherein Form A is characterized by a solid state ¹⁹F NMR spectrum having at least two peaks at chemical shifts selected from −74.5, −139.5, and −161.5 ppm.
   185. The compound, the composition, or the pharmaceutical composition of any one of embodiments 147 to 182, wherein Form A is characterized by a solid state ¹⁹F NMR spectrum having peaks at chemical shifts of −74.5, −139.5, and −161.5 ppm.
   186. The compound, the composition, or the pharmaceutical composition of any one of embodiments 147 to 185, wherein Form A is characterized by a solid state ¹³C NMR spectrum having at least one peak at a chemical shift selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   187. The compound, the composition, or the pharmaceutical composition of any one of embodiments 147 to 185, wherein Form A is characterized by a solid state ¹³C NMR spectrum having at least two peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   188. The compound, the composition, or the pharmaceutical composition of any one of embodiments 147 to 185, wherein Form A is characterized by a solid state ¹³C NMR spectrum having at least three peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   189. The compound, the composition, or the pharmaceutical composition of any one of embodiments 147 to 185, wherein Form A is characterized by a solid state ¹³C NMR spectrum having at least four peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   190. The compound, the composition, or the pharmaceutical composition of any one of embodiments 147 to 185, wherein Form A is characterized by a solid state ¹³C NMR spectrum having at least five peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   191. The compound, the composition, or the pharmaceutical composition of any one of embodiments 147 to 185, wherein Form A is characterized by a solid state ¹³C NMR spectrum having at least six peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   192. The compound, the composition, or the pharmaceutical composition of any one of embodiments 147 to 185, wherein Form A is characterized by a solid state ¹³C NMR spectrum having at least seven peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   193. The compound, the composition, or the pharmaceutical composition of any one of embodiments 147 to 185, wherein Form A is characterized by a solid state ¹³C NMR spectrum having at least eight peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   194. The compound, the composition, or the pharmaceutical composition of any one of embodiments 147 to 185, wherein Form A is characterized by a solid state ¹³C NMR spectrum having at least nine peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   195. The compound, the composition, or the pharmaceutical composition of any one of embodiments 147 to 185, wherein Form A is characterized by a solid state ¹³C NMR spectrum having at least ten peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   196. The compound, the composition, or the pharmaceutical composition of any one of embodiments 147 to 185, wherein Form A is characterized by a solid state ¹³C NMR spectrum having at least eleven peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   197. The compound, the composition, or the pharmaceutical composition of any one of embodiments 147 to 185, wherein Form A is characterized by a solid state ¹³C NMR spectrum having at least twelve peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   198. The compound, the composition, or the pharmaceutical composition of any one of embodiments 147 to 185, wherein Form A is characterized by a solid state ¹³C NMR spectrum having at least thirteen peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   199. The compound, the composition, or the pharmaceutical composition of any one of embodiments 147 to 185, wherein Form A is characterized by a solid state ¹³C NMR spectrum having at least fourteen peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   200. The compound, the composition, or the pharmaceutical composition of any one of embodiments 147 to 185, wherein Form A is characterized by a solid state ¹³C NMR spectrum having at least fifteen peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   201. The compound, the composition, or the pharmaceutical composition of any one of embodiments 147 to 185, wherein Form A is characterized by a solid state ¹³C NMR spectrum having at least sixteen peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   202. The compound, the composition, or the pharmaceutical composition of any one of embodiments 147 to 175, wherein Form A is characterized by a solid state ¹³C NMR spectrum having at least seventeen peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   203. The compound, the composition, or the pharmaceutical composition of any one of embodiments 147 to 185, wherein Form A is characterized by a solid state ¹³C NMR spectrum having at least eighteen peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   204. The compound, the composition, or the pharmaceutical composition of any one of embodiments 147 to 185, wherein Form A is characterized by a solid state ¹³C NMR spectrum having at least nineteen peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   205. The compound, the composition, or the pharmaceutical composition of any one of embodiments 147 to 185, wherein Form A is characterized by a solid state ¹³C NMR spectrum having at least twenty peaks at chemical shifts selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   206. The compound, the composition, or the pharmaceutical composition of any one of embodiments 147 to 185, wherein Form A is characterized by a solid state ¹³C NMR spectrum having chemical shifts of 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.
   207. The compound, the composition, or the pharmaceutical composition of any one of embodiments 147 to 206, wherein Form A is characterized by an orthorhombic crystal system, a P2₁2₁2₁ space group, and a unit cell having dimensions measured at 100(2) K on an x-ray diffractometer equipped with Cu Kα radiation (λ=1.54178 Å) of:

$$a = 7. ( 1 ) Å α = 90 °$$

$$b = 8.3 ( 1 ) Å ; β = 90 °$$

$$c = 38.8 ( 1 ) Å γ = 90 ° .$$

208. The compound, the composition, or the pharmaceutical composition of embodiment 207, wherein the x-ray diffractometer is a Bruker x-ray diffractometer.
209. A pharmaceutical composition comprising:
45-55 wt % of a solid dispersion comprising a polymer and a compound of formula (II):

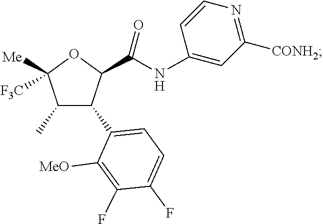 [SMILES: COc1c([C@H]2[C@H](C(=O)Nc3ccnc(C(N)=O)c3)O[C@@](C)(C(F)(F)F)[C@H]2C)ccc(F)c1F]

42-50 wt % of microcrystalline cellulose;
2-4 wt % of croscarmellose sodium; and
0.5-1.5 wt % of magnesium stearate.
   210. The pharmaceutical composition of embodiment 209, wherein the pharmaceutical composition is a tablet core composition.
   211. The pharmaceutical composition of embodiment 210, wherein the tablet core composition is coated with a tablet coating.
   212. The pharmaceutical composition of embodiment 211, wherein the tablet coating is Opadry Blue.
   213. The pharmaceutical composition of any one of embodiments 209 to 212, wherein the pharmaceutical composition comprises about 50 mg of the compound of formula (II).
   214. The pharmaceutical composition of any one of embodiments 209 to 213, wherein the polymer is HPMCAS.
   215. The pharmaceutical composition of any one of embodiments 209 to 214, wherein the solid dispersion comprises 70-80 wt % of the polymer and 20-30 wt % of the compound of formula (II).
   216. The pharmaceutical composition of embodiment 215, wherein the solid dispersion comprises about 75 wt % of the polymer and about 25 wt % of the compound of formula (II).
   217. The pharmaceutical composition of any one of embodiments 209 to 216, wherein the pharmaceutical composition comprises about 50 wt % of the solid dispersion; about 46 wt % of microcrystalline cellulose; about 3 wt % of croscarmellose sodium; and about 1.0 wt % of magnesium stearate.
   218. The pharmaceutical composition of any one of embodiments 209 to 217, wherein the pharmaceutical composition comprises an intragranular blend and extragranular blend.
   219. The pharmaceutical composition of embodiment 218, wherein the intragranular blend comprises about 200 mg of the solid dispersion, about 93.6 mg of microcrystalline cellulose, about 6.0 mg of croscarmellose sodium, and about 0.4 mg of magnesium stearate; and the extragranular blend comprises about 90.4 mg of microcrystalline cellulose, about 6.0 mg of croscarmellose sodium, and about 3.6 mg of magnesium stearate.

### EXAMPLES

General methods. ¹H NMR (400 MHz) spectra were obtained as solutions in an appropriate deuterated solvent such as dimethyl sulfoxide-d₆ (DMSO-d₆).

Unless otherwise noted, X-ray powder diffraction (XRPD) spectra were recorded at room temperature in reflection mode using a PANalytical Empyrean system equipped with a sealed tube source and a PIXcel 1D Medipix 3 detector (Malvern PANalytical Inc, Westborough, Massachusetts). The X-Ray generator operated at a voltage of 45 kV and a current of 40 mA with copper radiation (1.54060 Å). The powder sample was placed in a back filled sample holder and loaded into the instrument. The sample was scanned over the range of about 3° to about 40° 2θ with a step size of 0.0131° and 49.73s per step.

Unless otherwise noted, solid state NMR analysis was conducted on a Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 KHz.

### ABBREVIATIONS

Unless otherwise noted, or where the context dictates otherwise, the following abbreviations shall be understood to have the following meanings:

| Abbrevi- | |
|---|---|
| ation | Meaning |
| NMR | Nuclear magnetic resonance |
| ESI-MS | Electrospray mass spectrometry |
| LC/MS | Liquid chromatography-mass spectrometry |
| UPLC | Ultra performance liquid chromatography |
| HPLC/ | High performance liquid chromatography/tandem mass |
| MS/MS | spectrometry |
| IS | Internal standard |
| HPLC | High performance liquid chromatography |
| SFC | Supercritical fluid chromatography |
| ESI | Electrospray ionization |
| kg | Kilogram |
| g | Grams |
| mg | Milligrams |
| L | Liter(s) |
| mL | Milliliters |
| μL | Microliters |
| nL | Nanoliters |
| mol | Mole |
| mmol | Millimoles |
| hr, h | Hours |
| min | Minutes |
| ms | Millisecond |
| mm | Millimeters |
| μm | Micrometers |
| nm | Nanometer |
| MHz | Megahertz |
| Hz | Hertz |
| N | Normal (concentration) |
| M | Molar (concentration) |
| mM | Millimolar (concentration) |
| μM | Micromolar (concentration) |
| ppm | Parts per million |
| % w/v | Weight-volume concentration |
| % w/w | Weight-weight concentration |
| Ac₂O | Acetic anhydride |
| BnBr | Benzyl bromide |
| t-BuOH | Tert-butyl alcohol |
| CDI | 1,1′-Carbonyldiimidazole |
| DAST | (Diethylamino)sulfur trifluoride |
| DCM | Dichloromethane |
| DCE | Dichloroethane |
| DIAD | Diisopropyl azodicarboxylate |
| DIBAL | Diisobutylaluminium hydride |
| DIEA, | N,N-Diisopropyl ethyl amine |
| DIPEA | |
| DMA | N,N-Dimethylacetamide |
| DMAP | Dimethylaminopyridine |
| DMF | N,N-Dimethylformamide |
| DMSO | Dimethyl sulfoxide |
| DRG | Dorsal root ganglia |
| EtOH | Ethanol |
| EtOAc | Ethyl acetate |
| HATU | 1-[Bis(dimethylamino)methylene]-1H-1,2,3-triazolo[4,5- |
| | b]pyridinium 3-oxide hexafluorophosphate |
| EDCI | 1-Ethyl-3-(3-dimethylaminopropyl)carbodiimide |
| T3P | Propylphosphonic anhydride, i.e., 2,4,6-tripropyl- |
| | 1,3,5,2,4,6-trioxatriphosphinane 2,4,6-trioxide |
| mCPBA | Meta-Chloroperoxybenzoic acid |
| MeOH | Methanol |
| MsCl | Methanesulfonyl chloride |
| MTBE | Methyl tert-butyl ether |
| NCS | N-Chlorosuccinimide |
| NIS | N-Iodosuccinimide |
| NMP | N-Methylpyrrolidone |
| PdCl₂(dtbpf) | 1,1′-Bis(di-tert-butylphosphino)ferrocene palladium |
| | dichloride |
| PTSA | Para-toluenesulfonic acid |
| STAB | Sodium triacetoxyborohydride |
| TBAF | Tetrabutylammonium fluoride |
| TBSOTf | Tert-Butyldimethylsilyl trifluoromethanesulfonate |
| TCFH | Chloro-N,N,N′,N′-tetramethylformamidinium |
| | hexafluorophosphate |
| THF | Tetrahydrofuran |
| TEA | Triethylamine |
| Tf₂O | Trifluoromethanesulfonic anhydride |
| TFA | Trifluoroacetic acid |
| TMSCl | Trimethylsilyl chloride |
| TMSCN | Trimethylsilyl cyanide |
| RB | Round bottom (flask) |
| RT | Room temperature |
| ca. | Circa (approximately) |
| E-VIPR | Electrical stimulation voltage ion probe reader |
| HEK | Human embryonic kidney |
| KIR2.1 | Inward-rectifier potassium ion channel 2.1 |
| DMEM | Dulbecco's Modified Eagle's Medium |
| FBS | Fetal bovine serum |
| NEAA | Non-essential amino acids |
| HEPES | 2-[4-(2-hydroxyethyl)piperazin-1-yl]ethanesulfonic acid |
| DiSBAC₆(3) | Bis-(1,3-dihexyl-thiobarbituric acid) trimethine oxonol |
| CC2-DMPE | Chlorocoumarin-2-dimyristoyl phosphatidylethanolamine |
| VABSC-1 | Voltage Assay Background Suppression Compound |
| HS | Human serum |
| BSA | Bovine Serum Albumin |
| SDD | Spray-Dried Dispersion |
| HPMCAS | Hydroxypropyl methylcellulose acetate succinate |
| PTFE | Polytetrafluoroethylene |

### Example 1—Preparation of Compound (IV) and (VII)

Compound VII may be prepared by the process described below:

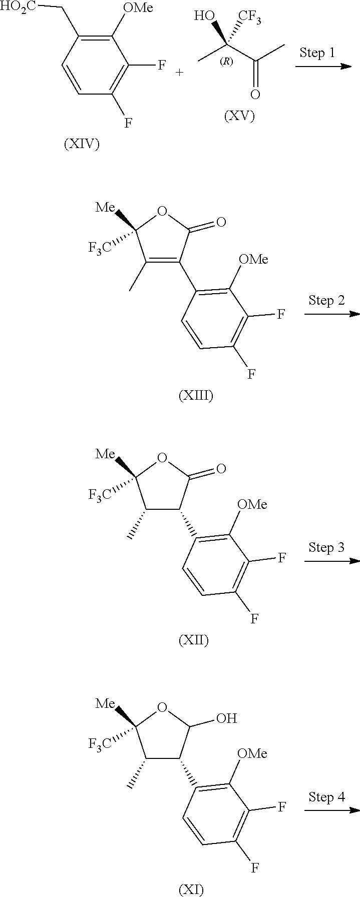

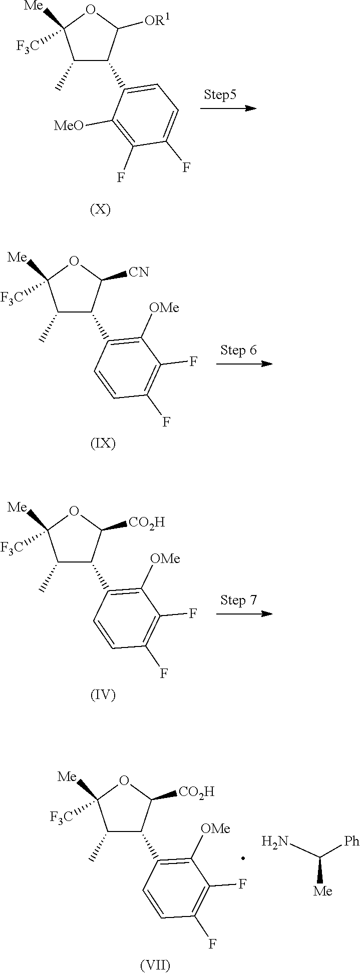

In some embodiments, R¹ is —C(O)CH₃, as described below in Step 4A. In some embodiments, R¹ is —C(O)(4-nitrophenyl), as described below in Step 4B.

### Step 1: Synthesis of (R)-3-(3,4-difluoro-2-methoxyphenyl)-4,5-dimethyl-5-(trifluoromethyl)furan-2 (5H)-one (XIII)

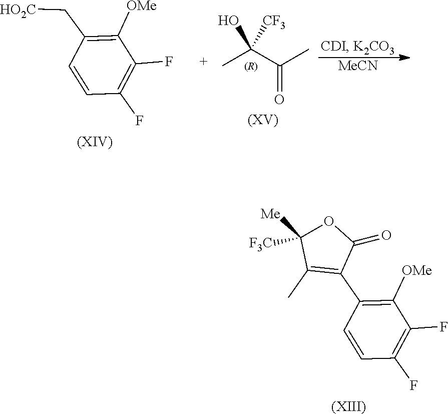

Compound XIV (Alichem, 3.832 kg, 18.955 mols) was added to a 140 L reactor that had been previously vacuum-dried and flushed with N₂ gas. Anhydrous acetonitrile, 18.85 L, was added, and the solution was cooled to −2° C. Carbonyldiimidazole (Chem Impex, 99.5%, 3.280 kg, 19.92 mols, 1.05 eq) was added in portions (from 4×820 g bottles, preweighed in a N₂ drybox. Each bottle of CDI was added 1-2 minutes apart to avoid a sudden gas evolution of CO₂. The temperature went to +2° C. during the addition. The solution was stirred at 0° to −2° C. for 1.5 hours. A solution of compound XV in THF (containing 4.028 kg of XII; 20.42 mols, 1.077 eq) was added fast with a metering pump. The pump and container were rinsed with 2 L of anhydrous acetonitrile, potassium carbonate, 325 mesh anhydrous (3.276 kg, 23.70 mols, 1.25 eq) was quickly added, and the reaction mixture was stirred at 35° C. for 5 hours, then cooled to 15° C. overnight. MTBE (24.5 L) was added followed by 62.4 kg of 0.62N H₂SO₄ and then a 5 L rinse of DI water. The aqueous layer (pH 8) was re-extracted with 20 L of MTBE. The total MTBE was vacuum concentrated to a dry solid, and re-concentrated with 10 L IPA to a dry solid.

For recrystallization, the solid was dissolved in 25.5 L of IPA and transferred to the 140 L reactor with a 5 L rinse of IPA; the solution was warmed to 35° C. A pump was set up for delivery of 47.1 kg of DI water; this was added to the IPA solution slowly over 2.5 hours. After stirring the slurry of crystals for another 2 hours, the slurry was ramped down over approx. 3 hours to 15° C. and continued stirring for another 12 hours. The slurry was filtered, washed with 2×6 L of 1:4 IPA:DI water, and dried with single pass heated N₂ gas (N₂ gas was heated to 75° C., the cake temperature was ca 50° C.) for 3 days to constant weight. Final weight of product was 5.205 kg, 85.2%. The product gave a proton NMR spectrum consistent with the structure of compound XIII.

### Step 2: Synthesis of (3S,4S,5R)-3-(3,4-difluoro-2-methoxyphenyl)-4,5-dimethyl-5-(trifluoromethyl)dihydrofuran-2 (3H)-one (XII)

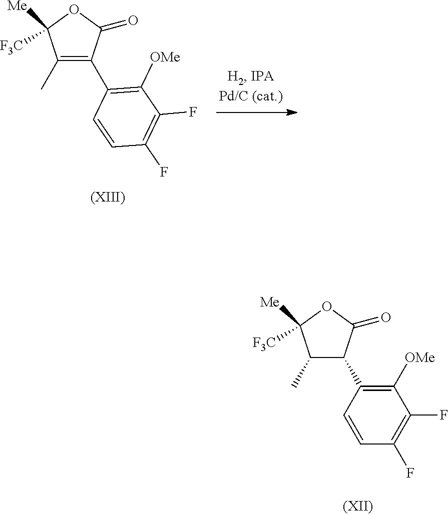

### Procedure 1:

Compound XIII, obtained from Step 1 above (1200 g, 3.72 mol), was added to the 11 L hydrogenation reactor (previously flushed with N₂ gas and set for a slow N₂ gas sweep). The catalyst (Pd/C, e.g., Johnson Matthey Catalog No. A503032-5 or A503014-5, 1200 g, 4.89% Pd, 63.8% water, corresponding to 21.2 g Pd, 0.0536 molar eq.) was added. Isopropanol (7.0 L) was added. The reactor was sealed, purged 3×N₂ gas/vacuum, then with 50 psi nitrogen/hydrogen, and finally adjusted to 225 psi hydrogen. The jacket was set to 30-31° C. and agitation was started. Agitation was continued for 30 hours; an NMR sample (0.2 ml+2.0 ml MTBE+1.0 ml of 5% KHCO₃; evaporation of 1.5 ml of the MTBE, and NMR); showed 2.8% starting material. Reaction was considered complete. The slurry was filtered (solka-floc) and washed with 10 L of IPA, then 3 L DCM. The filtrate was concentrated to an oil, and redissolved in 3 L of toluene. The toluene solution was re-concentrated to an oil, 1152 g, 3.553 mols, 95.5% yield. Proton NMR of the product was consistent with compound XII.

¹H-NMR CDCl₃: δ 6.93-6.80 (m, 2H); 4.48 (d, 1H, J=9.5 Hz); 4.03 (d, 3H, J=3.1 Hz); 2.89 (dq, 1 H, J=9.5, 7.5 Hz); 1.71 (d, 3H, J=1.2 Hz); 0.84-0.76 (m, 3H) ppm.

### Procedure 2:

To a reactor rated for hydrogen and pressure service, and equipped with a gas dosing unit and pressure controller, was charged Compound XIII (1 equiv, limiting reagent), 5% palladium on carbon (0.05 equiv, e.g., Johnson Matthey Catalog No. A503032-5 or A503014-5, corrected for water content and palladium assay), tetrahydrofuran (1.75 volumes), 2-propanol (5.25 volumes), and trifluoroacetic acid (0.05 equiv). The vessel was pressurized to 3 barg with nitrogen, and then vented to ambient pressure. This sequence was performed 3 times. The reactor contents were adjusted to 30° C. Alternatively, the reactor contents can be adjusted to 50° C. The vessel was then pressurized to 3 barg with hydrogen, and vented to ambient pressure. This sequence was performed 3 times. The reactor was then pressurized to operating pressure (40 barg) with hydrogen, and agitation was started at a sufficient speed to achieve gassing of the liquid from the head space.

The reaction mixture was stirred at these conditions until reaction completion (Less than 1% Compound XIII and its diastereomers by GC).

The hydrogen in the headspace was vented. The reactor was pressurized to 3 barg with nitrogen and the reactor was vented. This sequence was performed 3 times. Cyclohexene (0.2 volumes) was charged to the reactor, and the reaction was maintained at 30° C. with stirring under nitrogen for no less than 15 minutes.

The reaction mixture was filtered over a bed of diatomaceous earth to remove the catalyst. The filter cake was washed with 2-propanol (4 volumes). The filtrates from the primary filtration and the wash were combined.

In a well mixed vessel, the filtrate was concentrated under reduced pressure at no more than 40° C. to a total of 3 volumes. Toluene (7 volumes) was charged and distillation was resumed under reduced pressure at no more than 50° C. until reaching a total of 3 volumes. Toluene (7 volumes) was charged and distillation was resumed under reduced pressure at no more than 50° C. until reaching a total of 3 volumes. Toluene (5 volumes) was charged and the solution was mixed well. Proton NMR of the product was consistent with compound XII.

¹H-NMR CDCl₃: δ 6.93-6.80 (m, 2H); 4.48 (d, 1H, J=9.5 Hz); 4.03 (d, 3H, J=3.1 Hz); 2.89 (dq, 1 H, J=9.5, 7.5 Hz); 1.71 (d, 3H, J=1.2 Hz); 0.84-0.76 (m, 3H) ppm.

### Step 3: Synthesis of (2S,3S,4S,5R)-3-(3,4-difluoro-2-methoxyphenyl)-4,5-dimethyl-5-(trifluoromethyl)tetrahydrofuran-2-ol (XI)

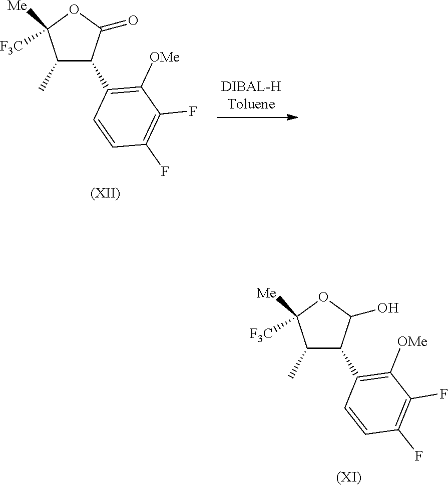

### Procedure 1:

The product of Step 2 above (compound XII, 1052 g, 3.2445 mols) was added to a 50 L jacketed reactor, and added 8.82 L of anhydrous toluene under N₂ gas. The resulting solution was cooled to −31° C. overnight under N₂ gas. Diisobutylaluminum hydride (1.96 kg of 25% solution in toluene, 3.445 mol, 1.056 eq) was slowly transferred to the reaction vessel through an addition funnel under N₂ gas. The hydride reagent was added to the reaction solution over 2 hours and the reactor temperature went from −31.6° to −27.4° C. during the addition. The solution was stirred at −26 to −27° C. for 90 minutes. A solution of 2.75 kg of potassium/sodium tartrate in 20 L of DI water was added over 2.5 hours. The reaction mixture was allowed to warm up until it reached 0° C., the cooler was turned to +24° C. after about 2 hours of addition. Toluene (5 L) was added and the mixture was stirred overnight at +20° C.

The reaction mixture was transferred to a separatory funnel and separated aqueous phase from the organic phase. The aqueous phase was re-extracted with 5 L of toluene. The two toluene solutions were combined, treated with magnesium sulfate, and filtered. The solid was washed with toluene, and the combined toluene solution was vacuum concentrated to an oil, wt. 1055 g, 3.2336 mol, 99.7% of crude lactol. The crude product was directly taken into the next step (acetylation is exemplified below). The proton NMR of the crude material was consistent with compound XI.

¹H-NMR CDCl₃: δ 7.30-7.26 (m, 1H); 7.20-7.18 (m, 1H); 5.81 (d, 1H, J=4 Hz); 4.00 (s, 3 H); 3.84-3.80 (m, 1H); 2.92-2.88 (m, 1H); 1.67 (s, 3H); 0.83 (d, 3H, J=8 Hz) ppm.

### Procedure 2:

A solution of Compound XII in toluene (about 8 vol) was added to a reactor. Alternatively, the compound of formula XII can be added to the reactor with a target of 5 vol. Agitation was begun and mixture was cooled to −25±5° C. A solution of diisobutylaluminum hydride (25% w/w toluene) was added to the reaction mixture while maintaining the batch temperature at no more than −20° C. Temperature was adjusted to −25±5° C. and the batch was stirred for no less than one hour. Upon reaction completion, a solution of acetone in toluene (0.3 equiv in 0.5 vol) was added to the reaction mixture, maintaining temperature at −25±5° C. and stirred for no less than 30 minutes. Reaction mixture was then warmed to 0±5° C. A 12 vol solution of 0.62 M citric acid was transferred to the reactor while maintaining the temperature at 20±5° C. and the biphasic mixture was stirred for no less than four hours. Alternatively, 8 vol of a 0.62 M citric acid can be added to the reactor while maintaining the temperature at 20±5° C. and the biphasic mixture can be stirred for no less than four hours. Phases were allowed to settle and the bottom, aqueous phase was drained off. A 12-vol solution of 0.62 M citric acid was added to the batch and the biphasic mixture was stirred at 20±5° C. for no less than 30 minutes. Alternatively, 8 vol of a 0.62 M citric acid can be added to the batch and the biphasic mixture can be stirred at 20±5° C. for no less than 30 minutes. Phases were allowed to settle and the bottom, aqueous layer was drained off. 5 vol of water was charged to the batch and the biphasic mixture was stirred at 20±5° C. for no less than 30 minutes. Phases were allowed to settle and the bottom, aqueous layer was drained off. The organic layer was distilled to a total of 5 vol while maintaining an internal temperature at or below 45° C. 5 vol of toluene was charged and the mixture was distilled to a total of 5 vol. Distillation continued until residual water was below 0.1%. The proton NMR of the isolated material was consistent with compound XI.

¹H-NMR CDCl₃: δ 7.30-7.26 (m, 1H); 7.20-7.18 (m, 1H); 5.81 (d, 1H, J=4 Hz); 4.00 (s, 3 H); 3.84-3.80 (m, 1H); 2.92-2.88 (m, 1H); 1.67 (s, 3H); 0.83 (d, 3H, J=8 Hz) ppm.

### Step 4A: Synthesis of (3S,4S,5R)-3-(3,4-difluoro-2-methoxyphenyl)-4,5-dimethyl-5-(trifluoromethyl)tetrahydrofuran-2-yl acetate (X-B)

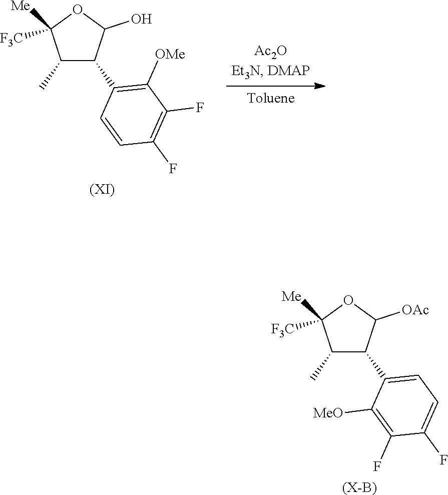

The crude product of Step 3 above (1055 g of compound XI, 3.23 mols) was transferred to a 25 L jacketed reactor pre-dried and kept under N₂ gas. Crude compound XI was rinsed in with anhydrous toluene, 6.8 L and the mixture stirred to ensure complete solution at 20° C. Triethylamine (466 ml, 3.343 mols, 1.04 eq) was added, followed by DMAP (3.58 g, 0.0293 mols, 0.01 eq). Acetic anhydride, (313 ml, 3.288 mols, 1.02 eq) was added over about 5-10 minutes using an addition funnel (T rose from 21.1° C. to a T=26.4° C. during addition of acetic anhydride). The reaction mixture was stirred at 25° C. for 100 minutes. Proton NMR of the reaction mixture indicated that the reaction was complete at this point.

The reaction mixture was extracted with 3.5 L of 25% ammonium chloride, then 1200 ml of 10% KHCO₃. The aqueous layers were re-extracted with 2 L of toluene and the combined toluene solution was dried over magnesium sulfate, filtered and washed the filtered solid with toluene. The resulting toluene solution was concentrated to dryness, initially with a 20 L RB flask and then using a 3 L jacketed reactor (1898 g transferred) for the final concentration. The solution was vacuum-distilling to a thick oil, placed it on high-vacuum at 25° C. with stirring until the oil converts to a crystalline mass (with a small amount of a viscous oil remaining). Hexane (900 ml) was added to the reaction vessel (containing solid and some remnant oil) and stirred the mixture at 20° C. overnight. The mixture was cooled to 10° C. and stirred for 2 hours, then cooled to 4° C. while stirring for 6 hours, before cooling the mixture to −10° C. overnight (while stirring), and finally to −14° C. over the weekend (36 to 48 hours) to obtain a crystal slurry. The slurry was filtered with a jacketed filter at −15° C., and then washed the solid with cold (−16° C.) hexane (2×150 ml, then 100 ml). The crystals were dried at RT under vacuum. The resulting solid was dissolved in toluene (total 1450 ml solution) and assayed to obtain 800.2 g in solution.

Additional product (that was stuck to the reactor) was dissolved off with toluene to give 425 ml of solution; assay gave 188.4 g product. The mother liquors were concentrated to 117 g of an oil. Proton NMR showed about 55% product, and 45% impurities.

The total yield (crystals filtered from reaction mixture and solids dissolved from the reactor surface) provided 988.6 g, 2.68 mols, 83.1% of compound X-B.

The product (988.6 g compound X-B) was used in the next reaction without any further purification.

### Step 4B: Synthesis of (2S,3S,4S,5R)-3-(3,4-difluoro-2-methoxyphenyl)-4,5-dimethyl-5-(trifluoromethyl)tetrahydrofuran-2-yl 4-nitrobenzoate (X-A)

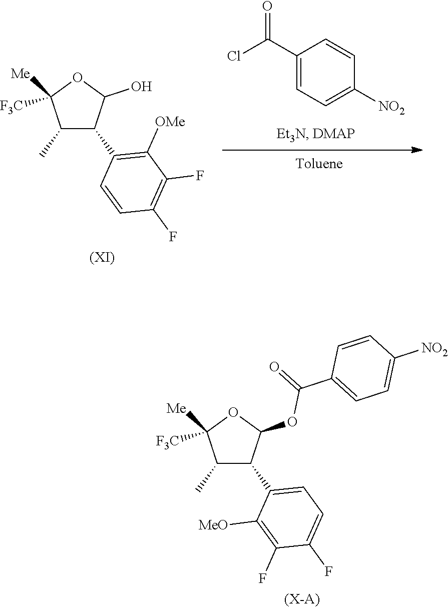

### Procedure 1:

Compound XI in a solution of 5 vol toluene was charged to the reactor. An additional 5 vol of toluene was charged bringing the total volume to 10 vol. Temperature was adjusted to 20±5° C. 4-nitrobenzoyl chloride was charged to the reactor as a solid. Temperature was adjusted to 0±5° C. Triethylamine was charged slowly to maintain temperature at 0±5° C. Temperature was adjusted to 20±5° C. and reaction mixture was stirred for no less than 3 h. Upon reaction completion, 5 vol of a 2 M aqueous NaOH solution was added to the reactor, maintaining a temperature of 20±5° C., and the biphasic mixture was stirred for no less than 1 hour. Agitation was stopped, the phases were allowed to settle and the bottom, aqueous phase was drained off. A 5 vol solution of saturated ammonium chloride was added the reactor and the biphasic mixture was stirred for no less than 30 minutes. Agitation was stopped, the phases were allowed to settle and the bottom, aqueous phase was drained off. A 5 vol solution of saturated ammonium chloride was added to the reactor and the biphasic mixture was stirred for no less than 30 minutes. 5 vol of water was charged to the reactor and the biphasic mixture was stirred for no less than 30 minutes. Phases were allowed to settle and the bottom, aqueous layer was drained off.

Organic phase was distilled to a total of 5 vol while maintaining an internal temperature at or below 45° C. 5 vol of toluene was charged and the mixture was distilled to 2.2 volumes. 1.2 vol of n-heptane was charged to the distilled toluene solution. Mixture was heated to an internal temperature of 70±5° C. and stirred for no less than 15 minutes and no more than 1 hour. Solution was cooled to 60±5° C. over 30 minutes. The clear solution was seeded with 0.010 w/w equiv of Compound X-A seeds and agitated for no less than 1 hour and no more than 2 h. 2.4 vol of n-heptane was charged at a linear rate over 5 hours. Slurry was cooled to 20±5° C. over 5 h. Slurry was aged for no less than 5 h. The solids were isolated via filtration. A 2 vol wash solution of 75:25 n-heptane: toluene was used to wash the wet cake. The wet cake was transferred to drying equipment and dried under vacuum at a temperature of 40±5° C. until a constant weight was observed. The proton NMR of the crude material was consistent with the 4-nitrobenzoate ester of compound X, compound X-A. The total yield from Step 2 to Step 4B is 65%.

¹H-NMR CDCl₃: δ 8.21 (d, 2H, J=8 Hz); 8.10 (d, 2H, J=8 Hz); 6.93-6.88 (m, 1H); 6.81-6.76 (m, 1H) 6.73 (d, 1H, J=4 Hz); 4.09 (d, 1H, J=4 Hz); 3.95-3.91 (m, 1H); 3.91 (s, 3H); 1.60 (s, 3 H); 0.86-0.84 (d, 3H, J=8 Hz) ppm.

### Procedure 2:

Compound XI (solution from above, 801 kg, 906.2 mol, 1.00 equiv) in a solution of 3 vol toluene was charged to the reactor. Temperature was adjusted to 20±5° C. Triethylamine (110.0 kg, 1087.4 mol, 1.20 equiv.) was charged, followed by 4-dimethylaminopyridine (DMAP, 1.66 kg, 13.6 mol, 0.015 equiv, 0.006 w/w equiv). A solution of 4-nitrobenzoyl chloride (185.0 kg, 996.8 mol, 1.10 equiv.) in 6.0v MTBE (1296.5 kg) was prepared, then charged to the reactor over no less than 1 hour at a temperature of 20±5° C. The reaction mixture was stirred for no less than 30 min. Upon reaction completion, 5 vol of a 2 M aqueous NaOH solution was added to the reactor, maintaining a temperature of 20±5° C., and the biphasic mixture was stirred for no less than 1 hour. Agitation was stopped, the phases were allowed to settle and the bottom, aqueous phase was drained off. A 5 vol solution of saturated ammonium chloride was added the reactor and the biphasic mixture was stirred for no less than 30 minutes. Agitation was stopped, the phases were allowed to settle and the bottom, aqueous phase was drained off. 5 vol of water was charged to the reactor and the biphasic mixture was stirred for no less than 30 minutes. Phases were allowed to settle and the bottom, aqueous layer was drained off.

Organic phase was distilled to a total of 3.45 vol while maintaining an internal temperature at or below 45° C. Toluene (2025 kg, 8.0 vol) was charged and the mixture was distilled to 3.45 volumes. n-Heptane (155.8 kg, 0.78 vol) was charged to the reactor and the mixture was heated to an internal temperature of 55±5° C. and stirred for no less than 30 minutes to fully dissolve. The solution was cooled to 47±3° C. over 30 minutes. The clear solution was seeded with Compound X-A (2.92 kg, 0.010 w/w equiv) seeds and agitated for no less than 1 hour. n-Heptane (747 kg, 3.7 vol) was charged at a linear rate over 1 hours, and the slurry was aged for 2 hours. The slurry was cooled to 5±5° C. over 140 min, then aged for no less than 12 h. The solids were isolated via filtration. A 2.4 vol wash solution of 75:25 n-heptane: toluene was used to wash the wet cake. The wet cake was transferred to drying equipment and dried under vacuum at a temperature of 40±5° C. until a constant weight was observed. The proton NMR of compound was consistent with the 4-nitrobenzoate ester of compound X, compound X-A.

¹H-NMR CDCl₃: δ 8.21 (d, 2H, J=8 Hz); 8.10 (d, 2H, J=8 Hz); 6.93-6.88 (m, 1H); 6.81-6.76 (m, 1H) 6.73 (d, 1H, J=4 Hz); 4.09 (d, 1H, J=4 Hz); 3.95-3.91 (m, 1H); 3.91 (s, 3H); 1.60 (s, 3 H); 0.86-0.84 (d, 3H, J=8 Hz) ppm.

### Recrystallization:

Compound X-A (258 kg, 543.8 mol, 1.00 equiv.) was charged to a reactor. Acetone (1264.6 kg, 6.24 vol) and water (315.4 kg, 1.22 vol) were added and agitation started. The slurry was heated to an internal temperature of 45±5° C. to fully dissolve. The solution was cooled to 30±3° C. at no more than 0.5° C./min. The clear solution was seeded with Compound X-A (1.29 kg, 0.0050 w/w equiv) seeds and agitated for no less than 2 hours. Water (224.9 kg, 0.87 vol) was charged at a linear rate over 2 hours, and the slurry was aged for 1 hour to grow the seed bed. The slurry was cooled to 20±3° C. at a linear rate of no more than 0.2° C./min, than aged for no less than 8 h. The solids were isolated via filtration. A 2.0 vol wash solution of 75:25 acetone: water was used to wash the wet cake. The wet cake was transferred to drying equipment and dried under vacuum at a temperature of 40±5° C. until a constant weight was observed. The proton NMR of the recrystallized compound was consistent with benzoate ester of compound X-A. The total yield from Step 2 to Step 4B is 65%.

¹H-NMR CDCl₃: δ 8.21 (d, 2H, J=8 Hz); 8.10 (d, 2H, J=8 Hz); 6.93-6.88 (m, 1H); 6.81-6.76 (m, 1H) 6.73 (d, 1H, J=4 Hz); 4.09 (d, 1H, J=4 Hz); 3.95-3.91 (m, 1H); 3.91 (s, 3H); 1.60 (s, 3 H); 0.86-0.84 (d, 3H, J=8 Hz) ppm.

### Step 5A: Synthesis of (2R,3S,4S,5R)-3-(3,4-difluoro-2-methoxyphenyl)-4,5-dimethyl-5-(trifluoromethyl)tetrahydrofuran-2-carbonitrile (IX)

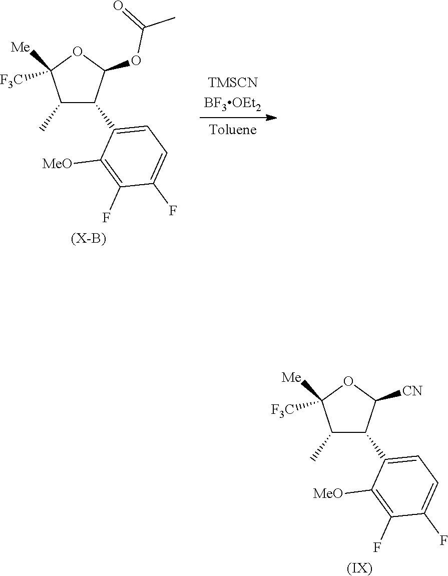

Transferred compound X-B (1.87 L toluene solution containing 987 g of compound X-B obtained in Step 4A; 2.68 mols) to a 50 L jacketed reactor predried with N₂ gas/vacuum 4 times under N₂ gas. Anhydrous toluene (7.0 L) was added, stirred and cooled to −31° C. TMS-CN (385 g; 3.7 mols; 1.4 eq) was added over 8 minutes. BF₃ diethyl etherate (380 g=330 ml; 2.68 mols) was added dropwise over 6 minutes (Temp. −31.5° C. increased to −27.6° C.). The bath temperature was set at −21.6° C., and the reaction mixture was stirred for 2.5 hours. Potassium hydroxide (3.5 L of 2.0 M solution) was added over about 5 minutes (temperature rose to +8° C.). The bath temperature was raised to +20° C., the reaction mixture was stirred at +20° C. for about 10 minutes. The layers were separated and the aqueous layer was re-extracted with 6 L of toluene. The toluene solutions were re-extracted with 1.5 L of 2M potassium hydroxide before vacuum concentrating them to about 900 g of oil. The oil was diluted with 5 L of methanol and re-concentrated to give 887 g (2.65 mols, 98.7% is pure) of final crude compound IX. Proton NMR of the solid was consistent with the expected structure. NMR did not detect any methanol or toluene, suggesting that the solid is not solvated.

The crude compound IX was purified using a preparative silica gel column packed in 85:15 hexane: MTBE. A solution of 0.78 kg of the crude product in the packing solvent mixture was transferred to the column and eluted with the packing solvent mixture. The fractions were split approximately in half and each half was reacted separately to prepare the next compound (compound IV). The early half of the fractions contained 333 g (or 993 mmols) of compound IX whereas the late half of the fractions contained 274 g (or 818 mmols) of compound IX. The example below (i.e. Step 6) shows the process of converting compound IX collected from the early half of the fractions. Compound IX obtained in the late half of the fractions was also converted to compound IV following an identical process except for the amounts of reagents and solvents.

¹H-NMR CDCl₃: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1 H, J=7.7 Hz); 1.64 (d, 3H, J=1.3 Hz); 0.80 (dq, 3 H, J=7.3, 2.3 Hz) ppm.

### Step 5B: Synthesis of (2R,3S,4S,5R)-3-(3,4-difluoro-2-methoxyphenyl)-4,5-dimethyl-5-(trifluoromethyl)tetrahydrofuran-2-carbonitrile (IX)

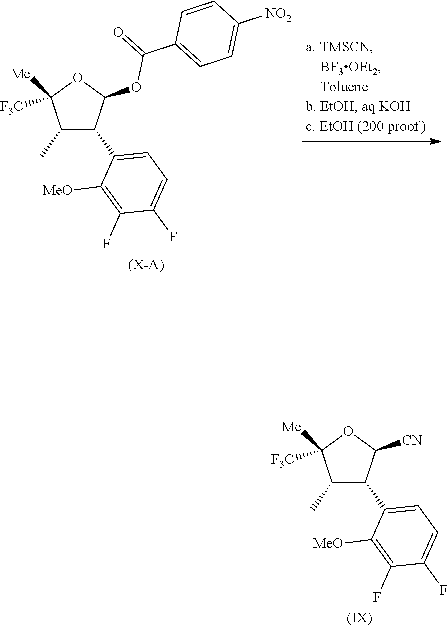

### Procedure 1:

Compound X-A solids were charged to a reactor. 10 vol of toluene was charged to the reactor, and temperature was controlled to 20±5° C. 40% trimethylsilyl cyanide solution in toluene (1.2 equiv of TMSCN) was added to the reactor while maintaining a temperature of 20±5° C. Batch was cooled to −20±5° C. 1.0 equiv of boron trifluoride diethyl etherate was added slowly while maintaining the temperature at −20±5° C. Batch was stirred at −20±5° C. for no less than 3 hours. Upon reaction completion batch was heated back to 20±5° C. 10 vol of 20% w/w aqueous potassium hydroxide solution was added to the batch, the biphasic mixture was stirred for no less than 1 h. 5 vol of ethanol was added to the batch and the biphasic mixture was stirred for no less than 12 h. Phases were allowed to settle, and the bottom aqueous layer was drained off. 10 vol of 20% w/w aqueous potassium hydroxide solution was added to the batch, the biphasic mixture was stirred for no less than 1 h. Phases were allowed to settle, and the bottom aqueous layer was drained off. 10 vol of 20% w/w aqueous potassium hydroxide solution was added to the batch, the biphasic mixture was stirred for no less than 30 minutes. Phases were allowed to settle, and the bottom aqueous layer was drained off. 10 vol of water was added to the batch and the biphasic mixture was stirred for no less than 30 minutes. Phases were allowed to settle, and the bottom aqueous layer was drained off. Top, organic phase was distilled to a total of 4 vol while maintaining internal temperature at or below 45° C. 7 vol of ethanol was charged and the mixture was distilled to a total of 4 vol. Another 7 vol of ethanol was charged and the mixture was distilled to a total of 4 vol. Distillation continued until residual toluene was no more than 1.0% w/w. The proton NMR of the crude product was consistent with compound IX.

¹H-NMR CDCl₃: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1 H, J=7.7 Hz); 1.64 (d, 3H, J=1.3 Hz); 0.80 (dq, 3 H, J=7.3, 2.3 Hz) ppm.

### Procedure 2:

Compound X-A solids (119.4 kg, 251.1 mol, 1.00 equiv.) were charged to a reactor. Toluene (517 kg, 5.0 vol) was charged to the reactor, and temperature was controlled to 20±5° C. 40% trimethylsilyl cyanide solution in toluene (376.8 mol, 1.5 equiv of TMSCN) was added to the reactor while maintaining a temperature of 20±5° C. Batch was cooled to −20=5° C. Boron trifluoride diethyl etherate (17.8 kg, 125.6 mol, 0.50 equiv.) was added slowly while maintaining the temperature at −20±5° C. Batch was stirred at −20±5° C. for no less than 3 hours. Upon reaction completion batch was heated back to 20±5° C. 10 vol of 20% w/w aqueous potassium hydroxide solution was added to the batch, the biphasic mixture was stirred for no less than 1 h. 5 vol of ethanol was added to the batch and the biphasic mixture was stirred for no less than 12 h. Phases were allowed to settle, and the bottom aqueous layer was drained off. 10 vol of 20% w/w aqueous potassium hydroxide solution was added to the batch, the biphasic mixture was stirred for no less than 1 h. Phases were allowed to settle, and the bottom aqueous layer was drained off. 10 vol of 20% w/w aqueous potassium hydroxide solution was added to the batch, the biphasic mixture was stirred for no less than 30 minutes. Phases were allowed to settle, and the bottom aqueous layer was drained off. 10 vol of water was added to the batch and the biphasic mixture was stirred for no less than 30 minutes. Phases were allowed to settle, and the bottom aqueous layer was drained off. Top, organic phase was distilled to a total of 4 vol while maintaining internal temperature at or below 45° C. 7 vol of ethanol was charged and the mixture was distilled to a total of 4 vol. Another 7 vol of ethanol was charged and the mixture was distilled to a total of 4 vol. Distillation continued until residual toluene was no more than 1.0% w/w. The proton NMR of the crude product was consistent with compound IX.

¹H-NMR CDCl₃: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1 H, J=7.7 Hz); 1.64 (d, 3H, J=1.3 Hz); 0.80 (dq, 3 H, J=7.3, 2.3 Hz) ppm.

### Procedure 3:

Compound X-A solids (1.00 equiv) were charged to a reactor. Toluene (5.0 vol, 4.34 w/w equiv) was charged to the reactor, and temperature was controlled to 20±5° C. 40% trimethylsilyl cyanide solution in toluene (1.55 equiv, 0.32 w/w equiv) was added to the reactor while maintaining a temperature of 20±5° C. Batch was cooled to −20±5° C. Boron trifluoride diethyl etherate (1.00 equiv, 0.30 w/w equiv) was added over no less than 1.0 h while maintaining the temperature at −20±5° C. Batch was stirred at −20±5° C. for no less than 1.0 h and no more than 1.5 h. Acctonc (1.00 equiv, 0.122 w/w equiv) was charged to quench the reaction while maintaining the temperature at −20±5° C. The batch was heated back to 10±5° C. and 10 vol of 5.9% w/w aqueous potassium hydroxide solution was added while the temperature was allowed to rise to 20±5° C. The biphasic mixture was stirred for no less than 2.0 h, then the phases were allowed to settle, and the bottom aqueous layer was drained off. 5.0 vol of 11.3% w/w aqueous potassium hydroxide solution was added to the batch, followed by ethanol (2.0 vol, 1.58 w/w equiv), and the biphasic mixture was stirred for no less than 2.0 h. Phases were allowed to settle, and the bottom aqueous layer was drained off. 5.0 vol of 11.3% w/w aqueous potassium hydroxide solution was added to the batch, followed by ethanol (2.0 vol, 1.58 w/w equiv), and the biphasic mixture was stirred for no less than 2.0 h. Phases were allowed to settle, and the bottom aqueous layer was drained off. The resulting organic layer was filtered through celite before being concentrated down to a total of 4.0 vol while maintaining internal temperature at or below 45° C. Ethanol (7.0 vol, 5.52 w/w equiv) was charged and the mixture was distilled to a total of 4.0 vol. Ethanol (7.0 vol, 5.52 w/w equiv) was charged and the mixture was distilled to a total of 4.0 vol. Distillation continued until residual toluene was no more than 5.0% w/w. The proton NMR of the crude product was consistent with compound IX.

¹H-NMR CDCl₃: δ 6.91-6.85 (m, 1H); 6.78-6.73 (m, 1H); 5.02 (d, 1H, J=9.0 Hz); 4.22 (t, 1H, J=8.6 Hz); 4.06 (d, 3H, J=3.1 Hz); 2.84 (p, 1 H, J=7.7 Hz); 1.64 (d, 3H, J=1.3 Hz); 0.80 (dq, 3 H, J=7.3, 2.3 Hz) ppm.

### Step 6: Synthesis of (2R,3S,4S,5R)-3-(3,4-difluoro-2-methoxyphenyl)-4,5-dimethyl-5-(trifluoromethyl)tetrahydrofuran-2-carboxylic acid (IV)

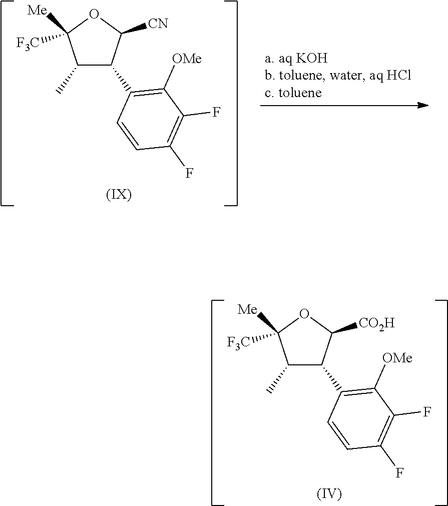

### Procedure 1:

Compound IX (333.06 g, 993.4 mmol) was dissolved in 2.60 L methanol and 2.60 L of 2.0M potassium hydroxide in a 25 L jacketed reactor under N₂ gas. The mixture was stirred at 55° C. for 20 hours. After overnight the slurry was a clear solution. Proton NMR indicated that the reaction complete. The reaction mixture was cooled to +15° C., and added 2925 ml of 2N HCl+5 L MTBE. After stirring for about 5 minutes, the layers were separated, and the aqueous was re-extracted with 5 L of MTBE. The MTBE solutions were combined and dried over magnesium sulfate, filtered and washed with MTBE. The combined MTBE solution was concentrated to an oil and re-concentrated with 4 L toluene, to give 360 g of an oily product, compound IV.

### Procedure 2:

To compound IX in 4 vol ethanol (245.3 kg, 168.3 mol, 1.00 equiv.) at 20° C., was charged a 10% w/w aqueous solution of KOH (192.8 kg, 2.4 vol, 2.38 equiv KOH). The reaction mixture was heated to 55° C. and stirred or 12 h. Upon completion of the reaction, the mixture was cooled down to 20° C. and toluene (242.8 kg, 3.5 vol) and water (400.0 kg, 5.0 vol) were charged. After 30 min of stirring, the phases were separated, and the organic phase was discarded. The aqueous phase was extracted with toluene (381.5 kg, 5.5 vol) and 7% HCl solution (537.2 kg, 6.5 vol). After 60 min of stirring the phases were separated and the aqueous phase was discarded. The organic phase was washed with water (400.0 kg, 5.0 vol), twice. The organic phase was distilled down to 4.0 volumes under vacuum at an internal temperature below 40° C. Toluene (277.4 kg, 4.0 vol) was charged and the solution was distilled down to 4.0 volumes under vacuum at an internal temperature below 40° C., to afford compound IV. The proton NMR of the crude compound was consistent with compound IV.

¹H-NMR d6-DMSO: δ 13.00 (s, 1H); 7.27-7.08 (m, 2H); 4.98 (d, 1H, J=10.5 Hz); 4.08 (dd, 1H, J=10.5, 7.6 Hz); 3.93 (d, 3H, J=2.1 Hz); 2.66 (p, 1 H, J=7.5 Hz); 1.53 (d, 3H, J=1.4 Hz); 0.73-0.64 (m, 3H) ppm.

### Step 7: Synthesis of (R)-(+)-α-methylbenzylamine salt of (2R,3S,4S,5R)-3-(3,4-difluoro-2-methoxyphenyl)-4,5-dimethyl-5-(trifluoromethyl)tetrahydrofuran-2-carboxylic acid (IV)

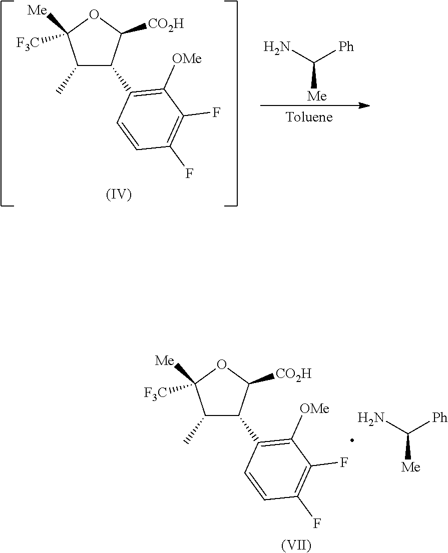

### Procedure 1:

Compound IV (2 g) was dissolved in 10 mL of toluene at 60° C. Separately, (R)-AMB (1.2 eq.) was dissolved in 2 mL of toluene and added to Compound IV solution over 1 hour. The resulting solution was then cooled to 50° C. over 1 hour and held for 1 hour for the self-nucleation to happen. Then the slurry was cooled to 20° C. over 5 hours and agitated at 20° C. for about 8 hours and filtered under vacuum. The resulting wet cake was washed with a 5 mL of toluene and dried under vacuum at 40° C. to provide about 70% yield compared to input Compound IV free form. The resulting solid was identified as (R)-(+)-α-methylbenzylamine salt of Compound IV Form A from XRPD, i.e. Form A of Compound VII.

Form A of Compound VII presents the following XRPD peaks in a standard X-Ray Powder Diffraction experiment.

TABLE 1

| No. | Pos. [°20] | Rel. Int. [%] |
|---|---|---|
| 1 | 10.32 | 28.04 |
| 2 | 11.78 | 11.26 |
| 3 | 12.81 | 15.58 |
| 4 | 13.44 | 57.18 |
| 5 | 14.01 | 5.72 |
| 6 | 14.76 | 6.18 |
| 7 | 15.91 | 18.58 |
| 8 | 17.00 | 5.45 |
| 9 | 17.45 | 100 |
| 10 | 18.25 | 36.72 |
| 11 | 18.53 | 11.02 |
| 12 | 18.95 | 63.98 |
| 13 | 21.96 | 11.86 |
| 14 | 24.07 | 24.65 |
| 15 | 24.36 | 8.98 |
| 16 | 24.62 | 7.83 |
| 17 | 24.69 | 7.32 |
| 18 | 25.09 | 8.2 |
| 19 | 29.69 | 7.8 |

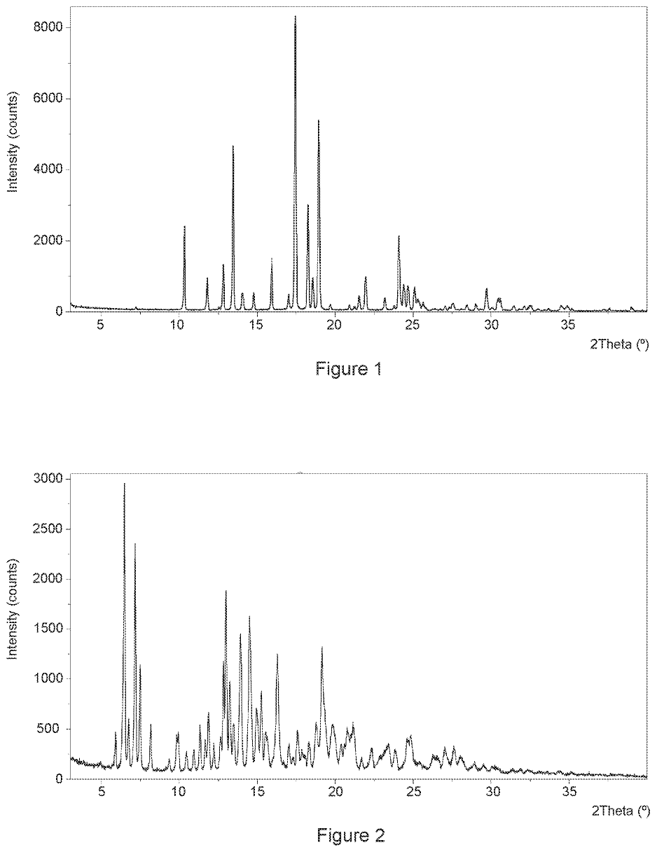

The skilled artisan would understand that the relative intensities of diffraction peaks can be affected by the experimental conditions. The overall diffraction pattern as shown in FIG. 1 may also be equally important for characterizing a solid form.

### Procedure 2:

The previously prepared solution of compound IV in 4.0 vol toluene (445.7 kg, 250.3 mol, 1.00 equiv) was charged to a reactor and the temperature was adjusted to 20±5° C. Separately, (R)-α-methylbenzylamine ((R)-AMB, 36.4 kg, 300.4 mol, 1.2 equiv.) was dissolved in toluene (51.6 kg, 0.50 vol). 40 vol % of the previously prepared solution was charged to the compound IV solution over 1 hour maintaining the temperature at 20±5° C., then the solution was stirred for no less than 60 minutes. Compound IV seeds (0.30 kg, 0.0025 w/w equiv.) were charged to the solution, and the slurry was stirred for no less than 1 hour. The remaining 60 vol % of the (R)-AMB solution was then charged to the slurry over no less than 1 hour, maintaining the temperature at 20±5° C. Then the slurry was aged at 20±5° C. for no less than 8 hours and filtered. The resulting wet cake was washed with toluene (206.3 kg, 2.0 vol) and dried under vacuum at 40° C. The resulting solid was identified as (R)-(+)-α-methylbenzylamine salt of Compound IV Form A from XRPD, i.e. Form A of Compound VII. The total yield from Step 5 to Step 7 is 80%.

### Example 2—Alternative Preparation of Compound (IV)

Compound IV may alternatively be prepared by the process described below:

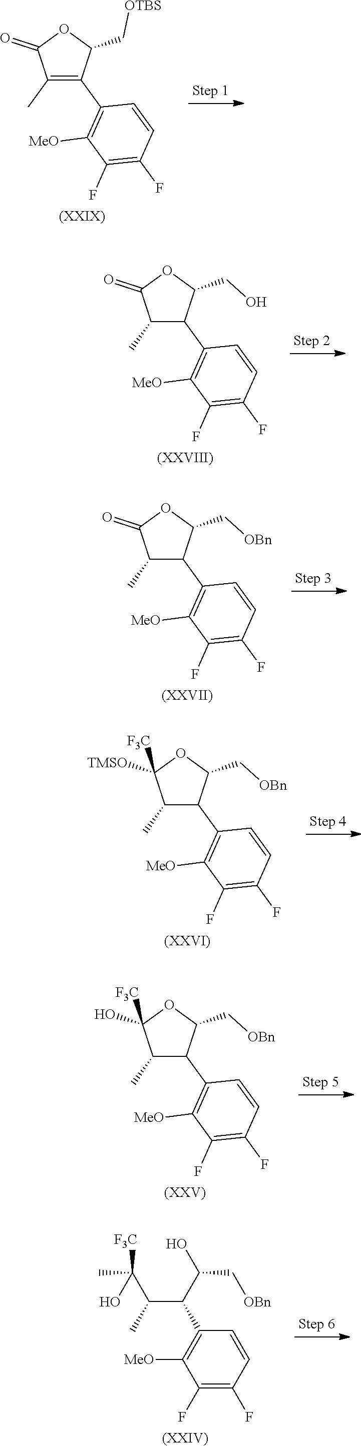

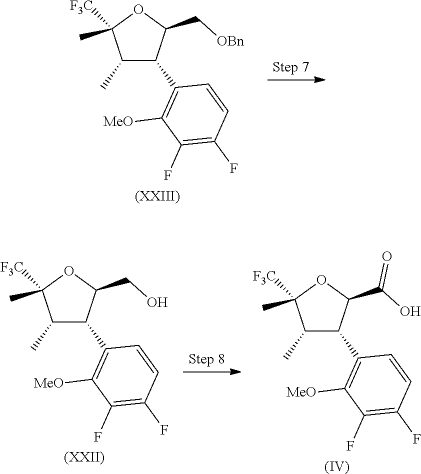

### Step 1: Synthesis of (3S,4S,5S)-4-(3,4-difluoro-2-methoxy-phenyl)-5-(hydroxymethyl)-3-methyl-tetrahydrofuran-2-one (XXVIII)

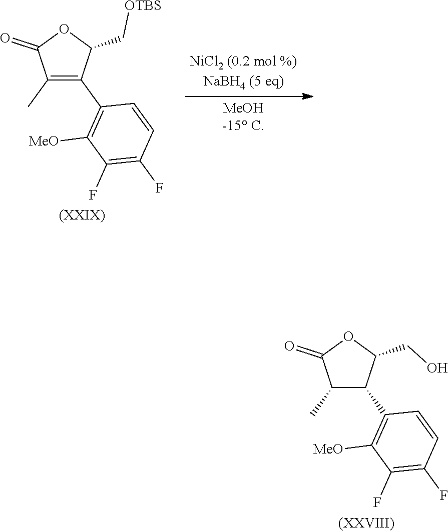

A solution of 5-(((tert-butyldimethylsilyl)oxy)methyl)-4-(3,4-difluoro-2-methoxyphenyl)-3-methylfuran-2(5H)-one XXIX (1.064 g, 3.937 mmol) and NiCl₂·6H₂O (112.0 mg, 0.4712 mmol) in methanol (40 mL) was cooled to −15° C. (NaCl/ice). Then NaBH₄ (730 mg, 19.30 mmol) was added portionwise over 10 mins and on complete addition was stirred for a further 20 mins. The reaction mixture was poured into cool NH₄Cl(sat) solution, the phases were separated and aqueous phase further extracted with DCM (×2). The combined organic phases were washed with water and brine. The crude product was purified by silica gel chromatography (eluting from 0% to 30% EtOAc in heptane) to afford (3S,4S,5S)-4-(3,4-difluoro-2-methoxy-phenyl)-5-(hydroxymethyl)-3-methyl-tetrahydrofuran-2-one XXVIII (501 mg, 47%).

¹H NMR (400 MHz, Chloroform-d) δ 6.81-6.69 (m, 1H), 6.64 (ddd, J=8.7, 5.6, 2.2 Hz, 1H), 4.74-4.62 (m, 1H), 3.91 (d, J=3.4 Hz, 3H), 3.52 (dd, J=12.1, 7.9 Hz, 1H), 3.28 (dd, J=12.1, 4.6 Hz, 1H), 3.14-2.92 (m, 1H), 1.84 (d, J=1.8 Hz, 1H), 0.93 (d, J=7.2 Hz, 3H) ppm.

ESI-MS m/z 273.4 (M+1)⁺; Retention time: 1.32 minutes.

### Step 2: Synthesis of (3S,4S,5S)-5-(benzyloxymethyl)-4-(3,4-difluoro-2-methoxy-phenyl)-3-methyl-tetrahydrofuran-2-one (XXVII)

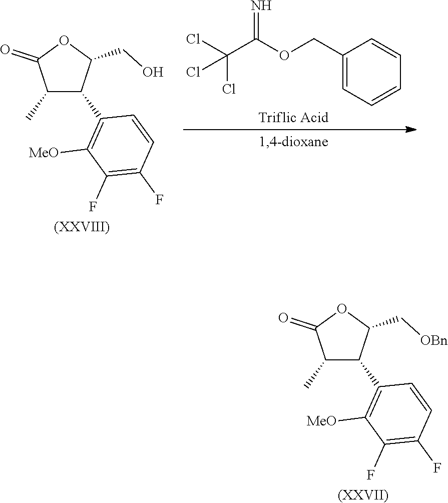

To a solution of (3S,4S,5S)-4-(3,4-difluoro-2-methoxy-phenyl)-5-(hydroxymethyl)-3-methyl-tetrahydrofuran-2-one XXVIII (17.7 g, 65.02 mmol) and benzyl 2,2,2-trichloroethanimidate (34 g, 134.6 mmol) in 1,4-dioxane (200 mL), trifluoromethanesulfonic acid (1 mL, 11.30 mmol) was added dropwise at room temperature (a mild exotherm develops even with slow addition, it reaches 28° C.) and the reaction cooled with an ice bath to keep the addition going. The mixture was stirred overnight at room temperature. The solution was diluted with TBME and quenched by addition of NaOH 1M. The phases were separated and the organic phase was washed twice more with NaOH 2M. The combined organic extracts were washed with water, brine, dried over MgSO₄, filtered and concentrated in vacuo affording crude product. The crude product was purified by silica gel chromatography (220 g column. Gradient: 0 to 20% EtOAc in heptane) to afford impure product. Product was dissolved in DCM and washed twice with NaOH 2 M, then water, dried (MgSO₄), filtered and concentrated in vacuo to provide (3S,4S,5S)-5-(benzyloxymethyl)-4-(3,4-difluoro-2-methoxy-phenyl)-3-methyl-tetrahydrofuran-2-one XXVII (18.7 g, 79%).

¹H NMR (400 MHz, Chloroform-d) δ 7.34-7.27 (m, 1H), 7.31-7.17 (m, 2H), 7.21-7.13 (m, 2H), 6.79 (td, J=9.1, 7.2 Hz, 1H), 6.70 (ddd, J=8.5, 5.7, 2.1 Hz, 1H), 4.87-4.78 (m, 1H), 4.44 (d, J=11.8 Hz, 1H), 4.28 (d, J=11.9 Hz, 1H), 3.89 (d, J=3.2 Hz, 3H), 3.51 (dd, J=10.3, 6.8 Hz, 1H), 3.25 (s, 1H), 3.04 (s, 1H), 0.97 (d, J=7.1 Hz, 3H) ppm.

ESI-MS m/z 363.4 (M+1)⁺; Retention time: 0.95 minutes.

### Steps 3 and 4: Synthesis of (2S,3S,4S,5S)-5-(benzyloxymethyl)-4-(3,4-difluoro-2-methoxy-phenyl)-3-methyl-2-(trifluoromethyl)tetrahydrofuran-2-ol (XXV)

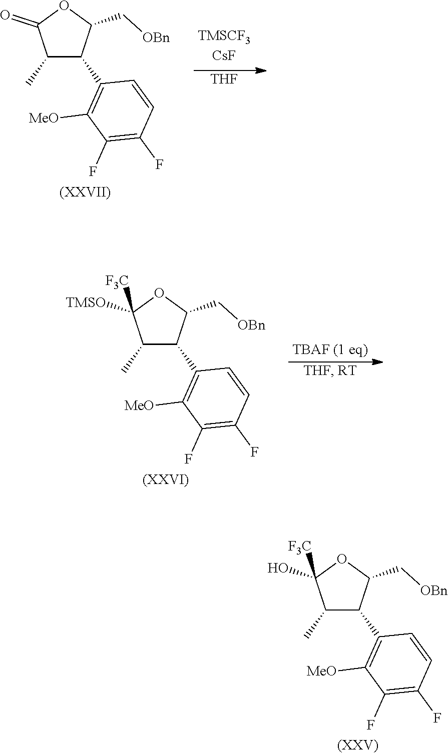

(3S,4S,5S)-5-(benzyloxymethyl)-4-(3,4-difluoro-2-methoxy-phenyl)-3-methyl-tetrahydrofuran-2-one XXVII (948 mg, 2.616 mmol) was loaded into a round-bottomed flask and backfilled with N₂ gas/vacuum 3 times. Trimethyl(trifluoromethyl) silane (1.7 mL, 11.50 mmol) was added using a syringe. THF (0.4 mL) was added followed by anhydrous CsF (97.5 mg, 0.6419 mmol) (oven dried). The mixture was backfilled with N₂ gas/vacuum three times. The resulting mixture was stirred at room temperature over the weekend to produce the compound XXVI. Additional THF (9.5 mL) was added followed by TBAF (2.6 mL of 1 M, 2.600 mmol) at room temperature and stirred for 10 minutes. The crude mixture was dissolved in DCM and wash with water (twice) and brine. The solvent was removed in vacuo to leave crude (2S,3S,4S,5S)-5-(benzyloxymethyl)-4-(3,4-difluoro-2-methoxy-phenyl)-3-methyl-2-(trifluoromethyl)tetrahydrofuran-2-ol XXV (367 mg, 32%).

¹H NMR (500 MHz, Chloroform-d) δ 7.25-7.12 (m, 3H), 7.12-7.02 (m, 2H), 6.72 (dtt, J=11.1, 7.0, 3.8 Hz, 2H), 4.47 (qd, J=6.1, 2.3 Hz, 1H), 4.35 (d, J=11.9 Hz, 1H), 4.22-4.14 (m, 1H), 3.98 (dd, J=8.5, 6.0 Hz, 1H), 3.76 (t, J=1.6 Hz, 3H), 3.46-3.35 (m, 1H), 3.20 (ddd, J=10.1, 5.8, 2.1 Hz, 1H), 2.95 (p, J=7.5 Hz, 1H), 1.27-1.11 (m, 3H), 0.81 (td, J=7.0, 2.1 Hz, 1H), 0.75 (dd, J=7.2, 1.7 Hz, 3H) ppm.

ESI-MS m/z 431.5 (M−1)⁻; Retention time: 1.01 minutes.

### Step 5: Synthesis of (2S,3S,4S,5S)-6-benzyloxy-4-(3,4-difluoro-2-methoxy-phenyl)-1,1,1-trifluoro-2,3-dimethyl-hexane-2,5-diol (XXIV)

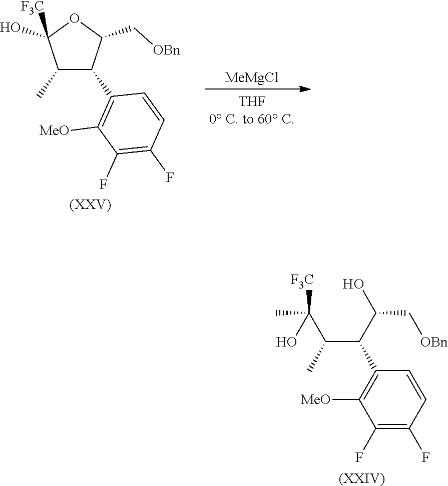

(2S,3S,4S,5S)-5-(benzyloxymethyl)-4-(3,4-difluoro-2-methoxy-phenyl)-3-methyl-2-(trifluoromethyl)tetrahydrofuran-2-ol XXV (415 mg, 0.9598 mmol) was dissolved in THF (8.5 mL) and cooled to 0° C. before adding MeMgCl (1.6 mL of 3 M, 4.800 mmol) dropwise. The reaction mixture was allowed to reach room temperature and then heated in the sealed vial to 60° C. until the reaction was complete. The reaction was allowed to cool to room temperature and quenched by addition of HCl 2M, extracted with EtOAc, dried over MgSO₄, filtered and concentrated in vacuo. The crude product was purified by silica gel chromatography (12 g column. Gradient: 0 to 20% EtOAc in heptane) to afford desired product, (2R,3S,4S,5S)-6-benzyloxy-4-(3,4-difluoro-2-methoxy-phenyl)-1,1,1-trifluoro-2,3-dimethyl-hexane-2,5-diol XXIV (300 mg, 70%).

¹H NMR (400 MHz, Chloroform-d) δ 7.30-7.12 (m, 7H), 6.74 (td, J=9.3, 7.5 Hz, 1H), 4.33 (d, J=3.2 Hz, 2H), 4.30-4.21 (m, 1H), 3.88 (d, J=2.7 Hz, 3H), 3.47 (dd, J=7.3, 2.8 Hz, 1H), 3.29 (dd, J=9.3, 3.7 Hz, 1H), 2.91 (t, J=8.9 Hz, 1H), 2.42 (s, 1H), 2.37-2.25 (m, 1H), 2.19 (q, J=6.1, 5.1 Hz, 1H), 1.28 (d, J=1.2 Hz, 3H), 1.24 (dq, J=7.3, 1.8 Hz, 3H) ppm.

ESI-MS m/z 447.5 (M+1)⁺; Retention time: 0.96 minutes.

### Step 6: Synthesis of (2R,3S,4S,5R)-5-(benzyloxymethyl)-4-(3,4-difluoro-2-methoxy-phenyl)-2,3-dimethyl-2-(trifluoromethyl)tetrahydrofuran (XXIII)

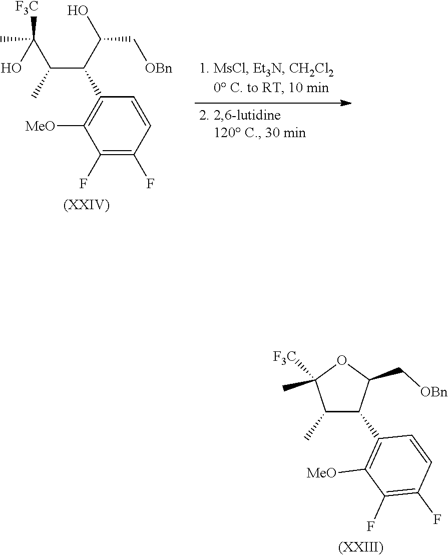

(2R,3S,4S,5S)-6-benzyloxy-4-(3,4-difluoro-2-methoxy-phenyl)-1,1,1-trifluoro-2,3-dimethyl-hexane-2,5-diol XXIV (218 mg, 0.4861 mmol) and Et₃N (0.34 mL, 2.439 mmol) were dissolved in DCM (2.5 mL) and cooled to 0° C. using an ice/water bath. Then mesyl chloride (0.12 mL, 1.521 mmol) was added dropwise and the reaction stirred at 0° C. until reaction deemed complete. The reaction was quenched by the addition of MeOH (1.5 mL). The cold mixture was allowed to warm to room temperature and water (20 mL) was added. The layers were separated and the aqueous layer was extracted with DCM (2×15 mL). The combined organic phases were dried over Na₂SO₄, filtered and concentrated under reduced pressure to give the crude mesylate intermediate as yellow oil, 264 mg. The crude mesylated product was diluted in 2,6-lutidine (5 mL) and heated at 120° C. for 1 hour. The mixture was diluted with water (20 mL) and DCM (20 mL). The layers were separated and the aqueous layer was extracted with DCM (3×20 mL). The combined organic phases were washed with saturated CuSO₄ aq. (20 mL×4), dried over Na₂SO₄, filtered and concentrated under reduced pressure and the crude product purified by silica gel chromatography using a gradient of 0 to 10% EtOAc in heptane then 20% EtOAc:80% heptane to provide (2R,3S,4S,5R)-5-(benzyloxymethyl)-4-(3,4-difluoro-2-methoxy-phenyl)-2,3-dimethyl-2-(trifluoromethyl)tetrahydrofuran XXIII (100 mg, 48%).

¹H NMR (500 MHz, Chloroform-d) δ 7.28-7.11 (m, 5H), 6.76-6.56 (m, 2H), 4.61-4.38 (m, 3H), 3.90-3.75 (m, 4H), 3.57 (ddd, J=11.0, 2.6, 1.4 Hz, 1H), 3.42 (ddd, J=11.1, 4.7, 1.4 Hz, 1H), 2.52 (pd, J=7.7, 1.4 Hz, 1H), 1.48-1.41 (m, 3H), 0.67 (dt, J=7.2, 2.2 Hz, 3H) ppm.

ESI-MS m/z 431.5 (M+1)⁺; Retention time: 1.21 minutes.

### Step 7: Synthesis of [(2R,3S,4S,5R)-3-(3,4-difluoro-2-methoxy-phenyl)-4,5-dimethyl-5-(trifluoromethyl)tetrahydrofuran-2-yl]methanol (XXII)

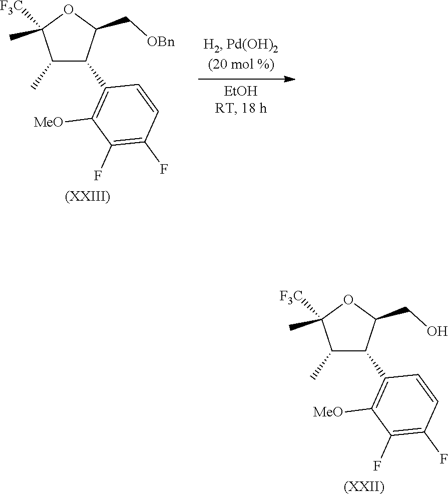

Pd(OH)₂ (Degussa, 23.7 mg, 0.03375 mmol) was loaded into a N₂ gas filled round bottomed flask. The flask was backfilled with N₂ gas three times. Then (2R,3S,4S,5R)-5-(benzyloxymethyl)-4-(3,4-difluoro-2-methoxy-phenyl)-2,3-dimethyl-2-(trifluoromethyl)tetrahydrofuran XXIII (76 mg, 0.1766 mmol) in EtOH (2 mL) was added and again the flask was backfilled with N₂ gas (×3). A balloon of Hydrogen was bubbled through the resulting solution until empty. The balloon was refilled and the mixture vigorously stirred overnight at room temperature. The reaction flask was flushed with N₂ gas three times (vacuum/N₂ gas cycles). The catalyst was then filtered-off by passing the mixture through pre-wetted celite cartridge with EtOH and solvent removed in vacuo to provide [(2R,3S,4S,5R)-3-(3,4-difluoro-2-methoxy-phenyl)-4,5-dimethyl-5-(trifluoromethyl)tetrahydrofuran-2-yl]methanol XXII (68 mg, quantitative yield based on ~94% purity).

¹H NMR (400 MHz, Chloroform-d) δ 6.76 (td, J=9.2, 7.3 Hz, 1H), 6.67 (ddd, J=8.6, 5.6, 2.1 Hz, 1H), 4.46 (ddd, J=11.3, 4.0, 2.5 Hz, 1H), 3.92 (d, J=2.5 Hz, 3H), 3.89-3.70 (m, 2H), 3.41 (dd, J=12.3, 4.0 Hz, 1H), 2.56 (p, J=7.7 Hz, 1H), 2.12-1.83 (m, 1H), 1.46 (d, J=1.4 Hz, 3H), 0.69 (dq, J=7.4, 2.4 Hz, 3H) ppm.

ESI-MS m/z 358.5 (M+1)⁺; Retention time: 0.94 minutes.

### Step 8: Synthesis of (2R,3S,4S,5R)-3-(3,4-difluoro-2-methoxyphenyl)-4,5-dimethyl-5-(trifluoromethyl)tetrahydrofuran-2-carboxylic acid (IV)

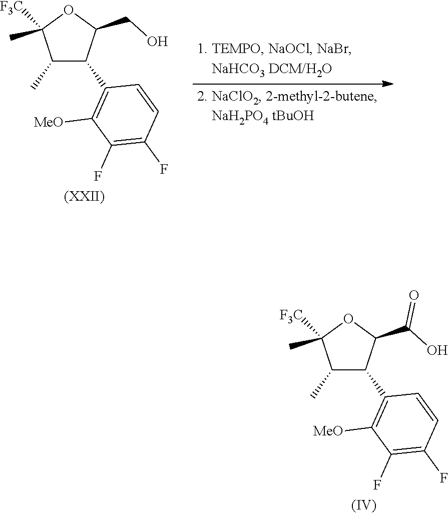

[(2R,3S,4S,5R)-3-(3,4-difluoro-2-methoxy-phenyl)-4,5-dimethyl-5-(trifluoromethyl)tetrahydrofuran-2-yl]methanol XXII (45 mg, 0.1322 mmol) was dissolved in CH₂Cl₂ (0.5 mL) and cooled to 0° C. NaBr (8.6 mg, 0.08358 mmol), tetrabutylammonium bromide (45.3 mg, 0.1405 mmol), TEMPO (7.7 mg, 0.04928 mmol) and NaHCO₃ aqueous sat. (0.4 mL) were then added. The resulting mixture was treated with NaOCl (9 μL, 0.1326 mmol) stirring vigorously and allowing to warm up to room temperature over 1 hour. HCl (0.13 mL of 1 M, 0.1300 mmol) was added until neutralizing pH to 6-7. Then tBuOH (2 mL), 2-methylbut-2-ene (0.5 mL of 2 M, 1.000 mmol) were added followed by an aqueous solution of NaClO₂ (12.6 mg, 0.1393 mmol) and NaH₂PO₄ (0.02 mL, 0.3192 mmol) and the mixture stirred at room temperature for 1-2 hours. The mixture was diluted with saturated aqueous NaH₂PO₄ solution (5 mL), and extracted with EtOAc (3×10 mL). The organic layers were combined and dried over MgSO₄, filtered and concentrated in vacuo to leave crude product. Product was purified by reverse-phase HPLC. Method: C18 Waters X-bridge column (19×150 mm, 5 micron), Gradient: MeCN in H₂O with 0.1% Ammonium hydroxide. 19 mL/min plus 1 mL/min MeCN at column dilution injection, Gradient 15.8% to 30.5% over 9 mins to afford (2R,3S,4S,5R)-3-(3,4-difluoro-2-methoxy-phenyl)-4,5-dimethyl-5-(trifluoromethyl)tetrahydrofuran-2-carboxylic acid IV (17 mg, 36%).

¹H NMR (400 MHz, Methanol-d₄) δ 7.11 (ddd, J=8.3, 5.6, 2.2 Hz, 1H), 6.96 (ddd, J=9.9, 8.9, 7.5 Hz, 1H), 4.96 (d, J=10.5 Hz, 1H), 4.16 (dd, J=10.5, 7.8 Hz, 1H), 3.99 (d, J=2.4 Hz, 3H), 2.73 (p. J=7.6 Hz, 1H), 1.59 (d, J=1.2 Hz, 3H), 0.86-0.68 (m, 3H) ppm.

ESI-MS m/z 353.4 (M−1)⁻; Retention time: 0.59 minutes.

### Example 3—Preparation of Compound (VI)

### Procedure 1:

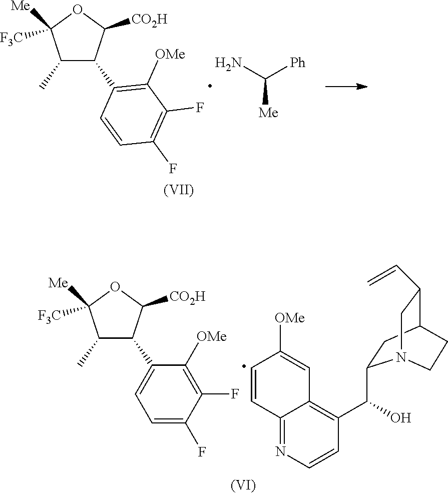

Isopropyl alcohol (27.8 L) was charged to a vessel and the solvent was saturated with N₂ gas. n-Heptane (47.2 L) was added to the vessel while continuously stirring the mixture and purging the vessel with N₂ gas. The n-heptane/isopropyl alcohol mixture was transferred to a clean drum. Compound VII, i.e. the (R)-(+)-α-Methylbenzylamine salt of Compound IV (10.42 Kg; product of Example 1, Step 7) was added to the solvent mixture while purging with N₂ gas. The reactor was sealed and three vacuum/N₂ gas cycles were conducted. Methylene chloride (80 L) was added to the reaction mixture before slowly starting the agitator to suspend and slurry the solids. Hydrochloric acid (full carboy of 2.0M) was added. Stirred the mixture for 15 minutes and let the phases separate (about 10 minutes). Compound IV was in the lower phase. Separated the phases and then returned the organic phase to the reactor. Repeated the wash procedure with two more portions (19 L each) of 2 M HCl. Dried the lower organic phase by stirring with anhydrous magnesium sulfate (1 kg) and then filtered off the supernatant. Distilled the mixture down to a volume of about 11 L, and then added isopropanol (10 L) to the reactor. Repeated the distillation and isopropanol addition two more times. Added quinine (7.11 kg) to the mixture. Diluted the remaining solution with a mixture (57 L total) of isopropanol and n-heptane and then heated the resulting mixture to about 60 to 65° C. to dissolve all solids. Cooled to 45° C. over 90 min. Added compound VI, i.e. the quinine salt of compound IV, seed material during the cooling period, when the mixture temperature was about 56-58° C. Cooled the mixture to 20° C. over 3 h. Stirred the mixture for 100 min and then filtered. Washed the filter cake with an isopropanol/n-heptane mixture (8 L) and then dried the solids at 75° C. The product was recrystallized from isopropanol/n-heptane to provide 8.7 kg of compound VI in 80% yield.

### Procedure 2:

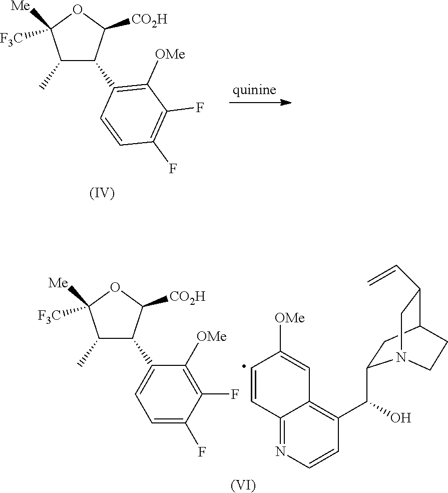

Quinine (1 eq.) was added to Compound IV (2 g; product of Example 1, Step 6) in about 4 mL of dichloromethane. The resulting slurry/solution was then solvent swapped to 2-propanol (4 mL) and heated to 70° C. to obtain complete dissolution. The solution was then cooled to 65° C. and n-heptane (12 mL) was added over one hour. During this heptane addition, self-nucleation was observed, and the resulting slurry was stirred at 65° C. for 1 hour. The slurry was then cooled to 20° C. over 3 hours and stirred at 20° C. for 2 hours. The slurry was filtered and washed with 3 mL of 75V % heptane in 2-propanol mixture. The wet solids were analyzed by XRPD and identified as the 2-propanol solvate of Compound IV quinine salt Form A (i.e., Compound VI). The wet cake was then dried at 50° C. for 24 hours to obtain Compound VI Form A in about 85% yield.

Form A of Compound VI may be characterized by the following XRPD peaks in a standard X-Ray Powder Diffraction experiment.

TABLE 2

| No. | Pos. [°2θ] | Rel. Int. [%] |
|---|---|---|
| 1 | 5.91 | 12.52 |
| 2 | 6.48 | 100 |
| 3 | 6.75 | 17.86 |
| 4 | 7.16 | 83.17 |
| 5 | 7.48 | 36.2 |
| 6 | 8.17 | 15.77 |
| 7 | 9.79 | 10.28 |
| 8 | 9.93 | 13.88 |
| 9 | 11.31 | 15.64 |
| 10 | 11.65 | 10.77 |
| 11 | 11.85 | 21.32 |
| 12 | 12.61 | 11.76 |
| 13 | 12.81 | 38.92 |
| 14 | 12.97 | 64.51 |
| 15 | 13.2 | 32.35 |
| 16 | 13.48 | 16.95 |
| 17 | 13.91 | 50.21 |
| 18 | 14.48 | 54.11 |
| 19 | 14.94 | 21.87 |
| 20 | 15.25 | 29.27 |
| 21 | 15.54 | 14.26 |
| 22 | 15.66 | 10.61 |
| 23 | 16.25 | 42.39 |
| 24 | 17.57 | 15.21 |
| 25 | 18.30 | 10.74 |
| 26 | 18.75 | 18.23 |
| 27 | 19.12 | 42.79 |
| 28 | 19.79 | 16.98 |
| 29 | 20.40 | 10.28 |
| 30 | 20.75 | 15.48 |
| 31 | 21.18 | 15.63 |
| 32 | 24.57 | 12.24 |
| 33 | 24.87 | 12.03 |

The skilled artisan would understand that the relative intensities of diffraction peaks can be affected by the experimental conditions. The overall diffraction pattern as shown in FIG. 2 may also be equally important for characterizing a solid form.

### Example 4—Preparation of Compound (V)

Compound V may be prepared by the process described below:

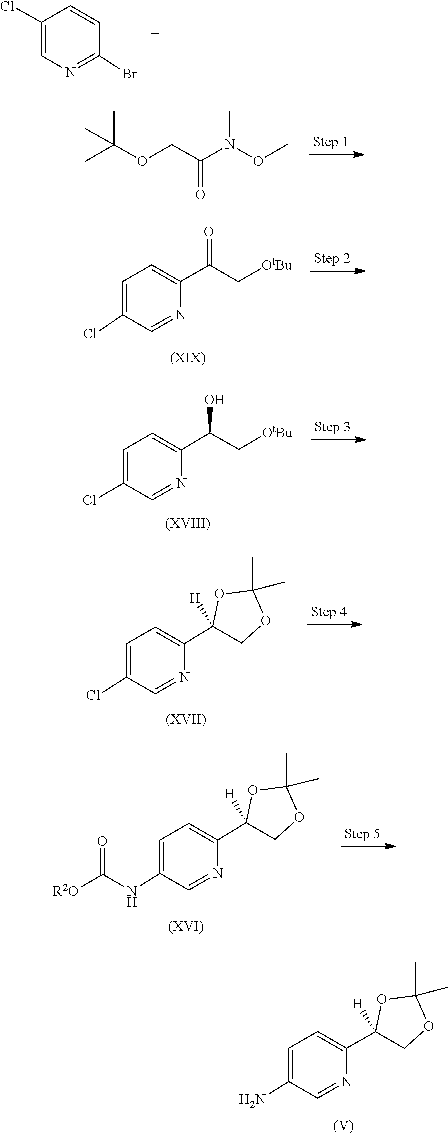

In some embodiments, R² is ethyl. In some embodiments, R² is tert-butyl.

### Step 1: Synthesis of 2-(tert-Butoxy)-1-(5-chloropyridin-2-yl)ethan-1-one (XIX)

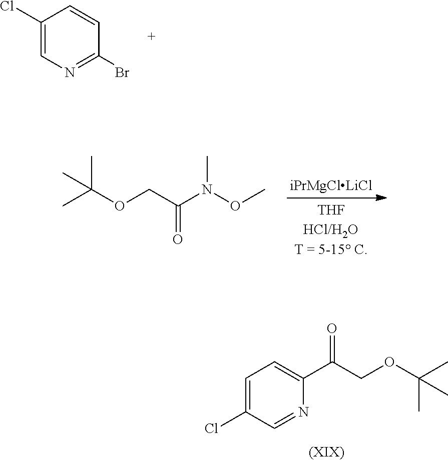

A solution of 2-bromo-5-chloropyridine (46.10 kg) and THF (100.00 kg) was prepared in a 500 L glass-lined reactor.

Under nitrogen protection, Isopropyl magnesium chloride lithium chloride complex tetrahydrofuran solution (180.00 kg) was added into a 500 L glass-lined reactor. The mixture was cooled to −5~10° C. The THF solution of 2-bromo-5-chloropyridine was added at 0~5° C. After complete activation, 2-(tert-butoxy)-N-methoxy-N-methylacetamide (35.00 kg) was charged to the reactor. The mixture was allowed to react at 10~20° C. After complete reaction, a mixture of purified water (320.00 kg) and [31%] Hydrochloric acid (42.00 kg), was charged keeping the temperature≤15° C. The mixture was warmed up to ambient temperature. n-Heptane (48.00 kg) was added, and the mixture was stirred for 0.5~1 h, settled and separated. The aqueous layer was back extracted with n-heptane (134.60 kg). The combined organic layers were washed with purified water (175.00 kg) followed by a wash with aqueous NaCl solution (sodium chloride (44.00 kg) dissolved in water (175.00 kg). The organic layer was concentrated under reduced pressure. n-Heptane (191.00 kg+191.50 kg) was added into the mixture concentrated under reduced pressure. The mixture was heated to 45~50° C. and stirred for 1~3 h. The mixture was cooled was cooled to −5~5° C. The product was isolated by filtration and dried under vacuum at <40° C. 2-(tert-Butoxy)-1-(5-chloropyridin-2-yl)ethan-1-one (XIX) was isolated as a light-yellow solid (35 kg, 77% yield).

¹H NMR (400 MHz, CDCl₃) δ 8.58 (d, J=2.4 Hz, 1H), 8.02 (d, J=8.4 Hz, 1H), 7.81 (dd, J=8.4, 2.4 Hz, 1H), 2.08 (s, 2H), 1.31 (s, 9H) ppm.

### Step 2: Synthesis of (R)-2-(tert-butoxy)-1-(5-chloropyridin-2-yl)ethan-1-ol (XVIII)

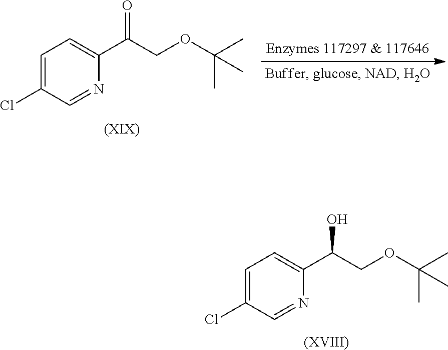

2-(tert-Butoxy)-1-(5-chloropyridin-2-yl)ethan-1-one (XIX, 33.40 kg) in methanol (78.00 kg) was prepared at 30~40° C. Purified water (871.00 kg), disodium phosphate dodecahydrate (50.00 kg) and sodium dihydrogenphosphate (7.80 kg) were charged into the 3000 L glass-lined reactor and stirred till a solution was obtained. D-Glucose monohydrate (87.40 kg) was added into the mixture to obtain a solution with a pH of 7.0~7.5. At 20~30° C. NAD (1.70 kg), Ketone reductase 117297 (51.02 kg) and Glucose dehydrogenase 117646 (33.62 kg) were charged to a 3000 L reactor. The methanol solution containing 2-(tert-butoxy)-1-(5-chloropyridin-2-yl)ethan-1-one (XIX) was added, and the mixture stirred at 25° C. at pH 7-7.5. After reaction completion, sodium chloride (200.00 kg) was added into the mixture. The pH was adjusted to 2~3 with hydrochloric acid (27.60 kg). Celite (32.50 kg) and MTBE (250.00 kg) were added into the mixture and stirred for 1-2 h. The mixture was filtered. MTBE (250.00 kg) was added to the filtrate and the pH adjusted to 7~8 with the sodium hydroxide solution (102.92 kg). The organic layer was separated, and the aqueous layer was back extracted with MTBE (250.00 kg). The filter cake was transferred into a reactor and stirred in MTBE (250.00 kg). The reactor content was filtered, and the filtrate combined with the organic layers. The filter cake was transferred into a reactor and stirred in MTBE (250.00 kg). The reactor content was filtered, and the filtrate combined with the organic layers. The combined organic layers were washed with a solution of sodium bicarbonate (17.00 kg) and purified water (318.00 kg). The organic layer was washed with a solution of sodium chloride (55.00 kg) and purified water (167.00 kg). The organic layer was concentrated under vacuum. n-Heptane (114.60 kg+114.6.00 kg) was added, and the mixture concentrated under vacuum. The mixture was heated to 45~50° C. and then cooled 0-10° C.

The product was isolated by filtration, the cake washed with n-Heptane (15.00 kg) and was dried under vacuum. (R)-2-(tert-butoxy)-1-(5-chloropyridin-2-yl)ethan-1-ol (XVIII) was isolated in 20.6 kg, 61.1% yield.

¹H NMR (400 MHz, CDCl₃) δ 8.49 (dd, J=2.4, 0.7 Hz, 1H), 7.67 (dd, J=8.4, 2.4 Hz, 1H), 7.47 (dt, J=8.5, 0.7 Hz, 1H), 4.82 (dd, J=7.0, 4.7 Hz, 1H), 3.63 (dd, J=9.0, 4.8 Hz, 1H), 3.55 (br s, 1H), 3.49 (dd, J=9.0, 7.0 Hz, 1H), 1.16 (s, 9H) ppm.

### Step 3: Synthesis of (R)-5-chloro-2-(2,2-dimethyl-1,3-dioxolan-4-yl)pyridine (XVII)

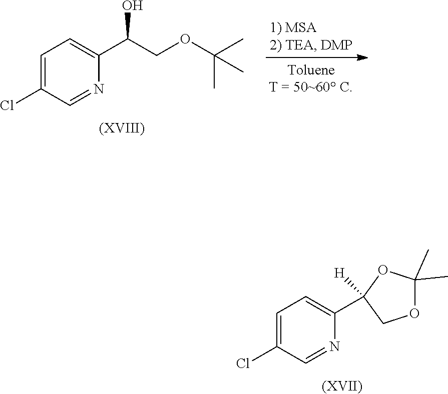

(R)-2-(tert-butoxy)-1-(5-chloropyridin-2-yl)ethan-1-ol (XVIII, 26.0 kg) and toluene (180.00 kg) were charged into the 1000 L glass-lined reactor 1. Methyl sulfonic acid (43.10 kg) was charged at T≤35° C. The mixture was heated to 50~60° C. After reaction completion, the reaction was cooled to 20° C. Triethylamine (40.60 kg) was added to adjust the pH to 5.0~6.5. 2,2-Dimethoxypropane (93.40 kg) was added into the mixture at 0-20° C. The mixture was heated to 45~55° C. (After reaction completion, the reaction was cooled to 0-10° C. and an aqueous solution of sodium bicarbonate (NaHCO₃ (11.30 kg) in purified water (144.20 kg) to adjust the pH to 7-8. The organic layer was separated and washed with an aqueous sodium chloride solution (sodium chloride) (15.40 kg) in purified water (62.20 kg). The organic layer was concentrated under vacuum. Toluene (54.00 kg) was added into the mixture. The mixture was concentrated under reduced pressure. The mixture was cooled to 15-25° C. (R)-5-chloro-2-(2,2-dimethyl-1,3-dioxolan-4-yl)pyridine (XVII) was isolated as a yellow toluene solution (142.4 kg in Toluene, assay: 11.89%)

¹H NMR (400 MHz, DMSO-d6) δ 8.58 (dd, J=2.5, 0.7 Hz, 1H), 7.95 (dd, J=8.4, 2.5 Hz, 1H), 7.52 (dt, J=8.4, 0.7 Hz, 1H), 5.12 (t, J=6.6 Hz, 1H), 4.37 (dd, J=8.3, 6.8 Hz, 1H), 3.86 (dd, J=8.3, 6.4 Hz, 1H), 1.42 (dd, J=11.5, 0.7 Hz, 6H) ppm.

### Step 4: Synthesis of ethyl (R)-(6-(2,2-dimethyl-1,3-dioxolan-4-yl)pyridin-3-yl)carbamate (XVI-A)

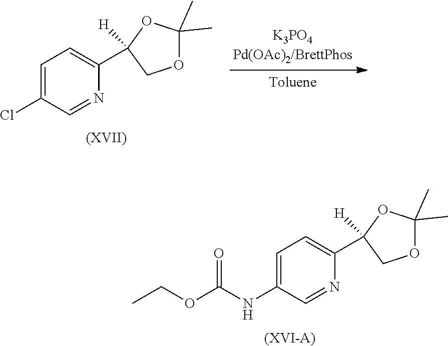

(R)-5-chloro-2-(2,2-dimethyl-1,3-dioxolan-4-yl)pyridine (XVII, 29.41 kg) was charged to a reactor. Toluene (217 kg, 10 vol) was charged, and the solution cooled to 10° C. Ethyl carbamate (16.50 kg) and anhydrous potassium phosphate (44.10 kg) were added. The reactor content was deoxygenated and palladium acetate (0.466 kg) and 2-(dicyclohexylphosphine)-3,6-dimethoxy-2′,4′,6′, tri-i-propyl-1,1′-biphenyl (brettphos) (2.24 kg) were added to the reactor. The reactor was deoxygenated and heated the mixture to 110° C. After reaction completion, the reaction was cooled and filtered through diatomaceous earth. The filter was rinsed with toluene (310.00 kg). The organic solution was treated with water (295.50 kg). The layers were separated, and the organic layer washed with an aqueous sodium chloride solution (74.00 kg sodium chloride dissolved in 295.00 kg water). The organic layer was concentrated to approximately 295.00~354.00 L. The concentrate was treated with silica gel (11.80 kg). The silica gel was filtered off and rinsed with toluene (290.0 kg). The filtrate was concentrated to approximately 250~300 L. n-Heptane (123.00 kg) was added and the reactor content cooled to 0-5° C. The product was filtered, and the cake washed with n-Heptane (31.50 kg). The material was dried under vacuum at <30° C. Ethyl (R)-(6-(2,2-dimethyl-1,3-dioxolan-4-yl)pyridin-3-yl)carbamate (XVI-A) was isolated as a white solid (32.1 kg, 55.7% yield).

¹H NMR (400 MHz, CDCl₃) δ 8.51 (d, J=2.6 Hz, 1H), 8.04 (s, 1H), 7.51 (d, J=8.6 Hz, 1H), 7.06 (s, 1H), 5.21 (t, J=6.7 Hz, 1H), 4.44 (dd, J=8.3, 6.7 Hz, 1H), 4.24 (q, J=7.1 Hz, 2H), 3.93 (dd, J=8.4, 6.7 Hz, 1H), 1.55-1.46 (m, 6H), 1.31 (t, J=7.1 Hz, 3H) ppm.

### Step 4′: Synthesis of tert-butyl (R)-(6-(2,2-dimethyl-1,3-dioxolan-4-yl)pyridin-3-yl)carbamate (XVI-B)

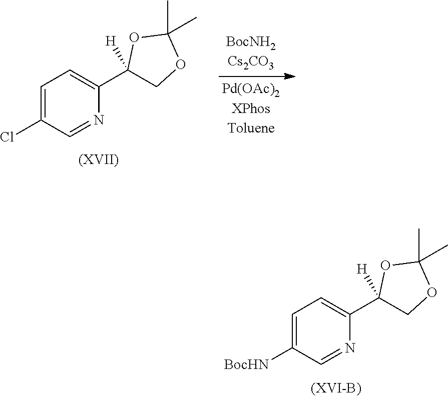

The reaction was performed in the 50 L reactor. The reactor was charged with Compound (XVII) (2,074 g, 9.707 mol), toluene (24 L), Cs₂CO₃ (3,529.3 g, 10.832 mol, 1.116 equiv.), tert-butyl carbamate (1,833.2 g, 15.648 mol, 1.612 equiv.) and XPhos (278.8 g, 584.8 mmol, 0.0603 equiv.). The thermostat was set to 60° C. and after 2 cycles of vacuum/nitrogen back-filling, about 2 L of solvent was distilled to remove any possible water present (took 20 min). To the stirred mixture, Pd(OAc)₂ (65.49 g, 291.7 mmol, 0.0301 equiv.) was added. Another cycle of vacuum/nitrogen back-filling was applied, and the thermostat was set to 112° C., at t=0 min. At t=255 min, the mixture was cooled to RT. To the mixture toluene (10 L) and water (30 L) were added. The mixture was stirred for 3-4 min and allowed to settle overnight. The clear phases were separated, and the turbid phase in between (about 2 L) was filtered over a pad of Celite (200 g). The aqueous phase was extracted with toluene (2×7 L). The combined organic phases were filtered over another pad of Celite (435 g) using an 8 L P2 glass filter (Ø=20 cm), took 1 h. The combined filtrates were washed with water (20 L), stirred for 2 min. The phases were allowed to settle overnight. After phase separation, the organic phase was washed once more with water (20 L) and stirred for 2 min. After phase separation, the organic phase was concentrated under reduced pressure (65-70° C., 26 mbar). tert-Butyl (R)-(6-(2,2-dimethyl-1,3-dioxolan-4-yl)pyridin-3-yl)carbamate (XVI-B) was isolated (3,341 g). ¹H-NMR showed 7.3 wt % XPhos, 9.6 wt % tert-butyl carbamate and 1.8 wt % toluene present. Calculated yield: 2,716 g (95.0%).

¹H NMR (400 MHz, CDCl₃) δ 8.38 (d, J=2.4 Hz, 1H), 7.94 (d, J=7.6 Hz, 1H), 7.44 (d, J=8.8 Hz, 1H), 6.95 (s, 1H), 5.15 (t, J=6.5 Hz, 1H), 4.40 (t, J=7.6 Hz, 1H), 3.92 (t, J=7.6 Hz, 1H), 1.51 (s, 9H) ppm.

### Step 5: Synthesis of (R)-6-(2,2-dimethyl-1,3-dioxolan-4-yl)pyridin-3-amine (V)

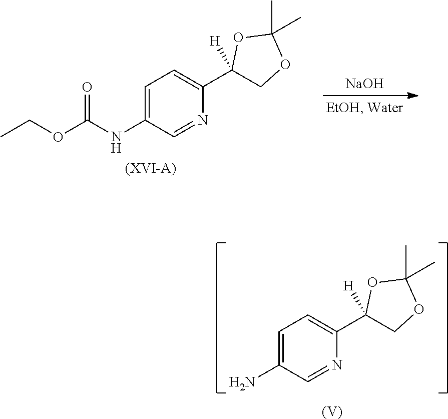

Charge ethyl (R)-(6-(2,2-dimethyl-1,3-dioxolan-4-yl)pyridin-3-yl)carbamate (XVI) (1.00 equiv., 100 g) to a reactor containing a mixture of EtOH (250 ml, 2.5 vol) and aqueous [2M] NaOH (37.6 g, 4.7 vol). Stir the contents at 50° C. for NLT 18 h. After reaction completion, concentrate to ~65% of original weight. Cool the contents to <10° C. and charge aqueous [6M] HCl to adjust the pH to 8-9. Adjust the temperature to 20-25° C. and charge aqueous [20% w/w] NaCl solution (200 ml, 2 vol). Charge EtOAc (500 ml, 5 vol), and stir for 15 minutes. Separate the layers and back extract the aqueous layer with EtOAc (500 ml, 5 vol). Combine the organic layers and concentrate the organics layer. Charge EtOAc (300 ml, 3 vol) and concentrate to ~300 ml (3 vols). Dilute the content with EtOAc (200 ml, 2 vol) to reactor. (R)-6-(2,2-dimethyl-1,3-dioxolan-4-yl)pyridin-3-amine (V) is stored as an ethyl acetate solution. HPLC retention time confirmed that the desired compound was formed.

¹H NMR (400 MHz, DMSO; isolated Compound V) δ 7.89 (dd, J=2.8, 0.7 Hz, 1H), 7.13 (d, J=8.3 Hz, 1H), 6.94 (dd, J=8.3, 2.7 Hz, 1H), 5.25 (s, 2H), 5.00-4.88 (m, 1H), 4.22 (dd, J=8.0, 6.4 Hz, 1H), 3.80 (dd, J=8.0, 7.4 Hz, 1H), 1.41 (d, J=0.7 Hz, 3H), 1.37 (d, J=0.7 Hz, 3H) ppm.

### Step 5′: Synthesis of (R)-6-(2,2-dimethyl-1,3-dioxolan-4-yl)pyridin-3-amine (V)

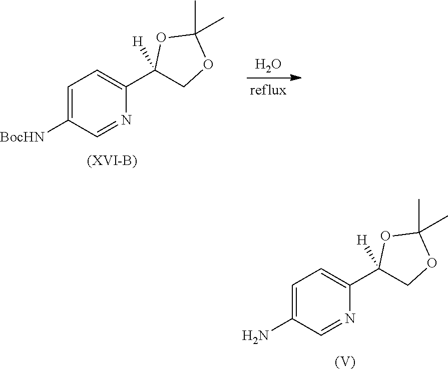

The reactor was loaded with tert-butyl (R)-(6-(2,2-dimethyl-1,3-dioxolan-4-yl)pyridin-3-yl)carbamate (XVI-B) (3,330 g, taken as 81.5% pure, 9.220 mol) and DME (16 L, 4.8 vol.). Celite (1235 g) was added to the brown solution. To the orange-brown suspension, 66° C. tap water (48 L, 14.4 vol.) was added, the internal temperature of the suspension was 59° C. The heating was set to 120° C., t=0. At t=1 h, reflux was reached (85.9° C. internal). The lower boiling DME was distilled off slowly and replaced by added hot tap water at about the same rate. After 24 hours, the heating was set to 130° C. and the mixture was distilled at 500 mbar. Then the temperature was set to −10° C. and under further reduced pressure (30 mbar) the mixture was cooled to 5° C. Citric acid (1,956.4 g, 10.183 mol, 1.104 equiv.) was added. The mixture was stirred for 35 min and filtered over wetted Celite. The filter cake was rinsed with water (4×2 L). The combined filtrates (~39 L) were washed with TBME (5 L). The aqueous phase was loaded into the reactor and made basic by adding NaHCO₃ (s) portion-wise. The mixture was heated to 25-30° C. and saturated with Na₂SO₄·10H₂O by adding Na₂SO₄ (s, 4.05 kg). TBME (24 L) was added, and the mixture was stirred vigorously for 20-30 min and then stirred slowly overnight. The stirring was halted, and the phases were allowed to settle. The organic phase was sucked from the aqueous phase. In this way the aqueous phase was extracted with TBME (24 L) twice more. The combined organic phases were dried over Na₂SO₄ and filtered. Removal of the solvent under reduced pressure (55° C., 10 mbar) yielded a thick amber oil which solidified upon cooling to ambient temperature. Yield: 1,700 g.

### Pd Removal and Crystallization:

1,655 g of the product was dissolved in TBME (5.5 L), SiliaMetS Thiol (Silicycle, 175 g, 1.32 mmol/g) was added and the mixture stirred was stirred overnight at RT. The solution was filtered over Celite. The filter cake was rinsed with TBME (1 L). The combined filtrates were filtered through 0.45 μm syringe filters. The filtered solution was transferred to a 20 L jacketed glass reactor (the container was rinsed with TBME (~300 mL)). Under reduced pressure TBME was distilled from the mixture leaving 3,300-3,400 mL (~2.0 vol) of TBME in the solution. The solution was cooled to 16° C. and slowly 12.5 L (~7.5 vol.) of n-heptane was added in a small stream. When ~1,300 mL of n-heptane was added, the solution was seeded with a few grains of authentic material. The suspension was stirred at 10° C. The suspension was filtered, and the filter was washed with a mixture of TBME/n-heptane (1:3.5, 2×1 L). The product (V) was dried further by suction overnight. Yield: 1,500.1 g (7.72 mol, 79.6%).

¹H NMR (400 MHz, DMSO-d6) δ 7.89 (dd, J=2.8, 0.7 Hz, 1H), 7.13 (d, J=8.3 Hz, 1H), 6.94 (dd, J=8.3, 2.7 Hz, 1H), 5.25 (s, 2H), 5.00-4.88 (m, 1H), 4.22 (dd, J=8.0, 6.4 Hz, 1H), 3.80 (dd, J=8.0, 7.4 Hz, 1H), 1.41 (d, J=0.7 Hz, 3H), 1.37 (d, J=0.7 Hz, 3H) ppm.

### Example 5—Preparation of Compound (III) from Compound (VII)

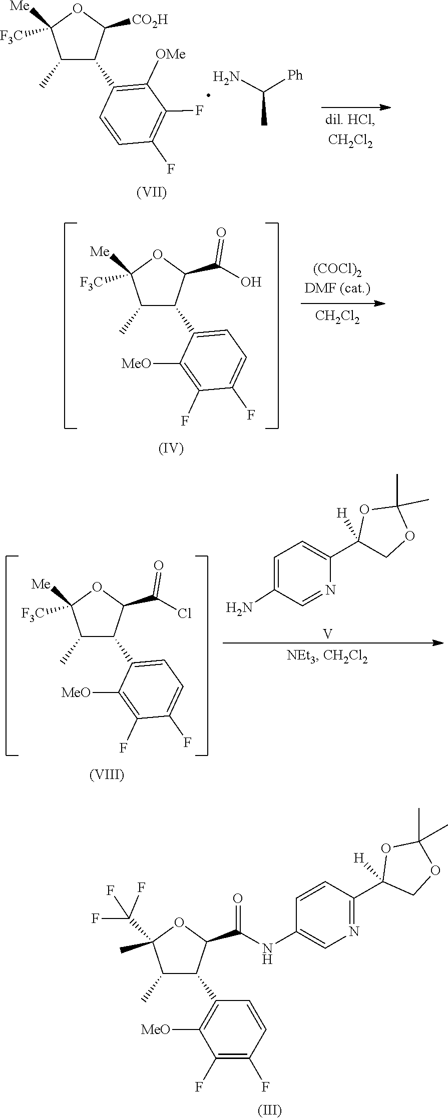

In a 300 mL jacketed reactor, Compound (VII) (16.56 g, 34.83 mmol) was suspended in dichloromethane (120 mL) and agitated at 200 rpm. The reactor contents were adjusted to 20° C. 2M HCl (35 mL, 70.00 mmol) was added over 5 min. The resulting biphase was stirred for 10 min. before halting stirring and allowing the phases to settle. The layers were separated, the lower organic layer returned to the reactor and the aqueous discarded. Stirring was restarted in the reactor. Further 2M HCl (35 mL, 70.00 mmol) was added over 5 min. The resulting biphase was stirred for 10 min. before halting stirring and allowing the phases to settle. The layers were separated, the lower organic layer returned to the reactor and the aqueous discarded. Stirring was restarted in the reactor. Further 2M HCl (35 mL, 70.00 mmol) was added over 5 min. The resulting biphase was stirred for 10 min. before halting stirring and allowing the phases to settle.

The layers were separated, and the lower organic layer was concentrated to approx. 50 mL at 350 mbar/40° C. Dichloromethane (100 mL) was added and the mixture concentrated again to give a volume of approx. 30 mL. The mixture was then diluted with dichloromethane to give a total volume of 80 mL (Compound (IV) dichloromethane solution).

In a clean and dry reactor, the Compound (IV) dichloromethane solution was stirred and the reactor contents adjusted to 20° C. N,N-Dimethylformamide (35 μL, 0.45 mmol) was added. Oxalyl chloride (4.7 mL, 53.88 mmol) was added over 30 mins. The reaction was stirred for a further 20 mins. HPLC analysis (acid chloride intermediate analyzed as its butylamine adduct employing a derivatization method) showed complete conversion. The reaction mixture was diluted with dichloromethane (50 mL) and concentrated under vacuum (350 mbar/40C) to <50 mL. Further distillation cycles were carried out by addition of dichloromethane (3×100 mL) and reducing the volume on each occasion to approx. 50 mL.

In a clean and dry reactor, Compound (V) (7.1 g, 36.55 mmol) was dissolved in dichloromethane (100 mL), agitated at 200 rpm, and treated with triethylamine (5.9 mL, 42.33 mmol). The reactor contents were adjusted to 10° C.

The acid chloride solution of Compound (VIII) was diluted with dichloromethane to 80 mL total volume then added over 1 h to the amine Compound (V) solution in the reactor, maintaining the contents at 10° C.

The reaction was stirred for 3.5 h then the contents warmed to 20° C., and stirred for a further 16 h.

The reaction was quenched by addition of water (65 mL) and the mixture stirred at 300 rpm for 20 mins.

Agitation was stopped and the phases allowed to separate. The organics were returned to the reactor and treated with a solution of citric acid monohydrate (7.1 g, 33.79 mmol) in water (65 mL). The mixture was stirred at 20° C./300 rpm for 1.5 h.

Agitation was stopped and the phases were separated. The organics were returned to the reactor and water (50 mL) was added. The mixture was stirred for 30 mins.

Agitation was stopped and the phases allowed to separate. The organic phase was concentrated to dryness under vacuum (40° C./300 mbar initially then 20 mbar) to give Compound (III) as a yellow/orange foam (16.4 g, 91% HPLC purity, 80.8% yield).

¹H NMR (400 MHz, DMSO-d6) δ 10.43 (s, 1H), 8.71 (dd, J=2.6, 0.7 Hz, 1H), 8.06 (dd, J=8.5, 2.5 Hz, 1H), 7.45 (d, J=8.5 Hz, 1H), 7.23-7.09 (m, 2H), 5.13-5.03 (m, 2H), 4.33 (dd, J=8.2, 6.7 Hz, 1H), 4.25 (dd, J=10.3, 7.6 Hz, 1H), 3.95 (d, J=2.1 Hz, 3H), 3.82 (dd, J=8.2, 6.7 Hz, 1H), 2.77 (p, J=7.5 Hz, 1H), 1.61 (s, 3H), 1.47-1.37 (m, 6H), 0.73 (dt, J=7.7, 2.5 Hz, 3H).

¹⁹F NMR (376 MHz, DMSO-d6) δ −73.36, −138.18 (dt, J=20.9, 7.3 Hz), −154.55-−155.25 (m).

### Example 6—Preparation of Compound (III) from Compound (VI)

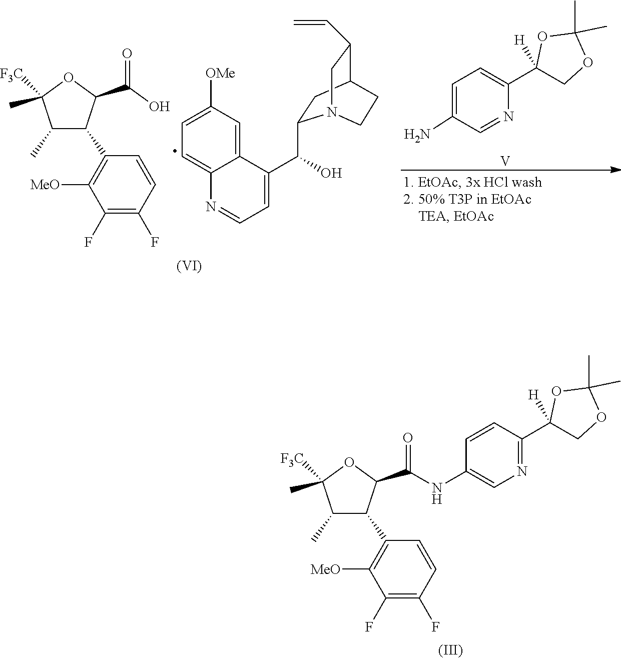

The quinine salt of compound IV (i.e. VI, 60 g, 1.00 eq.,) was charged to a reactor containing a mixture of EtOAc (480 ml, 8 vol) and [2N] HCl (180 ml, 3 vol). The mixture was stirred for 15 minutes and then allowed to settle. The aqueous was discarded and the organic layer was washed twice more with [2N] HCl (2×180 ml, 2×3 vol), and then with water (120 ml, 2 vol). The organic layer was then concentrated by distillation under vacuum down to ~120 ml, 2 volumes. EtOAc (360 ml, 6 vol) was added, and the mixture again distilled down to 120 ml, 2 volumes.

Compound V (18.03 g, 1.05 eq.) in EtOAc (300 ml, 5 vol) was treated with triethylamine (18.4 ml, 1.50 eq.). This solution was transferred into the free based solution of compound IV at a rate of 10 vol/h, while keepings contents temperature <25° C. Following completion of the addition, the mixture was stirred at 15° C. A [50% solution in EtOAc] T3P solution (78.1 ml, 1.50 eq.) was charged at a rate of 2.6 vol/h. Following completion of the addition, the mixture was stirred at 15° C. for over 1 hour.

After reaction completion, the reactor was cooled to 5° C. A solution of K₃PO₄ (41.28 g, 2.20 eq.) in water (456 ml,7.64 vol) was added to the contents, keeping temperature <25° C. The contents were stirred for NLT 15 min, allowed to settle and the aqueous then removed. The organic layer was washed with a solution of citric acid monohydrate (9.3 g, 0.50 eq) in water (240 ml, 4 vol), then a solution of K₂CO₃ (12.22 g, 1.00 eq.) in water (240 ml, 4 vol), and then with water (120 ml, 2 vol). The organics were concentrated under vacuum to 120 ml, 2 volumes. Isopropyl alcohol IPA (300 ml, 5 vol) was added, and the mixture concentrated under vacuum to 120 ml, 2 volumes. Isopropyl alcohol (IPA) (300 ml, 5 vol) was added, and the mixture concentrated under vacuum to 210 ml, 3.5 vol. The reactor content was heated to 60° C. Water (120 ml, 2 vol) was added over 30 min. The contents were stirred at 60° C. The mixture was cooled to 45° C., seeds of compound III (0.005 wt.) were added, and the mixture stirred for NLT 1 hour. Water (180 ml, 3 vol) was added over 4 hours. The contents were then cooled to 20° C. over 4 hours, then stirred for NLT 2 hours. The slurry was filtered, and the wet cake rinsed with 2:1 water: IPA (120 ml, 2 vol). The cake was de-liquored and the product dried to constant weight at 45° C. under vacuum to provide compound III in 85-95% yield.

¹H NMR (400 MHz, DMSO-d6) δ 10.42 (s, 1H), 8.70 (dd, J=2.6, 0.7 Hz, 1H), 8.06 (dd, J=8.5, 2.5 Hz, 1H), 7.44 (dt, J=8.5, 0.7 Hz, 1H), 7.22-7.08 (m, 2H), 4.32 (dd, J=8.2, 6.7 Hz, 1H), 4.24 (dd, J=10.3, 7.6 Hz, 1H), 3.94 (d, J=2.1 Hz, 3H), 3.90-3.77 (m, 1H), 2.76 (p, J=7.5 Hz, 1H), 1.60 (s, 3H), 1.47-1.34 (m, 7H), 0.73 (dd, J=7.4, 2.4 Hz, 3H) ppm.

### Example 7—Preparation of Compound (I) from Compound (III)

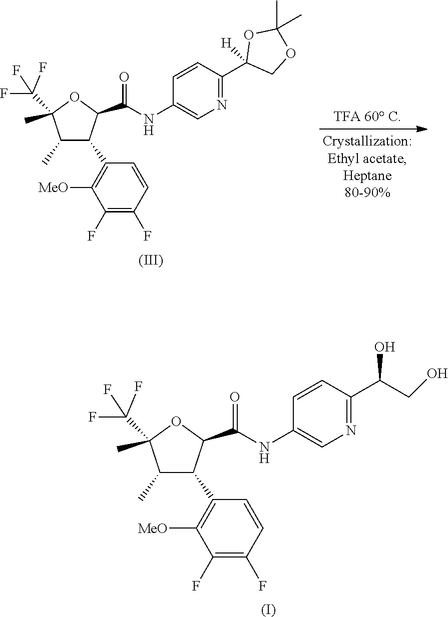

Compound III (42.2 g, 1.00 eq., 1.00 wt) was charged to a jacketed reactor set to 20° C. followed by a charge of THF (359 ml, 8.50 vol). Water (76 ml, 1.8 vol) and TFA (13.4 ml, 2.20 eq., 0.32 vol) were added at 20° C. The reactor contents were then heated to 60° C. for over 10 hours. After reaction completion, the reactor was cooled to 20° C. and the contents concentrated under vacuum to ~85 ml, 2 volumes. EtOAc (338 ml, 8 vol) and water (63 ml, 1.5 vol) were charged to reactor. The organic layer was separated and was washed with an aqueous K₂CO₃ solution (211 ml, 10% w/w, 5 vol) followed by water (169 ml, 4 vol). The organic layer was separated and diluted with EtOAc (211 ml, 5 vol). The solution was concentrated under vacuum to ~169 ml, 4 vol. EtOAc (338 ml, 8 vol) was charged, and the solution was filtered through a polish filter. The solution was concentrated 137 ml, ~3.25 vol.

The solution was heated to 70° C. and n-heptane (63 ml, 1.5 vol) was added over 1 hour. The solution was cooled to 60° C. and seeds of compound I (0.42 g, 1 wt %) were added. The reactor contents were stirred for 1 hour and n-heptane (190 ml, 4.5 vol) was added over 5 hours. The slurry was cooled to 20° C. over 5 hours and held for 8 hours at 20° C. The solids were isolated by filtration and the wet cake was rinsed with a mixture of ethyl acetate (32 ml, 0.75 vol) and n-heptane 95 ml, 2.25 vol). The wet cake was dried at 50° C. under vacuum. Compound I was obtained as a white solid in 85-90% yield, which is referred to herein as Form A.

¹H NMR (400 MHz, DMSO-d6) δ 10.37 (s, 1H), 8.66 (dd, J=2.6, 0.7 Hz, 1H), 8.01 (dd, J=8.5, 2.5 Hz, 1H), 7.43 (d, J=8.5 Hz, 1H), 7.23-7.09 (m, 2H), 5.33 (d, J=4.9 Hz, 1H), 5.09 (d, J=10.3 Hz, 1H), 4.63 (t, J=5.9 Hz, 1H), 4.55 (dt, J=6.8, 4.5 Hz, 1H), 4.25 (dd, J=10.3, 7.6 Hz, 1H), 3.95 (d, J=2.2 Hz, 3H), 3.63 (ddd, J=11.0, 6.0, 4.1 Hz, 1H), 3.45 (ddd, J=11.0, 6.8, 5.8 Hz, 1H), 2.77 (p, J=7.5 Hz, 1H), 1.61 (s, 3H), 0.73 (dt, J=7.6, 2.5 Hz, 3H) ppm.

### Compound (I)—Crystalline Solid Form A

Crystalline Form A of Compound (I) was characterized by XRPD, solid state NMR (¹³C and ¹⁹F), and single-crystal X-ray analysis.

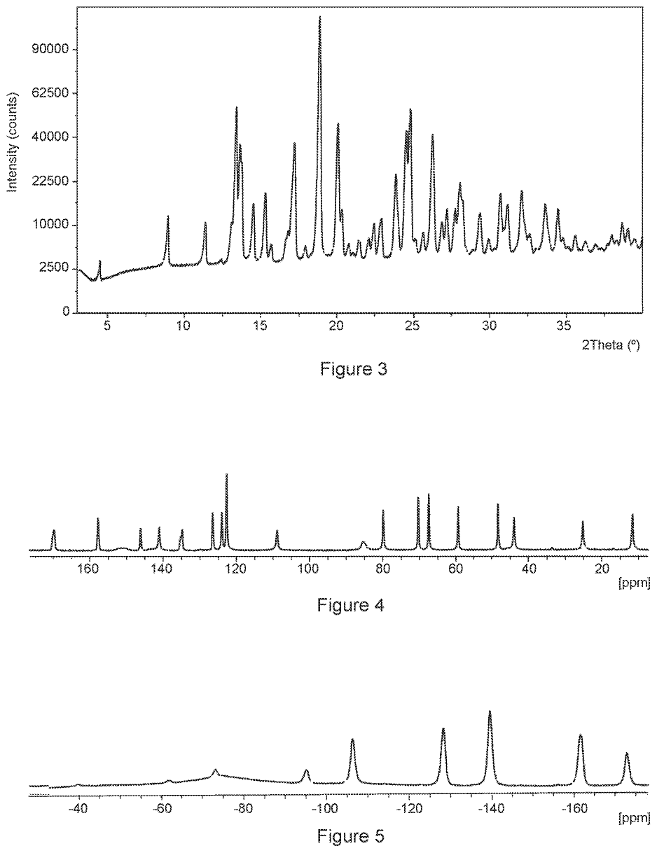

The XRPD pattern of Form A is depicted in FIG. 3, and the corresponding data are summarized in Table 3, below.

TABLE 3

| No. | Pos. [°2θ] | Rel. Int. [%] |
|---|---|---|
| 1 | 9.0 | 8.0 |
| 2 | 11.4 | 5.5 |
| 3 | 13.4 | 47.7 |
| 4 | 13.7 | 31.1 |
| 5 | 13.8 | 21.8 |
| 6 | 14.6 | 10.4 |
| 7 | 15.3 | 13.2 |
| 8 | 17.0 | 15.2 |
| 9 | 17.2 | 29.7 |
| 10 | 18.9 | 100 |
| 11 | 20.1 | 37.8 |
| 12 | 23.9 | 18.6 |
| 13 | 24.5 | 35.4 |
| 14 | 24.8 | 42.5 |
| 15 | 26.3 | 34.1 |
| 16 | 26.3 | 22.6 |
| 17 | 28.0 | 12.6 |
| 18 | 28.1 | 15.5 |
| 19 | 28.3 | 10.0 |
| 20 | 30.7 | 12.2 |
| 21 | 32.1 | 12.9 |

The skilled artisan would understand that the relative intensities of diffraction peaks can be affected by the experimental conditions. The overall diffraction pattern as shown in FIG. 3 may also be equally important for characterizing a solid form.

The solid state ¹³C NMR spectrum of Form A is depicted in FIG. 4, and the corresponding data are summarized in Table 4, below.

TABLE 4

| Peak # | Chem Shift [ppm] | Intensity [rel] |
|---|---|---|
| 1 | 169.8 | 27.9 |
| 2 | 157.7 | 43.1 |
| 3 | 151.3 | 3.3 |
| 4 | 146.1 | 29.4 |
| 5 | 142.8 | 2.2 |
| 6 | 141.0 | 30.7 |
| 7 | 135.3 | 17.5 |
| 8 | 134.8 | 27.1 |
| 9 | 126.4 | 50.3 |
| 10 | 123.9 | 50.4 |
| 11 | 122.6 | 100 |
| 12 | 108.9 | 26.2 |
| 13 | 85.1 | 10.7 |
| 14 | 79.8 | 53.3 |
| 15 | 70.2 | 69.2 |
| 16 | 67.3 | 73.8 |
| 17 | 59.3 | 56.6 |
| 18 | 48.4 | 61.0 |
| 19 | 44.0 | 42.4 |
| 20 | 25.1 | 37.8 |
| 21 | 11.6 | 46.2 |

The solid state ¹⁹F NMR spectrum of Form A is depicted in FIG. 5, and the corresponding data are summarized in Table 5, below.

TABLE 5

| Peak # | Chem Shift [ppm] | Intensity [rel] |
|---|---|---|
| 1 | −74.5 | 2.1 |
| 2 | −139.5 | 12.5 |
| 3 | −161.5 | 8.7 |

A single crystal having the Form A structure of Compound (I) was obtained from a hot solution in 1:1 heptane: ethyl acetate. X-ray diffraction data were acquired at 100K on a Bruker diffractometer equipped with Cu K_{α} radiation (λ=1.54178 Å) and a CMOS detector. The structure was solved and refined using SHELX programs (Sheldrick, G. M., Acta Cryst., (2008) A64, 112-122) and results are summarized in Table 6, below.

TABLE 6

| Crystal System: | Orthorhombic |
|---|---|
| Space Group: | P2₁2₁2₁ |
| a (Å) | 6.9511(10) |
| b (Å) | 8.2545(12) |
| c (Å) | 38.827(5) |
| α (°) | 90 |
| β (°) | 90 |
| γ (°) | 90 |
| V (Å³) | 2227.8(5) |
| Z/Z′ | 4/1 |
| Temperature (K) | 100(2) |

### Example 8—Preparation of Compound (XX) from Compound (VII)

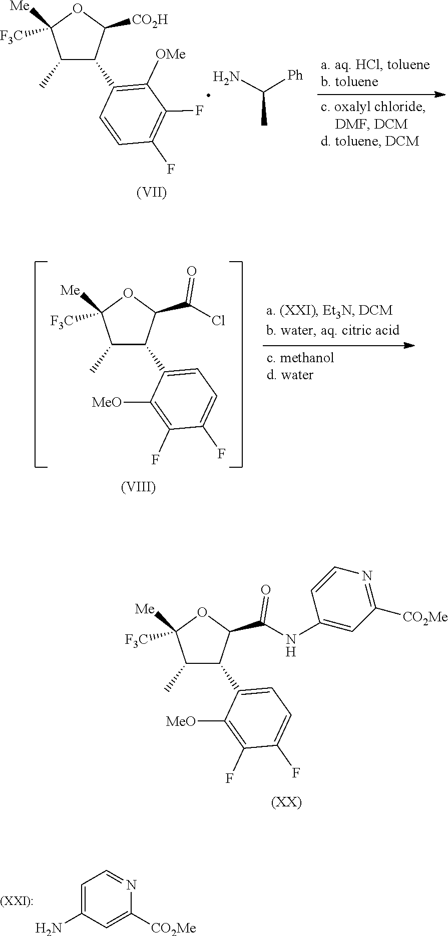

### Procedure 1:

Compound VII (205.6 kg, 432.4 mol, 1.00 equiv.) was dissolved with toluene (1070 kg, 6.0 vol) into a reactor and the temperature was adjusted to 20±5° C. A 2 M aqueous hydrochloric acid solution (637 kg, 3.0 vol) was charged, and the biphasic mixture was stirred for 30 minutes. The aqueous phase was separated out, then a 2 M aqueous hydrochloric acid solution (637 kg, 3.0 vol) was charged to the organic phase and the biphasic mixture was stirred for 30 minutes. The aqueous phase was separated out, then water (411.2 kg, 2.0 vol) was charged to the organic phase and the biphasic mixture was stirred for 30 minutes.

The resulting emulsion was phase separated and the organic phase was distilled down to 3.0 vol under vacuum at a maximum internal temperature of 45° C. Toluene (1070 kg, 6.0 vol) was charged and the solution was distilled down to 3.0 vol under vacuum at a maximum internal temperature of 45° C.

Dichloromethane (DCM, 817.3 kg, 3.0 vol) was charged to the previous solution, followed by N,N-dimethylformamide (DMF, 0.47 kg, 6.5 mol, 0.015 equiv.), and the resulting mixture was heated to 30±5° C. A previously prepared solution of oxalyl chloride (71.3 kg, 562.2 mol, 1.30 equiv.) in DCM (272.4 kg, 1.0 vol) was charged to the reaction mixture over no less than 1 hour while maintaining the temperature at 30±5° C. Reaction was allowed to progress for over 3 hours to form Compound VIII.

Post reaction, a series of put-take distillation cycles were performed (chasing with 10 vol toluene then 10 vol dichloromethane) to remove residual oxalyl chloride, resulting in a 3.0 vol solution of Compound VIII in dichloromethane. Dichloromethane (544.8 kg, 2.0 vol) was charged to make it a 5.0 vol solution.

Compound XXI (72.2 kg, 474.3 mol, 1.10 equiv.) was slurried into DCM (1086.5 kg, 4.0 vol) along with triethylamine (TEA, 52.4 kg, 517.4 mol, 1.20 equiv.) at a temperature of 25±5° C. The previous 5.0 vol solution of Compound VIII was charged in no less than 60 minutes, maintaining the temperature at 25±5° C., then the reaction mixture was stirred for no less than 1 hour.

After reaction completion, water (820.0 kg, 4.0 vol) was charged, the biphasic mixture was stirred for 30 minutes, allowed to settle for 30 min, then the aqueous phase was discarded. A 1 M citric acid aqueous solution (3.0 vol) was charged to the organic phase, the biphasic mixture was stirred for 30 minutes, allowed to settle for 30 min, then the aqueous phase was discarded. Water (410.0 kg, 2.0 vol) was charged, the biphasic mixture was stirred for 30 minutes, allowed to settle for 30 min, then the aqueous phase was discarded.

Solvent swap was performed via put-take distillation to exchange toluene with methanol, resulting in a 4.0 vol solution of Compound XX in methanol. A pre-mixed mixture of methanol (569.9 kg, 3.51 vol) and water (147.6 kg, 0.72 vol) was added to the latter solution. The batch was heated to 50±3° C. to fully dissolve. The batch was cooled to 40±3° C. before Compound XX seeds (1.03 kg, 0.0050 w/w equiv.) were added, and the seed bed was allowed to grow for 60 min at 40±3° C. Water (440.8 kg, 2.15 vol) was charged over no less than 3 hours, and the slurry was stirred at 40±3° C. for 30 min before being cooled to 20±3° C. over 5 hours and aged for 8 hours. The slurry was filtered and the resulting wet cake was washed with a previously prepared solution of methanol (324.7 kg, 2.0 vol) and water (176.3 kg, 0.86 vol) before drying at 40° C. to provide Compound XX. The isolated form is Form C in 85% yield.

### Procedure 2:

Compound VII (1.00 equiv) was slurried with toluene (6.0 vol, 5.19 w/w equiv) into a reactor and the temperature was adjusted to 20±5° C. A 2 M aqueous hydrochloric acid solution (3.0 vol) was charged, and the biphasic mixture was stirred for 30 minutes. The aqueous phase was separated out, then a 2 M aqueous hydrochloric acid solution (3.0 vol) was charged to the organic phase and the biphasic mixture was stirred for 30 minutes. The aqueous phase was separated out, then water (2.0 vol) was charged to the organic phase and the biphasic mixture was stirred for 30 minutes.

The resulting emulsion was phase separated and the organic phase was distilled down to 3.0 vol under vacuum at a maximum internal temperature of 45° C. Toluene (6.0 vol, 5.19 w/w equiv) was charged and the solution was distilled down to 3.0 vol under vacuum at a maximum internal temperature of 45° C.

Toluene (3.0 vol, 2.60 w/w equiv) was charged to the previous solution, followed by N,N-dimethylformamide (DMF, 0.015 equiv, 0.0023 w/w equiv), and the resulting mixture was heated to 30±5° C. A previously prepared solution of oxalyl chloride (1.30 equiv, 0.35 w/w equiv) in toluene (1.0 vol, 0.87 w/w equiv) was charged to the reaction mixture over no less than 1 hour while maintaining the temperature at 30±5° C. Reaction was allowed to progress for over 3 hours to form Compound VIII.

Post reaction, a series of put-take distillation cycles were performed (chasing with 10 vol toluene) to remove residual oxalyl chloride, resulting in a 3.0 vol solution of Compound VIII in toluene. Toluene (2.0 vol, 1.73 w/w equiv) was charged to make it a 5.0 vol solution.

Compound XXI (1.10 equiv, 0.35 w/w equiv) was slurried into toluene (4.0 vol, 3.47 w/w equiv) along with triethylamine (TEA, 1.20 equiv, 0.26 w/w equiv) at a temperature of 25±5° C. The previous 5.0 vol solution of Compound VIII was charged in no less than 1.0 h, maintaining the temperature at 20±5° C., then the reaction mixture was stirred for no less than 2.0 hour.

After reaction completion, ethyl acetate (EtOAc, 3.0 vol, 2.71 w/w equiv) and water (4.0 vol, 4.00 w/w equiv) were charged, the biphasic mixture was stirred for 30 minutes, allowed to settle for 30 min, then the aqueous phase was discarded. A 1 M citric acid aqueous solution (3.0 vol) was charged to the organic phase, the biphasic mixture was stirred for 30 minutes, allowed to settle for 30 min, then the aqueous phase was discarded. Water (2.0 vol) was charged, the biphasic mixture was stirred for 30 minutes, allowed to settle for 30 min, then the aqueous phase was discarded.

Solvent swap was performed via put-take distillation to exchange toluene and EtOAc with methanol, resulting in a 3.0 vol solution of Compound XX in methanol.

Methanol (4.5 vol, 3.56 w/w equiv) and water (0.95 vol, 0.95 w/w equiv) were added to the latter solution. The batch was heated to 60±3° C. to fully dissolve. The batch was cooled to 40±3° C. before Compound XX seeds (0.0050 w/w equiv) were added, and the seed bed was allowed to grow for 2.0 h at 40±3° C. Water (2.3 vol, 2.30 w/w equiv) was charged over no less than 5 hours, and the slurry was cooled to 20±3° C. over 2 hours and aged for no less than 1.0 h. The slurry was filtered and the resulting wet cake was washed with a previously prepared solution of methanol (2.8 vol, 2.22 w/w equiv) and water (1.2 vol, 1.20 w/w equiv) before drying at 45±5° C. to provide Compound XX. The isolated form is Form C in 85% yield.

### Example 9—Preparation of Compound (XX) from Compound (VI)

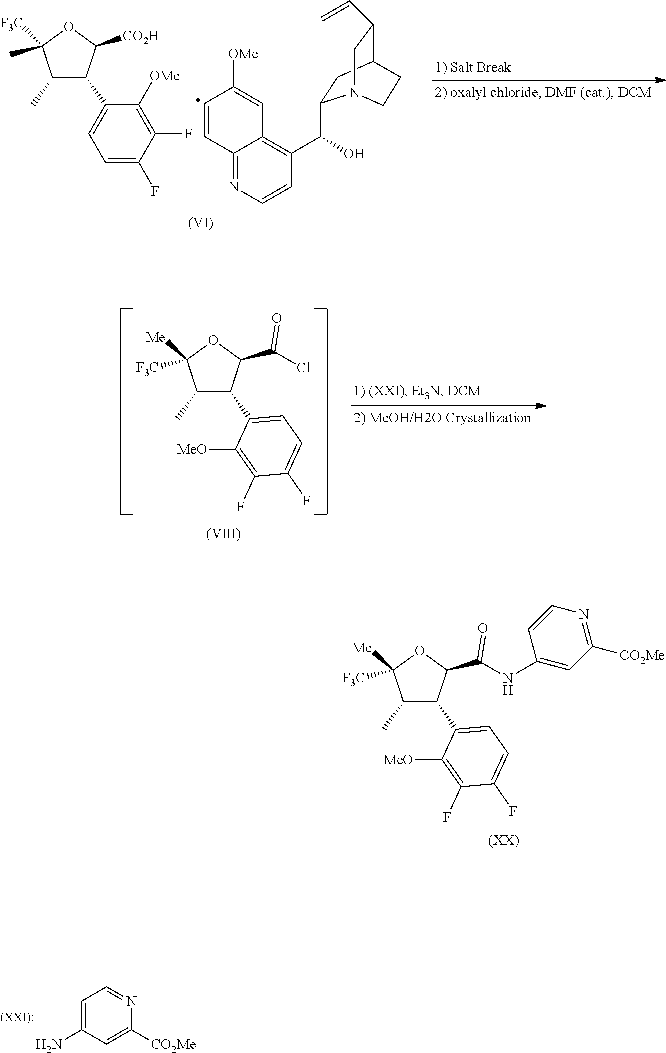

### Procedure 1:

Compound VI (quinine salt of compound IV, 2.8 kg) was dried in a tray dryer≤50° C. for at least 24 hours until the residual IPA content was ≤0.5% by ¹H NMR analysis. The dried quinine salt was charged to a 100 L jacketed reactor followed by addition of dichloromethane (DCM, 30.1 kg). A 2.5 M hydrochloric acid solution (9.0 kg) was charged and the mixture was agitated at 20±5° C. for at least 15 min. The layers were separated and the aqueous layer was discarded (this step was repeated two more times). The organic layer was washed with water (5.6 kg) and the layers were separated discarding the aqueous layer. A sample was pulled (IPC) from the organic layer in order to determine the amount of quinine. If the quinine was >1.0%, the organic layer was washed with aqueous hydrochloric acid and the layers were separated discarding the aqueous layer. The organic layer was transferred to a rotovap and distilled to 4 vol. DCM was charged to the 20 L rotovap round bottom flask (rbf) and the mixture was distilled to 4 vol. A sample was pulled (IPC) and analyzed for water content (KF) for information only.

The mixture was transferred to the 100 L jacketed reactor. N,N-Dimethylformamide (DMF, 33.1 g) was charged followed by oxalyl chloride (682.4 g) and the mixture was agitated at 20±5° C. for at least 3 h or until the amount of compound III present in the reaction mixture was <0.50%. The mixture was transferred to the 20 L rotovap rbf and distilled to 2 vol. DCM was charged to the rotovap rbf and the mixture was distilled to 3 vol (this step was repeated). A sample was pulled (IPC) and the amount of the oxalyl chloride was determined (derivative formation). When the amount of oxalyl chloride was determined to be >0.50%, DCM was charged to the mixture and the mixture was distilled to 3 vol (this step was repeated three times, if necessary, until the amount of oxalyl chloride was <0.50%).

Compound XXI (690.7 g) was charged to the 100 L jacketed reactor followed by DCM. The mixture was agitated and the temperature of the mixture was adjusted to 10±5° C. Triethylamine (TEA, 501.5 g) was charged to the 100 L jacketed reactor followed by transfer of the contents of the 20 L rbf containing the acid chloride. (i.e., compound V). The temperature of the reactor was adjusted to 20° C. over at least 30 min and agitated at 20±5° C. for at least 3 h. A sample was pulled (IPC) in order to determine the amount of compound IV. If compound IV was present in an amount >1.5%, the reaction was allowed to continue for at least 1 h at 20±5° C. The process of pulling a sample and determining compound IV content followed by stirring for at least 1 additional hour was repeated if necessary.

Water (11.2 kg) was charged to the reactor and the mixture was agitated for at least 15 min. The layers were allowed to separate and the aqueous layer was discarded. The organic layer was washed with an 18% aqueous citric acid solution (9.5 kg) followed by water (5.7 kg) and the aqueous layers discarded after each wash. The 100 L jacketed reactor was cleaned. The organic layer was transferred to the cleaned 100 L jacketed reactor and the mixture was distilled to 4 vol. Methanol was charged to the 100 L jacketed reactor and the mixture was distilled to 4 vol (this step was repeated). A sample was pulled (IPC) in order to determine the DCM content (¹H NMR) for information only. Additional Methanol (2.8 kg) was charged followed by water (4.9 kg). The temperature was adjusted to 60±5° C. and the mixture was agitated. The temperature of the mixture was adjusted to 55±5° C. and the mixture was agitated at 55±5° C. for at least 15 min. If needed Compound XX seed was charged and the mixture was agitated at 55±5° C. for at least 30 min. Water (4.9 kg) was charged over the course of at least 5 h. The mixture was agitated at 55±5° C. for at least 30 min followed by adjustment of the temperature to 20±5° C. over at least 5 h. The mixture was agitated at 20±5° C. for at least 8 h. The solids were collected by filtration and washed with water/methanol. The product (1.7 kg; 85% yield) was dried and packaged.

### Procedure 2:

Compound VI (quinine salt of compound IV; 10 g) was stirred with 60 mL toluene and 30 mL of aqueous hydrochloric acid solution (2 M) at 20° C. for over 30 minutes. The resulting emulsion was phase separated and organic phase (Compound IV) was stirred with 30 mL of aqueous hydrochloric acid solution (2 M) at 20° C. for over 30 minutes. The resulting emulsion was phase separated and organic phase (with Compound IV) was stirred with 20 mL of distilled water at 20° C. for over 30 minutes. The resulting emulsion was phase separated and organic phase (with compound IV) was distilled down (by chasing with 60 mL toluene) to about 30 mL. To the latter solution, 30 mL of dichloromethane was charged along with 200 μL of N,N-dimethylformamide and resulting mixture stirred at 30° C. To this solution, a separately prepared oxalyl chloride (1.6 mL) mixed with 10 mL of dichloromethane, was added in slowly over 1 hour. Reaction was allowed to progress for over 3 hours to form Compound VIII. Post reaction, a series of put-take distillation cycles need to be performed (chasing with 100 mL toluene then 100 mL dichloromethane) to remove residual oxalyl chloride. Resulting solution is 50 mL of Compound VIII in dichloromethane, to which is charged (over 1 hour) a separately prepared solution of Compound XXI (2.5 g) plus 40 mL dichloromethane plus 2.5 mL triethylamine. Reaction is allowed to happen at 25° C. for over 4 hours, after which a series of washes are performed [1) 40 mL water wash, 2) 26 mL citric acid solution wash, and 3) 20 mL water wash]. Solvent swap is performed via put-take distillation to exchange toluene with methanol, resulting in 70 mL of Compound XX solution in methanol. A pre-mixed mixture of 12.5 mL methanol and 5 mL water is added to the latter solution. The batch is heated to 35 to 40° C. and seeded with Compound XX crystals, following which a slow charge of 15 mL water over 3 hours and a cooldown to 20° C. for over 5 hours is carried out. The slurry is aged for over 8 hours and filtered under vacuum. The resulting wet cake was washed with a 30 vol % water in methanol solution and dried under vacuum at 40° C. to provide about 85 to 90% yield of Compound XX. The isolated form is Form C.

### Example 10—Synthesis of 4-((2R,3S,4S,5R)-3-(3,4-difluoro-2-methoxyphenyl)-4,5-dimethyl-5-(trifluoromethyl)tetrahydrofuran-2-carboxamido)picolinamide (II)

### Step 1: Synthesis of 4-((2R,3S,4S,5R)-3-(3,4-difluoro-2-methoxyphenyl)-4,5-dimethyl-5-(trifluoromethyl)tetrahydrofuran-2-carboxamido)picolinamide (II)

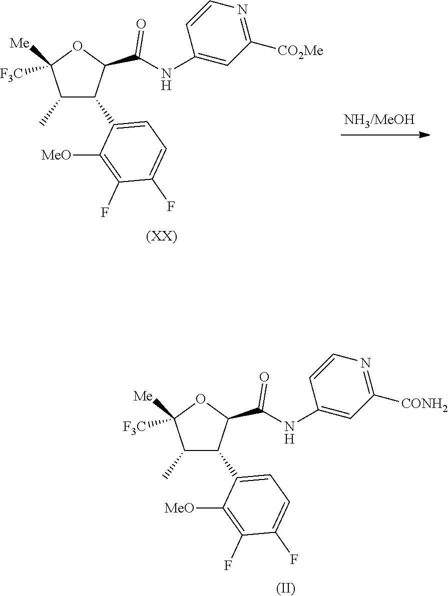

### Procedure 1:

Compound XX (1.6 kg) was charged to a 100 L reactor followed by a 7 M solution of ammonia in methanol (10.7 kg). The temperature of the mixture was adjusted to 20±5° C. The mixture was agitated at 20±5° C. for at least 24 h or until the amount of the starting material present was <0.5% (an additional amount of ammonia solution may be added if necessary to reduce compound XX to below 0.5%). The reaction mixture was transferred to a rotovap and distilled to 4 vol. Methanol was charged to the rotovap rbf and the mixture was distilled to 4 vol. The mixture was transferred to the 100 L reactor and methanol was charged. The temperature was adjusted to 55±5° C. and the mixture was agitated at 55±5° C. for at least 10 min. If a solution was not present in the reactor, the temperature of the mixture was adjusted to 60±5° C. and the mixture was agitated for at least 10 min followed by adjusting the temperature to 55±5° C. Water (7.9 kg) was charged over at least 2 hours to the rector while maintaining the temperature at 55±5° C. The mixture was agitated at 55±5° C. for at least 1 h followed by adjustment of the temperature to 20±5° C. over at least 12 h. The mixture was agitated at 20±5° C. for at least 5 hours and the solids were collected by filtration. The reactor was rinsed with methanol/water and the rinse was transferred through the filter cake. The solids were transferred to lined trays and dried in a tray drier at ≤45° C. for at least 12 h to yield 1.3 kg of crude compound (II) in 80% yield.

### Procedure 2:

Ammonia gas was bubbled into a 30 vol % tetrahydrofuran (385.8 kg, 3.10 vol) in methanol (798.3 kg, 7.20 vol) solution to result in about 6 M concentrated solution. Compound XX (140.0 kg, 286.6 mol, 1.00 equiv.) was charged to a sealed reactor, followed by the previously prepared ammonia solution, and solution was stirred at 20±5° C. for no less than 20 hours. The resulting Compound II solution was heated to 25±5° C., then water (175.0 kg, 1.25 vol) was charged over 1 hour at 25±5° C. Seeding was carried with Compound II crystals (0.70 kg, 0.0050 w/w equiv.) at 25±3° C. and aged for 1 hour. Water (1225.0 kg, 8.75 vol) was charged at 25±5° C. for over 4.5 hours. The slurry was aged for over 8 hours and filtered under vacuum. The resulting wet cake was washed with a methanol/tetrahydrofuran/water (35/15/50 volume ratio) and dried under vacuum at 40° C. to provide about 92 to 94% yield of Compound II. The isolated form is Form B in 90-93% yield.

### Procedure 3:

Ammonia was charged into a 30 vol % tetrahydrofuran (THF 3.0 vol, 2.67 w/w equiv) in methanol (7.0 vol, 5.54 w/w equiv) solution to result in no less than 5.2 M solution. Compound XX (1.00 equiv) was charged to a sealed reactor, followed by the previously prepared ammonia solution, and solution was stirred at 20±5° C. for no less than 24 hours.

The resulting Compound II solution was cooled to 18±3° C., then water (1.25 vol, 1.25 w/w equiv) was charged over 1 hour at 18±3° C. Compound II seeds (0.0050 w/w equiv) were charged at 18±3° C. and aged for 1 hour. Water (8.75 vol, 8.75 w/w equiv) was charged at 18±3° C. for over 4.5 hours. The slurry was aged for no less than 1 h and filtered. The resulting wet cake was washed with a previously prepared solution of methanol (1.05 vol, 0.83 w/w equiv), THF (0.45 vol, 0.40 w/w equiv), and water (1.50 vol, 1.50 w/w equiv), then dried under vacuum at 50±5° C. to provide dry Compound II. The isolated form is Form B in 92-94% yield.

### Step 2: Recrystallization of Compound (II)

### Procedure 1:

Crude compound II (1.23 kg) was charged to a 22 L rbf followed by acetone (5.01 kg). The mixture was agitated and the temperature was adjusted to 40±5° C. The mixture was polish filtered to a 20 L Jacketed Reactor. The temperature was adjusted to 35±5° C. and the mixture was agitated at 35±5° C. for at least 5 min. If the mixture was not a solution, the temperature was adjusted to 40±5° C. and the mixture was agitated at 40±5° C. for at least an additional 5 min. The temperature was adjusted to 35±5° C. and water (1.90 kg) was charged to the solution over the course of 1.5 h before seeding the solution. The mixture was agitated at 35±5° C. for at least 1 h. Water (2.49 kg) was charged to the reactor over at least 2 hours while maintaining a temperature of 35±5° C. and the mixture was agitated at 35±5° C. for at least 30 min. The temperature was adjusted to 20±5° C. over at least 5 h and the mixture was agitated at 20±5° C. for at least 5 h. The solid was collected by filtration and the reactor was rinsed with acetone/water sending the rinse through the filter cake. The solid was dried on the filter with N₂ gas for at least 30 min. The product was transferred to lined trays and dried in a tray drier to yield 1.10 kg of recrystallized compound (II).

### Procedure 2:

Compound II (120.0 kg, 253.5 mol, 1.00 equiv.) was charged to a reactor followed by methanol (665.3 kg, 7.0 vol) and tetrahydrofuran (320.0 kg, 3.0 vol) at 25±5° C. Polish filtration was then conducted. Water (150.0 kg, 1.25 vol) was charged over 1 hour at 25±3° C. Seeding was carried out with Compound II crystals (0.60 kg, 0.0050 w/w equiv.) at 25±3° C. and aged for 1 hour. Water (1050.0 kg, 8.75 vol) was charged at 25±3° C. for over 4.5 hours. The slurry was aged for over 8 hours and filtered under vacuum. The resulting wet cake was washed with a methanol/tetrahydrofuran/water (35/15/50 volume ratio) and dried under vacuum at 40° C. to provide about 95% yield of Compound II. The isolated form was Form B.

### Example 11—Preparation of Tablets Comprising Compound II

### Step 1: Preparation of Compound II Spray-Dried Dispersion (SDD)

About 2466.7 μm of dichloromethane and about 2466.7 μm of methanol were combined in a glass vessel. To the resulting mixture was added about 100 μm of Compound II (which can be prepared according to processes described herein) at room temperature. Upon complete dissolution of Compound II, about 300 gm of HPMCAS was added to the mixture at room temperature. Upon complete dissolution of the HPMCAS, the resulting mixture was spray-dried using a MicraSpray (MS-35) spray dryer with a 0.8 mm two fluid nozzle, 3 mm spacer, 2.6 mm air cap, 150 mesh PTFE inline filter, and a collection container attached to the cycle and pulseback filters. The process parameters of the MS-35 spray dryer are summarized in the following table:

### Spray Dryer Process Parameters

| Parameter | Target | (+/−) Range |
|---|---|---|
| Outlet Temp (° C.) | 48.0 | 45.0-51.0 |
| Process Gas Flow (kg/hr) | 35.0 | 33-37 |
| Nozzle Gas Flow (kg/hr) | 4.5 | 4.2-4.8 |
| Solution Feed Rate (kg/hr) | | |
| Solution Feed Rate (gm/min) | 30.0 | 27-33 |
| Estimated Spray time (hr) | 3.0 | |

To begin spray drying, the MS-35 was preheated, and when the target outlet temperature of 48° C. was reached an equilibrium solution was sprayed until all the parameters were stable and within the target range. Once all the parameters have stabilized and are within the target range the MS-35 spray dryer began spraying the solution containing Compound II and the polymer. The process parameters may be adjusted during the run to maintain it within the working range.

The wet SDD was transferred to appropriate size trays. Each tray was filled to a depth of about 1 inch of powder and placed in a vacuum oven at 40° C. with nitrogen purging. After 12-72 hours samples were pulled from the trays to check for residual solvent levels. Once the solvent levels were below the specifications (dichloromethane (<600 ppm) and methanol (<3000 ppm)) all powder from the trays were combined.

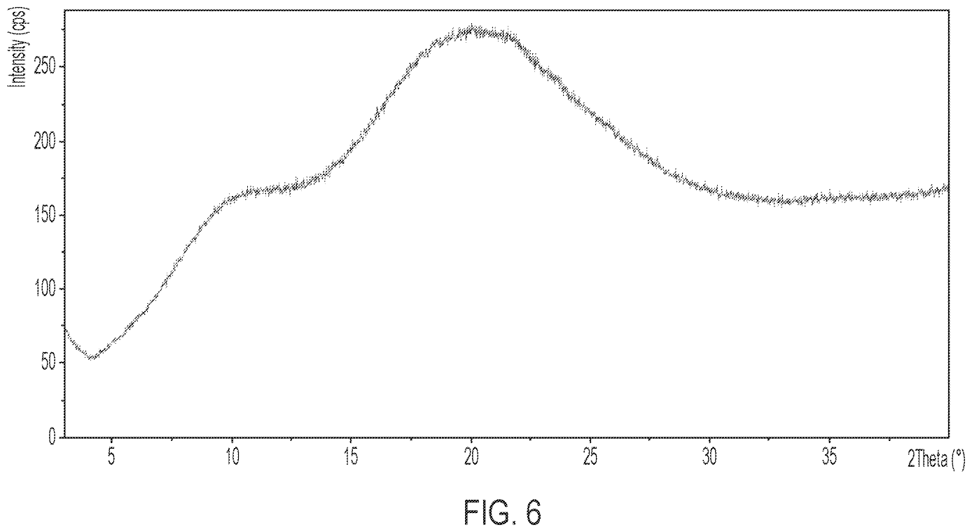

The SDD was transferred to a secondary drying chamber for further drying. Upon completion of drying, X-ray powder diffraction (XRPD) analysis was performed at room temperature in reflection mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed in a back-loading sample holder and loaded into the instrument. The sample was scanned over the range of about 3° to about 40°2θ with a step size of 0.0131° and 48.195 s per step. The obtained XRPD diffractogram is depicted in FIG. 6.

The composition of the SDD is summarized in Table 7:

TABLE 7
Composition of Compound II Spray-Dried Dispersion

| Component | Content (% w/w) |
|---|---|
| Compound II | 25.0 |
| Hydroxypropyl | 75.0 |
| Methylcellulose Acetate | |
| Succinate | |
| Total | 100.0 |

### Step 2: Preparation of Tablet Comprising Compound II SDD

Compound II SDD from Step 1, microcrystalline cellulose, lactose monohydrate, and croscarmellose sodium were each passed through a 20 mesh screen and combined in a 10 L Bohle blender. The mixture was blended for about 2.5 minutes at 32 rpm. Sodium stearyl fumarate was passed through a 60 mesh screen and added to the blended mixture and further blended for about 1.5 minutes at 32 rpm.

The resulting blended mixture was dry granulated using a GERTEIS® roller compactor and an in-line mill. The resulting milled granules were added to a 5 L Bohle blender along with microcrystalline cellulose and croscarmellose sodium that was passed through a 20 mesh screen. The resulting mixture was blended for 8.5 minutes at 32 rpm. After blending, sodium stearyl fumarate that was passed through a 60 mesh screen was added to the blended mixture and blended for an additional 2 minutes at 32 rpm. Process parameters for the roller compactor and in-line mill are summarized in Table 8:

TABLE 8
Process Parameters

| Parameter | Target | (+/−) Range |
|---|---|---|
| Roll Gap (mm) | 2.0 | 1.0-3.0 |
| Roll Pressure (kN/cm) | 9 | 6-12 |
| Roll Speed (rpm) | 2 | 1.5-2.5 |
| Gap Control | On | |
| Agitator speed (rpm) | 15 | |
| Granulation Speed cw/ccw (rpm) | 80/80 | 60-100/60-100 |
| Oscillation (cw/ccw) (deg) | 330-360 | 320-340/350-370 |
| PID “P value” | 3 | NA |
| PID “I value” | 3000 | 2000-7000 |
| PID “D value” | 0 | NA |
| Torque Control | off | |
| Tamp/Feed Ratio (%) | 165 | 110-500 |
| Feed Factor | 0.3 | 0.1-0.9 |
| Roll type | Smooth/Smooth | |
| Screen Size (mm) | 1.00 | |
| Rotor type | Pocketed rotor | |

The resulting mixture was then compressed into tablets using a PICCOLA® tablet press to yield tablets with each containing 10 mg of Compound II. The press had an 8 mm standard round concave tooling and paddle feeder. The turret speed was set to 30 rpm and the paddle speed was set to 25 rpm. The target weight and hardness of the tablets is summarized in Table 9:

TABLE 9
Target Weight and Hardness Specifications

| | Weight (mg) | Hardness (kP) |
|---|---|---|
| Target | 200 | 8.00 |
| Acceptance Limits | 180-220 | 6.0-10.0 |

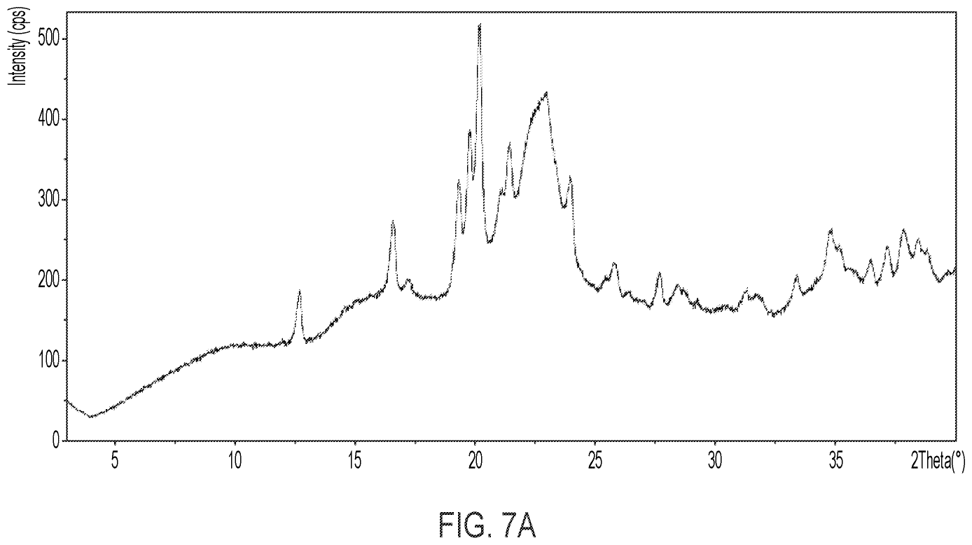

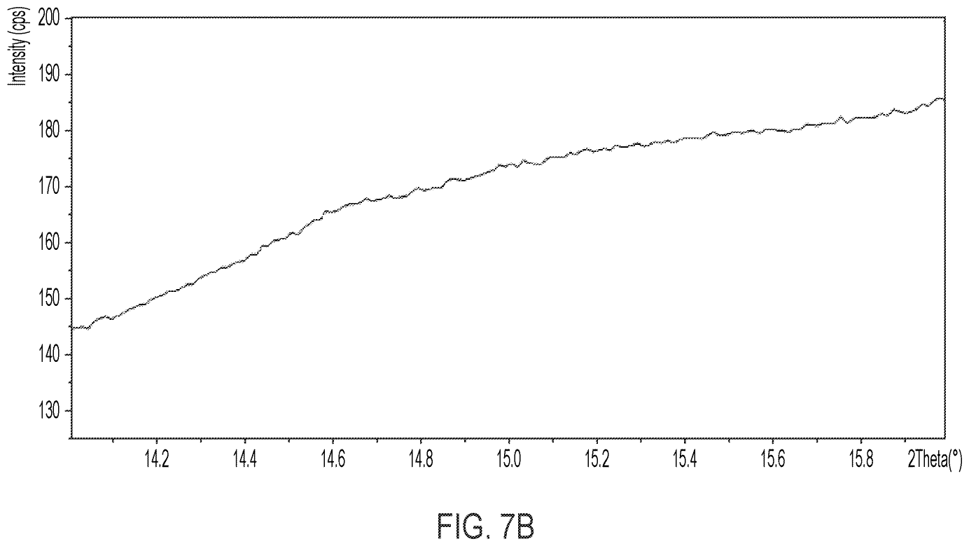

The resulting tablets were analyzed by X-ray powder diffraction (XRPD) and solid state nuclear magnetic resonance (ssNMR) after being ground into a uniform powder. XRPD analysis was performed at room temperature in reflection mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed in a back-loading sample holder and loaded into the instrument. The sample was scanned over the range of about 3° to about 40° 2θ with a step size of 0.0131° and 48.195s per step, and scanned over the range of about 14° to about 16° 2θ with a step size of 0.0131° and 1497.870s per step. The obtained XRPD diffractogram is depicted in FIGS. 7A and 7B.

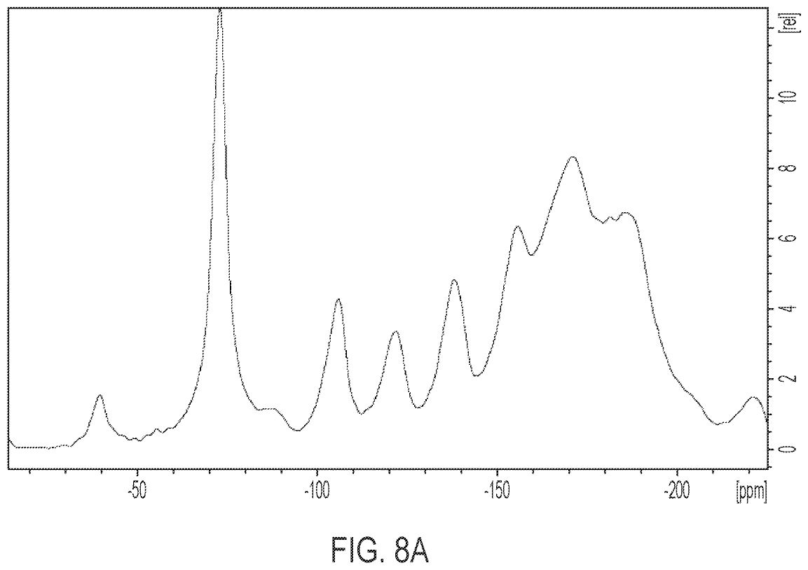

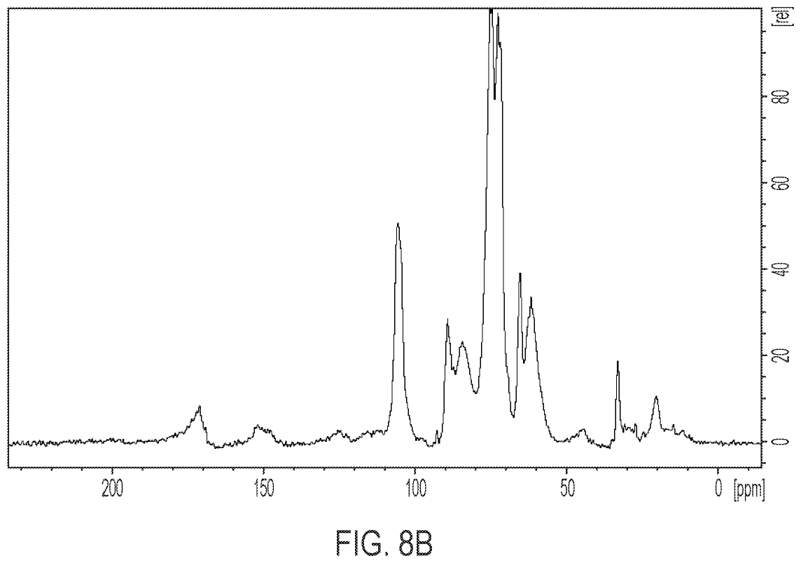

Solid state NMR analysis was conducted on a Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz. The obtained fluorine and carbon NMR spectra are depicted in FIGS. 8A and 8B, respectively, and the peak lists appear in Tables 10 and 11.

TABLE 10
¹⁹F MAS peak list:

| Peak # | δ [ppm] | Intensity |
|---|---|---|
| 1 | −40.1 | 1.51E0 |
| 2 | −73.2 | 1.23E1 |
| 3 | −105.9 | 4.26E0 |
| 4 | −121.8 | 3.33E0 |
| 5 | −137.9 | 4.80E0 |
| 6 | −155.6 | 6.33E0 |
| 7 | −171.1 | 8.30E0 |
| 8 | −220.6 | 1.49E0 |

TABLE 11
¹³C CPMAS peak list:

| Peak # | δ [ppm] | Intensity |
|---|---|---|
| 1 | 170.6 | 8.85E0 |
| 2 | 151.5 | 4.48E0 |
| 3 | 125.2 | 3.23E0 |
| 4 | 92.6 | 3.24E0 |
| 5 | 89.2 | 2.87E1 |
| 6 | 84.3 | 2.33E1 |
| 7 | 75.1 | 9.99E1 |
| 8 | 72.7 | 9.87E1 |
| 9 | 65.3 | 3.92E1 |
| 10 | 61.7 | 3.37E1 |
| 11 | 44.5 | 3.52E0 |
| 12 | 33.2 | 1.92E1 |
| 13 | 31.0 | 4.74E0 |
| 14 | 27.4 | 4.85E0 |
| 15 | 20.7 | 1.11E1 |

Table 12 describes exemplary tablet compositions produced using the method described in this example.

TABLE 12
Tablet Compositions (10 mg Compound II per tablet)

| Component | Content (% w/w) | Amount per Tablet (mg) |
|---|---|---|
| Compound II SDD | 20.0 | 40.0 |
| (25% drug load) | | |
| Microcrystalline Cellulose | 25.0 | 50.0 |
| (Aviccl PH101)* | | |
| Microcrystalline Cellulose | 22.5 | 45.0 |
| (Avicel PH200)** | | |
| Lactose Monohydrate | 25.0 | 50.0 |
| Croscarmellose Sodium | 4.5 | 9.0 |
| Sodium Stearyl Fumarate | 3.0 | 6.0 |
| Total | 100.0 | 200.0 |

*Avicel PH101 has a particle size of about 50 microns.
**Avicel PH200 has a particle size of about 180 microns.

### Example 12—Preparation of Tablets Comprising Compound II

### Step 1: Preparation of Compound II Spray-Dried Dispersion (SDD)

About 106532.8 μm of acetone was added to a stainless steel vessel. To the solvent was added about 4700.8 μm of Compound II (which can be prepared according to processes described herein) at room temperature. Upon complete dissolution of Compound II, about 14100.8 μm of HPMCAS was added to the mixture at room temperature. Upon complete dissolution of the HPMCAS, the resulting mixture was spray-dried using a MicraSpray (MS-75) spray dryer with a 0.8 mm two fluid nozzle, 3 mm spacer, 2.6 mm air cap, 150 mesh PTFE inline filter, and a collection container attached to the cyclone and pulseback filters. The process parameters of the MS-75 spray dryer are summarized in the following table:

### Spray Dryer Process Parameters

Spray Dryer Process Parameters

| Parameter | Target | (+/−) Range |
|---|---|---|
| Outlet Temp (° C.) | 45.0 | 35.0-55.0 |
| Process Gas Flow (kg/hr) | 75.0 | 60-77 |
| Nozzle Gas Flow (kg/hr) | 7.0 | 6.0-10.0 |
| Solution Feed Rate (kg/hr) | | |
| Solution Feed Rate (gm/min) | 70.0 | 60.0-80.0 |
| Estimated Spray time (hr) | 25 | |

To begin spray drying, the MS-75 was preheated, and when the target outlet temperature of 45° C. was reached an equilibrium solution was sprayed until all the parameters were stable and within the target range. Once all the parameters have stabilized and are within the target range the MS-75 spray dryer began spraying the solution containing Compound II and the polymer. The process parameters may be adjusted during the run to maintain it within the working range.

The wet SDD was transferred to appropriate size trays. Each tray was filled to a depth of about 1 inch of powder and placed in a vacuum oven at 40° C. with nitrogen purging. After 12-72 hours samples were pulled from the trays to check for residual solvent levels. Once the solvent levels were below the specifications (acetone (<5000 ppm) and cumene (<580 ppm)) all powder from the trays were combined.

Upon completion of drying, X-ray powder diffraction (XRPD) analysis was performed at room temperature in reflection mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed in a back-loading sample holder and loaded into the instrument. The sample was scanned over the range of about 3° to about 40° 2θ with a step size of 0.0131° and 48.195 s per step. The obtained XRPD diffractogram is depicted in FIG. 6.

The composition of the SDD is summarized in Table 13:

TABLE 13
Composition of Compound II Spray-Dried Dispersion

| Component | Content (% w/w) |
|---|---|
| Compound II | 25.0 |
| Hydroxypropyl | 75.0 |
| Methylcellulose Acetate | |
| Succinate | |
| Total | 100.0 |

### Step 2: Preparation of Tablet Comprising Compound II SDD

Compound II SDD from Step 1, microcrystalline cellulose and croscarmellose sodium were each passed through a 20 mesh screen and combined in a 100 L Bohle blender. The mixture was blended for about 205 minutes at 6 rpm. Magnesium stearate was passed through a 60 mesh screen and added to the blended mixture and further blended for about 11 minutes at 6 rpm.

The resulting blended mixture was dry granulated using a GERTEIS® roller compactor and an in-line mill. The resulting milled granules were added to a 100 L Bohle blender along with microcrystalline cellulose and croscarmellose sodium that was passed through a 20 mesh screen. The resulting mixture was blended for 20 minutes at 6 rpm. After blending, magnesium stearate that was passed through a 60 mesh screen was added to the blended mixture and blended for an additional 11 minutes at 6 rpm. Process parameters for the roller compactor and in-line mill are summarized in Table 14:

TABLE 14
Process Parameters

| Parameter | Target | (+/−) Range |
|---|---|---|
| Roll Gap (mm) | 2.0 | 1.0-4.0 |
| Roll Pressure (kN/cm) | 5.6 | 3.0-9.0 |
| Roll Speed (rpm) | 6.0 | 1.0-8.0 |
| Gap Control | On | |
| Agitator speed (rpm) | 40 | |
| Granulation Speed cw/ccw (rpm) | 80/80 | 60-100/60-100 |
| Oscillation (cw/ccw) (deg) | 330-360 | 320-340/350-370 |
| PID “P value” | 3 | NA |
| PID “I value” | 3000 | 2000-7000 |
| PID “D value” | 0 | NA |
| Torque Control | off | |
| Tamp/Feed Ratio (%) | 165 | 100-200 |
| Feed Factor | 0.3 | 0.05-1.0 |
| Roll type | Smooth/Smooth | |
| Screen Size (mm) | 1.00 | |
| Rotor type | Pocketed rotor | |

The resulting mixture was then compressed into tablets using a PICCOLA® tablet press to yield tablets with each containing 50 mg of Compound II. The press was equipped with 0.267×0.525 modified caplet tooling and a paddle feeder. The turret speed was set to 20 rpm and the paddle speed was set to 20 rpm. The target weight and hardness of the tablets is summarized in Table 15:

TABLE 15
Target Weight and Hardness Specifications

| | Weight (mg) | Hardness (kP) |
|---|---|---|
| Target | 400 | 14.00 |
| Acceptance Limits | 380-420 | 9.0-19.0 |

The resulting tablets were then film coated using a non-functional film coat, Opadry II Blue 85F105173. Approximately 1412.1 g of Opadry II Blue was suspended in approximately 5648.4 g of purified water. The Opadry II Blue was coated onto the tablets using a Labcoat 40 pan coater equipped with a 19 in pan and a single gun equipped with a 1.2 mm nozzle. The tablets were coated until a gain of ~3% by weight was achieved. The coating parameters are summarized in Table 16:

TABLE 16
Film Coating Process Parameters

| Parameter | Target | (+/−) Range |
|---|---|---|
| Atomizing Air Pressure (psi) | 30 | 15-45 |
| Pattern Air Pressure (psi) | 35 | 15-45 |
| Supply (Inlet) Air Flow Rate (cfm) | 190 | 150-350 |
| Pan Speed (rpm) | 15 | 8-20 |
| Inlet Temperature (° C.) | 65 | 55-75 |
| Exhaust Air Temperature (° C.) | 50 | 40-60 |
| Suspension Feed Rate (g/min) | 30 | 25-50 |

The resulting tablets were analyzed by X-ray powder diffraction (XRPD) and solid state nuclear magnetic resonance (ssNMR) after being ground into a uniform powder. XRPD analysis was performed at room temperature in reflection mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed in a back-loading sample holder and loaded into the instrument. The sample was scanned over the range of about 3° to about 40° 2θ with a step size of 0.0131° and 48.195s per step, and scanned over the range of about 14° to about 16° 2θ with a step size of 0.0131° and 1497.870s per step. The obtained XRPD diffractogram is depicted in FIGS. 7A and 7B.

Solid state NMR analysis was conducted on a Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz. The obtained fluorine and carbon NMR spectra are depicted in FIGS. 8A and 8B, respectively, and the peak lists appear in Tables 17 and 18.

TABLE 17
¹⁹F MAS peak list:

| Peak # | δ [ppm] | Intensity |
|---|---|---|
| 1 | −40.1 | 1.51E0 |
| 2 | −73.2 | 1.23E1 |
| 3 | −105.9 | 4.26E0 |
| 4 | −121.8 | 3.33E0 |
| 5 | −137.9 | 4.80E0 |
| 6 | −155.6 | 6.33E0 |
| 7 | −171.1 | 8.30E0 |
| 8 | −220.6 | 1.49E0 |

TABLE 18
¹³C CPMAS peak list:

| Peak # | δ [ppm] | Intensity |
|---|---|---|
| 1 | 170.6 | 8.85E0 |
| 2 | 151.5 | 4.48E0 |
| 3 | 125.2 | 3.23E0 |
| 4 | 92.6 | 3.24E0 |
| 5 | 89.2 | 2.87E1 |
| 6 | 84.3 | 2.33E1 |
| 7 | 75.1 | 9.99E1 |
| 8 | 72.7 | 9.87E1 |
| 9 | 65.3 | 3.92E1 |
| 10 | 61.7 | 3.37E1 |
| 11 | 44.5 | 3.52E0 |
| 12 | 33.2 | 1.92E1 |
| 13 | 31.0 | 4.74E0 |
| 14 | 27.4 | 4.85E0 |
| 15 | 20.7 | 1.11E1 |

Tables 19 and 20 describe exemplary tablet compositions produced using the method described in this example.

TABLE 19
Exemplary Tablet Compositions (50 mg Compound II per tablet)

| | | mg/tablet | mg/tablet |
|---|---|---|---|
| | Component | (50 mg, Ver #1) | (50 mg, Ver #2) |
| Intra- | Compound II SDD | 200.0 | 200.0 |
| granular | (25% drug load) | | |
| blend | Microcrystalline | 93.0 | 93.6 |
| | Cellulose | | |
| | (Avicel PH101) | | |
| | Croscarmellose | 6.0 | 6.0 |
| | sodium | | |
| | Magnesium | 1.0 | 0.4 |
| | stearate | | |
| Extra- | Microcrystalline | 91.0 | 90.4 |
| granular | Cellulose | | |
| blend | (Acivel PH102) | | |
| | Croscarmellose | 6.0 | 6.0 |
| | sodium | | |
| | Magnesium | 3.0 | 3.6 |
| | stearate | | |
| | Opadry II Blue | 12.0 | 12.0 |
| | Core Tablet Total | 400.0 | 400.0 |
| | Final Tablet Total | 412.0 | 412.0 |

TABLE 20
Exemplary Tablet Composition (50 mg Compound II per tablet)

| Component | mg/tablet |
|---|---|
| Compound II SDD | 200.0 |
| (25% drug load) | |
| Microcrystalline Cellulose | 184.0 |
| Croscarmellose sodium | 12.0 |
| Magnesium stearate | 4.0 |
| Opadry II Blue | 12.0 |
| Core Tablet Total | 400.0 |
| Final Tablet Total | 412.0 |

Many modifications and variations of the embodiments described herein may be made without departing from the scope, as is apparent to those skilled in the art. The specific embodiments described herein are offered by way of example only.

What is claimed is:

1. A process for preparing a compound of formula (I): 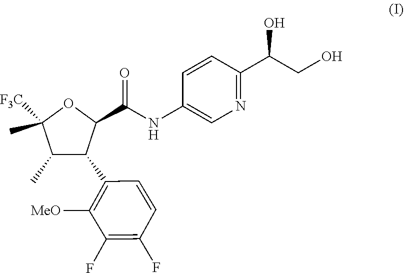 [SMILES: COc1c([C@H]2[C@H](C(=O)Nc3ccc([C@@H](O)CO)nc3)O[C@@](C)(C(F)(F)F)[C@H]2C)ccc(F)c1F]
comprising transforming a compound of formula (IV): 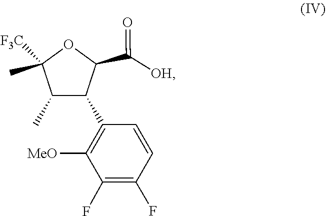 [SMILES: COc1c([C@H]2[C@H](C(=O)O)O[C@@](C)(C(F)(F)F)[C@H]2C)ccc(F)c1F]
or a salt thereof, to the compound of formula (I).

2. The process of claim 1, wherein transforming the compound of formula (IV) to the compound of formula (I) comprises preparing a compound of formula (VI):

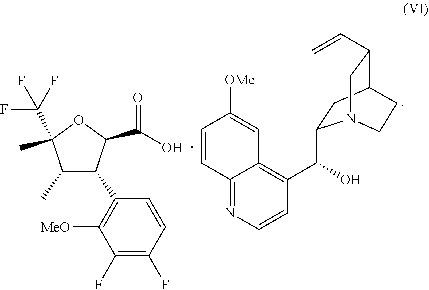

3. The process of claim 2, wherein preparing the compound of formula (VI) comprises contacting the compound of formula (IV) or a compound of formula (VII): 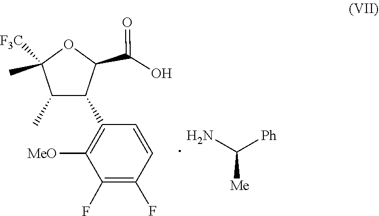
with quinine.

4. The process of claim 1, wherein transforming the compound of formula (IV) to the compound of formula (I) comprises preparing a compound of formula (VII):

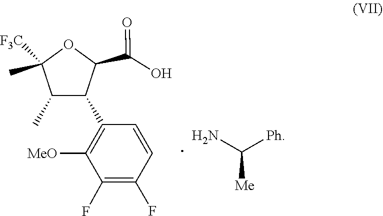

5. The process of claim 3 or 4, wherein the compound of formula (VII) is obtained by contacting the compound of formula (IV) with (R)-α-methylbenzylamine.

6. The process of any one of claims 1 to 5, wherein transforming a compound of formula (IV) to the compound of formula (I) comprises contacting the compound of formula (IV), the compound of formula (VI), or the compound of formula (VII) with a compound of formula (V): 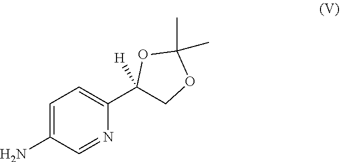
to afford a compound of formula (III):

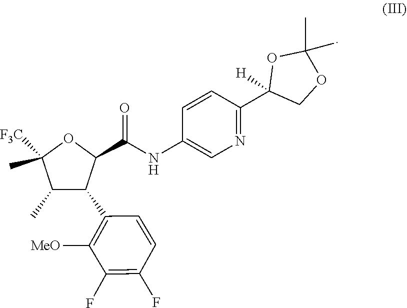

7. The process of any one of claims 1 to 5, wherein transforming the compound of formula (IV) to the compound of formula (I) comprises transforming the compound of formula (IV), the compound of formula (VI), or the compound of formula (VII) to a compound of formula (VIII):

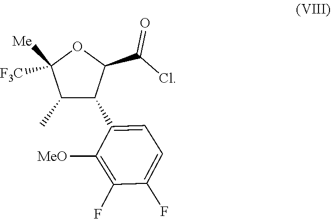 [SMILES: COc1c([C@H]2[C@H](C(=O)Cl)O[C@@](C)(C(F)(F)F)[C@H]2C)ccc(F)c1F]

8. The process of claim 7, wherein transforming a compound of formula (IV) to the compound of formula (I) comprises contacting the compound of formula (VIII) with a compound of formula (V): 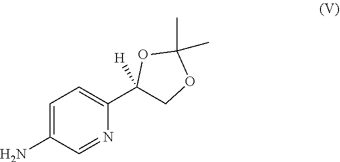
to afford a compound of formula (III):

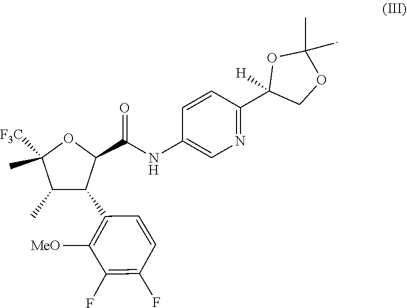

9. The process of claim 6 or 8, wherein transforming a compound of formula (IV) to the compound of formula (I) further comprises transforming the compound of formula (III) to the compound of formula (I).

10. The process of any one of claims 1 to 9, further comprising recrystallizing the compound of formula (I) from a suitable solvent to afford crystalline Form A of the compound of formula (I).

11. A process for preparing a compound of formula (II): 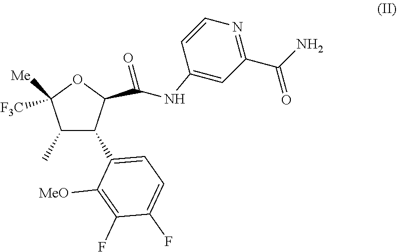 [SMILES: COc1c([C@H]2[C@H](C(=O)Nc3ccnc(C(N)=O)c3)O[C@@](C)(C(F)(F)F)[C@H]2C)ccc(F)c1F]
comprising transforming a compound of formula (VII): 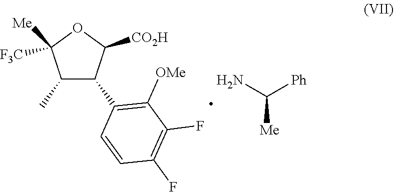 [SMILES: COc1c([C@H]2[C@H](C(=O)O)O[C@@](C)(C(F)(F)F)[C@H]2C)ccc(F)c1F ; C[C@@H](N)c1ccccc1]
to the compound of formula (II).

12. The process of claim 11, wherein transforming the compound of formula (VII) to the compound of formula (II) comprises contacting the compound of formula (VII) with a compound of formula (XXI): 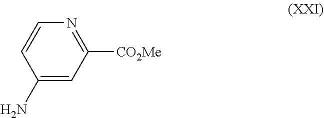 [SMILES: COC(=O)c1cc(N)ccn1]
to afford a compound of formula (XX):

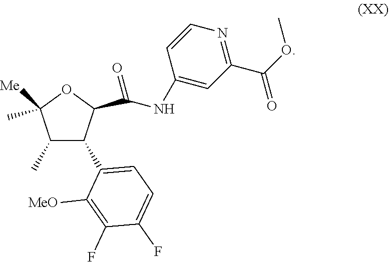

13. The process of claim 11, wherein transforming the compound of formula (VII) to the compound of formula (II) comprises transforming the compound of formula (VII) to a compound of formula (VIII):

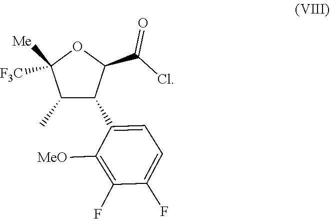 [SMILES: COc1c([C@H]2[C@H](C(=O)Cl)O[C@@](C)(C(F)(F)F)[C@H]2C)ccc(F)c1F]

14. The process of claim 12, wherein transforming a compound of formula (VII) to the compound of formula (II) further comprises transforming the compound of formula (XX) to the compound of formula (II).

15. The process of claim 14, wherein transforming the compound of formula (XX) to the compound of formula (II) comprises treating the compound of formula (XX) with ammonia to afford the compound of formula (II).

16. The process of any one of claims 11 to 15, further comprising transforming a compound of formula (IV): 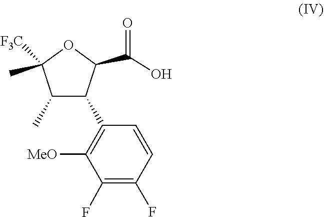 [SMILES: COc1c([C@H]2[C@H](C(=O)O)O[C@@](C)(C(F)(F)F)[C@H]2C)ccc(F)c1F]
to the compound of formula (VII).

17. The process of claim 16, wherein transforming the compound of formula (IV) to the compound of formula (VII) comprises contacting the compound of formula (IV) with (R)-α-methylbenzylamine.

18. The process of any one of claim 1 to 10, 16, or 17, further comprising transforming a compound of formula (IX): 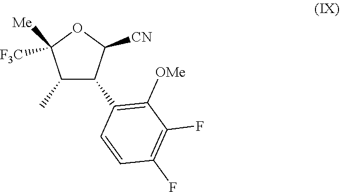 [SMILES: COc1c([C@H]2[C@H](C#N)O[C@@](C)(C(F)(F)F)[C@H]2C)ccc(F)c1F]
to the compound of formula (IV).

19. The process of claim 18, further comprising transforming a compound of formula (X): 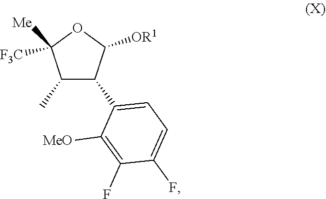
to the compound of formula (IX),
wherein:
R¹ is —C(O)—Z, —C(O)OZ, —C(O)CH═CH—Z, or —P(O)Z₂; and
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl.

20. The process of claim 19, wherein the compound of formula (X) has formula (X′):

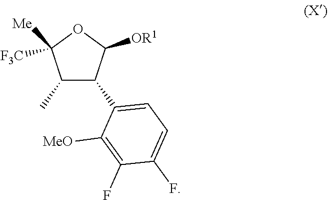

21. The process of claim 19 or 20, further comprising transforming a compound of formula (XI): 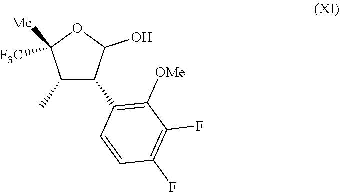 [SMILES: COc1c([C@H]2C(O)O[C@@](C)(C(F)(F)F)[C@H]2C)ccc(F)c1F]
to the compound of formula (X).

22. The process of claim 21, wherein the compound of formula (XI) has formula (XI′):

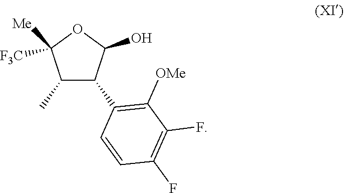 [SMILES: COc1c([C@H]2[C@H](O)O[C@@](C)(C(F)(F)F)[C@H]2C)ccc(F)c1F]

23. The process of claim 21 or 22, further comprising transforming a compound of formula (XII): 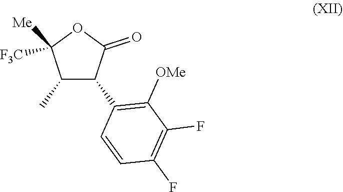 [SMILES: COc1c([C@H]2C(=O)O[C@@](C)(C(F)(F)F)[C@H]2C)ccc(F)c1F]
to the compound of formula (XI).

24. The process of claim 23, further comprising transforming a compound of formula (XIII): 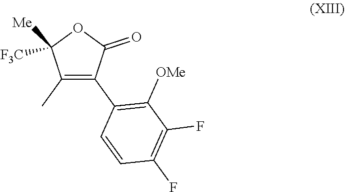 [SMILES: COc1c(C2=C(C)[C@](C)(C(F)(F)F)OC2=O)ccc(F)c1F]
to the compound of formula (XII).

25. The process of claim 24, further comprising contacting a compound of formula (XIV): 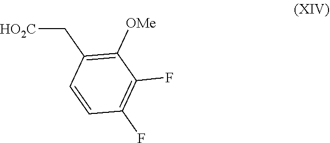 [SMILES: COc1c(CC(=O)O)ccc(F)c1F]
with a compound of formula (XV): 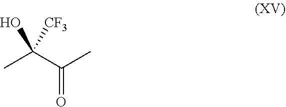
to afford the compound of formula (XIII).

26. A compound of formula: 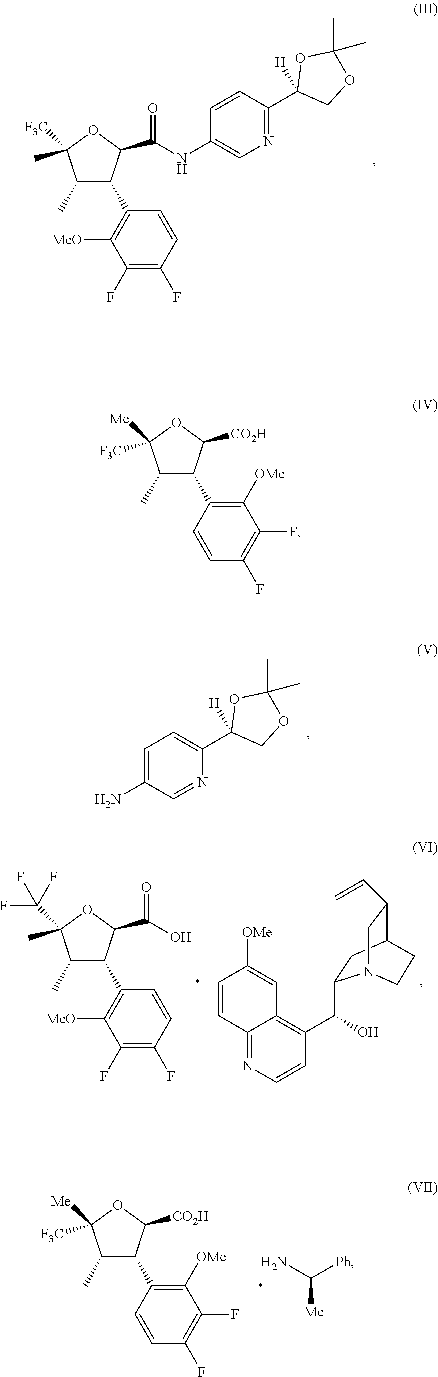 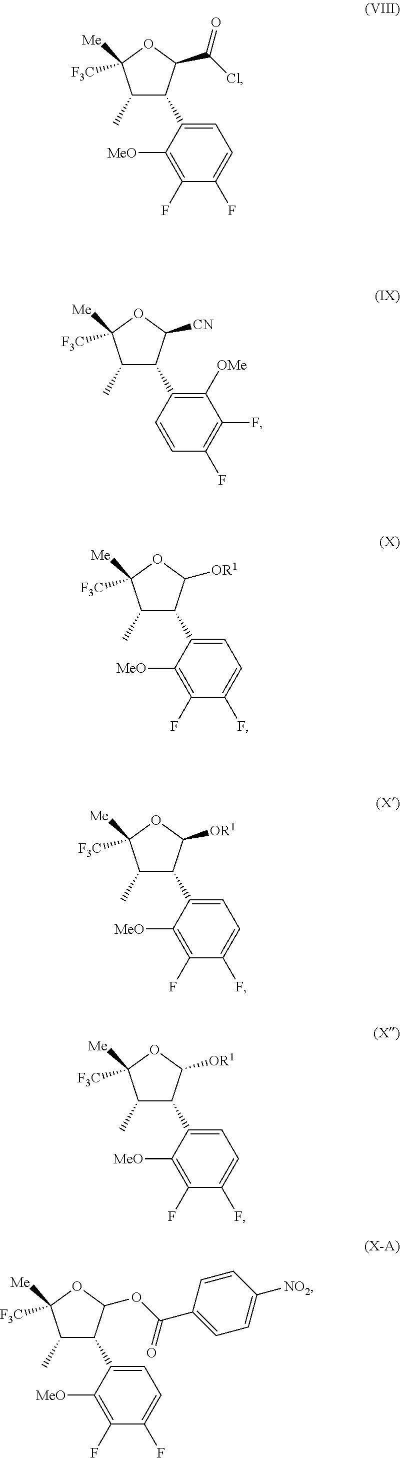 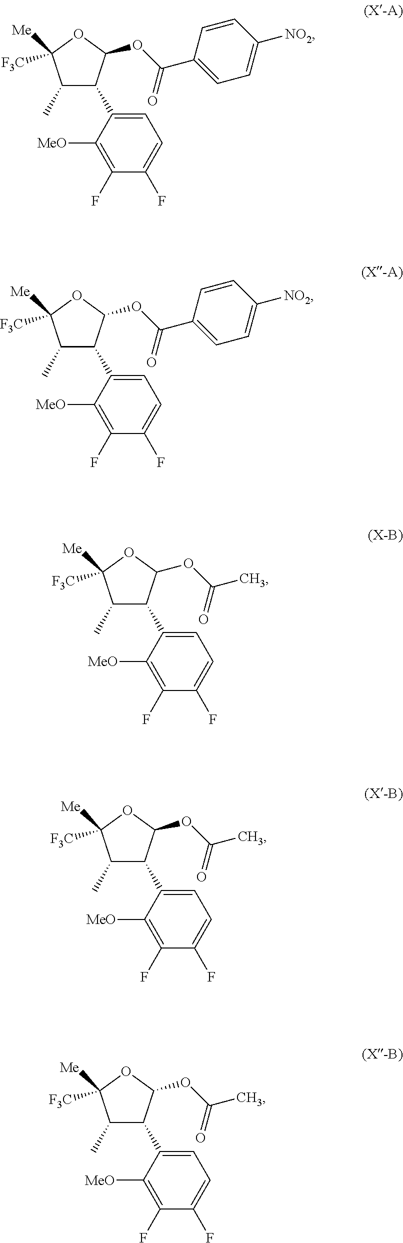 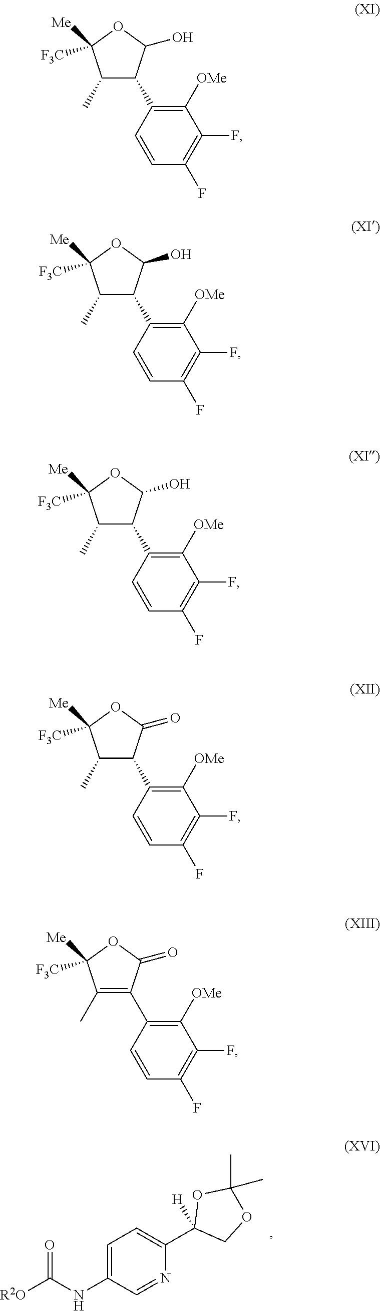 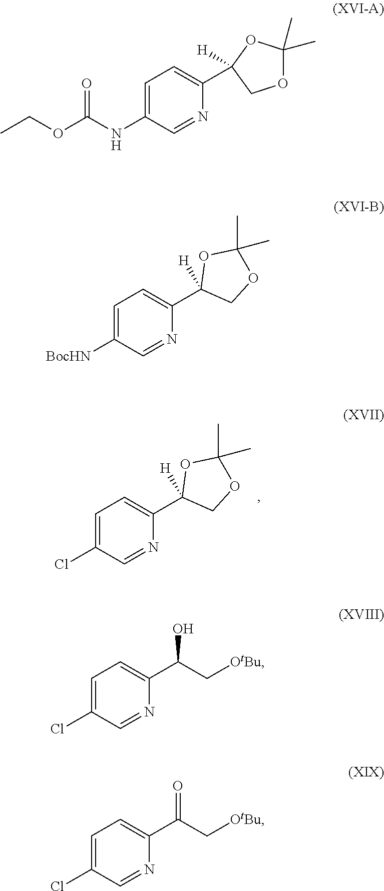 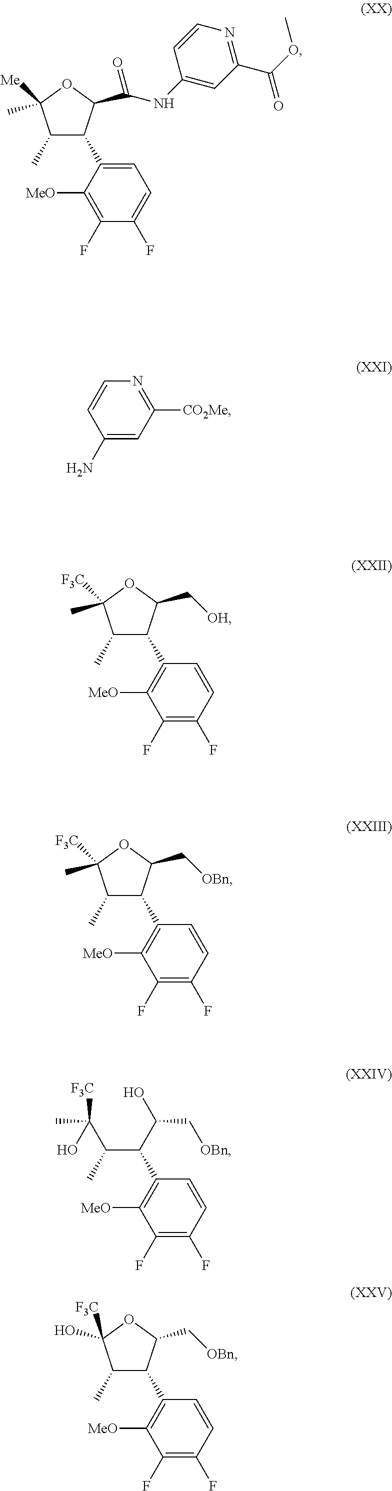 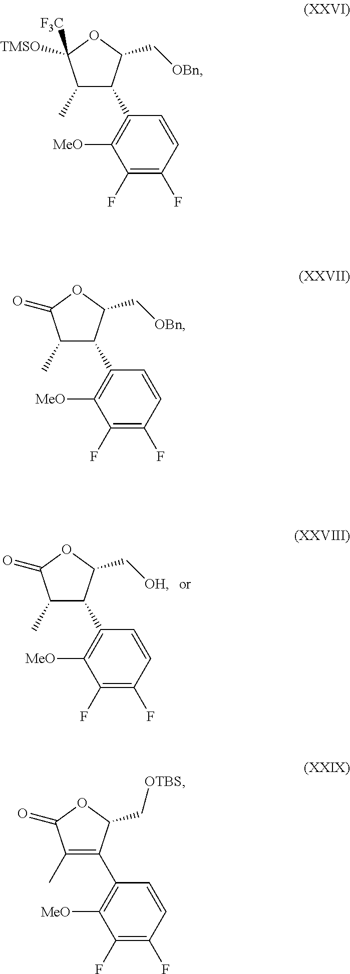
wherein:
R¹ is —C(O)—Z, —C(O)OZ, —C(O)CH═CH—Z, or —P(O)Z₂;
Z is selected from C₆-C₁₀ aryl optionally substituted with CN, halo, NO₂, C₁-C₄ alkyl, C₁-C₄ alkoxy, C₁-C₄ haloalkyl, and/or C₁-C₄ haloalkoxy; C₁-C₄ alkyl; and C₁-C₄ haloalkyl; and
R² is C₁-C₆ alkyl.

27. A compound of formula:

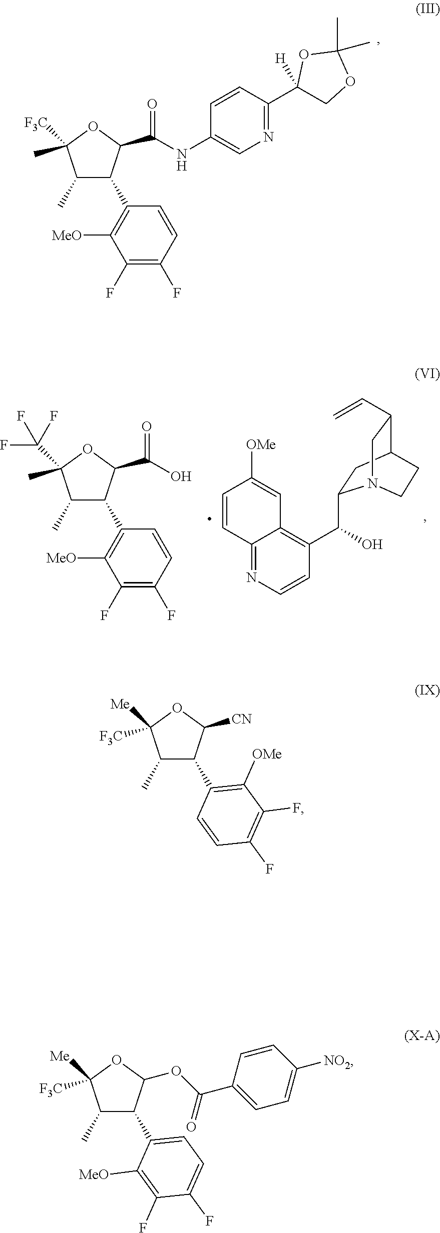

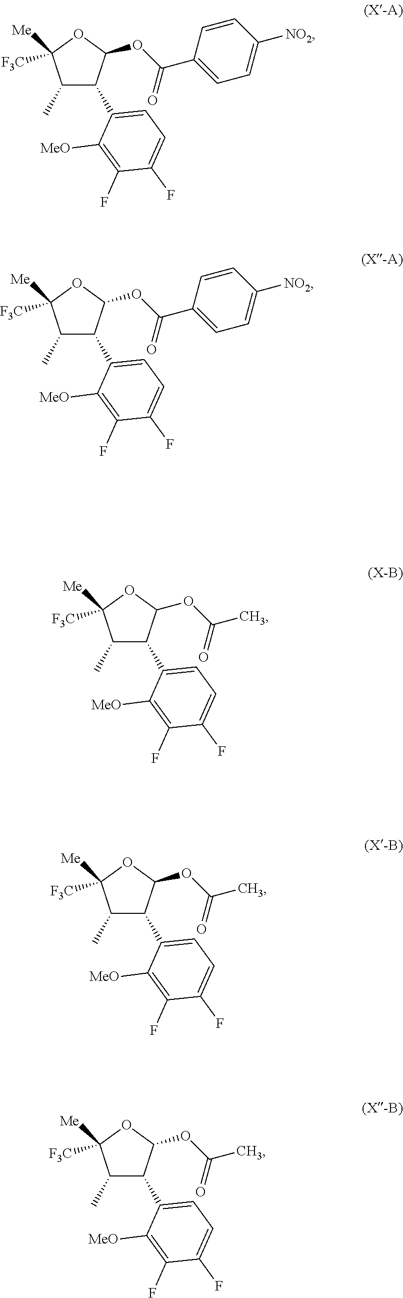 [SMILES: COc1c([C@H]2[C@H](OC(=O)c3ccc([N+](=O)[O-])cc3)O[C@@](C)(C(F)(F)F)[C@H]2C)ccc(F)c1F ; COc1c([C@H]2[C@@H](OC(=O)c3ccc([N+](=O)[O-])cc3)O[C@@](C)(C(F)(F)F)[C@H]2C)ccc(F)c1F ; COc1c([C@H]2C(OC(C)=O)O[C@@](C)(C(F)(F)F)[C@H]2C)ccc(F)c1F ; COc1c([C@H]2[C@H](OC(C)=O)O[C@@](C)(C(F)(F)F)[C@H]2C)ccc(F)c1F ; COc1c([C@H]2[C@@H](OC(C)=O)O[C@@](C)(C(F)(F)F)[C@H]2C)ccc(F)c1F]

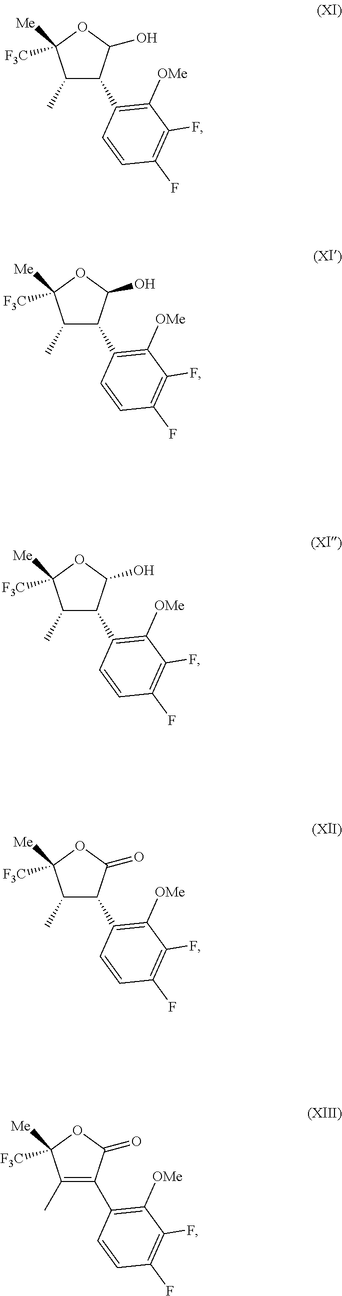 [SMILES: COc1c([C@H]2C(O)O[C@@](C)(C(F)(F)F)[C@H]2C)ccc(F)c1F ; COc1c([C@H]2[C@H](O)O[C@@](C)(C(F)(F)F)[C@H]2C)ccc(F)c1F ; COc1c([C@H]2[C@@H](O)O[C@@](C)(C(F)(F)F)[C@H]2C)ccc(F)c1F ; COc1c([C@H]2C(=O)O[C@@](C)(C(F)(F)F)[C@H]2C)ccc(F)c1F ; COc1c(C2=C(C)[C@](C)(C(F)(F)F)OC2=O)ccc(F)c1F]

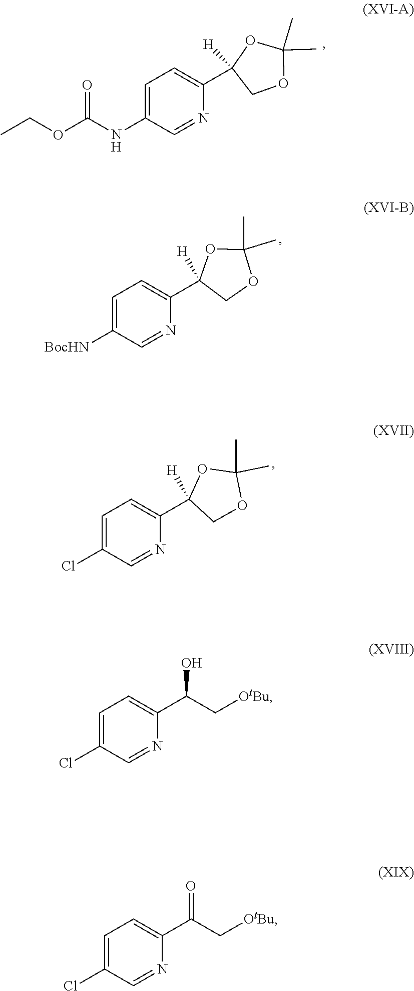 [SMILES: CCOC(=O)Nc1ccc([C@@H]2COC(C)(C)O2)nc1 ; CC(C)(C)OC(=O)Nc1ccc([C@@H]2COC(C)(C)O2)nc1 ; CC1(C)OC[C@@H](c2ccc(Cl)cn2)O1 ; CC(C)(C)OC[C@H](O)c1ccc(Cl)cn1 ; CC(C)(C)OCC(=O)c1ccc(Cl)cn1]

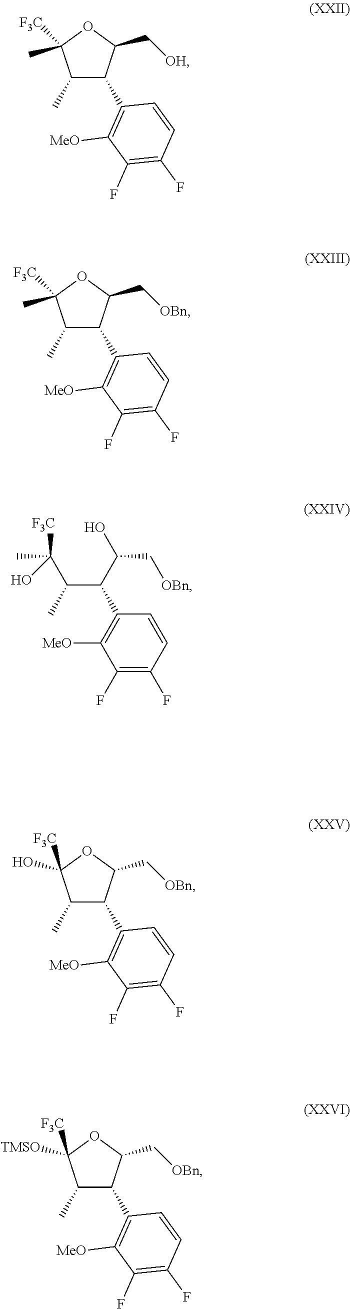

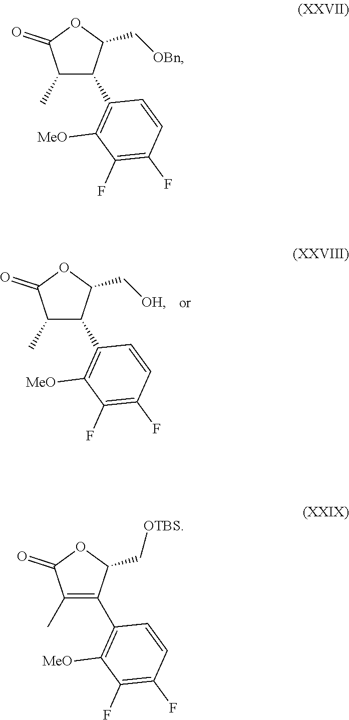 [SMILES: COc1c([C@@H]2[C@H](C)C(=O)O[C@@H]2COCc2ccccc2)ccc(F)c1F ; COc1c([C@@H]2[C@H](C)C(=O)O[C@@H]2CO)ccc(F)c1F ; COc1c(C2=C(C)C(=O)O[C@@H]2CO[Si](C)(C)C(C)(C)C)ccc(F)c1F]

28. A compound of formula (I): 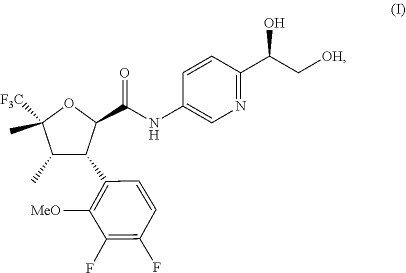 [SMILES: COc1c([C@H]2[C@H](C(=O)Nc3ccc([C@@H](O)CO)nc3)O[C@@](C)(C(F)(F)F)[C@H]2C)ccc(F)c1F]
wherein the compound is in a crystalline solid form.

29. A composition comprising a compound of formula (I): 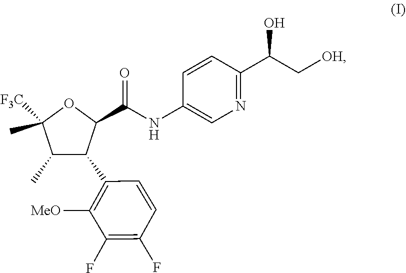 [SMILES: COc1c([C@H]2[C@H](C(=O)Nc3ccc([C@@H](O)CO)nc3)O[C@@](C)(C(F)(F)F)[C@H]2C)ccc(F)c1F]
wherein the compound of formula (I) is in a crystalline solid form.

30. The composition of claim 29, wherein at least 85% of the compound of formula (I) present in the composition is in a crystalline solid form.

31. A pharmaceutical composition comprising a compound of formula (I): 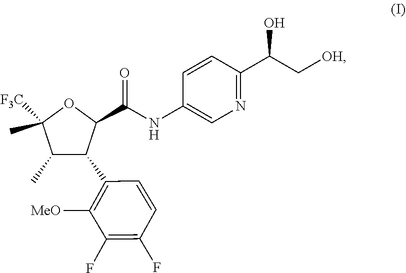 [SMILES: COc1c([C@H]2[C@H](C(=O)Nc3ccc([C@@H](O)CO)nc3)O[C@@](C)(C(F)(F)F)[C@H]2C)ccc(F)c1F]
and one or more pharmaceutically acceptable carriers or vehicles, wherein the compound of formula (I) is in a crystalline solid form.

32. The pharmaceutical composition of claim 31, wherein at least 85% of the compound of formula (I) present in the pharmaceutical composition is in a crystalline solid form.

33. The compound of claim 28, the composition of claim 29 or 30, or the pharmaceutical composition of claim 31 or 32, wherein the crystalline solid form is Form A.

34. The compound, the composition, or the pharmaceutical composition of claim 33, wherein Form A is characterized by an X-ray powder diffraction pattern (XRPD) pattern comprising at least one approximate peak position (degrees 2-theta±0.2) when measured using Cu K_{α} radiation, selected from 9.0, 11.4, 13.4, 13.7, 13.8, 14.6, 15.3, 17.0, 17.2, 18.9, 20.1, 23.9, 24.5, 24.8, 26.3, 28.0, 28.1, 28.3, 30.7, and 32.1.

35. The compound, the composition, or the pharmaceutical composition of claim 33, wherein Form A is characterized by an X-ray powder diffraction pattern (XRPD) pattern comprising at least one approximate peak position (degrees 2-theta±0.2) when measured using Cu K_{α} radiation, selected from 13.4, 17.2, and 18.9.

36. The compound, the composition, or the pharmaceutical composition of claim 33, wherein Form A is characterized by an X-ray powder diffraction pattern (XRPD) pattern comprising:
at least one approximate peak position (degrees 2-theta±0.2) when measured using Cu K_{α} radiation, selected from 13.4, 17.2, and 18.9; and
at least one approximate peak position (degrees 2-theta±0.2) when measured using Cu K_{α} radiation, selected from 9.0, 11.4, and 20.1.

37. The compound, the composition, or the pharmaceutical composition of claim 33, wherein Form A is characterized by an X-ray powder diffraction pattern (XRPD) pattern comprising:
at least one approximate peak position (degrees 2-theta±0.2) when measured using Cu K_{α} radiation, selected from 13.4, 17.2, and 18.9;
at least one approximate peak position (degrees 2-theta±0.2) when measured using Cu K_{α} radiation, selected from 9.0, 11.4, and 20.1; and
at least one approximate peak position (degrees 2-theta±0.2) when measured using Cu K_{α} radiation, selected from 13.7, 13.8, and 24.8.

38. The compound, the composition, or the pharmaceutical composition of any one of claims 33 to 37, wherein Form A is characterized by a solid state ¹⁹F NMR spectrum having at least one peak at a chemical shift selected from −74.5, −139.5, and −161.5 ppm.

39. The compound, the composition, or the pharmaceutical composition of any one of claims 33 to 38, wherein Form A is characterized by a solid state ¹³C NMR spectrum having at least one peak at a chemical shift selected from 169.8, 157.7, 151.3, 146.1, 142.8, 141.0, 135.3, 134.8, 126.4, 123.9, 122.6, 108.9, 85.1, 79.8, 70.2, 67.3, 59.3, 48.4, 44.0, 25.1, and 11.6 ppm.

39. The compound, the composition, or the pharmaceutical composition of any one of claims 33 to 38, wherein Form A is characterized by an orthorhombic crystal system, a P2₁2₁2₁ space group, and a unit cell having dimensions measured at 100(2) K on an x-ray diffractometer equipped with Cu K_{α} radiation (λ1.54178 Å) of:
$$a = 7. ( 1 ) Å α = 90 °$$
$$b = 8.3 ( 1 ) Å ; β = 90 °$$
$$c = 38.8 ( 1 ) Å γ = 90 ° .$$

40. A pharmaceutical composition comprising:
45-55 wt % of a solid dispersion comprising a polymer and a compound of formula (II): 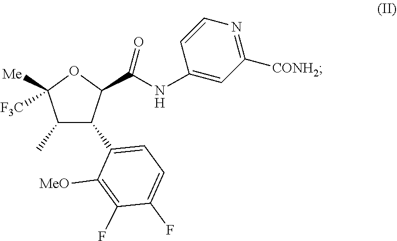 [SMILES: COc1c([C@H]2[C@H](C(=O)Nc3ccnc(C(N)=O)c3)O[C@@](C)(C(F)(F)F)[C@H]2C)ccc(F)c1F]
42-50 wt % of microcrystalline cellulose;
2-4 wt % of croscarmellose sodium; and
0.5-1.5 wt % of magnesium stearate.

41. The pharmaceutical composition of claim 40, wherein the pharmaceutical composition is a tablet core composition.

42. The pharmaceutical composition of claim 41, wherein the tablet core composition is coated with a tablet coating.

43. The pharmaceutical composition of any one of claims 40 to 42, wherein the pharmaceutical composition comprises about 50 mg of the compound of formula (II).

44. The pharmaceutical composition of any one of claims 40 to 43, wherein the pharmaceutical composition comprises an intragranular blend and extragranular blend.
